SEQUENCE LISTING (197 sequences)

SEQ ID NO: 1 - DNA, 606 bp, Danio rerio
atggagcagactgaagtcatgaagcccgagactctggaggatctgatcaaaactctgcatcagatcttccagagcgactccatcaatgtggaggaggtgcagaacctgatggagtcctaccagagcaacccgcaggactggatgaagttcgccaagttcgaccagtacaggtacaccaggaacctcgtggatgaaggaaacggaaagttcaacctgatgatcctgtgctggggtgaaggacacggcagcagcatccatgaccacacagactcgcactgcttcctgaagctgctgcagggtcagctgaaggagacgctgttcgactggcccgaccgcaagctgcagagcggcatgaagccccgcggccagagcgtgctgcaggagaaccagtgcgcgtacatcaacgactctctgggactccaccgtgtggagaatgtgagccacacagagccggccgtgagtctgcacctttacagtcctccgttccagagctgccgcacgtttgaccagcgcaccggacaccacaacaccgtcaagatgaccttctggagcaaatatggcgagaggacgccctatgagctgagcgtctcgcaggagaataactga

SEQ ID NO: 2 - protein, 201 aa, Danio rerio
MEQTEVMKPETLEDLIKTLHQIFQSDSINVEEVQNLMESYQSNPQDWMKFAKFDQYRYTRNLVDEGNGKFNLMILCWGEGHGSSIHDHTDSHCFLKLLQGQLKETLFDWPDRKLQSGMKPRGQSVLQENQCAYINDSLGLHRVENVSHTEPAVSLHLYSPPFQSCRTFDQRTGHHNTVKMTFWSKYGERTPYELSVSQENN

SEQ ID NO: 3 - DNA, 810 bp, Chlamydomonas reinhardtii
atgtcttctatcatcgctatgcctatcaacgaggacggtgtcgttgtggtcgaccgcaagctgctgggcaacgaggtcgagagcaaggcccgctgcgcggacaccgcctgcaccgcggctgcgcccgccccgcccgccacggcggccgcgcccacctccatgccggagctgttgcaggcgttgcagcgcgccattgacgaggagaaggccactggccaggtcgccatcaacgctgtggaccagacgcccgagtccgctgcgcggctgagcgcccgcgtgcaggctctactctcggcctacaccagctccaactcgggcgactggcgacgctacgccatgttcaacgacatccactacgtgcgcaacctggtggatgccaatgaggactttgaactaattgttctttgttggaagcgcgggcaagtcagccgcgtgcacaaccacgccaacgcgcactgctggctggcggtgctggacggcgagatgcgcgagacgcagttccagcgcgcgtccgcgccgcccggctgccccgcgcccgcggcctcggagcacgatggcagcactgtgtacgtggagcccacacaggtgtccgacatgcgagtgggtgacgccggctacatcaacgactccatggcgctgcacaacgtggggtgttgcatgcccgccctggccgctggcgaggagggccccgagggcggggtgacgctgcactgctacgcccccccgattcgccgcgtcaagatctatgaggacagcaaggtcacggagcgcgtgcccggctactactccaagggcggagtgcgcgtttga

SEQ ID NO: 4 - protein, 269 aa, Chlamydomonas reinhardtii
MSSIIAMPINEDGVVVVDRKLLGNEVESKARCADTACTAAAPAPPATAAAPTSMPELLQALQRAIDEEKATGQVAINAVDQTPESAARLSARVQALLSAYTSSNSGDWRRYAMFNDIHYVRNLVDANEDFELIVLCWKRGQVSRVHNHANAHCWLAVLDGEMRETQFQRASAPPGCPAPAASEHDGSTVYVEPTQVSDMRVGDAGYINDSMALHNVGCCMPALAAGEEGPEGGVTLHCYAPPIRRVKIYEDSKVTERVPGYYSKGGVRV

SEQ ID NO: 5 - DNA, 606 bp, Pseudomonas aeruginosa
atgtcatccatcctgcgcctcgaccgcctgcgccagttcatcggcgagctggcgacactgctcgacagccgtcccgacgaatccaccctgctcgcccaagcccaccccctgctggccgagctggtgcgccaggacgactggctgccggaagactgcgcccgccccgatccacagcgctaccaacagtacctgctgcatatcgactcacggcagcgcttctcggtggtcagcttcgtctgggggccgggccagatcacaccggtacacgatcatcgggtctggggcctgatcggcatgctccgcggggccgaatactcgcagccgtacgccttcgatgcgggggggcgtccgcatcccagcggagcccgtcgacgcctggagcccggcgaggtcgaagcgctgtcgccacgcattggcgacgtgcaccaggtgagcaacgccttcagcgaccgaacatccatcagtatccacgtctacggcgccaatatcggtgcggtacggcgtgccgtgttcagcgccgaaggtgaggaaaaacccttcatttccagctattccaacagccgcttgcccaatatctgggacctgtcgaaagagaaccccgcatga

SEQ ID NO: 6 - protein, 201 aa, Pseudomonas aeruginosa
MSSILRLDRLRQFIGELATLLDSRPDESTLLAQAHPLLAELVRQDDWLPEDCARPDPQRYQQYLLHIDSRQRFSVVSFVWGPGQITPVHDHRVWGLIGMLRGAEYSQPYAFDAGGRPHPSGARRRLEPGEVEALSPRIGDVHQVSNAFSDRTSISIHVYGANIGAVRRAVFSAEGEEKPFISSYSNSRLPNIWDLSKENPA

SEQ ID NO: 7 - DNA, 639 bp, Bradyrhizobium guangzhouense
atgacgcctacagccgcaggcagccggacctcaaccgagtttcaacaaggtcggaggactgaggtgagccaaaagctgcttgcatttgtggccgaggtgacacgctgtgtcgaaaacgttcaggacgaagtcatagtcgtttcgcgcgtcaggaaggcgatgagcgagcttgtgtcaacggatgactggttgcctgagacgatggcgctgtccgatcccaaattctatcagcaacatctgctatatggagaccctcttgatcgcttttcggtcgtgagcttcgtttggggaccgggccagcagacgccggtccatgaccattccgtgtggggtgtcatcggcatgttgcgaggatccgagatcaaccagaggtacggccggacggcctatgggatggacccgatcggtggttccgagcgactggagccaggcgacgtggaaatcgtgtcggaagcggttggcgacattcatcaagtgcgaaatgcgttcgatgaccgcgtgtccattagtatccactgctacggtgggaatatcgggagaatccgacgtcatgtcttcaaagatctcggtgagacccaagagtttgtctcgggttattccaatgctctcactcccaacctctgggcgccagcgcagtga

SEQ ID NO: 8 - protein, 191 aa, Bradyrhizobium guangzhouense
MSQKLLAFVAEVTRCVENVQDEVIVVSRVRKAMSELVSTDDWLPETMALSDPKFYQQHLLYGDPLDRFSVVSFVWGPGQQTPVHDHSVWGVIGMLRGSEINQRYGRTAYGMDPIGGSERLEPGDVEIVSEAVGDIHQVRNAFDDRVSISIHCYGGNIGRIRRHVFKDLGETQEFVSGYSNALTPNLWAPAQ

SEQ ID NO: 9 - DNA, 1449 bp, Danio rerio
atggacgagtctgatgggaagctgttccttactgaggctttcaacataatcatggaagaaattcttaacaaaggaagggacttgaaggagaaggtttgtgagtggaaagatccagatcagctgagatctctcctggacctcgaacttcgggatcatggagaatgtcatgagaagctgctgcagagggttcgagatgtggccaaatacagcgtaaaaacttgtcatcctcggttcttcaatcagctgtttgctggcgtggactatcatgcactgacaggacggctcatcactgaaaccctcaataccagccaatacacctatgaagtggctccagtgtttgtcctgatggaggaggaagtgatcagtaagcttcgctctctggttggctggtcagaaggagatgggatcttttgtcctggaggatccatgtctaacatgtatgccattaacgtcgctcggtactgggcttttcctcaagtgaagacaaaaggcttgtgggccgcaccacggatggctatatttacatcacaacagagtcattactccgtgaaaaaaggagctgcgtttcttggtattggaacagaaaatgttttcattgtgcaagtggatgagagcggcagcatgataccagaagacctggaggcaaaaattgtgcaggcaaaatcccaagacgctgttccgtttttcgtaaacgccacagccggaaccacagtgcagggagcctttgaccctctgaagcgcatagctgacatatgtgaaagaaacggcatgtggatgcatgttgacgccgcatggggaggaagcgtgctgttttccaaaaagcacagacatctggttgcaggaatagaaagagcaaactcggtgacttggaatcctcacaaaatgcttctgacgggactgcagtgctctgtgattttgttcagagatactacgaatttgctcatgcactgtcacagtgccaaagccacatacttgttccagcaagacaagttctacgacacaagtctggacacgggcgacaaatccatccagtgtggccggaaggtggattgcctcaagctctggctcatgtggaaggcaatcggagctagtggtctttcacagcgtgtcgataaggcctttgccctcactaggtatttagttgaagaaatggagaaacgggagaatttccagctggtctgtaaggggccgtttgtgaacgtttgcttctggtttattccacccagtctgaaaggaaaggagaacagcccagattaccaggaaagactatccaaggtggcgccagtcattaaagagaggatgatgaagcgaggaacgatgatggtgggatatcagccaatggatgaacacgtcaacttcttccgcatggtggttgtttctccacagctcacaaccaaagacatggatttcttccttgatgagatggagaaactcgggaaggatctatga

SEQ ID NO: 10 - protein, 482 aa, Danio rerio
MDESDGKLFLTEAFNIIMEEILNKGRDLKEKVCEWKDPDQLRSLLDLELRDHGECHEKLLQRVRDVAKYSVKTCHPRFFNQLFAGVDYHALTGRLITETLNTSQYTYEVAPVFVLMEEEVISKLRSLVGWSEGDGIFCPGGSMSNMYAINVARYWAFPQVKTKGLWAAPRMAIFTSQQSHYSVKKGAAFLGIGTENVFIVQVDESGSMIPEDLEAKIVQAKSQDAVPFFVNATAGTTVQGAFDPLKRIADICERNGMWMHVDAAWGGSVLFSKKHRHLVAGIERANSVTWNPHKMLLTGLQCSVILFRDTTNLLMHCHSAKATYLFQQDKFYDTSLDTGDKSIQCGRKVDCLKLWLMWKAIGASGLSQRVDKAFALTRYLVEEMEKRENFQLVCKGPFVNVCFWFIPPSLKGKENSPDYQERLSKVAPVIKERMMKRGTMMVGYQPMDEHVNFFRMVVVSPQLTTKDMDFFLDEMEKLGKDL

SEQ ID NO: 11 - DNA, 1629 bp, Guillardia theta
atggtgccccccgccttgcatgaagggttctgcagccctcgaggcaggacttgttgctctcaggtgggacacgtggagttgttggagagctgggaaacgcaggggaacaagctgagatgcgagcaagacctcctgctggccaaggttccctctcgcttccaccaccttgaggaagtggccgagctggatgatatcttcagggaggtgtatcctctgatccggcagtacgagacggagaacgcgctagcagacgagcacaaggtgctggagttcaggacgccagcggagctgaaggaggaggtggacgtggggctgcctgaggagggatctgtggagaaatttgtcgagggatgcagaagctctatgaagtacagcgtccgaacgagtcacccgcgcttcatgaaccagctctatgctggcagcgacccggcagggcaggtggcagagctgctcagtgctgtgctgaacaccaccatccacacgtacggggcagctcccttcttctccgtgctggagcggcaggtgatcgagaagctggggaggatgctggggtttcaggagcatgtcgacggcgtctttgcccccggaggctcgtacgcgaacatggtggcgctgatagttgcgaggaaccagcacttccctcatgtgcgggagcatggctggaggagcgacgacaaacctgttatcttcacttcttcccatgctcactactctgtcgccaaggctgccatgatcacggggatggggtcgaatcaagtggtcgctgtgcctacggacgagcagggaagaatgcagcctgcagcgctggaggaggagattatgcgagcaaaggagagcggacggaagcctttctacgtgagctgcacggcagggacgacagtgactggggcgtttgacccgattgacgagatctgtcagatatgtagaaggcatgagatgtggctgcacacggatggcgcgtggggaggagctgcaatattctcggaggagcacagaaatcttctacgaggagttgagggcgtcgatagcttctgcttgaatccgcacaagatgctgggggtcccgatgcagtgctccgtgctcatcctcaacaaccacgaggggcgctcgagaggagcaacagaggaagagagcttggatctcgggcagaagtcgctgcagtgcggaaggaaacctgattgcctaaagctctggctctgctggaagcgacatggaacccgcgggtttgcaaggagggtagatcgcgcgtataccttctcgcagaagttcgcagaaatggtcagaagggaccccaggttctacctgctgatggacccgatctcctgcaacgtctgcttcttctacctccctccctccctccggcagcagctggtggacagaaacctcaacgacttggaaaaggaggaggcgcagcggcagctcaaggagttccatgctcgactcggtcaggttactcagatcatctacaggaggatgcagaaagacggcaagatgctcatcaacttcagccctcttaaagacagagatctgcctcacttcttccgagccgtcatgatccagcagagagtaacggaagacgatcttgttttcatcctcgatcattttgaacatctgggaaaggacctctag

SEQ ID NO: 12 - protein, 542 aa, Guillardia theta
MVPPALHEGFCSPRGRTCCSQVGHVELLESWETQGNKLRCEQDLLLAKVPSRFHHLEEVAELDDIFREVYPLIRQYETENALADEHKVLEFRTPAELKEEVDVGLPEEGSVEKFVEGCRSSMKYSVRTSHPRFMNQLYAGSDPAGQVAELLSAVLNTTIHTYGAAPFFSVLERQVIEKLGRMLGFQEHVDGVFAPGGSYANMVALIVARNQHFPHVREHGWRSDDKPVIFTSSHAHYSVAKAAMITGMGSNQVVAVPTDEQGRMQPAALEEEIMRAKESGRKPFYVSCTAGTTVTGAFDPIDEICQICRRHEMWLHTDGAWGGAAIFSEEHRNLLRGVEGVDSFCLNPHKMLGVPMQCSVLILNNHEGRSRGATEEESLDLGQKSLQCGRKPDCLKLWLCWKRHGTRGFARRVDRAYTFSQKFAEMVRRDPRFYLLMDPISCNVCFFYLPPSLRQQLVDRNLNDLEKEEAQRQLKEFHARLGQVTQIIYRRMQKDGKMLINFSPLKDRDLPHFFRAVMIQQRVTEDDLVFILDHFEHLGKDL

SEQ ID NO: 13 - DNA, 1425 bp, Spirula spirula
atgttcaacgccgccgactattccgacttgctgcaccaacttgaaaccttttttgcccaatcaaccccatcccaccctgttagccagcctcttgaccccgctcaactacgcgatcgcctccccctcaacctccctgctgagggcaaacccctagccgacctgaaaattgagaccgcggcgtacttacaccatgccgttaaaactgctcaccctagttatttcaatcagctttggggtggtgtcaatggcgcttgtctgatgggagaactgctcactagcgccaccaatacctcaatgtatacctatgaggttgctcccgtggcgaccctgattgagcaagccttgatcaccaagctgggtgaactgatcggcttcacccaccctgacggacaattcaccacagggggcagcaatggcaacctgatggcgatggcgatcgcccgtcatcgtgccatgccgacgcttaagcaagacggcatgctcaatgcccccccgttaattgcgtttgtttcggctgattctcactactcctttgccaaagccgcccatcttttaggtttaggaaccaaccaactctgggctgtgcccactgatgcccagggcaaactgaatgtagcagaattagaatcgctcatcaccaaagcccacagccttggggcgcagccgttttttgtggctggtacagccgggacaaccgtgcgcggagcctatgaccccttaaccgaaattgcagccgtggcccaacgggaggggctatggttccatgtggacggcgcttggggggcatcggtgctcttgagccctaaacaccgtgagctgatgcggggcgtggagctagccgactctgtcgtttgggatgcgcacaagatgatggggatgacgctgatgtgttcagtgctgctggtgaagcagcgggggcagctactcaataccttctcgacccaaggaacagactatattttccatgcggatgaatccgaacccgtggatttggggccgagcacgatgcattgcggacggcgggtagatgcagtgaagctctggttagcgtggcatcatctgggcgatcgcggttgggcagccctgattaaccattactttgagttggcagcctatgctgagtgtcttatccgtaaccagccgtatctggaattggtgactccccgcgaatcgctgaacctctgtttccgagtgttgcctcaaagcccagaccaggatgccaatcagctcaccttacgcgttcgacaagtcttgttgcagaaggggttggcgatggtgaactacgcccgtattggtaagcacatcattttccggctggtgatttgcaacaaccaaacccaatgcaccgacctcgatcgcctgtttgagcagatcggtgcgatagctcaccacttacaagcagaatttccacaggagaaaacttga

SEQ ID NO: 14 - protein, 474 aa, Spirula spirula
MFNAADYSDLLHQLETFFAQSTPSHPVSQPLDPAQLRDRLPLNLPAEGKPLADLKIETAAYLHHAVKTAHPSYFNQLWGGVNGACLMGELLTSATNTSMYTYEVAPVATLIEQALITKLGELIGFTHPDGQFTTGGSNGNLMAMAIARHRAMPTLKQDGMLNAPPLIAFVSADSHYSFAKAAHLLGLGTNQLWAVPTDAQGKLNVAELESLITKAHSLGAQPFFVAGTAGTTVRGAYDPLTEIAAVAQREGLWFHVDGAWGASVLLSPKHRELMRGVELADSVVWDAHKMMGMTLMCSVLLVKQRGQLLNTFSTQGTDYIFHADESEPVDLGPSTMHCGRRVDAVKLWLAWHHLGDRGWAALINHYFELAAYAECLIRNQPYLELVTPRESLNLCFRVLPQSPDQDANQLTLRVRQVLLQKGLAMVNYARIGKHIIFRLVICNNQTQCTDLDRLFEQIGAIAHHLQAEFPQEKT

SEQ ID NO: 15 - DNA, 1776 bp, Danio rerio
atggcggcgtctgcaccctcctcctcctcttctggcggcgttccggatcccaactcgacaaatttacagccaccttcctcaaactacgactggagtggagtcgctcatggatgtacaaggaagcttggaatgaagatatgtgggttcttgcagaagaacaataatgttgacgacaagggtcgaattgtcgggttgtttaacgaccagcagcccaggagtattttaacccgggacaacgagcgagattcccgcttcagacgcacagagacggacttctccaatctgtatgcaagagatctgcttcctgctaaaaatggcgaggagtacaccatgcagttcctgctggaggtggtggagatcctcactaactacgtgcgcaagaccttcgacagatccaccaaagtgctggacttccaccatccacaccagctgctggaaggcatggagggcttcaacctggagctgtgtgaccagcccgagagtctggagcagatcctggtggactgcagggacactctcaaatatggagtccggacaggtcacccaaggttttttaaccagctgtcttcaggactagacatcatcggtttagctggagaatggttgacctccactgccaacaccaacatgttcacgtatgagattgcgccagtgtttgtcctgatggagcagctcacactgaagaagatgcgagagattgtcggctggccgaacggagaaggagatggcattttctcaccaggaggagccatctccaacatgtacagcgtgatggtggctcgatataaacactatcctgagattaaaatcaaaggcatggcggcggctcccagactggtgctgttcacctcagaacacagtcactactctataaagaaggccagtgcagtgttgggtttcggcacagagaatctgatcctgctgagaacggatgaaagaggtcgagttattccagctgatttggaggccaaagtcattgacgccaagcagaagggctttgtgccgatgtttgtgaacgcaacggctggatctacagtgtacggagccttcgacccaatcaatgagatcgccgacatctgtgagaaatacaacatgtggcttcacgtagatggagcgtggggtggaggtttgctgatgtctagaaaacacaaacacaagctcagtggcattgagagagcaaactccgtcacctggaacccacacaagatgatgggtgttcctctacagtgctccgccattctggtccgggagaagggtcttctacagggctgtaattccatgtgcgctggatatctctttcagccggataagcagtatgacgtcacctatgacacgggggacaaggccatacagtgtggccgtcatgtagacatcttcaaattctggctcatgtggaagtcaaagggcactactggttttgagaagcacattgacaggtgtctggagctgtcggagtatctctaccacaagatcaagaacagagaaggatatgagatggtgtttcaaggggagccacagcacacaaatgtatgtttctggtacattcctccaagcctgcggcttctgccagatggagaggagaaacgacatcggcttcataaggtcgccccaaagatcaaggcactgatgatggagtgcgggacaacaatggtgggctaccagcctcagggtgagaaggttaacttcttcaggatggtggtctccaatccggcggttaccaggtctgacattgacttcctgatcgatgagatagaaagactgggacaggatttatag

SEQ ID NO: 16 - protein, 591 aa, Danio rerio
MAASAPSSSSSGGVPDPNSTNLQPPSSNYDWSGVAHGCTRKLGMKICGFLQKNNNVDDKGRIVGLFNDQQPRSILTRDNERDSRFRRTETDFSNLYARDLLPAKNGEEYTMQFLLEVVEILTNYVRKTFDRSTKVLDFHHPHQLLEGMEGFNLELCDQPESLEQILVDCRDTLKYGVRTGHPRFFNQLSSGLDIIGLAGEWLTSTANTNMFTYEIAPVFVLMEQLTLKKMREIVGWPNGEGDGIFSPGGAISNMYSVMVARYKHYPEIKIKGMAAAPRLVLFTSEHSHYSIKKASAVLGFGTENLILLRTDERGRVIPADLEAKVIDAKQKGFVPMFVNATAGSTVYGAFDPINEIADICEKYNMWLHVDGAWGGGLLMSRKHKHKLSGIERANSVTWNPHKMMGVPLQCSAILVREKGLLQGCNSMCAGYLFQPDKQYDVTYDTGDKAIQCGRHVDIFKFWLMWKSKGTTGFEKHIDRCLELSEYLYHKIKNREGYEMVFQGEPQHTNVCFWYIPPSLRLLPDGEEKRHRLHKVAPKIKALMMECGTTMVGYQPQGEKVNFFRMVVSNPAVTRSDIDFLIDEIERLGQDL

SEQ ID NO: 17 - DNA, 2958 bp, Micromonas pusilla
atgtccgcggcgacgggatcattatccctacccctactcgggcatctcgcgacctcgcgtaacgcacgcgcgcgtcggaaccgcgccgccgcggccatccccggcgtctccctcgggaaatcgacctcggttttcactccgcgaggtcctaagcgcatcgcgcgcgtcgtcacctcgaaggcgggcccgcattcgaaccctccgagggcgatatcgaccgtcgacgacgtcctcgcgttcaccgtgcccaccgacgagcccgcggccgagaccgcctcccccgccgacagcgactgcgaaggcgagttctgcgacatgaaggagagctcgtgcacgacgagggacctcatcggcagcacgccgctgctcgatctgagcgcgtactccctgaaccccaccgtgaagatcctcgcgaagtgcgagtacctcaacccgtccgggtccatcaaagaccgcatcgcgacgcacatcctggacaaggcgatcaagagcggcgatctcaagcccgggatgaccgtcgtcgcggcgacgtccgggaacaccggcgccgcgatcgcgatggcgtgcgcgttgcgcgggtacgactacatcgtcatcaccaacgagaagacgtccaaggagaaggtggacgcgatgagagcgtacggcggcgaggtgatcgtctccccgtccggggtgtccccggacgacccacagcactaccagaacatcgagaacaagctgtgcgaggagaaccccgggacgtactacggcgtggatcagtataacaacccgtacaacgcggacgcgtacgaggcgacgctcgggccggagatttggcgtcagagcgtgggcgcggtgacgcacttcatcgtcggcggcagcaccggcggcacggtcagcggcacggggaggtacttgaagcaagagaacccggacgtgaggatcgtcctcgcggacccgagagggagcgtgttctgggaccacgtcgtcaacggcgtcgccgccgacgacgtcaaggtgtccaagtcgtgggagacggagggcgtcggcaaggattccatccccgggtgcctcgacgtctcgatcgtggacgggatggtgcgcgcgacggacgagcaggcgttcggcgtgtgccgcgagctcgcgagcagcgacggcctcctcgtcggcggcagcagcggtctgaacctccacgcctcgcgcgtgttatccggcgacgtcgcggacgacagcgtcatcgtcacggtgttcccggacaacggcgtgaagtacctgtcgaagatttacaacgacgactggctcgactcgaagaagatgggcggcgcaaagaactcggacgggaacgcggagagagccgcggagtgcgaggtgtactggcgcccggacgcgctctcgttcgcggagcgaaaggcggcggcggacgccgccgccgccgccgccgtcgagggcgacaacctctggcccgaggacgagaccgagcgcgagctcaagttcctggaggaactcgcgccgaagctgacgcagtaccacagagactccatcaagggcgacgagcgcgtgcacagcaagctccagtccccggaggagctcgcggcgacgttcgccgccgcgggggcgcccatcgacctcgcggagggcgacgcccccgcgacggaggagcaactcgcgctcgcggtgcaggcggtcatggacaactcggtccgctcctcgcacccgatgttcttgaaccagctgtacgccggcgtcgacgtcgtcgcgctcgcgggggagtggaccgcgagcgcgttgaacgccaacgtgcacacgtttgaagtcgcgccggtgctcacggagattgagaaagccgtcctcgcgaaaaccgcgcggatgtggctgaacaagcccgggtctaagacgacgccgccgcacgacggtctgctcgtccccggcgggtccctggcgaacatgtactcgatgatcctcgcgcgcgatcgcgcggagccggaggcgaagaccaagggcgcgagcggcaacctcgtcgcgttttgctcggagcagtcgcactactcgtacaaaaagtccgcgatggtcatgggcctcgggatggacaacatgatcaaggtgaagtgcgaccagtccggcgcgatgatcccggcggagctcgagaaggcggttcaggaggccaagtcccggggcaaggtgccgttctacgtcggcaccaccgcggggtccaccgtgctcggcgcctttgacgactacgaaggctgcgcggacgtctgcgaaaagcacgacatgtggatgcacgtcgacggcgcgtggggcggcgccgcggcgctgtccccgacgagaaggcacaatctccagggcgcgaacagagcggactcgttctgctggaacccgcacaagatgctcgggttgccgctccagtgctccatcttcgtgacgaagcaacccggggcgctgtccaaggcgaacgccgcgcaggcggactacttgttccagccggacaagaacaacgccgccgcggacctcggcgaccgcacgattcagtgcggacgcaaggcggacgccctcaagatctggctcgcgtggaaggcgcgcggagacgaaggctgggcgaatctcgtggaccgctcctttggcctcgcggagtacgtcgaggcgtcggtgcgcgagcggtgcgaaaaagacggctcgttcgtcctcgccgcgcccgcgcagtgcgcgaacatcgggttctggtacgtgcccccgcgcctgaggccgttcgatgtcgagtccgcgaccgcggaccagctcacggagattgggttcgtcgccccgaagctgaaggaccggatgcaacggaccggggacgcgatgatcgggttccagccgatcgactcgatgaaccttccaaactttttccgactcgtgcttccaaactcgaggcacctgtcgaagaacgcgctcgacgctatgctcgatcgcatggacgacatgggcaaagacctgtga

SEQ ID NO: 18 - protein, 985 aa, Micromonas pusilla
MSAATGSLSLPLLGHLATSRNARARRNRAAAAIPGVSLGKSTSVFTPRGPKRIARVVTSKAGPHSNPPRAISTVDDVLAFTVPTDEPAAETASPADSDCEGEFCDMKESSCTTRDLIGSTPLLDLSAYSLNPTVKILAKCEYLNPSGSIKDRIATHILDKAIKSGDLKPGMTVVAATSGNTGAAIAMACALRGYDYIVITNEKTSKEKVDAMRAYGGEVIVSPSGVSPDDPQHYQNIENKLCEENPGTYYGVDQYNNPYNADAYEATLGPEIWRQSVGAVTHFIVGGSTGGTVSGTGRYLKQENPDVRIVLADPRGSVFWDHVVNGVAADDVKVSKSWETEGVGKDSIPGCLDVSIVDGMVRATDEQAFGVCRELASSDGLLVGGSSGLNLHASRVLSGDVADDSVIVTVFPDNGVKYLSKIYNDDWLDSKKMGGAKNSDGNAERAAECEVYWRPDALSFAERKAAADAAAAAAVEGDNLWPEDETERELKFLEELAPKLTQYHRDSIKGDERVHSKLQSPEELAATFAAAGAPIDLAEGDAPATEEQLALAVQAVMDNSVRSSHPMFLNQLYAGVDVVALAGEWTASALNANVHTFEVAPVLTEIEKAVLAKTARMWLNKPGSKTTPPHDGLLVPGGSLANMYSMILARDRAEPEAKTKGASGNLVAFCSEQSHYSYKKSAMVMGLGMDNMIKVKCDQSGAMIPAELEKAVQEAKSRGKVPFYVGTTAGSTVLGAFDDYEGCADVCEKHDMWMHVDGAWGGAAALSPTRRHNLQGANRADSFCWNPHKMLGLPLQCSIFVTKQPGALSKANAAQADYLFQPDKNNAAADLGDRTIQCGRKADALKIWLAWKARGDEGWANLVDRSFGLAEYVEASVRERCEKDGSFVLAAPAQCANIGFWYVPPRLRPFDVESATADQLTEIGFVAPKLKDRMQRTGDAMIGFQPIDSMNLPNFFRLVLPNSRHLSKNALDAMLDRMDDMGKDL

SEQ ID NO: 19 - DNA, 747 bp, Danio rerio
atgccgaggaacaacaagacctcgctcatccagaaaatagcgagccaggcttatgcgactttcacaaactgctcttcctccgccattgaggataataataaggtgtttttagagcaccaggcagacttggtcgccctgctctccgacatcagggctgcggatctgaagattgcgccaccaacgaaagtctccacatcctccccttcggtccctccagtcacttacatgcacatctgcgagactgatgtcttcagcatgggggtgtttctcctgaaatctggcgcttccatccctctgcacgaccatcccgggatgcacggcatgctgaaggtcctgtacggtaaagtcagcatcaggtgttatgacaagttggataaagccgagagtgacactgagaggcactttgatcctccattgttggctttccagggagatgatgtgaggagagcagcgctgaggtcttcggggcagttttcggaactaagtggcccttgtgttttgagcccttttaaagacaaccttcatgagattgatgcagtggatggagcggctgcgtttcttgatatactggcaccgccgtatgaccctgacgatggacgggattgtcactactacagagttttacagactgctgggaaaaagtcagagcagagcggtgatgatgaagcttggcttttagagatcccccagccggatgatttctggtgtgggggtgagccgtaccctggtcctgaagtgactatttag

SEQ ID NO: 20 - protein, 249 aa, Danio rerio
MTSTVQKIAKQALATFRNPSVIGEHNKVFLENQSKLKSLLAEVRAADLKIAARTPESAPVPHQRIAPPVTYMHICETDSFSMGVFLLKTGASIPLHDHPGMYGMLKVIYGKVRISCFDRLDKPRDGASGVQFNPPLMPFQRGSLRPSVLKSVGEFTEDSSPCVLSPQQDNIHQIDAVDGPTAFLDILAPPYDPDEGRDCHYYKVLQAHSEAADKKSEVQDQGDVWLMEIPQPSEFWCGGEPYPGPKVTL

SEQ ID NO: 21 - DNA, 1200 bp, Gallus gallus
atggagggaggttacttgcggcccttcgcgcacttcggcacgcaggccatccacgccgggcaggagcccgagcagtggcgttcgggggctttggtgccgccggtctcgctctccaccacgttcaagcagcaggcgcccggcgagcacgcgggttatgattacagccggtgtggaaaccctacccgaacctgcctggagaaggctgtggctgtgctggatggagcgaaatactgtttggcctatgcttctggcttagctgctactgtgaatattactcacctgttaaaggcaggagatacaattatctgtatggatgatgtgtatggaggtacgaacagatacttccagcaaatagccaagaaaatgggtttaaaaataatttttgtggactgtacaaaattgaagtgcctggaagctgcaattacacctgagactaagcttgtttggattgaaacgcccacaaaccccacactgaaggtcattgatattcagggctgtgctgatgtgatccataagcacaaagatattctgctggcagtagacaatagttttatgtctgcgtatttccagcgtcctttgtccctgggggctgacatctgtatgtactctgcaaccaagtacatgaacgggcacagtgatgttgtaatgggactcgtttcagtaaatcgtgatgatgtgtatgaaaggctcaaattcttacaaggctctcttggagccattccatctccctttgactgctacctctgcaaccggggtttgaagactctgcagctgcggatgaatcaacacttccgcaatgcattggctgtgggtcgattccttgagtcaaatccccgcgtggagaaagtcatttaccctggattgccttcgcacccccagcacgagctggccaagcggcagtgcactggctgtcctgggatgctgtcattttacattaaaggaaatctcaaacatgctaccatctttctgaagagcataaaggtgttcacgctggctgagagcctgggaggatatgaaagcttagcagagcttccggctgtcatgactcatgcttcagtgcctaaagaagagagagaagctcttggaatcagcgatacattaattcgcctgtcggttggtttggaagatgaagaagatttactggaagacctagatcaagctctgaaagctgcaatcccagaccctaaggctttgaaatga

SEQ ID NO: 22 - protein, 399 aa, Gallus gallus
MEGGYLRPFAHFGTQAIHAGQEPEQWRSGALVPPVSLSTTFKQQAPGEHAGYDYSRCGNPTRTCLEKAVAVLDGAKYCLAYASGLAATVNITHLLKAGDTIICMDDVYGGTNRYFQQIAKKMGLKIIFVDCTKLKCLEAAITPETKLVWIETPTNPTLKVIDIQGCADVIHKHKDILLAVDNSFMSAYFQRPLSLGADICMYSATKYMNGHSDVVMGLVSVNRDDVYERLKFLQGSLGAIPSPFDCYLCNRGLKTLQLRMNQHFRNALAVGRFLESNPRVEKVIYPGLPSHPQHELAKRQCTGCPGMLSFYIKGNLKHATIFLKSIKVFTLAESLGGYESLAELPAVMTHASVPKEEREALGISDTLIRLSVGLEDEEDLLEDLDQALKAAIPDPKALK

SEQ ID NO: 23 - DNA, 1401 bp, Chlamydomonas reinhardtii
atgcgcagtgtttactctcctgccgcggagatgcccgctggcgtctctatgcgggcgcctgttcccgctgctcttaggtcagcggcccgcagcggcctgcacctgcgcaatgcgtctttccacacgcaccggccgagcgtggtggccccggcggctgccaaggacctggagcccacttctgcgagcagcaacggagcccccaagaacaacggcgtgcagttcactaagctgtcgaccactgccgtccacggcggcgagcgcggtggccgtccccgcgtggccgacgcgctgaccacgccgattgttcaaacctccacctatcacttccgcaacactgcggagctgattgagtacaatgagggtcggttcgattcgtatgagtatgggcgctatggcaacccgaccgcccaggcatgcgagaacaagatcaaggccctggagggcgctgaggactgcctggtttcggcgtcgggcatgaacgcggtgaccagcatgctgctgtcgctggtgccggcgggcggccacatcgtgaccacgtccgattgttactggcgcacccgccagttcatgcagaacttcctgcccaagatgaacatcggcgtcagcgtgattaagcccaacgatctggaggctctgcaggaggcgctggacaagcacagcgtgacgctgttcttctcggagtcgcccaccaacccctacctgcgctgcgtggacatcctcgccatctcgcgcctgtgccacgccaagggcgccttcgtgtgcgtcgactccaccttcgccacgcccatcaacacccgcgccctagagctgggcgccgacctggtgctgcactcggccaccaagtacctggccggccacaacgacgtgctggccggcgcgctgtgcggcaagaaggagctggtggagaaggtgcgcgagttccaccacatcatgggcggcgtggtggacccgcacgccgcctacctgctgctgcgcggcctcaagacgctggagctgcgcgtcacgcgccacaacgccagcgcaatggagatcgcgcggcggctggagagccaccccaaggtggtccgcgtctggtaccccggcctggagtcgcaccccgaccacgccatcgccaagcgccagatgagcggcttcggcggcgtggtgtcgttcgagacgcgcggcggtctgaacgagtgcatcagcttcatcgacaacgtgaagctgccgtacattgcgccgtcgctgggcggcgtggagtcgttgatagagatgccggcggtgcagagctactggggctttggaccggagcgccgcgcgcagattggcatcaaggagaacctgatccgcttcagcatcggcgtggaggacgtggaggacatctgggccgacctggtgcaggcgctggagtacgtgccgtaa

SEQ ID NO: 24 - protein, 466 aa, Chlamydomonas reinhardtii
MRSVYSPAAEMPAGVSMRAPVPAALRSAARSGLHLRNASFHTHRPSVVAPAAAKDLEPTSASSNGAPKNNGVQFTKLSTTAVHGGERGGRPRVADALTTPIVQTSTYHFRNTAELIEYNEGRFDSYEYGRYGNPTAQACENKIKALEGAEDCLVSASGMNAVTSMLLSLVPAGGHIVTTSDCYWRTRQFMQNFLPKMNIGVSVIKPNDLEALQEALDKHSVTLFFSESPTNPYLRCVDILAISRLCHAKGAFVCVDSTFATPINTRALELGADLVLHSATKYLAGHNDVLAGALCGKKELVEKVREFHHIMGGVVDPHAAYLLLRGLKTLELRVTRHNASAMEIARRLESHPKVVRVWYPGLESHPDHAIAKRQMSGFGGVVSFETRGGLNECISFIDNVKLPYIAPSLGGVESLIEMPAVQSYWGFGPERRAQIGIKENLIRFSIGVEDVEDIWADLVQALEYVP

SEQ ID NO: 25 - DNA, 1260 bp, Methanosarcina acetivorans
atgatagcaatgggaagattcatattaaaatgtctgaaatgcggcagagaatacagccaggaatacaggctgacctgcgagaatgacgactcctttttgcgggcggaataccttgaaaaaaaacttgagctgagaaagcagccagggataggaagatttcactcatggcttccggttcaggaagagcttactaccgaagccgggcccatcacgtacaaaagcgaagctcttgcgagggaacttgggctttcgaatctgtacatagggttcagcgggtactggcccgagaaaggagcttttatcaagacctgcagtttcaaagaactcgaagcccatcctacgatgcagcttctcaaggaatccgggggaaaagccatagtccttgcctctgcagggaatacggggagggcttttgcacatgtttcggcacttaccggaaccgatgtttatatcgtggttcccgactcaggcatccctaaactctggctgcctgaagaaccgaccgattccattcaccttatcagcatgactccggggaacgattacaccgatgctatcaaccttgcaggaagaattgcaaagcttcctggaatggtccctgaaggaggagccagaaacgttgccagaagagaaggaatgggtactgtaatgcttgatgcagccgtaaccataggaaagatgcctgatcactacttccaggctgtcggaagcgggacgggaggaatctcagcctgggaagcttctctgcgcctcagagaggacgggcgttttggttccaaacttccaaagctccagcttacccagaatctccccttcgttcccatgtataatgcatggcaagaaggcaggagggatataattcccgaaattgacatgaaagatgcaaagaagcggatcgaagagacctacgccactgtacttaccaaccgagcaccaccttactccgtgacaggcgggctctatgacgcacttgtcgatacggacgggataatgtatgcagtaagcaaagaagaagcccttgacgcaaaagcgctttttgagtcccttgaaggaatagatatccttcccccatctgccgttgctgctgcttccctcttaaaagccgtggaagccggaaatgtcggaaaggacgacactatcctcctgaacattgcaggcggaggtttcaaacggctgaaggaagacttcacactattccagattgaacctgaaattactgtctcgaacccggatgtgccgcttgaggaactgaagctctga

SEQ ID NO: 26 - protein, 419 aa, Methanosarcina acetivorans
MIAMGRFILKCLKCGREYSQEYRLTCENDDSFLRAEYLEKKLELRKQPGIGRFHSWLPVQEELTTEAGPITYKSEALARELGLSNLYIGFSGYWPEKGAFIKTCSFKELEAHPTMQLLKESGGKAIVLASAGNTGRAFAHVSALTGTDVYIVVPDSGIPKLWLPEEPTDSIHLISMTPGNDYTDAINLAGRIAKLPGMVPEGGARNVARREGMGTVMLDAAVTIGKMPDHYFQAVGSGTGGISAWEASLRLREDGRFGSKLPKLQLTQNLPFVPMYNAWQEGRRDIIPEIDMKDAKKRIEETYATVLTNRAPPYSVTGGLYDALVDTDGIMYAVSKEEALDAKALFESLEGIDILPPSAVAAASLLKAVEAGNVGKDDTILLNIAGGGFKRLKEDFTLFQIEPEITVSNPDVPLEELKL

SEQ ID NO: 27 - DNA, 1314 bp, Euryarchaeota archaeon
atgacatacggaaaaacaataggttatggatcgaatactgttcattacacgggggattcgaaatatgagcttgtatgcgcagaatgttcaaacatttttactgatgacagattgaaatgctgcaacgatggagcgctcctgcgtgcaaattacaggaataaacagctcatgttaaaggacgtccccgggatgggtaagttccacgactggctgccgatacatgagataatgaccacgaattcaggtcctgtcacctacaggagcactgaacttgcagcagaacttgggcttaacaacctgtacatctctttcaatggctactggcctgaaaaaggtgcgtacataaagacttgcagtttcaaggaacttgaggcttttcccacggtccagaaaaccagacgtgcaaaccgctcgctggtacttgcatcagcgggaaataccgcgcgcgctttttcccatgtatcagcgatgacaggacaggaagtgtatatagtggtgccttcaagcgggatgacacggatgtggctgcctgaagaacccacggattccatacacattatacaaatgggtaataactgtgattatacagattctatacatcttgcagacaggatcgccgcgctccccggtatgcaggcagaaggcggcgcaaggaacgtggcgcgaagggacggtatggggacggtgatgctggatgccgctgtgactatgaagcgcatgcctgaccattatttccaggctatagggagcgggacaggcggaatagcagcatgggaagcatcgctgcggctgctgggggatggaaggttcgcaggcggactgccgcagcttcatctttcacagaacctgccttttgccccaatgttcaatgcatggaaagcaggacggcgcgaaataatcagggacattgatatgcctgacccgaaaaaactcatacacgagatgtatgcagacatcctttcaaacagggagccgccatattcagtagctggcggcgtctatgacgcgctttgctctacacagggtacgatgtacgggatatccaatagcgaagcgaaaaaagcgcagcagttgttcgaacggtacgaagggatagatatatttgcacccgcaggagttgctgttgcgtcgctcatcgaagcggtaaatgaacataaagtcgatgcagaagatcatattgtgttaaatatcactggcggaggagtgaagcgccttgaaaaggaccataccctctcgaaaatcgagccagagtcgatcgttgaaaatgcgaatgttgcgcttgaagaggtaattaaaagaaaggtatttgcttga

SEQ ID NO: 28 - protein, 437 aa, Euryarchaeota archaeon
MTYLKTISSGTNTILHTWDPKYDLVCAGCSSVFTEDRLKCSNDGALLRANYRNKQLILKDVPGMGKFHDWLPIHEIMTTESGPVTYRSTELAAELGLNNLYISFNGYWPEKGAYIKTCSFKELEAFPTVQKTTGKNRSLVLASAGNTARAFSHVSAMTGQEVYIVVPSSGMLRMWLPEKPTDSIHIIQMGKNCDYTDSIHLADRIAALPGMQAEGGARNVARRDGMGTVMLDAAVTMKRMPDHYFQAIGSGTGGIAAWEAALRLREDGRFPGGLPQLHLSQNLPFAPMYNAWKAGRREIIKDIDMPDPKKLIHEMYADILSNREPPYSVAGGVYDALSSTKGTMYGISNNEAKKAQQLFERYEGIDIFAPAGVAVASLIEAVNEHKVDSEDHILLNITGGGVKRLEQDHTLSKIEPESIAENANVPLEEIIKRKVLA

SEQ ID NO: 29 - DNA, 1545 bp, Escherichia coli
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtggatgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgagtttccggaatcaccgggcgcgttcctgcgcttcctcaacacgctgggtacgtactggaacatttctttgttccactatcgcagccacggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 30 - protein, 514 aa, Escherichia coli
MADSQPLSGAPEGAEYLRAVLRAPVYEAAQVTPLQKMEKLSSRLDNVILVKREDRQPVHSFKLRGAYAMMAGLTEEQKAHGVITASAGNHAQGVAFSSARLGVKALIVMPTATADIKVDAVRGFGGEVLLHGANFDEAKAKAIELSQQQGFTWVPPFDHPMVIAGQGTLALELLQQDAHLDRVFVPVGGGGLAAGVAVLIKQLMPQIKVIAVEAEDSACLKAALDAGHPVDLPRVGLFAEGVAVKRIGDETFRLCQEYLDDIITVDSDAICAAMKDLFEDVRAVAEPSGALALAGMKKYIALHNIRGERLAHILSGANVNFHGLRYVSERCELGEQREALLAVTIPEEKGSFLKFCQLLGGRSVTEFNYRFADAKNACIFVGVRLSRGLEERKEILQMLNDGGYSVVDLSDDEMAKLHVRYMVGGRPSHPLQERLYSFEFPESPGAFLRFLNTLGTYWNISLFHYRSHGTDYGRVLAAFELGDHEPDFETRLNELGYDCHDETNNPAFRFFLAG

SEQ ID NO: 31 - DNA, 903 bp, Oceanospirillales bacterium
atgactgaatctaagtttcctgtgaagacgagagagtttcacaaccaccattttgattcgacggtctggaatgattttgtattccgcgacgacgatattatcatttccacctatgcaaaatccggcacgacctggatgcagcaaattgtgtcccagttgctgttttccggagaggagggggtggaggttgctgagatgtcaccctggatggatttgcgagttcctccgaaagaagtcaagttgccggcggttgaggcgcagacgcaccgtcgatttttaaaaactcacttacctgtggatgcgctggtcttttcagaaaaagcaaagtacatctacatcggtcgggacgggcgtgatgttgtctggagtctttataatcatcatcttaatgccaacggagcctggtatgatgctctcaacaatacgccaggcagggttggcccggagatagggcctccctgcgcgtctgtctcgcagtatttcgacgattggctggaaaaagacggctatccattctggtcgctgtgggacaacgcccgcacctggtgggcgattcggcatttgccgaatgtgcacatggttcactttgcaaatctgaagtcagacatgcctggtgagattcgccgcattgcagaattcattggcacgcccgttgacgaatcgaaatgggagagcgtattaaagcattgtagcttcgactatatgaaggccaatgcgacaaagagtgttccacttggtggggcgttctgggatggcggcgcacaaacctttattcataaaggccagaatggccggtggcgtgatgtgctttcagccgaacagaataagcagtatgagcagcgtgcgcagtcagagcttggctccgactgtgcccattggtttgccactggtgagctcagataa

SEQ ID NO: 32 - protein, 300 aa, Oceanospirillales bacterium
MTESKFPVKTREFHNHHFDSTVWNDFVFRDDDIIISTYAKSGTTWMQQIVSQLLFSGEEGVEVAEMSPWMDLRVPPKEVKLPAVEAQTHRRFLKTHLPVDALVFSEKAKYIYIGRDGRDVVWSLYNHHLNANGAWYDALNNTPGRVGPEIGPPCASVSQYFDDWLEKDGYPFWSLWDNARTWWAIRHLPNVHMVHFANLKSDMPGEIRRIAEFIGTPVDESKWESVLKHCSFDYMKANATKSVPLGGAFWDGGAQTFIHKGQNGRWRDVLSAEQNKQYEQRAQSELGSDCAHWFATGELR

SEQ ID NO: 33 - DNA, 969 bp, Ostreococcus tauri
atgccgcgcggatggacgaagacgcgcgcgtacgactcgcatcactttgacgccgacgcgtggtcggtggtgacgcctcgagcgggtgacgtcatcatcgccaccgcgtataaatctggcacgacgtggatgcaacagatcgtgtcgcaactcgtgttcgagggcgcggccccggcggcgttgggggagctctcgccgtgggtggatctgcgcgtgcccccgcgggaggtgaagcgagggatgatcgagggattgccctcgccccggatcttgaagacgcatcttccgacgacggggttggaatacgacgaaaacgcgaagtacatttacgtcgcgcgggacggccgcgacgcgttcatgtctttgatgaaccactataagaacggtaatgaagcgttttacggcgcgctgaacggccctgggttaaagggcgcacctttgcctacgtgggaagaggcgtgcgaaggcgagggcgacgagaaacttcgcgcgctttttgacaagtggctcaacacgccgtggggccagcacccgtgggaagaagacgggtggcctttctggtctctgttctataacatgaagacgtggtgggacgcgcgcgaatccaagaacatcatcttcgtgcatttttcggatttgaagaaggatttgaagggtcagatgcgacgcattgcgaagtttttgaacgccccgatcgatgaaagcaaattcgatgcgcaagtcacagcgtgcacgttcgagagcatgaagggtaacgccgcgagcgtcgcgcctctcggtggcgcgctgtggaagggcggtgcggagacgttcattaacaaaggtaccaacggccggtggaggaacgttctaaccaaggaacaagtcaagcagtacgagcaggtggctgagaaacggctgggtaaggactgcgcaaagtggctcgccaacggcggcgatatgaacggccgtgggtgcgtgatcatgtga

SEQ ID NO: 34 - protein, 322 aa, Ostreococcus tauri
MPRGWTKTRAYDSHHFDADAWSVVTPRAGDVIIATAYKSGTTWMQQIVSQLVFEGAAPAALGELSPWVDLRVPPREVKRGMIEGLPSPRILKTHLPTTGLEYDENAKYIYVARDGRDAFMSLMNHYKNGNEAFYGALNGPGLKGAPLPTWEEACEGEGDEKLRALFDKWLNTPWGQHPWEEDGWPFWSLFYNMKTWWDARESKNIIFVHFSDLKKDLKGQMRRIAKFLNAPIDESKFDAQVTACTFESMKGNAASVAPLGGALWKGGAETFINKGTNGRWRNVLTKEQVKQYEQVAEKRLGKDCAKWLANGGDMNGRGCVIM

SEQ ID NO: 35 - DNA, 1218 bp, Corynebacterium glutamicum
atggctgttaagaccctcaaggacttgctcgacgaaggcgtagacggacgccacgtcatcgttcgatctgacttcaatgttcccctcaacgatgaccgcgagatcaccgataagggccgaatcattgcctccctaccaacccttaaagcactgagcgaaggtggcgcaaaggtcatcgtcatggctcaccttggccgcccaaagggcgaggtcaacgagaagtactccctcgcacctgtcgctgaggcactctccgatgagcttggccagtacgttgcacttgccgcagacgttgttggcgaagacgcacacgagcgcgcaaacggcctgaccgagggcgacatcctgctcctggagaacgtgcgcttcgacccacgcgaaacctccaaggacgaggcagagcgcaccgctttcgctcaggagctcgcagctcttgcagcagacaacggcgcattcgtttctgacggcttcggtgttgtccaccgcgcacagacctccgtctacgacattgcaaagttgctgccacactacgctggcggactggtagagaccgagatttccgttctggaaaagatcgcagaatcaccagaggcaccatacgtagtggttctcggtggatccaaggtctctgacaagatcggtgttattgaggcgctggctgccaaggctgacaagatcatcgtcggtggcggcatgtgctacaccttcctcgcagctcagggacacaacgttcagcagtccctcctgcaggaagaaatgaaggctacctgcaccgacctgctcgcacgcttcggtgacaagatcgttctcccagttgacctggttgcagcatccgaatttaacaaggacgcagagaagcagatcgttgacctggactccatcccagaaggctggatgtctcttgacatcggaccagagtccgtcaagaacttcggtgaggttctcagcaccgctaagaccatcttctggaacggcccaatgggcgtgttcgagttcgcagcattctctgaaggcacccgcggcatcgcccaggccatcatcgatgcaactgcaggcaacgacgcattctccgttgttggcggtggcgactccgcagcatccgttcgcgtgctcggcctgaacgaagacggcttctcccacatctccaccggtggtggcgcatccctcgagtaccttgaaggcaaggaactcccaggcgttgcaattctcgctcagtaa

SEQ ID NO: 36 - protein, 405 aa, Corynebacterium glutamicum
MAVKTLKDLLDEGVDGRHVIVRSDFNVPLNDDREITDKGRIIASLPTLKALSEGGAKVIVMAHLGRPKGEVNEKYSLAPVAEALSDELGQYVALAADVVGEDAHERANGLTEGDILLLENVRFDPRETSKDEAERTAFAQELAALAADNGAFVSDGFGVVHRAQTSVYDIAKLLPHYAGGLVETEISVLEKIAESPEAPYVVVLGGSKVSDKIGVIEALAAKADKIIVGGGMCYTFLAAQGHNVQQSLLQEEMKATCTDLLARFGDKIVLPVDLVAASEFNKDAEKQIVDLDSIPEGWMSLDIGPESVKNFGEVLSTAKTIFWNGPMGVFEFAAFSEGTRGIAQAIIDATAGNDAFSVVGGGDSAASVRVLGLNEDGFSHISTGGGASLEYLEGKELPGVAILAQ

SEQ ID NO: 37 - DNA, 1002 bp, Corynebacterium glutamicum
atgagccagaatggccgtccggtagtcctcatcgccgataagcttgcgcagtccactgttgacgcgcttggagatgcagtagaagtccgttgggttgacggacctaaccgcccagaactgcttgatgcagttaaggaagcggacgcactgctcgtgcgttctgctaccactgtcgatgctgaagtcatcgccgctgcccctaacttgaagatcgtcggtcgtgccggcgtgggcttggacaacgttgacatccctgctgccactgaagctggcgtcatggttgctaacgcaccgacctctaatattcactccgcttgtgagcacgcaatttctttgctgctgtctactgctcgccagatccctgctgctgatgcgacgctgcgtgagggcgagtggaagcggtcttctttcaacggtgtggaaattttcggaaaaactgtcggtatcgtcggttttggccacattggtcagttgtttgctcagcgtcttgctgcgtttgagaccaccattgttgcttacgatccttacgctaaccctgctcgtgcggctcagctgaacgttgagttggttgagttggatgagctgatgagccgttctgactttgtcaccattcaccttcctaagaccaaggaaactgctggcatgtttgatgcgcagctccttgctaagtccaagaagggccagatcatcatcaacgctgctcgtggtggccttgttgatgagcaggctttggctgatgcgattgagtccggtcacattcgtggcgctggtttcgatgtgtactccaccgagccttgcactgattctcctttgttcaagttgcctcaggttgttgtgactcctcacttgggtgcttctactgaagaggctcaggatcgtgcgggtactgacgttgctgattctgtgctcaaggcgctggctggcgagttcgtggcggatgctgtgaacgtttccggtggtcgcgtgggcgaagaggttgctgtgtggatggatctggcttaa

SEQ ID NO: 38 - protein, 333 aa, Corynebacterium glutamicum
MSQNGRPVVLIADKLAQSTVDALGDAVEVRWVDGPNRPELLDAVKEADALLVRSATTVDAEVIAAAPNLKIVGRAGVGLDNVDIPAATEAGVMVANAPTSNIHSACEHAISLLLSTARQIPAADATLREGEWKRSSFNGVEIFGKTVGIVGFGHIGQLFAQRLAAFETTIVAYDPYANPARAAQLNVELVELDELMSRSDFVTIHLPKTKETAGMFDAQLLAKSKKGQIIINAARGGLVDEQALADAIESGHIRGAGFDVYSTEPCTDSPLFKLPQVVVTPHLGASTEEAQDRAGTDVADSVLKALAGEFVADAVNVSGGRVGEEVAVWMDLA

SEQ ID NO: 39 - DNA, 1341 bp, Corynebacterium glutamicum
atgtcgtgttccgcgctcagacatgagacaattgttgccgtgactgaactcatccagaatgaatcccaagaaatcgctgagctggaagccggccagcaggttgcattgcgtgaaggttatcttcctgcggtgatcacagtgagcggtaaagaccgcccaggtgtgactgccgcgttctttagggtcttgtccgctaatcaggttcaggtcttggacgttgagcagtcaatgttccgtggctttttgaacttggcggcgtttgtgggtatcgcacctgagcgtgtcgagaccgtcaccacaggcctgactgacaccctcaaggtgcatggacagtccgtggtggtggagctgcaggaaactgtgcagtcgtcccgtcctcgttcttcccatgttgttgtggtgttgggtgatccggttgatgcgttggatatttcccgcattggtcagaccctggcggattacgatgccaacattgacaccattcgtggtatttcggattaccctgtgaccggcctggagctgaaggtgactgtgccggatgtcagccctggtggtggtgaagcgatgcgtaaggcgcttgctgctcttacctctgagctgaatgtggatattgcgattgagcgttctggtttgctgcgtcgttctaagcgtctggtgtgcttcgattgtgattccacgttgatcactggtgaggtcattgagatgctggcggctcacgcgggcaaggaagctgaagttgcggcagttactgagcgtgcgatgcgcggtgagctcgatttcgaggagtctctgcgtgagcgtgtgaaggcgttggctggtttggatgcgtcggtgatcgatgaggtcgctgccgctattgagctgacccctggtgcgcgcaccacgatccgtacgctgaaccgcatgggttaccagaccgctgttgtttccggtggtttcatccaggtgttggaaggtttggctgaggagttggagttggattatgtccgcgccaacactttggaaatcgttgatggcaagctgaccggcaacgtcaccggaaagatcgttgaccgcgctgcgaaggctgagttcctccgtgagttcgctgcggattctggcctgaagatgtaccagactgtcgctgtcggtgatggcgctaatgacatcgatatgctctccgctgcgggtctgggtgttgctttcaacgcgaagcctgcgctgaaggagattgcggatacttccgtgaaccacccattcctcgacgaggttttgcacatcatgggcatttcccgcgacgagatcgatctggcggatcaggaagacggcactttccaccgcgttccattgaccaatgcctaa

SEQ ID NO: 40 - protein, 446 aa, Corynebacterium glutamicum
MSCSALRHETIVAVTELIQNESQEIAELEAGQQVALREGYLPAVITVSGKDRPGVTAAFFRVLSANQVQVLDVEQSMFRGFLNLAAFVGIAPERVETVTTGLTDTLKVHGQSVVVELQETVQSSRPRSSHVVVVLGDPVDALDISRIGQTLADYDANIDTIRGISDYPVTGLELKVTVPDVSPGGGEAMRKALAALTSELNVDIAIERSGLLRRSKRLVCFDCDSTLITGEVIEMLAAHAGKEAEVAAVTERAMRGELDFEESLRERVKALAGLDASVIDEVAAAIELTPGARTTIRTLNRMGYQTAVVSGGFIQVLEGLAEELELDYVRANTLEIVDGKLTGNVTGKIVDRAAKAEFLREFAADSGLKMYQTVAVGDGANDIDMLSAAGLGVAFNAKPALKEIADTSVNHPFLDEVLHIMGISRDEIDLADQEDGTFHRVPLTNA

SEQ ID NO: 41 - DNA, 1131 bp, Corynebacterium glutamicum
atgaccgacttccccaccctgccctctgagttcatccctggcgacggccgtttcggctgcggaccttccaaggttcgaccagaacagattcaggctattgtcgacggatccgcatccgtcatcggtacctcacaccgtcagccggcagtaaaaaacgtcgtgggttcaatccgcgagggactctccgacctcttctcccttccagaaggctacgagatcatcctttccctaggtggtgcgaccgcattctgggatgcagcaaccttcggactcattgaaaagaagtccggtcacctttctttcggtgagttctcctccaagttcgcaaaggcttctaagcttgctccttggctcgacgagccagagatcgtcaccgcagaaaccggtgactctccggccccacaggcattcgaaggcgccgatgttattgcatgggcacacaacgaaacctccactggcgccatggttccagttcttcgccccgaaggctctgaaggctccctggttgccattgacgcaacctccggcgctggtggactgccagtagacatcaagaactccgatgtttactacttctccccacagaagtgcttcgcatccgacggtggcctgtggcttgcagcgatgagcccagcagctctcgagcgcatcgagaagatcaacgcttccgatcgcttcatccctgagttcctcaacctgcagaccgcagtggataactccctgaagaaccagacctacaacaccccagctgttgctaccttgctgatgctggacaaccaggtcaagtggatgaactccaacggcggcctggatggaatggttgctcgcaccacagcaagctcctccgccctgtacaactgggctgaggctcgcgaggaggcatccccatacgtggcagatgcagctaagcgctccctcgttgtcggcaccatcgacttcgatgactccatcgacgcagcagtgatcgctaagatactgcgcgcaaacggcatcctggacaccgagccttaccgcaagctgggacgcaaccagctgcgcatcggtatgttcccagcgatcgattccaccgatgtggaaaagctcaccggagcaatcgacttcatcctcgatggcggttttgcaaggaagtaa

SEQ ID NO: 42 - protein, 376 aa, Corynebacterium glutamicum
MTDFPTLPSEFIPGDGRFGCGPSKVRPEQIQAIVDGSASVIGTSHRQPAVKNVVGSIREGLSDLFSLPEGYEIILSLGGATAFWDAATFGLIEKKSGHLSFGEFSSKFAKASKLAPWLDEPEIVTAETGDSPAPQAFEGADVIAWAHNETSTGAMVPVLRPEGSEGSLVAIDATSGAGGLPVDIKNSDVYYFSPQKCFASDGGLWLAAMSPAALERIEKINASDRFIPEFLNLQTAVDNSLKNQTYNTPAVATLLMLDNQVKWMNSNGGLDGMVARTTASSSALYNWAEAREEASPYVADAAKRSLVVGTIDFDDSIDAAVIAKILRANGILDTEPYRKLGRNQLRIGMFPAIDSTDVEKLTGAIDFILDGGFARK

SEQ ID NO: 43 - DNA, 822 bp, Escherichia coli
atgtcgtgtgaagaactggaaattgtctggaacaatattaaagccgaagccagaacgctggcggactgtgagccaatgctggccagtttttaccacgcgacgctactcaagcacgaaaaccttggcagtgcactgagctacatgctggcgaacaagctgtcatcgccaattatgcctgctattgctatccgtgaagtggtggaagaagcctacgccgctgacccggaaatgatcgcctctgcggcctgtgatattcaggcggtgcgtacccgcgacccggcagtcgataaatactcaaccccgttgttatacctgaagggttttcatgccttgcaggcctatcgcatcggtcactggttgtggaatcaggggcgtcgcgcactggcaatctttctgcaaaaccaggtttctgtgacgttccaggtcgatattcacccggcagcaaaaattggtcgcggtatcatgcttgaccacgcgacaggcatcgtcgttggtgaaacggcggtgattgaaaacgacgtatcgattctgcaatctgtgacgcttggcggtacgggtaaatctggtggtgaccgtcacccgaaaattcgtgaaggtgtgaggattggcgcgggcgcgaaaatcctcggcaatattgaagttgggcgcggcgcgaagattggcgcaggttccgtggtgctgcaaccggtgccgccgcataccaccgccgctggcgttccggctcgtattgtcggtaaaccagacagcgataagccatcaatggatatggaccagcatttcaacggtattaaccatacatttgagtatggggatgggatctaa

SEQ ID NO: 44 - protein, 273 aa, Escherichia coli
MSCEELEIVWNNIKAEARTLADCEPMLASFYHATLLKHENLGSALSYMLANKLSSPIMPAIAIREVVEEAYAADPEMIASAACDIQAVRTRDPAVDKYSTPLLYLKGFHALQAYRIGHWLWNQGRRALAIFLQNQVSVTFQVDIHPAAKIGRGIMLDHATGIVVGETAVIENDVSILQSVTLGGTGKSGGDRHPKIREGVRIGAGAKILGNIEVGRGAKIGAGSVVLQPVPPHTTAAGVPARIVGKPDSDKPSMDMDQHFNGINHTFEYGDGI

SEQ ID NO: 45 - DNA, 972 bp, Escherichia coli
atgagtaagatttttgaagataactcgctgactatcggtcacacgccgctggttcgcctgaatcgcatcggtaacggacgcattctggcgaaggtggaatctcgtaaccccagcttcagcgttaagtgccgtatcggtgccaacatgatttgggatgccgaaaagcgcggcgtgctgaaaccaggcgttgaactggttgaaccgaccagcggtaataccgggattgcactggcctatgtagctgccgctcgcggttacaaactcaccctgaccatgccagaaaccatgagtattgaacgccgcaagctgctgaaagcgttaggtgcaaacctggtgctgacggaaggtgctaaaggcatgaaaggcgcaatccaaaaagcagaagaaattgtcgccagcaatccagagaaatacctgctgctgcaacaattcagcaatccggcaaaccctgaaattcacgaaaagaccaccggtccggagatatgggaagataccgacggtcaggttgatgtatttattgctggcgttgggactggcggtacgctgactggcgtcagccgctacattaaaggcaccaaaggcaagaccgatcttatctctgtcgccgttgagccaaccgattctccagttatcgcccaggcgctggcaggtgaagagattaaacctggcccgcataaaattcagggtattggcgctggttttatcccggctaacctcgatctcaagctggtcgataaagtcattggcatcaccaatgaagaagcgatttctaccgcgcgtcgtctgatggaagaagaaggtattcttgcaggtatctcttctggagcagctgttgccgcggcgttgaaactacaagaagatgaaagctttaccaacaagaatattgtggttattctaccatcatcgggtgagcgttatttaagcaccgcattgtttgccgatctcttcactgagaaagaattgcaacagtaa

SEQ ID NO: 46 - protein, 323 aa, Escherichia coli
MSKIFEDNSLTIGHTPLVRLNRIGNGRILAKVESRNPSFSVKCRIGANMIWDAEKRGVLKPGVELVEPTSGNTGIALAYVAAARGYKLTLTMPETMSIERRKLLKALGANLVLTEGAKGMKGAIQKAEEIVASNPEKYLLLQQFSNPANPEIHEKTTGPEIWEDTDGQVDVFIAGVGTGGTLTGVSRYIKGTKGKTDLISVAVEPTDSPVIAQALAGEEIKPGPHKIQGIGAGFIPANLDLKLVDKVIGITNEEAISTARRLMEEEGILAGISSGAAVAAALKLQEDESFTNKNIVVILPSSGERYLSTALFADLFTEKELQQ

SEQ ID NO: 47 - DNA, 2943 bp, Escherichia coli
atggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaagatgaacaccgcacttgcacaacaaatcgccaatgaaggcggcgtcgaagcctggatgattgcgcaacaacataaaagcctgctgcgttttctgacctgtggtagcgtcgatgacggcaaaagtactctgattggtcgtctgctgcacgatacccgccaaatctacgaagatcagctctcatcgctgcataacgacagtaagcgtcacggcacccagggcgaaaagctggatctggctctgctggtggacggcctgcaagctgagcgcgaacagggcatcaccattgacgtggcctaccgctatttctctaccgagaagcgtaaatttattatcgccgacaccccagggcacgagcagtacacccgcaatatggcgactggcgcatcgacatgtgaactggcgatcttactgatcgatgcccgtaaaggcgtgctcgatcaaacccgtcgtcacagttttatctccacactgttggggatcaaacatctggtcgtggcgatcaacaaaatggatctggtggattacagtgaagagacgttcacccgtattcgtgaagattatttgacctttgccgggcagctgccgggtaatctggatatccgctttgtgccgctctctgcactggaaggcgacaacgtggcatcgcaaagtgaaagtatgccgtggtacagcggtccgacactgctcgaagtgctggaaaccgtggagatccagcgagtggtggatgctcagccaatgcgcttcccggtgcagtacgttaatcgcccgaatctcgattttcgtggttacgccggaacgctggcatccggtcgcgtggaagtcgggcaacgtgtcaaagtgctgccctctggtgtggaatcaaacgtcgcgcggatcgtgacttttgatggtgatcgcgaagaagcctttgccggagaagcgatcaccctggtgctgacggatgagatcgacatcagccgtggcgatctgctgctggcggcagacgaagcgttaccggcggtgcagagcgcgtcggtggatgtggtatggatggcggaacagccgctttctccagggcagagttacgacatcaaaattgccggtaagaagacgcgcgcgcgtgttgatggcattcgctatcaggttgatattaataaccttacccagcgtgaagttgaaaacctgccactgaatgggatcggcctcgtggatctcacttttgacgagccgctggtgttagatcgttatcaacaaaatccggtgacgggtgggctgatttttatcgatcgcctgagcaatgtgaccgtgggtgccggtatggtgcacgagccagttagccaggcaactgctgcgccatctgaattcagtgcattcgaactggaattgaatgctctggttcgtcgccactttccgcactggggcgcgcgcgatttgctgggggataaataatggcgctgcatgacgaaaacgtcgtctggcatagccatccggtcactgtgcaacaacgcgagctacaccacggtcatcgtggtgtagtgctgtggtttaccggcctctccgggtccggtaaatcaacggtcgccggggcgctggaggaggcgttacataaactcggcgtcagtacgtatctgctggatggcgacaatgttcgccacggattatgcagcgatctcggttttagcgatgccgatcgtaaagagaatatccgtcgcgtcggtgaagtggcgaatttgatggttgaagccggactggtggtgctgaccgcatttatctcgccacaccgcgccgaacgccagatggttcgcgaacgcgtaggagaagggcgctttatcgaagtgtttgtcgatacgccgctggcgatttgcgaagcccgcgatcccaaaggcttatataagaaagcgcgtgccggtgaactgcgcaactttacgggaatagattccgtttacgaagcgcctgaatcggcagaaattcatctcaatggtgaacaattagtaacaaatttggtacagcaattattagatctgttgagacagaacgatattatcagatcctga

SEQ ID NO: 48 - protein, 302 aa, Escherichia coli
MDQIRLTHLRQLEAESIHIIREVAAEFSNPVMLYSIGKDSSVMLHLARKAFYPGTLPFPLLHVDTGWKFREMYEFRDRTAKAYGCELLVHKNPEGVAMGINPFVHGSAKHTDIMKTEGLKQALNKYGFDAAFGGARRDEEKSRAKERIYSFRDRFHRWDPKNQRPELWHNYNGQINKGESIRVFPLSNWTEQDIWQYIWLENIDIVPLYLAAERPVLERDGMLMMIDDNRIDLQPGEVIKKRMVRFRTLGCWPLTGAVESNAQTLPEIIEEMLVSTTSERQGRVIDRDQAGSMELKKRQGYF

SEQ ID NO: 49 - protein, 475 aa, Escherichia coli
MNTALAQQIANEGGVEAWMIAQQHKSLLRFLTCGSVDDGKSTLIGRLLHDTRQIYEDQLSSLHNDSKRHGTQGEKLDLALLVDGLQAEREQGITIDVAYRYFSTEKRKFIIADTPGHEQYTRNMATGASTCELAILLIDARKGVLDQTRRHSFISTLLGIKHLVVAINKMDLVDYSEETFTRIREDYLTFAGQLPGNLDIRFVPLSALEGDNVASQSESMPWYSGPTLLEVLETVEIQRVVDAQPMRFPVQYVNRPNLDFRGYAGTLASGRVEVGQRVKVLPSGVESNVARIVTFDGDREEAFAGEAITLVLTDEIDISRGDLLLAADEALPAVQSASVDVVWMAEQPLSPGQSYDIKIAGKKTRARVDGIRYQVDINNLTQREVENLPLNGIGLVDLTFDEPLVLDRYQQNPVTGGLIFIDRLSNVTVGAGMVHEPVSQATAAPSEFSAFELELNALVRRHFPHWGARDLLGDK

SEQ ID NO: 50 - protein, 201 aa, Escherichia coli
MALHDENVVWHSHPVTVQQRELHHGHRGVVLWFTGLSGSGKSTVAGALEEALHKLGVSTYLLDGDNVRHGLCSDLGFSDADRKENIRRVGEVANLMVEAGLVVLTAFISPHRAERQMVRERVGEGRFIEVFVDTPLAICEARDPKGLYKKARAGELRNFTGIDSVYEAPESAEIHLNGEQLVTNLVQQLLDLLRQNDIIRS

SEQ ID NO: 51 - DNA, 741 bp, Escherichia coli
atgttagatcaagtatgccagcttgcacggaatgcaggcgatgccattatgcaggtctacgacgggacgaaaccgatggacgtcgtcagcaaagcggacaattctccggtaacggcagcggatattgccgctcacaccgttatcatggacggtttacgtacgctgacaccggatgttccggtcctttctgaagaagatcctcccggttgggaagtccgtcagcactggcagcgttactggctggtagacccgctggatggtactaaagagtttattaaacgtaatggcgaattcaccgttaacattgcgctcattgaccatggcaaaccgattttaggcgtggtgtatgcgccggtaatgaacgtaatgtacagcgcggcagaaggcaaagcgtggaaagaagagtgcggtgtgcgcaagcagattcaggtccgcgatgcgcgcccgccgctggtggtgatcagccgttcccatgcggatgcggagctgaaagagtatctgcaacagcttggcgaacatcagaccacgtccatcggctcttcgctgaaattctgcctggtggcggaaggacaggcgcagctgtacccgcgcttcggaccaacgaatatttgggacaccgccgctggacatgctgtagctgcagctgccggagcgcacgttcacgactggcagggtaaaccgctggattacactccgcgtgagtcgttcctgaatccggggttcagagtgtctatttactaa

SEQ ID NO: 52 - protein, 246 aa, Escherichia coli
MLDQVCQLARNAGDAIMQVYDGTKPMDVVSKADNSPVTAADIAAHTVIMDGLRTLTPDVPVLSEEDPPGWEVRQHWQRYWLVDPLDGTKEFIKRNGEFTVNIALIDHGKPILGVVYAPVMNVMYSAAEGKAWKEECGVRKQIQVRDARPPLVVISRSHADAELKEYLQQLGEHQTTSIGSSLKFCLVAEGQAQLYPRFGPTNIWDTAAGHAVAAAAGAHVHDWQGKPLDYTPRESFLNPGFRVSIY

SEQ ID NO: 53 - DNA, 735 bp, Escherichia coli
atgtccaaactcgatctaaacgccctgaacgaactgccgaaggtagatcgcattctggcgctggcggaaactaacgccgaactggaaaaactggacgctgaaggccgcgtagcctgggcgctggataatctgcccggtgaatatgtactttcttccagctttggcattcaggcggcggtgagcctgcatctggtgaatcaaattcgcccggatattccggtgatcctcaccgatacgggttacttgttcccggaaacctaccgctttattgacgagttaacggacaaactcaagctcaacctgaaagtgtaccgtgctaccgaaagcgcagcctggcaggaagcacgctacggaaaactgtgggagcagggcgttgaaggcattgaaaagtacaatgacatcaacaaagtcgaaccgatgaaccgggctctgaaagaactgaatgcgcaaacctggtttgctggcctgcgccgtgaacaatccggcagtcgtgccaatttaccggtgctggcaattcagcgtggcgtatttaaagtgctgccgattatcgactgggataaccgaactatttatcagtacctgcaaaaacatggcctgaaatatcacccattatgggatgaaggatatttatcggtgggcgatacccatacaacccgtaaatgggaacccggcatggcggaagaagaaacgcgtttctttggcttaaaaagggaatgtgggttacacgaagggtaa

SEQ ID NO: 54 - protein, 244 aa, Escherichia coli
MSKLDLNALNELPKVDRILALAETNAELEKLDAEGRVAWALDNLPGEYVLSSSFGIQAAVSLHLVNQIRPDIPVILTDTGYLFPETYRFIDELTDKLKLNLKVYRATESAAWQEARYGKLWEQGVEGIEKYNDINKVEPMNRALKELNAQTWFAGLRREQSGSRANLPVLAIQRGVFKVLPIIDWDNRTIYQYLQKHGLKYHPLWDEGYLSVGDTHTTRKWEPGMAEEETRFFGLKRECGLHEG

SEQ ID NO: 55 - DNA, 3512 bp, Escherichia coli
atgacgacacaggtcccaccttccgcgttgcttccgttgaacccggagcaactggcacgccttcaggcggccacgaccgatttaactcccacccagcttgcctgggtttctggctatttctggggcgtactcaatcagcagcctgctgcgcttgcagcgacgccagcgccagccgcagaaatgccgggtataactattatctccgcctcgcaaaccggcaatgcgcgccgggttgctgaagcattacgtgatgatttattagcagcaaaactgaacgttaagctggtgaacgcgggcgactataaattcaaacaaatcgccagcgaaaaactgctcatcgtagtgacgtcaacgcaaggggaaggggaaccgccggaagaagccgtcgcgctgcataagttcctgttctccaaaaaagcgccaaagctggaaaacaccgcgtttgccgtgtttagcctcggcgatagctcttatgaatttttctgccagtccgggaaagatttcgacagcaagctggcggaactgggtggtgaacgcctgctcgaccgtgtcgatgccgatgttgaataccaggctgctgccagcgagtggcgcgcccgcgtggttgatgcgcttaaatcgcgtgcgcctgtcgcggcaccttcgcaatccgtcgctactggcgcggtaaatgaaatccacaccagcccgtacagcaaagacgcgccgctggtggctagcctctctgttaaccagaaaattaccgggcgtaactctgaaaaagacgttcgccatatcgaaattgacttaggtgactcgggcatgcgttaccagccgggtgacgcgctgggcgtctggtatcagaacgatccggcactggtgaaagaacttgtcgaactgctgtggctgaaaggcgatgaacctgtcaccgtcgagggcaaaacgttgcctctgaacgaagcgctacagtggcacttcgaactgaccgtcaacaccgccaacattgttgagaattacgccacgcttacccgcagtgaaacactgctgccgctggtgggcgataaagcgaagttacagcattacgccgcgacgacgccgattgttgacatggtgcgtttctccccggcacagcttgatgccgaagcgctaattaatctgctgcgcccgctgacgccgcgtctgtattccatcgcctcctcgcaggcggaagtcgagaacgaagtacacgtcaccgttggtgtggtgcgttacgacgtggaaggccgcgcccgtgccggtggtgcctccagcttcctcgctgaccgcgtggaagaagagggcgaagtccgcgtatttatcgaacataacgataacttccgcctgccagccaatccagaaaccccggtgattatgattggcccaggcaccggtattgcgccgttccgcgcctttatgcagcaacgcgccgccgacgaagcgccaggtaaaaactggctgttctttggtaatccgcactttacggaagacttcctgtaccaggtggagtggcagcgctacgtcaaagatggcgtgctgacacgtatcgatcttgcctggtcgcgcgatcaaaaagaaaaagtttacgtacaagacaaactgcgcgaacagggcgcggagctgtggcgctggatcaatgatggtgcccacatttatgtctgcggcgacgctaatcgcatggcgaaagacgttgagcaggcacttctggaagtgattgccgaatttggtggcatggacaccgaagcggcggatgaatttttaagtgagctgcgcgtagagcgccgttatcagcgagatgtctactaatgagcgaaaaacatccagggcctttagtggtcgaaggaaaactgacagacgccgagcgcatgaagcatgaaagcaactacctgcgcggcaccattgcggaagatttaaacgacggtctgaccggcggctttaagggcgacaacttcctgctgattcgcttccacggcatgtatcagcaggatgaccgcgacatccgcgccgaacgtgctgaacagaagctggagccgcgccacgcgatgctgcttcgctgtcgtctgccgggtggggtgattaccactaaacagtggcaggcgatcgacaaatttgccggtgaaaacaccatctatggcagcattcgcctgaccaaccgccagacgtttcagttccacggcattctgaaaaagaacgtcaaaccggtgcaccagatgctgcactcggtcggtcttgatgcgctggcgacagctaacgacatgaaccgtaacgtactctgcacctcgaacccttacgagtcgcagctgcacgcggaagcgtacgagtgggcgaagaagatttctgagcatctgttgcctcgtacccgcgcgtatgcggagatctggctcgaccaggaaaaagtcgccactactgatgaagaaccgatcctcggccagacctacctgccgcgtaaattcaaaaccacggtagtgatcccgccacagaacgatatcgatctgcacgccaacgacatgaacttcgtggcgatcgccgaaaacggcaagctggtgggctttaacctgttggtgggcggtgggctttccatcgaacacggcaacaagaaaacctacgcccgcaccgcgagtgagtttggctatctgccgctggagcatacgctggcggtggccgaagccgtcgtgacaactcagcgtgactggggtaaccgaaccgatcgtaaaaatgccaaaaccaaatacacgctggagcgcgtgggggttgagacgtttaaagcggaagtggagcgtcgcgcggggatcaaatttgaaccgatccgtccatatgagttcaccggacgaggcgatcgtattggctgggttaagggcattgatgataactggcacctgacgctgtttatcgaaaatggtcgcatccttgattatccggcgcgtccgctgaaaaccggcctgctggagatcgcgaagatccacaaaggcgatttccgcattacggcgaaccagaatctgatcatcgccggtgtaccggaaagcgagaaagcgaagatcgagaagatcgccaaagagagcgggttaatgaatgccgtcacgccgcagcgtgaaaactcgatggcttgcgtgtcattcccgacttgcccgctggcgatggcggaagcagagcgtttcctgccgtcttttatcgacaacatcgataatttaatggcgaaacatggtgtcagcgatgagcatatcgtgatgcgtgtaacaggctgcccgaacggttgtggtcgcgcgatgctggcggaagtgggcctggtgggtaaagcgccgggtcgctacaacctgcatcttggcggcaaccgcattgggacacgtatcccacggatgtataaagaaaacatcaccgagccggaaatcctggcgtcgcttgatgaactgatagggcgctgggcgaaagagcgcgaagcgggtgaaggcttcggcgactttacggtgcgtgcgggcatcattcgcccggtgctcgatccggcgcgtgatttgtgggattaa

SEQ ID NO: 56 - protein, 599 aa, Escherichia coli
MTTQVPPSALLPLNPEQLARLQAATTDLTPTQLAWVSGYFWGVLNQQPAALAATPAPAAEMPGITIISASQTGNARRVAEALRDDLLAAKLNVKLVNAGDYKFKQIASEKLLIVVTSTQGEGEPPEEAVALHKFLFSKKAPKLENTAFAVFSLGDSSYEFFCQSGKDFDSKLAELGGERLLDRVDADVEYQAAASEWRARVVDALKSRAPVAAPSQSVATGAVNEIHTSPYSKDAPLVASLSVNQKITGRNSEKDVRHIEIDLGDSGMRYQPGDALGVWYQNDPALVKELVELLWLKGDEPVTVEGKTLPLNEALQWHFELTVNTANIVENYATLTRSETLLPLVGDKAKLQHYAATTPIVDMVRFSPAQLDAEALINLLRPLTPRLYSIASSQAEVENEVHVTVGVVRYDVEGRARAGGASSFLADRVEEEGEVRVFIEHNDNFRLPANPETPVIMIGPGTGIAPFRAFMQQRAADEAPGKNWLFFGNPHFTEDFLYQVEWQRYVKDGVLTRIDLAWSRDQKEKVYVQDKLREQGAELWRWINDGAHIYVCGDANRMAKDVEQALLEVIAEFGGMDTEAADEFLSELRVERRYQRDVY

SEQ ID NO: 57 - protein, 570 aa, Escherichia coli
MSEKHPGPLVVEGKLTDAERMKHESNYLRGTIAEDLNDGLTGGFKGDNFLLIRFHGMYQQDDRDIRAERAEQKLEPRHAMLLRCRLPGGVITTKQWQAIDKFAGENTIYGSIRLTNRQTFQFHGILKKNVKPVHQMLHSVGLDALATANDMNRNVLCTSNPYESQLHAEAYEWAKKISEHLLPRTRAYAEIWLDQEKVATTDEEPILGQTYLPRKFKTTVVIPPQNDIDLHANDMNFVAIAENGKLVGFNLLVGGGLSIEHGNKKTYARTASEFGYLPLEHTLAVAEAVVTTQRDWGNRTDRKNAKTKYTLERVGVETFKAEVERRAGIKFEPIRPYEFTGRGDRIGWVKGIDDNWHLTLFIENGRILDYPARPLKTGLLEIAKIHKGDFRITANQNLIIAGVPESEKAKIEKIAKESGLMNAVTPQRENSMACVSFPTCPLAMAEAERFLPSFIDNIDNLMAKHGVSDEHIVMRVTGCPNGCGRAMLAEVGLVGKAPGRYNLHLGGNRIGTRIPRMYKENITEPEILASLDELIGRWAKEREAGEGFGDFTVRAGIIRPVLDPARDLWD

SEQ ID NO: 58 - DNA, 975 bp, Escherichia coli
atgaaattacaacaacttcgctatattgttgaggtggtcaatcataacctgaatgtctcatcaacagcggaaggactttacacatcacaacccgggatcagtaaacaagtcagaatgctggaagacgagctaggcattcaaattttttcccgaagcggcaagcacctgacgcaggtaacgccagcagggcaagaaataattcgtatcgctcgcgaagtcctgtcgaaagtcgatgccataaaatcggttgccggagagcacacctggccggataaaggttcactgtatatcgccaccacgcatacccaggcacgctacgcattaccaaacgtcatcaaaggctttattgagcgttatcctcgcgtttctttgcatatgcaccagggctcgccgacacaaattgctgatgccgtctctaaaggcaatgctgatttcgctatcgccacagaagcgctgcatctgtatgaagatttagtgatgttaccgtgctaccactggaatcgggctattgtagtcactccggatcacccgctggcaggcaaaaaagccattaccattgaagaactggcgcaatatccgttggtgacatataccttcggctttaccggacgttcagaactggatactgcctttaatcgcgcagggttaacgccgcgtatcgttttcacggcaacggatgctgacgtcattaaaacttacgtccggttagggctgggggtaggggtcattgccagcatggcggtggatccggtcgccgatcccgaccttgtgcgtgttgatgctcacgatatcttcagccacagtacaaccaaaattggttttcgccgtagtactttcttgcgcagttatatgtatgatttcattcagcgttttgcaccgcatttaacgcgtgatgtcgttgatgcggctgtcgcattgcgctctaatgaagaaattgaggtcatgtttaaagatataaaactgccggaaaaataa

SEQ ID NO: 59 - protein, 324 aa, Escherichia coli
MKLQQLRYIVEVVNHNLNVSSTAEGLYTSQPGISKQVRMLEDELGIQIFSRSGKHLTQVTPAGQEIIRIAREVLSKVDAIKSVAGEHTWPDKGSLYIATTHTQARYALPNVIKGFIERYPRVSLHMHQGSPTQIADAVSKGNADFAIATEALHLYEDLVMLPCYHWNRAIVVTPDHPLAGKKAITIEELAQYPLVTYTFGFTGRSELDTAFNRAGLTPRIVFTATDADVIKTYVRLGLGVGVIASMAVDPVADPDLVRVDAHDIFSHSTTKIGFRRSTFLRSYMYDFIQRFAPHLTRDVVDAAVALRSNEEIEVMFKDIKLPEK

SEQ ID NO: 60 - DNA, 387 bp, Roseobacter denitrificans
atgaccaaaacactgacagctcaggacttgtccgacacctttgacgccttcaatcgccatgacgttgatggcgtcatgacacatttcgccgatgattgcgtgttctacaccgtgggcggggatgaagcctatggcgccaaagtcgaaggcgcagaagcgattgccaaagcattctctgccgtctgggcgggcatgaaggacgcccattgggatcatcacagccactttgtgcatggggatcgcgccgtatccgaatggacgttctccggaactggcgcggacggcatgcgcatcgaagcacagggcgctgacctctttaccctgcgcgacggcaagatcatcgtgaaacaggccctgcgcaaatcccgcccgcccttcaaggcttaa

SEQ ID NO: 61 - protein, 128 aa, Roseobacter denitrificans
MTKTLTAQDLSDTFDAFNRHDVDGVMTHFADDCVFYTVGGDEAYGAKVEGAEAIAKAFSAVWAGMKDAHWDHHSHFVHGDRAVSEWTFSGTGADGMRIEAQGADLFTLRDGKIIVKQALRKSRPPFKA

SEQ ID NO: 62 - DNA, 1395 bp, Roseobacter denitrificans
atgccacatagaccaaagcactggcccaaggccagctacgatcccaaatacgatcctatcgtcgacgcgggtcccggtcacaaccgggaccacgcaccgacctattggattggtacggcggggacgccacctgaagatgacgggccggtgtcgggtgacatcgatgcggatgtcgtcgttgtcggctctggctatacaggtctgtctaccgcaatccacctggcgaaggaccacggcatcaaggcgcatgtccttgaagccaacacagtcgcctggggctgttccacccgcaatggcgggcaggcacagatttcttccggtcgtctcaagcggtcggagtggatcaagcggtggggcgtggatgtcgccaaaggcatgcacgccgaggtctgtgaagccttcgaactgttcaatgatctgatcgggtcagatgacattgattgcgacccgcaaaccgggggccatttctatattgcccaccgcgaaaaggtcatggcgaagctggaaaaggaatgtgccgtcctgaacgacacgtttggctatggctctcgcattctgtcgcgcgacgaactacacgaaaaatacgtgcgggatcaggaagcacacggtgccctttgggaaccggacgggacctcgatccacgcggcaaaactggccttcagctacgtgcgtcttgcgcgcaaactcggcgccaagatccacacggccagcccggtcatggggtggaagaccgtgaacggtgtgcatcacctcaccacgcccggtggcacggtgcgcgcacgtgccgtggccttggcgacagcgggctacacaccgccggggctgaacgaaaagaccaagcaccggctcatgccgatcctgtcaaactccatcgtgacgcgtccgctgagcgatgaggaaaaggcgggatgcggttttcaggtgaaatctccgctgactgacacgcgcaccttgcggcactactaccgctatctgcccgacggacgggtccagatcggcagccgcagtgcgattacaggtcgagacgcagagaaccccagacatctggagcttctgcagaaaggtctctatcgcaagttccccgtgctcgaaggcattgaactggattactcctggtggggatgggtggatgtcagccatgacatgatgccacgcattttccagccaaacccgaagcaaacaatcttttatgcgatgggctacggcggcaacggggtgatgtattccgcacaggccggcaagcgcatggcgcaaatggttgcgggcgaaggcaaggacctcaaacttccgatcttcacctcgcaactgccaagccacggtgttctgacacccttccgcaggttgggccagcgcatggcctacccctactactaccttcgcgatgaaattctctga

SEQ ID NO: 63 - protein, 464 aa, Roseobacter denitrificans
MPHRPKHWPKASYDPKYDPIVDAGPGHNRDHAPTYWIGTAGTPPEDDGPVSGDIDADVVVVGSGYTGLSTAIHLAKDHGIKAHVLEANTVAWGCSTRNGGQAQISSGRLKRSEWIKRWGVDVAKGMHAEVCEAFELFNDLIGSDDIDCDPQTGGHFYIAHREKVMAKLEKECAVLNDTFGYGSRILSRDELHEKYVRDQEAHGALWEPDGTSIHAAKLAFSYVRLARKLGAKIHTASPVMGWKTVNGVHHLTTPGGTVRARAVALATAGYTPPGLNEKTKHRLMPILSNSIVTRPLSDEEKAGCGFQVKSPLTDTRTLRHYYRYLPDGRVQIGSRSAITGRDAENPRHLELLQKGLYRKFPVLEGIELDYSWWGWVDVSHDMMPRIFQPNPKQTIFYAMGYGGNGVMYSAQAGKRMAQMVAGEGKDLKLPIFTSQLPSHGVLTPFRRLGQRMAYPYYYLRDEIL

SEQ ID NO: 64 - DNA, 852 bp, Escherichia coli
atgagtgaacgtctgagcattaccccgctggggccgtatatcggcgcacaaatttcgggtgccgacctgacgcgcccgttaagcgataatcagtttgaacagctttaccatgcggtgctgcgccatcaggtggtgtttctacgcgatcaagctattacgccgcagcagcaacgcgcgctggcccagcgttttggcgaattgcatattcaccctgtttacccgcatgccgaaggggttgacgagatcatcgtgctggatacccataacgataatccgccagataacgacaactggcataccgatgtgacatttattgaaacgccacccgcaggggcgattctggcagctaaagagttaccttcgaccggcggtgatacgctctggaccagcggtattgcggcctatgaggcgctctctgttcccttccgccagctgctgagtgggctgcgtgcggagcatgatttccgtaaatcgttcccggaatacaaataccgcaaaaccgaggaggaacatcaacgctggcgcgaggcggtcgcgaaaaacccgccgttgctacatccggtggtgcgaacgcatccggtgagcggtaaacaggcgctgtttgtgaatgaaggctttactacgcgaattgttgatgtgagcgagaaagagagcgaagccttgttaagttttttgtttgcccatatcaccaaaccggagtttcaggtgcgctggcgctggcaaccaaatgatattgcgatttgggataaccgcgtgacccagcactatgccaatgccgattacctgccacagcgacggataatgcatcgggcgacgatccttggggataaaccgttttatcgggcggggtaa

SEQ ID NO: 65 - protein, 283 aa, Escherichia coli
MSERLSITPLGPYIGAQISGADLTRPLSDNQFEQLYHAVLRHQVVFLRDQAITPQQQRALAQRFGELHIHPVYPHAEGVDEIIVLDTHNDNPPDNDNWHTDVTFIETPPAGAILAAKELPSTGGDTLWTSGIAAYEALSVPFRQLLSGLRAEHDFRKSFPEYKYRKTEEEHQRWREAVAKNPPLLHPVVRTHPVSGKQALFVNEGFTTRIVDVSEKESEALLSFLFAHITKPEFQVRWRWQPNDIAIWDNRVTQHYANADYLPQRRIMHRATILGDKPFYRAG

SEQ ID NO: 66 - DNA, 1392 bp, Roseobacter denitrificans
atggacggcaatttcaatgaaaatgatatctcccgcgtcgtcgaagcagaccgcgcgcatatctggcaccatctgagccagcacaaaccttacgagacaacagacccgcgcatcattgtcgaaggcaagggcatgaaggtttgggaccagaagggcaaagagcatcttgatgccgtctccggtggggtctggaccgtcaatgtcggctatggccgcgaacgcatcgccaacgccgtgcgggaccagttggtcaagttgaactatttcgccggctccgcaggctccatccccggtgccatgttcgccgagcgtctgatcgagaagatgccggggctgagccgcgtttattactgcaattccggctccgaggcgaatgaaaaagccttcaagatggtccgccagatcgcgcacaaacgctatggcggcaaaaagcacaaggtgctttatcgcgagcgtgactatcacggcaccaccatttccgccctttccgcaggcgggcaggacgaacggaacgcacaatatggccccttcacgcccggtttcgtgcgcgtgccccattgccttgaataccgcgcctttgaacaggaaggggcgccacaggaaaactacggtgtctgggcggcggatcagatcgaaaaggtaatcctcgccgaagggcccgataccgtgggcggcctgtgccttgaaccggtcactgcaggtggcggggtgatcacgccccccgatggctactgggagcgtgtgcaggaaatctgccacaaatacgacatcctgctgcatatcgacgaggtcgtatgcggcgtcggtcggaccggcacatggttcggctatcagcactacggcatccagccggatatggtcacgatggccaagggtgtcgcgtccggttacgcggcgatcgcctgccttgtgaccaatgaaaaagtcttcgacatgttcaaggatgacgcctcggatccgctgaactacttccgcgacatctcgacctttgggggctgcacggcgggtccggcagctgcgctggaaaacctgtcgatcatcgaagaagaaggcctgctggacaacaccacggaacagggggcctatatgctcgactgtctgggcggcttgatggacaagcacaagatcatcggccaggtgcgcggcaaggggctgttcctcggtgccgaactggtcgaggatcgcgacacgcgcaaaccggttgacgaaaggctcgcgcaagcggtggtcgcggactgcatgcaacagggtgtgatcatcggcgtgaccaaccgctctctgccgggcaagaacaacacgctgtgtttctcgcccgccctgatcgccagcaaggatgacattgaccacatctgcgacgcggtggacggtgcgctgtcgcgcgttttcggctaa

SEQ ID NO: 67 - protein, 463 aa, Roseobacter denitrificans
MDGNFNENDISRVVEADRAHIWHHLSQHKPYETTDPRIIVEGKGMKVWDQKGKEHLDAVSGGVWTVNVGYGRERIANAVRDQLVKLNYFAGSAGSIPGAMFAERLIEKMPGLSRVYYCNSGSEANEKAFKMVRQIAHKRYGGKKHKVLYRERDYHGTTISALSAGGQDERNAQYGPFTPGFVRVPHCLEYRAFEQEGAPQENYGVWAADQIEKVILAEGPDTVGGLCLEPVTAGGGVITPPDGYWERVQEICHKYDILLHIDEVVCGVGRTGTWFGYQHYGIQPDMVTMAKGVASGYAAIACLVTNEKVFDMFKDDASDPLNYFRDISTFGGCTAGPAAALENLSIIEEEGLLDNTTEQGAYMLDCLGGLMDKHKIIGQVRGKGLFLGAELVEDRDTRKPVDERLAQAVVADCMQQGVIIGVTNRSLPGKNNTLCFSPALIASKDDIDHICDAVDGALSRVFG

SEQ ID NO: 68 - DNA, 3415 bp, Escherichia coli
atggcaatttcatcgcgtaacacacttcttgccgcactggcattcatcgcttttcaggcacaggcggtgaacgtcaccgtggcgtatcaaacctcagccgaaccggcgaaagtggctcaggccgacaacacctttgctaaagaaagcggagcaaccgtggactggcgtaagtttgacagcggagccagcatcgtgcgggcgctggcttcaggcgacgtgcaaatcggcaacctcggttccagcccgttagcggttgcagccagccaacaggtgccgattgaagtcttcttgctggcgtcaaaactgggtaactccgaagcgctggtggtaaagaaaactatcagcaaaccggaagatctgattggcaaacgcatcgccgtaccgtttatctccaccacccactacagcctgctggcggcactgaaacactggggcattaaacccgggcaagtggagattgtgaacctgcagccgcccgcgattatcgctgcctggcagcggggagatattgatggtgcttatgtctgggcaccggcggttaacgccctggaaaaagacggcaaggtgttgaccgattctgaacaggtcgggcagtggggcgcgccaacgctggacgtctgggtggtgcgcaaagattttgccgagaaacatcctgaggtcgtgaaagcgttcgctaaaagcgccatcgatgctcagcaaccgtacattgctaacccagacgtgtggctgaaacagccggaaaacatcagcaaactggcgcgtttaagcggcgtgcctgaaggtgacgttccggggctggtgaaggggaatacctatctgacgccgcagcaacaaacggcagaactgaccggaccggtgaacaaagcgatcatcgacaccgcgcagtttttgaaagagcagggcaaggtcccggctgtagcgaatgattacagccagtacgttacctcgcgcttcgtgcaataaaaggaggcgcagatgctgcaaatctctcatctttacgccgattatggcggcaaaccggcactggaagatatcaacctgacgctggaaagcggcgagctactggtggtgctggggccgtccggctgcggtaaaaccaccctgctgaatctgattgccggttttgtgccttatcagcatggcagcattcaactggcgggtaagcgtattgagggaccgggagcagagcgtggcgtagtttttcagaatgaagggctactaccgtggcgcaatgtacaggacaacgtggcgttcggcctgcaattggcaggtatagagaaaatgcagcgactggaaatcgcgcaccagatgctgaaaaaagtggggctggaaggcgcagaaaaacgctacatctggcagctttccggtggtcaacgtcagcgggtggggattgctcgtgcgctggcggcgaatccccagctgttattactcgacgaaccgtttggtgcgctggacgccttcacccgcgaccagatgcaaaccctgctgctgaaactctggcaggagacgggcaagcaggtgctgttgattacccacgatatagaagaagcggtgtttatggcgactgaactggttctgctttcatccggccctggccgtgtgctggagcggctgccgctcaactttgctcgccgctttgttgcgggagagtcgagccgcagcatcaagtccgatccacaattcatcgccatgcgcgaatatgttttaagccgcgtatttgagcaacgggaggcgttctcatgagtgtgctcattaatgaaaaactgcattcgcggcggctgaaatggcgctggccgctctcgcgtcaggtgaccttaagcattggcacgttagcggttttactcaccgtatggtggacggtggcgacgctgcaactgattagcccgctatttttgccgccgccgcaacaggtactggaaaaactactcaccattgccggaccgcaaggctttatggacgccacgctgtggcagcatctggcagccagtctgacgcgcattatgctggcgctatttgcagcggtgttgttcggtattccggtcgggatcgcgatgggacttagccctacggtacgcggcattctggatccgataatcgagctttatcgtccggtgccgccgctggcttatttgccgctgatggtgatctggtttggtattggtgaaacctcgaagatcttactgatctatttagcgatttttgcaccggtggcgatgtcggcgctggcgggggtgaaaagcgtgcagcaggttcgcattcgtgccgcccagtcgctgggtgccagccgtgcgcaggtgctgtggtttgtcattttgcccggtgcgctgccggaaatcctcaccggattacgtattggtctgggggtgggctggtctacgctggtggcggcggagctgattgccgcgacgcgcggtttaggatttatggttcagtcagcgggtgaatttctcgcaactgacgtggtgctggcggggatcgcggtgattgcgattatcgcctttcttttagaactgggtctgcgcgcgttacagcgccgcctgacgccctggcatggagaagtacaatgagtgaacgtctgagcattaccccgctggggccgtatatcggcgcacaaatttcgggtgccgacctgacgcgcccgttaagcgataatcagtttgaacagctttaccatgcggtgctgcgccatcaggtggtgtttctacgcgatcaagctattacgccgcagcagcaacgcgcgctggcccagcgttttggcgaattgcatattcaccctgtttacccgcatgccgaaggggttgacgagatcatcgtgctggatacccataacgataatccgccagataacgacaactggcataccgatgtgacatttattgaaacgccacccgcaggggcgattctggcagctaaagagttaccttcgaccggcggtgatacgctctggaccagcggtattgcggcctatgaggcgctctctgttcccttccgccagctgctgagtgggctgcgtgcggagcatgatttccgtaaatcgttcccggaatacaaataccgcaaaaccgaggaggaacatcaacgctggcgcgaggcggtcgcgaaaaacccgccgttgctacatccggtggtgcgaacgcatccggtgagcggtaaacaggcgctgtttgtgaatgaaggctttactacgcgaattgttgatgtgagcgagaaagagagcgaagccttgttaagttttttgtttgcccatatcaccaaaccggagtttcaggtgcgctggcgctggcaaccaaatgatattgcgatttgggataaccgcgtgacccagcactatgccaatgccgattacctgccacagcgacggataatgcatcgggcgacgatccttggggataaaccgttttatcgggcggggtaa

SEQ ID NO: 69 - DNA, 4236 bp, Escherichia coli
atgcgtgtcatcaccctggcgggtagtcctcgctttccttctcgctccagctccttgctggaatatgcgcgggaaaaactaaatggcctggatgtagaggtttatcactggaatctgcaaaacttcgccccggaagatctactttatgctcgtttcgatagtccggcactcaagaccttcaccgaacagctgcaacaggccgatgggctgattgtcgccacgcctgtgtataaagccgcctattccggtgcgttgaaaaccctgctcgacctgctgccagaacgcgctttgcaaggcaaagtggtgctaccgctggcgacgggcggtaccgtggcccatctgctggcggtcgattatgcccttaaaccagttttaagcgcactgaaagctcaggagatcctgcacggcgtgtttgccgatgactcacaagtaattgattaccatcacagaccccagttcacgccaaatctgcaaacccgtcttgataccgcgctagaaactttctggcaggcattgcaccgccgcgatgttcaggttcctgaccttctgtctctgcgaggtaatgcccatgcgtaacatcattaaactggcgctggcgggattgcttagcgtctctacttttgcggttgctgcagaatcctcgcctgaagcgttacgtataggctatcagaaaggcagtattggtatggtgctggcaaaaagccaccagttactggaaaaacgctatccggaatcaaaaatctcctgggtggagttccccgcgggtccgcaaatgttggaagcgttaaacgttggcagtattgatctcggcagtaccggggatattccgccaatctttgcccaggctgccggggctgatttggtgtacgtgggcgtcgagccaccgaagcccaaagccgaagtgattctggtggcagaaaacagcccgatcaaaaccgtagccgatcttaaaggtcacaaagttgcctttcagaaaggttccagttcacacaaccttttactgcgtgcactgcgtcaggccggacttaagtttaccgatatccaacccacttacctgacgcccgctgatgcccgcgccgcgttccagcaaggtaacgttgacgcctgggctatctgggatccctactactctgctgcattattacagggcggcgtgcgggtgctgaaagacggcaccgatctcaatcaaactggatcgttttatctggcagctcgtccctatgcagaaaaaaacggcgcttttattcagggcgtactggcaacctttagtgaggccgatgcgttaacccgcagccagcgcgagcaaagcatcgctttactggcaaaaacgatgggcttaccggcaccggtgattgcctcttacttagatcatcgccctcctaccaccatcaaaccggttaacgccgaggttgccgccttacagcagcaaacggcagatctgttttatgaaaatcgtctggtgccgaaaaaagtcgatattcgccagcgcatctggcagcccactcaactggaaggaaaacaattatgagtctgaatatgttctggtttttaccgacccacggtgacgggcattatctgggaacggaagaaggttcacgcccggttgatcacggttatctgcaacaaattgcgcaagcggcggatcgtcttggctataccggtgtgctaattccaacggggcgctcctgcgaagatgcgtggctggttgccgcatcgatgatcccggtgacgcagcggctgaagtttcttgtcgccctgcgtcccagcgtaacctcacctaccgttgccgcccgccaggccgccacgcttgaccgtctctcaaatggacgtgcgttgtttaacctggtcacaggcagcgatccacaagagctggcaggcgacggagtgttccttgatcatagcgagcgctacgaagcctcggcggaatttacccaggtctggcggcgtttattgcagagagaaaccgtcgatttcaacggtaaacatattcatgtgcgcggagcaaaactgctcttcccggcgattcaacagccgtatccgccactttactttggcggatcgtcagatgtcgcccaggagctggcggcagaacaggttgatctctacctcacctggggcgaaccgccggaactggttaaagagaaaatcgaacaagtgcgggcgaaagctgccgcgcatggacgcaaaattcgtttcggtattcgtctgcatgtgattgttcgtgaaactaacgacgaagcgtggcaggccgccgagcggttaatctcgcatcttgatgatgaaactatcgccaaagcacaggccgcattcgcccggacggattccgtagggcaacagcgaatggcggcgttacataacggcaagcgcgacaatctggagatcagccccaatttatgggcgggcgttggcttagtgcgcggcggtgccgggacggcgctggtgggcgatggtcctacggtcgctgcgcgaatcaacgaatatgccgcgcttggcatcgacagttttgtgctttcgggctatccgcatctggaagaagcgtatcgggttggcgagttgctgttcccgcttctggatgtcgccatcccggaaattccccagccgcagccgctgaatccgcaaggcgaagcggtggcgaatgattttatcccccgtaaagtcgcgcaaagctaaggaagaaataatggcaacgccagtgaagaagtggttattgcgcgttgccccctggtttttaccggtgggcatcgtggcggtgtggcaactggcctcctcggttggctggctgtcgacgcgtattttgccttcaccggaaggagtggtgacggcgttctggacgctctccgccagcggcgaactgtggcagcatctggcgatcagctcctggcgggcgctgattggcttttcaattggcggatcgctggggttgattttgggactgattagcgggctatctcgctggggagaacggctgctggatacctcaattcagatgttgcgcaacgtgccgcatctggcgctgatcccgctggtgattttgtggtttggcattgatgaatccgcaaaaatctttctggtggcgctcggtacgctattccccatttatatcaacacctggcatggcatccgtaatatcgatcgcgggttggtggagatggcgcgtagctatggattatccggcataccgctgtttatccatgtgatcctgcctggtgccctgccctcaattatggtcggcgtgcgttttgcgttgggcctgatgtggctgacgctgattgttgccgaaaccatttctgccaattcaggcattggttatctggcgatgaatgcgcgggagtttttgcaaacggacgtggtggtagtcgctattattctttacgccctgctcggcaaactggctgacgtcagcgcgcagctgttagagcgcctctggttgcgctggaacccggcttatcatttgaaggaggccacggtatgaatactgctcgtctgaaccagggcacgccattgttgctcaatgcagtaagcaaacattacgcggaaaatatcgtcctgaaccaactggatttacatattccggcaggtcagtttgtggcggtggtgggccgcagcggtggtggcaaaagtaccctgctgcgcctgctggcaggtctggaaacgccaaccgcaggcgatgtgttagcgggcaccacaccgctggctgaaattcaggaagacacgcgaatgatgtttcaggatgcgcgtctgctaccgtggaaatcggtgattgataacgttgggttaggccttaaaggccagtggcgcgatgccgcacgtcgagcgttggctgcggtaggactggagaatcgcgcaggggaatggcctgccgcactttctggcgggcagaaacagcgagtggcgctggcaagagcgttgattcatcgaccgggattattgttgcttgatgaaccgctcggggcgctggacgccttaacgcgactcgagatgcaggatttgattgtgtcactttggcaggagcacggctttaccgtactgttggtgacgcatgatgtgagcgaagcggtagcgatggctgaccgggtgctgttaattgaagagggaaaaattggtctggatttgacagtggatatcccccgtccgcgtcgattagggtcggtgcgattagcggagctggaagcggaggtgttgcaacgggtgatgcaacgtggtgaatctgaaactcgcctgagaaaacaggggtaa

SEQ ID NO: 70 - DNA, 1146 bp, Escherichia coli
atgagtctgaatatgttctggtttttaccgacccacggtgacgggcattatctgggaacggaagaaggttcacgcccggttgatcacggttatctgcaacaaattgcgcaagcggcggatcgtcttggctataccggtgtgctaattccaacggggcgctcctgcgaagatgcgtggctggttgccgcatcgatgatcccggtgacgcagcggctgaagtttcttgtcgccctgcgtcccagcgtaacctcacctaccgttgccgcccgccaggccgccacgcttgaccgtctctcaaatggacgtgcgttgtttaacctggtcacaggcagcgatccacaagagctggcaggcgacggagtgttccttgatcatagcgagcgctacgaagcctcggcggaatttacccaggtctggcggcgtttattgcagagagaaaccgtcgatttcaacggtaaacatattcatgtgcgcggagcaaaactgctcttcccggcgattcaacagccgtatccgccactttactttggcggatcgtcagatgtcgcccaggagctggcggcagaacaggttgatctctacctcacctggggcgaaccgccggaactggttaaagagaaaatcgaacaagtgcgggcgaaagctgccgcgcatggacgcaaaattcgtttcggtattcgtctgcatgtgattgttcgtgaaactaacgacgaagcgtggcaggccgccgagcggttaatctcgcatcttgatgatgaaactatcgccaaagcacaggccgcattcgcccggacggattccgtagggcaacagcgaatggcggcgttacataacggcaagcgcgacaatctggagatcagccccaatttatgggcgggcgttggcttagtgcgcggcggtgccgggacggcgctggtgggcgatggtcctacggtcgctgcgcgaatcaacgaatatgccgcgcttggcatcgacagttttgtgctttcgggctatccgcatctggaagaagcgtatcgggttggcgagttgctgttcccgcttctggatgtcgccatcccggaaattccccagccgcagccgctgaatccgcaaggcgaagcggtggcgaatgattttatcccccgtaaagtcgcgcaaagctaa

SEQ ID NO: 71 - protein, 381 aa, Escherichia coli
MSLNMFWFLPTHGDGHYLGTEEGSRPVDHGYLQQIAQAADRLGYTGVLIPTGRSCEDAWLVAASMIPVTQRLKFLVALRPSVTSPTVAARQAATLDRLSNGRALFNLVTGSDPQELAGDGVFLDHSERYEASAEFTQVWRRLLQRETVDFNGKHIHVRGAKLLFPAIQQPYPPLYFGGSSDVAQELAAEQVDLYLTWGEPPELVKEKIEQVRAKAAAHGRKIRFGIRLHVIVRETNDEAWQAAERLISHLDDETIAKAQAAFARTDSVGQQRMAALHNGKRDNLEISPNLWAGVGLVRGGAGTALVGDGPTVAARINEYAALGIDSFVLSGYPHLEEAYRVGELLFPLLDVAIPEIPQPQPLNPQGEAVANDFIPRKVAQS

SEQ ID NO: 72 - DNA, 1146 bp, Corynebacterium glutamicum
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaa

SEQ ID NO: 73 - protein, 381 aa, Corynebacterium glutamicum
MTLTFHWFLSTSGDSRGIIGGGHGAEKSGTSRELSHSYLKQLALAAETNGFESVLTPTGTWCEDAWITDASLIEATKRLKFLVALRPGQIGPTLSAQMASTFQRLSGNRLLINVVTGGEDAEQRAFGDFLNKEERYARTGEFLDIVSRLWRGETVTHHGEHLQVEQASLAHPPEIIPEILFGGSSPAAGEVAARYADTYLTWGETPDQVAQKINWINELAAQRGRELRHGIRFHVITRDTSEEAWVVAEKLISGVTPEQVAKAQAGFATSKSEGQRRMAELHSKGRAFTSGSTARDLEVYPNVWAGVGLLRGGAGTALVGSHEEVADRIEEYAALGLDQFVLSGYPNLEEAFHFGEGVIPELLRRGVDIKNQESRVLEPVG

SEQ ID NO: 74 - DNA, 576 bp, Escherichia coli
atgcgtgtcatcaccctggcgggtagtcctcgctttccttctcgctccagctccttgctggaatatgcgcgggaaaaactaaatggcctggatgtagaggtttatcactggaatctgcaaaacttcgccccggaagatctactttatgctcgtttcgatagtccggcactcaagaccttcaccgaacagctgcaacaggccgatgggctgattgtcgccacgcctgtgtataaagccgcctattccggtgcgttgaaaaccctgctcgacctgctgccagaacgcgctttgcaaggcaaagtggtgctaccgctggcgacgggcggtaccgtggcccatctgctggcggtcgattatgcccttaaaccagttttaagcgcactgaaagctcaggagatcctgcacggcgtgtttgccgatgactcacaagtaattgattaccatcacagaccccagttcacgccaaatctgcaaacccgtcttgataccgcgctagaaactttctggcaggcattgcaccgccgcgatgttcaggttcctgaccttctgtctctgcgaggtaatgcccatgcgtaa

SEQ ID NO: 75 - protein, 191 aa, Escherichia coli
MRVITLAGSPRFPSRSSSLLEYAREKLNGLDVEVYHWNLQNFAPEDLLYARFDSPALKTFTEQLQQADGLIVATPVYKAAYSGALKTLLDLLPERALQGKVVLPLATGGTVAHLLAVDYALKPVLSALKAQEILHGVFADDSQVIDYHHRPQFTPNLQTRLDTALETFWQALHRRDVQVPDLLSLRGNAHA

SEQ ID NO: 76 - DNA, 924 bp, Corynebacterium glutamicum
atgacgtccccgcataattttgtcagtggtgctattgatctgggtgaggtgaaagcgcgtgcggatgcgcgccagaaggcccatgagcaggggccggtaactcagggcattgctagttcccttgatgtgaccatggagaacctggagaatgaggtgctgcgtcgttccacgcaggttccggtgattgttctcgtgggtaccccgcgcagccctgattcggagcagttgaagtcggatctgaccacgcttgctgctgaaagtggcaggaagttcattttcggttatgtcaatgctgataccgatgctgatgtggcccaggtgtttggggtgcagggcttgccgtcggtgattgctgtggcagcgggacgccctctggctgatttccagggcggacagccagcggatgcactaaagcagtggactgatcaggtggttcaggctgtgggtggacagctggaaggactgccagaggaggccacagacggcgaacaagaagacgctcctgtggaagacccccgcttcgatgctgccactgatgctctaaaccgtggcgctttcgatgaggcgattgcggtttatgagtccattttggcgcaggagccaaacaacgctgatgcgaagcaggcacgcgataccgcaaagctgttgggccggcttgccacggtggatccttcggtggatgttgtcgctgctgcagatgctgatccaacaaacgttgatctggcctacacagcagctgacgcggctgttgttgcgggtgatcctgaggctgcctttgatcgtttaattgctctgctgaccatcagcgctggcgatcagaagaatcaggtgaaggaacgtttgctggagctgtttggcatgtttgagaccgccgatccccgtgtgctgcaggcgcgaggaaagatggccagcgcgctgttctaa

SEQ ID NO: 77 - protein, 307 aa, Corynebacterium glutamicum
MTSPHNFVSGAIDLGEVKARADARQKAHEQGPVTQGIASSLDVTMENLENEVLRRSTQVPVIVLVGTPRSPDSEQLKSDLTTLAAESGRKFIFGYVNADTDADVAQVFGVQGLPSVIAVAAGRPLADFQGGQPADALKQWTDQVVQAVGGQLEGLPEEATDGEQEDAPVEDPRFDAATDALNRGAFDEAIAVYESILAQEPNNADAKQARDTAKLLGRLATVDPSVDVVAAADADPTNVDLAYTAADAAVVAGDPEAAFDRLIALLTISAGDQKNQVKERLLELFGMFETADPRVLQARGKMASALF

SEQ ID NO: 78 - DNA, 2727 bp, Ostreococcus tauri
atgcggacatacacccgatcttccgagggtccccgcccattccacgagagcgcgccgtcgccagtgcgcgcctccccagtacgccgcgccgttcacatcgccgacgcgagcgccggtgctgtcacttccgatcactaccggcgcgctcgctcgaacggcgccgcgcgcgccgtgcgcggcgccgacgagacgtcgtcttcagggtttgattcaacagaggcgtcgctcgagtccgtcgacgacgccctcgagagcgatctccagcgacgccgacgacctggaggagctcctgaacttagattacccggtggacatgccggagttcgagaacgcgctgccgacgataagcactggcggtcgacgcacgcgcgaatcgcaaacggagcggtggtgccacagcagctcatcggtgggacgccgatgatcgatcttagtgagtttagtgcgaacccaaaggtgaagatctatgggaagtgcgagtacatgaatccgagtgggagcattaaagatcggattgcgcaggagattttgactcgggcgctggagacgggcgagttgaaacccgggatgacggtcgtggcggcaacgagtgggaacaccggggcggcgatcgcgatggcgtgcgcgattcgtgggtttgattacatcgtgatcacgaataagaagacgagtaaggagaagattgacgccatgaaggcgtacggggggcaagtcatcgtcgcggagagtggggtcccggcagatcatccggatcattatcagaacatcgagacgacgatgtgcgcgcagaacccgaactattacggcgtaaatcagtacgataatccgtacaacgcggatgcgtacgagaagactcttggtcccgagatttggtcgcaaaccaagggggcagtgacgcacttcatcgcgggcggttccacaggaggcaccatcactggtaccggtcgctacttgaagagcgtagatccaacgatcaaaatcatgttagctgatcccaagggtagcgttttgtgggactatttcgtcaatgacgtgcccgaagaggatcttgtggcgaagagttgggaagtcgagggcgtcggcaaggactccattccgggtgttttgcagacagaatacatcgacggtgccgtgaaaggttgcgacgcgagctcattccgaatttgccgaatggtggccgaatcttcgggcatcttgctcggcggtagctccggtctgaacctgcacgccgctcgagtgctctcgagccagatcaaggagggtgttatagtcacggttttgtgcgacagcggtgtcaagtatttgtcaaagatcttcaacgacgaatggctcgaatcgaagaatttgaatcagccattgtcggatgtcaagaacttccaagtcgcttggaaaaaggaccagtctgaggcgagtgacgacgaagacgcagaccacggtctgtggagtcgcgacgatgaggagaaggagcttcgctttctagacgaaatcgcgacgcacatggttgagtactaccgcaactccgcgcgcgccgccgacccggtcagtacgtacaactctccgctcgcccttcacgaaaagttcaaggaggtgggtattccgttggccatcggtacgggtgaggagccggtctcgatgagcttactcaccaccgcgatgaacacggtgatccagaacagcgctcgcacctcgcacccaatgttaggaggaacgcgcggaggaagagacggaggaaggatgaggcggatgggtgagttgtcgtgggagctcggggcggcgggtgaggaggaggaggaggaagagcaggggcaggagggcgaggaaatctctcgagaggaggtgctggcgctctcggtggagctcatcgaatccatcgcttccgggaaggaaccgctcgatgcggcgcgtcttgggagcttgctttggactttgagcaacgcgctgttggaggatttgacggacagggacgatttacgcgttccggggaactcgctcgagacgtttccggtggagctggtgcgaaacgtcatgggcgccgtcgaaaccctggtcgttgcgctgacgttcgagccggaagatgcggtgacgcaacgaagtgggcacgcaattccggccatcggcacacatcgcgtcgctgcggcggagatcatcgccgtgctcttgcaaatcgggtgccaagacattgacgagcgcatcgcaaagctcaagctgccgaacgacggtcagttcgtcctcgtgtcgctcgtacgcatgtttttcaagtattcttggagttcggcgctgcacgcgaccgtggtgagactgatcttggccgcgctggtgagcccacacgagccgctctgggcacccatgtttgagggcggggacgagagtcttcagggctctctggcagcgtccatgaagacggcgctcgcgacgaagcccatctccacgagagacggtaacgtcgggagcgtgattattctcgccaacgctctgcacgagctcgaaacttgcgatgacgtcgagcgccagagcgtgcggacgacgttgcaggaggatgcaacgtggcgagccgccattgacggtgaggatagcccgttggcgaacctcaacaacgaacaggctggcggactttgtggaccaaagccgcagaagtcgcctgttttcatggactccggcatgggcgccaacgtcatcagcagccaagagctgctcaggatgttgcagcacatctcgcttggtcaatga

SEQ ID NO: 79 - protein, 908 aa, Ostreococcus tauri
MRTYTRSSEGPRPFHESAPSPVRASPVRRAVHIADASAGAVTSDHYRRARSNGAARAVRGADETSSSGFDSTEASLESVDDALESDLQRRRRPGGAPELRLPGGHAGVRERAADDKHWRSTHARIANGAVVPQQLIGGTPMIDLSEFSANPKVKIYGKCEYMNPSGSIKDRIAQEILTRALETGELKPGMTVVAATSGNTGAAIAMACAIRGFDYIVITNKKTSKEKIDAMKAYGGQVIVAESGVPADHPDHYQNIETTMCAQNPNYYGVNQYDNPYNADAYEKTLGPEIWSQTKGAVTHFIAGGSTGGTITGTGRYLKSVDPTIKIMLADPKGSVLWDYFVNDVPEEDLVAKSWEVEGVGKDSIPGVLQTEYIDGAVKGCDASSFRICRMVAESSGILLGGSSGLNLHAARVLSSQIKEGVIVTVLCDSGVKYLSKIFNDEWLESKNLNQPLSDVKNFQVAWKKDQSEASDDEDADHGLWSRDDEEKELRFLDEIATHMVEYYRNSARAADPVSTYNSPLALHEKFKEVGIPLAIGTGEEPVSMSLLTTAMNTVIQNSARTSHPMLGGTRGGRDGGRMRRMGELSWELGAAGEEEEEEEQGQEGEEISREEVLALSVELIESIASGKEPLDAARLGSLLWTLSNALLEDLTDRDDLRVPGNSLETFPVELVRNVMGAVETLVVALTFEPEDAVTQRSGHAIPAIGTHRVAAAEIIAVLLQIGCQDIDERIAKLKLPNDGQFVLVSLVRMFFKYSWSSALHATVVRLILAALVSPHEPLWAPMFEGGDESLQGSLAASMKTALATKPISTRDGNVGSVIILANALHELETCDDVERQSVRTTLQEDATWRAAIDGEDSPLANLNNEQAGGLCGPKPQKSPVFMDSGMGANVISSQELLRMLQHISLGQ

SEQ ID NO: 80 - DNA, 387 bp, Escherichia coli
atgagcaaaactatcgcgacggaaaatgcaccggcagctatcggtccttacgtacagggcgttgatctgggcaatatgatcatcacctccggtcagatcccggtaaatccgaaaacgggcgaagtaccggcagacgtcgctgcacaggcacgtcagtcgctggataacgtaaaagcgatcgtcgaagccgctggcctgaaagtgggcgacatcgttaaaactaccgtgtttgtaaaagatctgaacgacttcgcaaccgtaaacgccacttacgaagccttcttcaccgaacacaacgccaccttcccggcacgttcttgcgttgaagttgcccgtctgccgaaagacgtgaagattgagatcgaagcgatcgctgttcgtcgctaa

SEQ ID NO: 81 - protein, 128 aa, Escherichia coli
MSKTIATENAPAAIGPYVQGVDLGNMIITSGQIPVNPKTGEVPADVAAQARQSLDNVKAIVEAAGLKVGDIVKTTVFVKDLNDFATVNATYEAFFTEHNATFPARSCVEVARLPKDVKIEIEAIAVRR

SEQ ID NO: 82 - DNA, 390 bp, Escherichia coli
atgaaaaagattatcgaaacgcaacgtgccccaggcgcaatcggcccttatgttcagggcgttgatttaggcagcatggtcttcacctccgggcaaattccggtttgcccacagaccggtgagatcccggctgatgtgcaagatcaggcgcgtttaagcctcgaaaacgtcaaagcgatcgtggttgctgccgggctgagtgtgggcgatatcatcaagatgaccgtgttcatcaccgatctgaatgattttgccaccatcaatgaagtctataagcagttcttcgatgaacatcaggcgacctacccgacccggagctgtgtgcaggtcgcgcgtttgccgaaagatgtgaagctggaaattgaagccgtcgcagtacgtagtgcgtaa

SEQ ID NO: 83 - protein, 129 aa, Escherichia coli
MKKIIETQRAPGAIGPYVQGVDLGSMVFTSGQIPVCPQTGEIPADVQDQARLSLENVKAIVVAAGLSVGDIIKMTVFITDLNDFATINEVYKQFFDEHQATYPTRSCVQVARLPKDVKLEIEAVAVRSA

SEQ ID NO: 84 - DNA, 387 bp, Escherichia coli
atgccaaaatccgtaattattcccgctggcagcagcgcaccgctggcccccttcgttcccggcacgctggctgatggcgtggtgtatgtctccggtacgctggcttttgatcaacataataacgtgctgtttgccgatgacccaaaggcgcaaacccgccacgttctggaaactatccgcaaggtgatcgagacggcgggtggcacgatggcggatgtgaccttcaacagcatctttattaccgactggaaaaattacgccgcgattaacgaaatctacgccgagttttttccgggtgataaaccggcgcgattctgcattcagtgcggactggtaaaacctgacgcgctggtggaaatcgccacaattgcgcatatcgccaagtga

SEQ ID NO: 85 - protein, 128 aa, Escherichia coli
MPKSVIIPAGSSAPLAPFVPGTLADGVVYVSGTLAFDQHNNVLFADDPKAQTRHVLETIRKVIETAGGTMADVTFNSIFITDWKNYAAINEIYAEFFPGDKPARFCIQCGLVKPDALVEIATIAHIAK

SEQ ID NO: 86 - DNA, 1017 bp, Roseovarius sp. EC-HK134
atgcatcttgcccgctatccccgtcgttttctcgcccatctgccgactccgctggaacggctggaccgtttgaccaaggaattgaacggtcctgaaatctggatcaagcgggatgattgcaccggccttagcaccggcggcaacaagacccgcaagctggaattcctgatggcagaggccgaattgcagggcgcggatatggtgatgacccaaggggccacgcaatccaaccatgcgcgtcaaacggcggcctttgccgccaaactgggaatggattgccacctgctgcttgaggatcgcacgggttcgaacaacgccaattacaacaatggcggcaacgttctgttggatcacatgcacggggcgaccacggaaaagcgccccggtggcggcgacatgaatgccgagatggaaaaggtcgcggacaagttccgtgccgatgggcgcaatgtctataccattcccggtggtggctcgaacccgacaggcgcgcttggctatgttaactgcgccttcgagatgctggcgcagttcaatgatcgggcgctcaaggtggatcacattgttcatgctaccggcagtgctggcacgcaggcggggctgatcaccggtctcaaggcgatgaacgcgcaaattccgctcttgggcattggcgtccgcgcgcccaaggccaagcaagaagagaacgtctataacctcgcctgcgcgaccgctgaaaagctgggctgcgccggagtggtcaagcgtgaggatgtggttgccaataccgattatgttggcgaaggctatggcattcccacggaatccggcatcgaagccattcacatgtttgccgaattggaggcaatcctgctcgatccggtgtattcggccaaaggggctgcgggctttatcgacctgatccgcaagggtcatttcaagaaaggtgagcgcgtcgtctttctgcataccggggggtctgtcgcgctgtttggctatgacagcgcctttgatttctcgggccgctggaaaaagtaa

SEQ ID NO: 87 - protein, 338 aa, Roseovarius sp. EC-HK134
MHLARYPRRFLAHLPTPLERLDRLTKELNGPEIWIKRDDCTGLSTGGNKTRKLEFLMAEAELQGADMVMTQGATQSNHARQTAAFAAKLGMDCHLLLEDRTGSNNANYNNGGNVLLDHMHGATTEKRPGGGDMNAEMEKVADKFRADGRNVYTIPGGGSNPTGALGYVNCAFEMLAQFNDRALKVDHIVHATGSAGTQAGLITGLKAMNAQIPLLGIGVRAPKAKQEENVYNLACATAEKLGCAGVVKREDVVANTDYVGEGYGIPTESGIEAIHMFAELEAILLDPVYSAKGAAGFIDLIRKGHFKKGERVVFLHTGGSVALFGYDSAFDFSGRWKK

SEQ ID NO: 88 - DNA, 951 bp, Escherichia coli
atgaatttccaacaactaaagataatccgcgaggctgcacgtcaggattacaacctgacagaggttgcgaatatgctttttacctcacagtcaggcgtcagccgtcatattcgggaactggaggatgaacttggcatcgaaatatttgttcgacgaggtaagcgactgctgggcatgactgaaccgggcaaagcattactggtcattgcagaacgtattctgaatgaagccagtaatgttcgtcggcttgcagacctgtttaccaacgatacgtctggcgttctcactattgcaacgacgcatactcaggcacgttatagcttgccagaggtcattaaagcttttcgcgaacttttcccggaggttcggctcgagctaatccaggggacgccacaggaaattgcgacattgttgcaaaatggcgaagctgatattggtatcgccagcgagcgtttgagtaatgacccgcagctcgtcgccttcccgtggtttcgttggcaccatagtttgcttgttccacacgatcatcccttgacgcaaatttcaccattgacgctggaatcaatagcgaagtggccgttaatcacttaccgacaggggattacggggcgctcacgtattgatgacgcatttgcccgcaaaggtttgctggcagatattgtattaagtgcgcaggattctgatgtcattaaaacctatgttgctcttgggcttgggatcggattagttgccgagcaatccagtggcgaacaagaggaagagaatttaatccgcctggatacgcggcatctttttgatgctaatactgtctggttgggactgaagcgaggacaacttcagcgtaactatgtctggcgctttctggaactttgtaatgcaggactgtcagttgaggatatcaagcggcaggtgatggaaagcagtgaagaggaaattgattatcagatatag

SEQ ID NO: 89 - protein, 316 aa, Escherichia coli
MNFQQLKIIREAARQDYNLTEVANMLFTSQSGVSRHIRELEDELGIEIFVRRGKRLLGMTEPGKALLVIAERILNEASNVRRLADLFTNDTSGVLTIATTHTQARYSLPEVIKAFRELFPEVRLELIQGTPQEIATLLQNGEADIGIASERLSNDPQLVAFPWFRWHHSLLVPHDHPLTQISPLTLESIAKWPLITYRQGITGRSRIDDAFARKGLLADIVLSAQDSDVIKTYVALGLGIGLVAEQSSGEQEEENLIRLDTRHLFDANTVWLGLKRGQLQRNYVWRFLELCNAGLSVEDIKRQVMESSEEEIDYQI

SEQ ID NO: 90 - DNA, 927 bp, Corynebacterium glutamicum
atggacaacgacggcggagacatgcgaatcgacgacctacgcagcttcatttcagtcgcccaatcaggccacctcaccgaaaccgccgaaagattaggcatcccgcagcccacactttccagacgaatcagccgagtggaaaaacacgcaggcaccccacttttcgaccgcgccggccgcaaactcgtcctcaaccaacgaggccacgccttcctcaaccacgccagcgccatcgtcgcagaattcaactccgccgcaactgaaatcaaacgcctcatggacccagaaaaaggcacaatccgactggacttcatgcattccttgggcacttggatggtccccgaacttatccgaacattccgcgccgaacaccccaatgtagaattccaactccaccaagcggcagcaatgctcctggtagatcgtgttttggctgatgaaactgacctcgcattagttggccccaaacctgccgaggttggtacctctttagggtgggcgccactgcttcgtcaacgacttgccctagctgttcccgcagatcaccggcttgcctctttttctggccaaggagaattgccgttgattagtgcgacggaagaacctttcgtggcgatgcgagcaggtttcggcacccgactcctcatggatgcattagccgaagaagccggttttgttcccaatgtggttttcgaatccatggagctcaccaccgtcgcagggcttgtcagcgcaggtctcggcgttggtgtggttccgatggatgatccgtaccttcccacagtgggaatcgtgcaacgcccacttagtccacccgcatatagggaactcggtctggtatggaggcttaacgcgggacctgcaccggccgtggataacttccggaagttcgtggcgggatcgagatatgcattagaagagggctga

SEQ ID NO: 91 - protein, 308 aa, Corynebacterium glutamicum
MDNDGGDMRIDDLRSFISVAQSGHLTETAERLGIPQPTLSRRISRVEKHAGTPLFDRAGRKLVLNQRGHAFLNHASAIVAEFNSAATEIKRLMDPEKGTIRLDFMHSLGTWMVPELIRTFRAEHPNVEFQLHQAAAMLLVDRVLADETDLALVGPKPAEVGTSLGWAPLLRQRLALAVPADHRLASFSGQGELPLISATEEPFVAMRAGFGTRLLMDALAEEAGFVPNVVFESMELTTVAGLVSAGLGVGVVPMDDPYLPTVGIVQRPLSPPAYRELGLVWRLNAGPAPAVDNFRKFVAGSRYALEEG

SEQ ID NO: 92 - DNA, 1362 bp, Corynebacterium glutamicum
atgcttgccgaccttcccatcgccttaaacccacacgaaccaacatccatccccacgcagctcacagaacagatccgtcgtctcgtggcgaggggaattctcaccccaggagacccgcttcccagcagtcgctcactatccacccaattgggggtatcccgcggcagtgtggtgaccgcttatgaccaattggccggtgaaggctacctcagcaccgcccgcggttccggtacaacgatcaacccagatctgcatttgttgaagcctgtggaaattgagaagaaggagacgtcgagaagcgtcccgcccccgctgctcaacctgagccccggcgtgcccgataccgcgacgctcgccgattccgcatggcgcgctgcgtggcgcgaagcctgcgccaagccacccacgcactcccctgagcagggacttttgaggctgcggatcgagatcgccgaccacctgcgccagatgcgtggcctcatggtcgagccggagcagatcatcgtcaccgccggcgcgcgcgaggggctgagtctgctgctgcgcaccatggatgcgcctgcccgcatcggcgtcgaatcgcccggctaccccagcctgcgccgcatcccgcaggtgcttggccatgagacgatcgatgtgccgaccgacgaatccggcctcgtaccccgcgcgctgccccacgacctcaacgcgctactggtaacccctagccatcaatatccctacggcggctcgctgcccgccgatcgccgcaccgcgctagtcgcgtgggctgaggcaaacgatgcgttgcttattgaagacgacttcgattctgagctgcgctacgtcggtatgccgcttccgccgctgcgtgcgctggcgcccgatcgcacgattctgctcggcacgttttcctccgtgatcacaccacaagtcgcctgcggatacctcatcgcgccgacgccccaggcgcgcgtgctcgccacgcttcgcgggattctcggccagccagtcggcgccatcacccaacacgcgctcgcgtcctacctcgcctcaggcgctttacgacgccgcacccaacgtttgcggcgcctttaccgacaccgccgctccatcgtccaagacaccctcggtgacctcccgaatacgcagcttcgccccatcaacggtggcctccacgcagttctcctttgcgacaaaccccaagacctcgtcgtcaccacactcgcctcccgaggccttaacgtcaccgcgctttcccactactggggcggcaccggcgcagacaacggcatcgtcttcggcttcggctcccacgacgaagacaccctcagatgggtgcttgctgagatcagcgatgcggtgtctctaggctaa

SEQ ID NO: 93 - protein, 453 aa, Corynebacterium glutamicum
MLADLPIALNPHEPTSIPTQLTEQIRRLVARGILTPGDPLPSSRSLSTQLGVSRGSVVTAYDQLAGEGYLSTARGSGTTINPDLHLLKPVEIEKKETSRSVPPPLLNLSPGVPDTATLADSAWRAAWREACAKPPTHSPEQGLLRLRIEIADHLRQMRGLMVEPEQIIVTAGAREGLSLLLRTMDAPARIGVESPGYPSLRRIPQVLGHETIDVPTDESGLVPRALPHDLNALLVTPSHQYPYGGSLPADRRTALVAWAEANDALLIEDDFDSELRYVGMPLPPLRALAPDRTILLGTFSSVITPQVACGYLIAPTPQARVLATLRGILGQPVGAITQHALASYLASGALRRRTQRLRRLYRHRRSIVQDTLGDLPNTQLRPINGGLHAVLLCDKPQDLVVTTLASRGLNVTALSHYWGGTGADNGIVFGFGSHDEDTLRWVLAEISDAVSLG

SEQ ID NO: 94 - DNA, 1464 bp, Rhodobacteraceae bacterium
atggcgatttcccccgaaaccttctttctcgcctccgaagccgagggcacgctccagacccggatccgccagatggtggccgaggggatcctgaccggccgcttccgcccgggcgagaaactgccctcctcgcgcaagctcgccgcgcatctgggcgtcagccggatcaccgtgacactcgcctataccgaacttcaggccgacgattacatcacctcgcgcggccggtcgggctattacgtgtccgacaacgcgcccgaaccgccgtcctttcccgcgcgcgcacccgggcagtcgtcggtcgactggtcgcgcgccatcggccagcggtttcgcggcaccgaaccgcattcgaaacccggcaactgggccgatttccgctatccgttcatctatggccaggccgatccgaccctgttcgacgcggccaattggcggctctgtgccctccaggccctggggcgcaaggatttcgccgcgctgaccaccgattacaacgacagcgacgatcccgaattgctggatttcatcgcccgccagatcctgccccgccgcggcatcctggccgggccggacgaaatcctgctgacgctgggcgcccagaacgcgctgtggctgaccgcccaggtgctgctgacccagcgccggaccgcggcgatcgaggatccctgctacccggccctgcgcggcatcctgacccaggcgcgctgccacctgcacgcggtcccggtggatcgcgacgggttgccgcccgaggcgatccccgacggcaccaacgtggtgttctgcacccccagccaccaatgcccgaccaccgcgaccatgccgatgtcgcgccgccatgccctgctggaacgcgccgaggccgaggatttcctgatcgtcgaggatgattacgaattcgagatgtcgttcctcaaatccccctcgccggcgctgaaatcgctcgaccggcacgggcgggtgatctacgtcggctctttctccaaatcgctgtttccgggcctgcgcctgggctatctggtcggccccgagccgttcatccgggaggcccgcgccctgcgtgccagcgtgctgcgccacccgccgggccatatccagcgcaccgtcacctatttcctgtctcttggccattacgacgccctgatccgccgcatgggccgggcctatcacgaccggcgccggatcatggaccgcgccctgcacgatcacgggctgaccgtcgccggatcgggctccttcggcggctcgtccttctggatgcgcgcgcccgcgggcgtcgatacggccgagctggcccgccgcctgtccgccgacagcgtgctgatcgaaccgggccagccgttctttgccggcaccgcgccgcccgggcggttctaccgcctggcctatagttcgatctctggcccgcgcatccccgacgggatcgcgcggatcgccgccgcgctggagaactggtcctag

SEQ ID NO: 95 - protein, 487 aa, Rhodobacteraceae bacterium
MAISPETFFLASEAEGTLQTRIRQMVAEGILTGRFRPGEKLPSSRKLAAHLGVSRITVTLAYTELQADDYITSRGRSGYYVSDNAPEPPSFPARAPGQSSVDWSRAIGQRFRGTEPHSKPGNWADFRYPFIYGQADPTLFDAANWRLCALQALGRKDFAALTTDYNDSDDPELLDFIARQILPRRGILAGPDEILLTLGAQNALWLTAQVLLTQRRTAAIEDPCYPALRGILTQARCHLHAVPVDRDGLPPEAIPDGTNVVFCTPSHQCPTTATMPMSRRHALLERAEAEDFLIVEDDYEFEMSFLKSPSPALKSLDRHGRVIYVGSFSKSLFPGLRLGYLVGPEPFIREARALRASVLRHPPGHIQRTVTYFLSLGHYDALIRRMGRAYHDRRRIMDRALHDHGLTVAGSGSFGGSSFWMRAPAGVDTAELARRLSADSVLIEPGQPFFAGTAPPGRFYRLAYSSISGPRIPDGIARIAAALENWS

SEQ ID NO: 96 - DNA, 642 bp, Corynebacterium glutamicum
atggctgctagcgcttcaggcaagagtaaaacaagtgccggggcaaaccgtcgtcgcaatcgaccaagcccccgacagcgtctcctcgatagcgcaaccaaccttttcaccacagaaggtattcgcgtcatcggtattgatcgtatcctccgtgaagctgacgtggcgaaggcgagcctctattcccttttcggatcgaaggacgccttggttattgcatacctggagaacctcgatcagctgtggcgtgaagcgtggcgtgagcgcaccgtcggtatgaaggatccggaagataaaatcatcgcgttctttgatcagtgcattgaggaagaaccagaaaaagatttccgcggctcgcactttcagaatgcggctagtgagtaccctcgccccgaaactgatagcgaaaagggcattgttgcagcagtgttagagcaccgcgagtggtgtcataagactctgactgatttgctcactgagaagaacggctacccaggcaccacccaggcgaatcagctgttggtgttccttgatggtggacttgctggatctcgattggtccacaacatcagtcctcttgagacggctcgcgatttggctcggcagttgttgtcggctccacctgcggactactcaatttag

SEQ ID NO: 97 - protein, 213 aa, Corynebacterium glutamicum
MAASASGKSKTSAGANRRRNRPSPRQRLLDSATNLFTTEGIRVIGIDRILREADVAKASLYSLFGSKDALVIAYLENLDQLWREAWRERTVGMKDPEDKIIAFFDQCIEEEPEKDFRGSHFQNAASEYPRPETDSEKGIVAAVLEHREWCHKTLTDLLTEKNGYPGTTQANQLLVFLDGGLAGSRLVHNISPLETARDLARQLLSAPPADYSI

SEQ ID NO: 98 - DNA, 912 bp, Escherichia coli
atgagtacattagaacaaacaataggcaatacgcctctggtgaagttgcagcgaatggggccggataacggcagtgaagtgtggttaaaactggaaggcaataacccggcaggttcggtgaaagatcgtgcggcactttcgatgatcgtcgaggcggaaaagcgcggggaaattaaaccgggtgatgtcttaatcgaagccaccagtggtaacaccggcattgcgctggcaatgattgccgcgctgaaaggctatcgcatgaaattgctgatgcccgacaacatgagccaggaacgccgtgcggcgatgcgtgcttatggtgcggaactgattcttgtcaccaaagagcagggcatggaaggtgcgcgcgatctggcgctggagatggcgaatcgtggcgaaggaaagctgctcgatcagttcaataatcccgataacccttatgcgcattacaccaccactgggccggaaatctggcagcaaaccggcgggcgcatcactcattttgtctccagcatggggacgaccggcactatcaccggcgtctcacgctttatgcgcgaacaatccaaaccggtgaccattgtcggcctgcaaccggaagagggcagcagcattcccggcattcgccgctggcctacggaatatctgccggggattttcaacgcttctctggtggatgaggtgctggatattcatcagcgcgatgcggaaaacaccatgcgcgaactggcggtgcgggaaggaatattctgtggcgtcagctccggcggcgcggttgccggagcactgcgggtggcaaaagctaaccctgacgcggtggtggtggcgatcatctgcgatcgtggcgatcgctacctttctaccggggtgtttggggaagagcattttagccagggggcggggatttaa

SEQ ID NO: 99 - protein, 303 aa, Escherichia coli
MSTLEQTIGNTPLVKLQRMGPDNGSEVWLKLEGNNPAGSVKDRAALSMIVEAEKRGEIKPGDVLIEATSGNTGIALAMIAALKGYRMKLLMPDNMSQERRAAMRAYGAELILVTKEQGMEGARDLALEMANRGEGKLLDQFNNPDNPYAHYTTTGPEIWQQTGGRITHFVSSMGTTGTITGVSRFMREQSKPVTIVGLQPEEGSSIPGIRRWPTEYLPGIFNASLVDEVLDIHQRDAENTMRELAVREGIFCGVSSGGAVAGALRVAKANPDAVVVAIICDRGDRYLSTGVFGEEHFSQGAGI

SEQ ID NO: 100 - DNA, 1365 bp, Escherichia coli
atgattagtctattcgacatgtttaaggtggggattggtccctcatcttcccataccgtagggcctatgaaggcaggtaaacagttcgtcgatgatctggtcgaaaaaggcttactggatagcgttactcgcgttgccgtggacgtttatggttcactgtcgctgacgggtaaaggccaccacaccgatatcgccattattatgggtcttgcaggtaacgaacctgccaccgtggatatcgacagtattcccggttttattcgcgacgtagaagagcgcgaacgtctgctgctggcacagggacggcatgaagtggatttcccgcgcgacaacgggatgcgttttcataacggcaacctgccgctgcatgaaaacggtatgcaaatccacgcctataacggcgatgaagtcgtctacagcaaaacttattattccatcggcggcggttttatcgtcgatgaagaacactttggtcaggatgctgccaacgaagtaagcgtgccgtatccgttcaaatctgccaccgaactgctcgcgtactgtaatgaaaccggctattcgctgtctggtctcgctatgcagaacgaactggcgctgcacagcaagaaagagatcgacgagtatttcgcgcatgtctggcaaaccatgcaggcatgtatcgatcgcgggatgaacaccgaaggtgtactgccaggcccgctgcgcgtgccacgtcgtgcgtctgccctgcgccggatgctggtttccagcgataaactgtctaacgatccgatgaatgtcattgactgggtaaacatgtttgcgctggcagttaacgaagaaaacgccgccggtggtcgtgtggtaactgcgccaaccaacggtgcctgcggtatcgttccggcagtgctggcttactatgaccactttattgaatcggtcagcccggacatctatacccgttactttatggcagcgggcgcgattggtgcattgtataaaatgaacgcctctatttccggtgcggaagttggttgccagggcgaagtgggtgttgcctgttcaatggctgctgcgggtcttgcagaactgctgggcggtagcccggaacaggtttgcgtggcggcggaaattggcatggaacacaaccttggtttaacctgcgacccggttgcagggcaggttcaggtgccgtgcattgagcgtaatgccattgcctctgtgaaggcgattaacgccgcgcggatggctctgcgccgcaccagtgcaccgcgcgtctcgctggataaggtcatcgaaacgatgtacgaaaccggtaaggacatgaacgccaaataccgcgaaacctcacgcggtggtctggcaatcaaagtccagtgtgactaa

SEQ ID NO: 101 - protein, 454 aa, Escherichia coli
MISLFDMFKVGIGPSSSHTVGPMKAGKQFVDDLVEKGLLDSVTRVAVDVYGSLSLTGKGHHTDIAIIMGLAGNEPATVDIDSIPGFIRDVEERERLLLAQGRHEVDFPRDNGMRFHNGNLPLHENGMQIHAYNGDEVVYSKTYYSIGGGFIVDEEHFGQDAANEVSVPYPFKSATELLAYCNETGYSLSGLAMQNELALHSKKEIDEYFAHVWQTMQACIDRGMNTEGVLPGPLRVPRRASALRRMLVSSDKLSNDPMNVIDWVNMFALAVNEENAAGGRVVTAPTNGACGIVPAVLAYYDHFIESVSPDIYTRYFMAAGAIGALYKMNASISGAEVGCQGEVGVACSMAAAGLAELLGGSPEQVCVAAEIGMEHNLGLTCDPVAGQVQVPCIERNAIASVKAINAARMALRRTSAPRVSLDKVIETMYETGKDMNAKYRETSRGGLAIKVQCD

SEQ ID NO: 102 - DNA, 1350 bp, Corynebacterium glutamicum
atggctatcagtgttgttgatctatttagcatcggtatcggaccatcatcctcacataccgtcggccccatgagagccgccctcacgtatatctctgaatttcccagctcacatgtcgatatcacgttgcacggatcccttgccgccaccggtaaaggccactgcactgaccgggcggtattactgggtctggtgggatgggaaccaacgatagttcccattgatgctgcaccctcacccggcgcgccgattcctgcgaaaggttctgtgaacgggccaaagggaacggtgtcgtattccctgacgtttgatcctcatcctcttccagaacaccccaatgccgttacctttaaaggatcaaccacaaggacttatttgtcggtgggtggtgggttcattatgacgttggaggatttccggaagctggacgatatcggatcaggtgtgtcaaccattcatccagaggcagaggtgccttgtccttttcagaagagttcccaattactcgcatatgggcgcgattttgcggaggtcatgaaggataatgagcgcttaatccacggggatcttggcacagtggatgcccatttggatcgagtgtggcagattatgcaggagtgcgtggcacaaggcatcgcaacgcctgggattttaccgggtgggttgaatgtgcaacgtcgggcgccgcaggtacacgcgctgattagcaacggggatacgtgtgagctgggtgctgatcttgatgctgtggagtgggtgaatctgtacgccttggcggtgaatgaagaaaacgccgctggtggtcgtgtggttactgctccgactaatggtgctgcggggattattccggcggtgatgcactatgcgcgggattttttgacaggttttggggcggagcaggcgcggacgtttttgtataccgcgggtgcggtgggcatcatcattaaggaaaatgcctcgatctctggcgcggaggtggggtgtcagggtgaggttggttcagcgtccgcgatggcggctgccgggttgtgtgcagtcttaggtggttctccgcaacaggtggaaaacgccgcggagattgcgttggagcacaatttgggattgacgtgcgatccggtgggcgggttagtgcagattccgtgtattgaacgcaacgctattgctgccatgaagtccatcaatgcggcaaggcttgcccggattggtgatggcaacaatcgcgtgagtttggatgatgtggtggtcacgatggctgccaccggtcgggacatgttgaccaaatacaaggaaacgtcccttggtggtttggcaaccaccttgggcttcccggtgtcgatgacggagtgttag

SEQ ID NO: 103 - protein, 449 aa, Corynebacterium glutamicum
MAISVVDLFSIGIGPSSSHTVGPMRAALTYISEFPSSHVDITLHGSLAATGKGHCTDRAVLLGLVGWEPTIVPIDAAPSPGAPIPAKGSVNGPKGTVSYSLTFDPHPLPEHPNAVTFKGSTTRTYLSVGGGFIMTLEDFRKLDDIGSGVSTIHPEAEVPCPFQKSSQLLAYGRDFAEVMKDNERLIHGDLGTVDAHLDRVWQIMQECVAQGIATPGILPGGLNVQRRAPQVHALISNGDTCELGADLDAVEWVNLYALAVNEENAAGGRVVTAPTNGAAGIIPAVMHYARDFLTGFGAEQARTFLYTAGAVGIIIKENASISGAEVGCQGEVGSASAMAAAGLCAVLGGSPQQVENAAEIALEHNLGLTCDPVGGLVQIPCIERNAIAAMKSINAARLARIGDGNNRVSLDDVVVTMAATGRDMLTKYKETSLGGLATTLGFPVSMTEC

SEQ ID NO: 104 - DNA, 1254 bp, Escherichia coli
atgttaaagcgtgaaatgaacattgccgattatgatgccgaactgtggcaggctatggagcaggaaaaagtacgtcaggaagagcacatcgaactgatcgcctccgaaaactacaccagcccgcgcgtaatgcaggcgcagggttctcagctgaccaacaaatatgctgaaggttatccgggcaaacgctactacggcggttgcgagtatgttgatatcgttgaacaactggcgatcgatcgtgcgaaagaactgttcggcgctgactacgctaacgtccagccgcactccggctcccaggctaactttgcggtctacaccgcgctgctggaaccaggtgataccgttctgggtatgaacctggcgcatggcggtcacctgactcacggttctccggttaacttctccggtaaactgtacaacatcgttccttacggtatcgatgctaccggtcatatcgactacgccgatctggaaaaacaagccaaagaacacaagccgaaaatgattatcggtggtttctctgcatattccggcgtggtggactgggcgaaaatgcgtgaaatcgctgacagcatcggtgcttacctgttcgttgatatggcgcacgttgcgggcctggttgctgctggcgtctacccgaacccggttcctcatgctcacgttgttactaccaccactcacaaaaccctggcgggtccgcgcggcggcctgatcctggcgaaaggtggtagcgaagagctgtacaaaaaactgaactctgccgttttccctggtggtcagggcggtccgttgatgcacgtaatcgccggtaaagcggttgctctgaaagaagcgatggagcctgagttcaaaacttaccagcagcaggtcgctaaaaacgctaaagcgatggtagaagtgttcctcgagcgcggctacaaagtggtttccggcggcactgataaccacctgttcctggttgatctggttgataaaaacctgaccggtaaagaagcagacgccgctctgggccgtgctaacatcaccgtcaacaaaaacagcgtaccgaacgatccgaagagcccgtttgtgacctccggtattcgtgtaggtactccggcgattacccgtcgcggctttaaagaagccgaagcgaaagaactggctggctggatgtgtgacgtgctggacagcatcaatgatgaagccgttatcgagcgcatcaaaggtaaagttctcgacatctgcgcacgttacccggtttacgcataa

SEQ ID NO: 105 - protein, 417 aa, Escherichia coli
MLKREMNIADYDAELWQAMEQEKVRQEEHIELIASENYTSPRVMQAQGSQLTNKYAEGYPGKRYYGGCEYVDIVEQLAIDRAKELFGADYANVQPHSGSQANFAVYTALLEPGDTVLGMNLAHGGHLTHGSPVNFSGKLYNIVPYGIDATGHIDYADLEKQAKEHKPKMIIGGFSAYSGVVDWAKMREIADSIGAYLFVDMAHVAGLVAAGVYPNPVPHAHVVTTTTHKTLAGPRGGLILAKGGSEELYKKLNSAVFPGGQGGPLMHVIAGKAVALKEAMEPEFKTYQQQVAKNAKAMVEVFLERGYKVVSGGTDNHLFLVDLVDKNLTGKEADAALGRANITVNKNSVPNDPKSPFVTSGIRVGTPAITRRGFKEAEAKELAGWMCDVLDSINDEAVIERIKGKVLDICARYPVYA

SEQ ID NO: 106 - DNA, 1305 bp, Corynebacterium glutamicum
atgaccgatgcccaccaagcggacgatgtccgttaccagccactgaacgagcttgatcctgaggtggctgctgccatcgctggggaacttgcccgtcaacgcgatacattagagatgatcgcgtctgagaacttcgttccccgttctgttttgcaggcgcagggttctgttcttaccaataagtatgccgagggttaccctggccgccgttactacggtggttgcgaacaagttgacatcattgaggatcttgcacgtgatcgtgcgaaggctctcttcggtgcagagttcgccaatgttcagcctcactctggcgcacaggctaatgctgctgtgctgatgactttggctgagccaggcgacaagatcatgggtctgtctttggctcatggtggtcacttgacccacggaatgaagttgaacttctccggaaagctgtacgaggttgttgcgtacggtgttgatcctgagaccatgcgtgttgatatggatcaggttcgtgagattgctctgaaggagcagccaaaggtaattatcgctggctggtctgcataccctcgccaccttgatttcgaggctttccagtctattgctgcggaagttggcgcgaagctgtgggtcgatatggctcacttcgctggtcttgttgctgctggtttgcacccaagcccagttccttactctgatgttgtttcttccactgtccacaagactttgggtggacctcgttccggcatcattctggctaagcaggagtacgcgaagaagctgaactcttccgtattcccaggtcagcagggtggtcctttgatgcacgcagttgctgcgaaggctacttctttgaagattgctggcactgagcagttccgtgaccgtcaggctcgcacgttggagggtgctcgcattcttgctgagcgtctgactgcttctgatgcgaaggccgctggcgtggatgtcttgaccggtggcactgatgtgcacttggttttggctgatctgcgtaactcccagatggatggccagcaggcggaagatctgctgcacgaggttggtatcactgtgaaccgtaacgcggttcctttcgatcctcgtccaccaatggttacttctggtctgcgtattggtactcctgcgctggctacccgtggtttcgatattcctgcattcactgaggttgcagacatcattggtactgctttggctaatggtaagtccgcagacattgagtctctgcgtggccgtgtagcaaagcttgctgcagattacccactgtatgagggcttggaagactggaccatcgtctaa

SEQ ID NO: 107 - protein, 420 aa, Corynebacterium glutamicum
MTDAHQADDVRYQPLNELDPEVAAAIAGELARQRDTLEMIASENFVPRSVLQAQGSVLTNKYAEGYPGRRYYGGCEQVDIIEDLARDRAKALFGAEFANVQPHSGAQANAAVLMTLAEPGDKIMGLSLAHGGHLTHGMKLNFSGKLYEVVAYGVDPETMRVDMDQVREIALKEQPKVIIAGWSAYPRHLDFEAFQSIAAEVGAKLWVDMAHFAGLVAAGLHPSPVPYSDVVSSTVHKTLGGPRSGIILAKQEYAKKLNSSVFPGQQGGPLMHAVAAKATSLKIAGTEQFRDRQARTLEGARILAERLTASDAKAAGVDVLTGGTDVHLVLADLRNSQMDGQQAEDLLHEVGITVNRNAVPFDPRPPMVTSGLRIGTPALATRGFDIPAFTEVADIIGTALANGKSADIESLRGRVAKLAA

SEQ ID NO: 108 - DNA, 1416 bp, Escherichia coli
atggaaaactttaaacatctccctgaaccgttccgcattcgtgttattgagccagtaaaacgtaccactcgcgcttatcgtgaagaggcaattattaaatccggtatgaacccgttcctgctggatagcgaagatgtttttatcgatttactgaccgacagcggcaccggggcggtgacgcagagcatgcaggctgcgatgatgcgcggcgacgaagcctacagcggcagtcgtagctactatgcgttagccgagtcagtgaaaaatatctttggttatcaatacaccattccgactcaccagggccgtggcgcagagcaaatctatattccggtactgattaaaaaacgcgagcaggaaaaaggcctggatcgcagcaaaatggtggcgttctctaactatttctttgataccacgcagggccatagccagatcaacggctgtaccgtgcgtaacgtctatatcaaagaagccttcgatacgggcgtgcgttacgactttaaaggcaactttgaccttgagggattagaacgcggtattgaagaagttggtccgaataacgtgccgtatatcgttgcaaccatcaccagtaactctgcaggtggtcagccggtttcactggcaaacttaaaagcgatgtacagcatcgcgaagaaatacgatattccggtggtaatggactccgcgcgctttgctgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtacaccgagtgcagaaccctttgcgtggtgcaggaaggcttcccgacatatggcggcctggaaggcggcgcgatggagcgtctggcggtaggtctgtatgacggcatgaatctcgactggctggcttatcgtatcgcgcaggtacagtatctggtcgatggtctggaagagattggcgttgtctgccagcaggcgggcggtcacgcggcattcgttgatgccggtaaactgttgccgcatatcccggcagaccagttcccggcacaggcgctggcctgcgagctgtataaagtcgccggtatccgtgcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaaggattaacctttacgtacgaaccgaaagtattgcgtcacttcaccgcaaaacttaaagaagtttaa

SEQ ID NO: 109 - protein, 471 aa, Escherichia coli
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKAMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 110 - DNA, 3811 bp, Escherichia coli
atggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcgcggcctgggcgatgtgctgattagcttcgaatcggaagtgaacaacatccgtaaacagtatgaagcgcagggctttgaagtggtgattccgaaaaccaacattctggcggaattcccggtggcgtgggttgataaaaacgtgcaggccaacggtacggaaaaagccgccaaagcctatctgaactggctctatagcccgcaggcgcaaaccatcatcaccgactattactaccgcgtgaataacccggaggtgatggacaaactgaaagacaaattcccgcagaccgagctgttccgcgtggaagacaaatttggctcctggccggaagtgatgaaaacccacttcaccagcggcggcgagttagacaagctgttagcggcggggcgtaactgatgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacggaagtgacgtcgctgatgatttttgtgcgcttacaggagtttgattacccggcagcgagcgcgattgcttcggtgatcctcgcggcatctctgctgctgctgttctcaattaacactctgcaaagtcgctttggtcggcgtgtggtaggtcattaatggcggaagttacccaattgaagcgttatgacgcgcgcccgattaactggggcaaatggtttctgattggcatcgggatgctggtttcggcgttcatcctgctggtgccgatgatttacatcttcgtgcaggcattcagcaaggggctgatgccggttttacagaatctggccgatccggacatgctgcacgccatctggctgacggtgatgatcgcgctgattgccgtaccggtaaacctggtgttcggcattctgctggcctggctggtgacgcgctttaacttccctggacgccagttactgctgacgctactggacattccgtttgccgtatcgccggtggttgccggtctggtgtatttgctgttctacggctctaacggcccgctcggcggttggctcgacgagcataacctgcaaattatgttctcctggccgggaatggtgctggtcaccatcttcgtgacgtgtccgtttgtggtgcgcgaactggtgccggtgatgttaagccagggcagccaggaagacgaagcggcgattttgcttggcgcgtccggctggcagatgttccgtcgcgtcacattaccgaacatccgctgggcgctgctttatggcgtggtgttgaccaacgcccgcgcaattggcgagtttggcgcggtgtcggtggtttccggctcgattcgcggcgaaaccctgtcgctgccgttacagattgaattgctggagcaggactacaacaccgtcggctcctttaccgctgcggcgctgttaacgctgatggcgattatcaccctgtttttaaaaagtatgttgcagtggcgcctggagaatcaggaaaaacgcgcacagcaggaggaacatcatgagcattgagattgccaatattaagaagtcgtttggtcgcacccaggtgctgaacgatatctcactggatattccttcaggtcagatggtcgcgttgctggggccgtccggttccgggaaaaccacgctgctgcgcattatcgccgggctggagcatcaaaccagcgggcatattcgcttccacggcaccgacgtgagccgcctgcacgcacgtgatcgtaaagtcggtttcgtgttccagcattacgcgctgttccgccatatgacggtgttcgacaatatcgcttttggcctgacggtgctgccgcgtcgcgagcgcccgaatgccgcagccatcaaagcgaaagtgacaaaattgctggaaatggtccagcttgcccatctggcggatcgttatccggcgcagctttccggcggccagaaacagcgcgtggcgctggcgcgcgcgctggctgtggaaccgcaaattctgctgcttgatgaaccgtttggcgcgctggatgcgcaggtgcgtaaagagctgcgtcgctggctgcgtcaactccatgaagaactaaaattcaccagcgtttttgtgacccacgatcaggaagaagcgaccgaagtagctgatcgtgtagttgtgatgagccagggcaatattgaacaggctgacgcgccggatcaggtatggcgcgaaccggcgacccgttttgtgctcgaatttatgggcgaagtgaaccgcctgcagggaaccattcgcggcgggcagttccatgttggcgcgcatcgctggccgctgggctacacacctgcgtatcaggggccggtggatctcttcctgcgcccttgggaagtggatatcagccgccgtaccagcctcgattcgccgctgccggtacaggtactggaagccagcccgaaaggtcactacacccaattagtggtgcagccgctggggtggtacaacgaaccgctgacggtcgtgatgcatggcgacgatgccccgcagcgtggcgagcgtttattcgttggtctgcaacatgcgcggctgtataacggcgacgagcgtatcgaaacccgcgatgaggaacttgctctcgcacaaagcgcctga

SEQ ID NO: 111 - protein, 338 aa, Escherichia coli
MAVNLLKKNSLALVASLLLAGHVQATELLNSSYDVSRELFAALNPPFEQQWAKDNGGDKLTIKQSHAGSSKQALAILQGLKADVVTYNQVTDVQILHDKGKLIPADWQSRLPNNSSPFYSTMGFLVRKGNPKNIHDWNDLVRSDVKLIFPNPKTSGNARYTYLAAWGAADKADGGDKGKTEQFMTQFLKNVEVFDTGGRGATTTFAERGLGDVLISFESEVNNIRKQYEAQGFEVVIPKTNILAEFPVAWVDKNVQANGTEKAAKAYLNWLYSPQAQTIITDYYYRVNNPEVMDKLKDKFPQTELFRVEDKFGSWPEVMKTHFTSGGELDKLLAAGRN

SEQ ID NO: 112 - protein, 277 aa, Escherichia coli
MFAVSSRRVLPGFTLSLGTSLLFVCLILLLPLSALVMQLAQMSWAQYWEVITNPQVVAAYKVTLLSAFVASIFNGVFGLLMAWILTRYRFPGRTLLDALMDLPFALPTAVAGLTLASLFSVNGFYGEWLAKFDIKVTYTWLGIAVAMAFTSIPFVVRTVQPVLEELGPEYEEAAETLGATRWQSFCKVVLPELSPALVAGVALSFTRSLGEFGAVIFIAGNIAWKTEVTSLMIFVRLQEFDYPAASAIASVILAASLLLLFSINTLQSRFGRRVVGH

SEQ ID NO: 113 - protein, 291 aa, Escherichia coli
MAEVTQLKRYDARPINWGKWFLIGIGMLVSAFILLVPMIYIFVQAFSKGLMPVLQNLADPDMLHAIWLTVMIALIAVPVNLVFGILLAWLVTRFNFPGRQLLLTLLDIPFAVSPVVAGLVYLLFYGSNGPLGGWLDEHNLQIMFSWPGMVLVTIFVTCPFVVRELVPVMLSQGSQEDEAAILLGASGWQMFRRVTLPNIRWALLYGVVLTNARAIGEFGAVSVVSGSIRGETLSLPLQIELLEQDYNTVGSFTAAALLTLMAIITLFLKSMLQWRLENQEKRAQQEEHHEH

SEQ ID NO: 114 - protein, 365 aa, Escherichia coli
MSIEIANIKKSFGRTQVLNDISLDIPSGQMVALLGPSGSGKTTLLRIIAGLEHQTSGHIRFHGTDVSRLHARDRKVGFVFQHYALFRHMTVFDNIAFGLTVLPRRERPNAAAIKAKVTKLLEMVQLAHLADRYPAQLSGGQKQRVALARALAVEPQILLLDEPFGALDAQVRKELRRWLRQLHEELKFTSVFVTHDQEEATEVADRVVVMSQGNIEQADAPDQVWREPATRFVLEFMGEVNRLQGTIRGGQFHVGAHRWPLGYTPAYQGPVDLFLRPWEVDISRRTSLDSPLPVQVLEASPKGHYTQLVVQPLGWYNEPLTVVMHGDDAPQRGERLFVGLQHARLYNGDERIETRDEELALAQSA

SEQ ID NO: 115 - DNA, 1444 bp, Artificial Sequence
  Synthetic Construct
tttgttacggtagtatcacaatcttttaattcaattggcacatcaatttgcacgccagaaacctgaccattggtttgcaacttcgtggcactaacatctggcaaagtcaccacggaatctttcgtcgcagctaccagggtacaagctggagcaataactttaccattaaatgttactgagccatccgtgatagcattatcatcagcagccataacgctggacgttgcacataccacagttataaacgccgtcaatacactttttttcatcgttatcatcctgatctcttcgtaaatatgataatgcctttaaatgacattagacttaataataacaataattgtccgccatttaaatcaagcatactaaatccaataagtgacaaatctttaatttatagatttcatgaatatttaaatattaactgaacgagtaattcattgattcaacatctaatgactatccaataaataaatatgcgtgatatataaagccctttctttagtttattttcagaaaaagatacacaaaatttatatttggaattttcttgtctctccgacagactgaccgaattgttatcaatgttaacaaaaaaagaacaattggttataaggagagagtattccccgaatggcggcgctattgccgccattcggttatttatcaagccagtgctttagttatcttctcgtacagatcgccagagagattttccagccctttcaactgttccagcgccgcgcgcattttctcctgacgtttggcatcgtaacgtttcaggcgaatcagcggttcaatcagacgtgaagccacctgcgggttacggctgttgaggtcggtaagcatttccaccaggaacaggtaaccgctgccatcttcggcatggaacgctgccggattgctgcccgcaaacgcgccaatcaacgaacgaatacggttcgggttgctcatggtaaatgagcgatgctgcaacaggccgcgcaccgtctccagcacattcgccgccgggctggtggcttgcaggataaaccatttatccatcaccagaccgttctgatgccacttgtcgtcgtactcctgcatcagcgcgtcacggcaaggcagctgtgcggcaaccgccgcagaaagcgccgccagcgcatcagtcatattgtttgcttcgtggaactgcttgctcaccagcacatcagccagatgcgtttcaccaaaagcgaggaaacgcaggcaggcattacgcagagtgcgttttgcaatatcttcatgctcaacacggtactcgctctggtaattcgcgttgtaaatagccagtagctcatccgccagttcagtcgccagagtacgagtgagtgcttcgcgtacttcggcaatagcaatcgggtcgatgatatcgaacaattcagccatttcattgacagaaggcagcgtcaggatttctgccgcc

SEQ ID NO: 116 - DNA, 854 bp, Artificial Sequence
  Synthetic Construct
aagtgtttgtatgtttggtatgtatagttattttgttttatacattgtatttagatgtgatttagtttgtcaattaattgcatttaaaaaatatgttctgtgaacaagcattgtttatatacattatgtgaatgtaatatgcgagtgattgagaatgatacagtgatatatacaatgcgaatataatagttttattatatgtattgatattgatagaaataatgaagtaataaatctcgtaatgtggttgtttatgcatcactaaaatgaagtgtagtaattttcccaattgttagaacggagtaattgcatatttaatctttccttagccgtttttttgctaagaataaaatcatctgtgcgataacgactaattcttttaatgaatgtttttattcctgaatactgctcccataacaagacaggggagcagacaatcatggcaatttcatcgcgtaacaccggtcccctcgcccccttggggagagggttagggtgagggggcgttcaccgtactttcaacaggttaactccccctttctgagaggaaacaaaattaacgcagaatcttcttctcagccaaatccagcgcaaagtagctgaaaatcagatccgcacccgcacgcttaatcgaacctaagctttcgagcacgactttctcttcatctatagcacccgccagcgcggcgaacttaatcatcgcatactcaccgctcacctgatacgcgccaatcggcaattcagtacgttcacgcagctcacgcacgatgtcgaggtacgctccagcaggtttaaccatcaggcagtctgcgccctgggcttcatccagcagtgattcacgaatcgcctcacga

SEQ ID NO: 117 - DNA, 1636 bp, Artificial Sequence
  Synthetic Construct
ctcagcgcctaatttcccttcctcttcgccgctggtcgccacaaatcgaataccatactcggtaggcgtatttttcaggcgttctgccaattccagcatgacacctaaacctgcggcgttatcatccattccttgtaacgtcagcccgccgagattggcatcggcgtcagcatcgctcagcggggcgtaagtatccagatgcgccataatgatgatctgctgcggcgctttgccttcatgagcggcaatcaccgtacttcccgtcacgttatgccagctcttacgattatcgcgggcggtataaatataccgactattaaatgtccgaatatcactgcgataacccatttgctgaaactgttggcgaatataatcggcagataacatttctgcaggagttccggtcatgcgtcccggaaagaaagtagcaatatgtcgtgcctgagtattagcaaaatcgccaggtttaggtgacgaggcgtgtacggggagaataaagcatacgccgagcgccagggcagcggtacggtggcgcaatgcggaaaacatagtgagtccttaaataccatgcaaatttttttaccgccatagtatgaaactgccgctgcgctaaaacaatttcaaatcttcctaaacgcccgaaatccggtgccttgacaattaatcatccggctcgtataatgtgtggaattgtgagcggataacaatttcacacaggaaacagaccatggatcaaatacgacttactcacctgcggcaactggaggcggaaagcatccacattattcgcgaggtggcggcagaattctcaaatccggtgatgctctactctatcggtaaagattccagcgtcatgctgcatctggcgcgcaaggcgttttatccaggtacgctgcctttcccgttgctgcatgtcgataccggctggaaattccgcgagatgtatgagttccgcgatcgtactgctaaagcctacggctgcgaactgctggtgcataaaaacccggaaggcgtggcgatggggattaatccattcgtgcacggcagcgcgaaacataccgatattatgaaaactgaaggcctgaaacaggcgctgaacaaatacggttttgatgccgccttcggtggtgcgcgccgtgacgaagagaaatcccgcgctaaagagcgaatttactctttccgtgaccgcttccatcgctgggatccgaaaaatcagcgcccggagctgtggcacaactacaacgggcaaattaacaaaggcgaaagcatccgcgtcttcccgctctctaactggaccgagcaggatatctggcaatacatctggctggaaaatatcgacattgttccgctatatctcgctgcggaacgtccggttctggaacgcgacggtatgttgatgatgattgatgacaaccgtatcgacctgcaaccgggcgaagtgattaaaaaacggatggtgcgtttccgtacgctgggctgctggccgctgaccggtgcggtggagtcaaatgcacaaacactgccggaaatcatcgaagagatgctggtttccaccaccagtgaacgtcagggccgcgtgattgaccgcgaccaggcggggtctatggagctgaaaaaacgtcaggggtatttttaa

SEQ ID NO: 118 - DNA, 1325 bp, Artificial Sequence
  Synthetic Construct
attagacctcagcgacccggcgtcggtagccgcagctatcaaacgcgcgaaggaaaaagaagggcgcattgatatcctggtgaataacgcaggcgtttgtcgtctgggcagtttcctcgatatgagcgatgacgatcgcgatttccatattgacatcaatattaaaggcgtatggaacgtcacgaaggcggtgctgccggagatgattgcccgcaaagatggtcgcattgtgatgatgtcttcagtcactggtgatatggtggccgatcctggcgaaacggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggttagcgtcggtatctgattgacaattaatcatccggctcgtataatgtgtggaattgtgagcggataacaatttcacacaggaaacagaccatggccgttaacttactgaaaaagaactcactcgcgctggtcgcttctctgctgctggcgggccatgtacaggcaacggaactgctgaacagttcttatgacgtctcccgcgagctgtttgccgccctgaatccgccgtttgagcaacaatgggcaaaagataacggcggcgacaaactgacgataaaacaatctcatgccgggtcatcaaaacaggcgctggcgattttacagggcttaaaagccgacgttgtcacttataaccaggtgaccgacgtacaaatcctgcacgataaaggcaagctgatcccggccgactggcagtcgcgcctgccgaataatagctcgccgttctactccaccatgggcttcctggtgcgtaagggtaacccgaagaatatccacgattggaacgacctggtgcgctccgacgtgaagctgattttcccgaacccgaaaacgtcgggtaacgcgcgttatacctatctggcggcatggggcgcagcggataaagctgacggtggtgacaaaggcaaaaccgaacagtttatgacccagttcctgaaaaacgttgaagtgttcgatactggcggtcgtggcgcgaccaccacttttgccgagcggcgatcgcatattat

SEQ ID NO: 119 - DNA, 2000 bp, Artificial Sequence
  Synthetic Construct
tggtgacctgaaatatggccgcaccgttcactccctgactcaggcgttagcgaagttcgacggcaaccgtttttacttcatcgcgccggacgcgctggcaatgccgcaatacattctggatatgctcgatgaaaaagggatcgcatggagtctgcacagctctattgaagaagtgatggcggaagtagacatcctgtacatgacccgcgtgcaaaaagagcgtctggacccgtccgagtacgccaacgtgaaagcgcagtttgttcttcgcgccagcgatctccacaacgccaaagccaatatgaaagtgctgcatccgctgccgcgtgttgatgagattgcgacggatgttgataaaacgccacacgcctggtacttccagcaggcaggcaacgggattttcgctcgccaggcgttactggcactggttctgaatcgcgatctggtactgtaaggggaaatagagatgacacacgataataaattgcaggttgaagctattaaacgcggcacggtaattgaccatatccccgcccagatcggttttaagctgttgagtctgttcaagctgaccgaaacggatcagcgcatcaccattggtctgaacctgccttctggcgagatgggccgcaaagatctgatcaaaatcgaaaatacctttttgagtgaagatcaagtagatcaactggcattgtatgcgccgcaagccacggttaaccgtatcgacaactatgaagtggtgggtaaatcgcgcccaagtctgccggagcgcatcgacaatgtgctggtctgcccgaacagcaactgtatcagccatgccgaaccggtttcatccagctttgccgtgcgaaaacgcgccaatgatatcgcgctcaaatgcaaatactgtgaaaaagagttttcccataatgtggtgctggccaattaattgcggttggtaataaaagtctggctccctataatgagccagactttttaccgctgtaataaaggagaaatctcttgatggaaatccgggctatcatgcccggattaagtctgatgacaaacgcaaaatcgcctgatgcgctacgcttatcaggcctacgtgattcctgcaatttattgaatttgttggccggataaggcatttacgccgcatccggcatgaacaaaactcactttgtctacaatctgaatcggggctatcgtgcccagtttattctttattgccagccgtaacgacggctatagaaccctttcaccaactgggttaatgtcatataccctgccagaatcgcaaccagccacgggaaatagcttaacggcagcgcctgtaattgcagataactggccagcggtgaaaacggcaatgcgatcccgacaatcatcacgatcacggtcatgatcattaacggccacgatgcacagctctgaataaacggcacacggcgggtgcggatcatatgcacaatcagcgtttgcgacagtaagcccaccacaaaccatcccgactggaacagcgtttgcgtttccggcgtgttggcatggaatacccaccacatcaggcaaaacgtcaaaatatcgaagatcgagctgatcggtccgaagaagatcataaagcgccccagatccgccggattccaacgctgcggcttttgaatttgctcgtcgtcgacgttatcaaacgggatcgccacctgtgacacatcgtacagcaggttctgaataagcaagtgtaacggcaacatcggcaggaagggcaagaaagcactcgctaccagcacgctgaacacattaccgaagttagagctcgccgtcattttgatgtatttcagcatgttggcgaaagtgcgacgtccctcaataaccccctcttccagcaccatcaggcttttttccagcaggatgatatcagccgcttcacgggcaatatctaccgcgccgtccacagaaatgccgatatccgcagcgcgtaaggcgggcgcatcattaataccatcgcccataaagccaaccac

SEQ ID NO: 120 - DNA, 2000 bp, Artificial Sequence
  Synthetic Construct
tttggtgcacgcgctaacctggcgaaaacgctgctctacgcaattaacggcggggtggacgagaagctgaagattcaggtcgggccgaaaacagcaccgctgatggacgacgtgctggattacgacaaagtgatggacagcctcgatcacttcatggactggctggcggtgcagtacatcagcgcgctgaatatcattcactacatgcacgacaagtacagctacgaagcttcgctgatggcgctgcacgatcgtgatgtctatcgcactatggcatgcggcatcgcgggcctgtcggtggcgacggactccctgtctgccatcaaatatgcccgcgtgaaaccaatccgtgacgaaaacggcctggcggtggactttgaaatcgacggtgaatatccgcagtacggcaacaacgacgagcgcgtagacagcattgcctgcgacctggttgaacgctttatgaagaaaattaaagcgctgccaacctatcgcaacgccgtccctacccagtcgattctgactatcacttctaacgtggtgtacggccagaaaaccggtaatacgccggacggtcgtcgcgccggaacaccgttcgcgccgggcgctaacccgatgcatggtcgtgaccgcaaaggtgccgtggcctcattgacgtcggtggcgaaactgccgttcacctacgccaaagatgggatctcgtacaccttctcaatcgttcctgcggcgctgggcaaagaagatccagtacgtaaaaccaaccttgtcggcctgctggatgggtatttccaccacgaagcggatgtcgaaggcggtcaacacctcaacgtcaacgtaatgaatcgggaaatgctgctggatgccatcgagcacccggaaaaatatcctaacctgacaatccgtgtctctggctacgccgtgcgcttcaacgcactgacccgtgaacagcaacaggatgttatttcacgtacctttacccaggcgctctgacgccggaggatgtagcctcgtgagcgggacggtcgtaaggtcgttccgctccacttcactgaacggcaatccgagggtgtggatatgattagtgcattcgatattttcaaaattgggattggtccctccagttcgcataccgtggggccaatgaatgccggaaaaagttttattgatcggctggaaagtagcggcttattaaccgcgacgagccatattgtggtcgatctgtacgggtcgttgtcactgacgggcaaaggccatgccacggatgtcgccatcatcatgggactggcaggaaacagtccgcaggatgttgtcattgatgagatccctgcatttatagagttagtaacgcgcagcgggcggctgccagtggcatctggtgcgcatattgttgattttcctgtagcaaagaacattatcttccatcccgaaatgttgcctcgccatgagaacggaatgcggatcactgcctggaagggacaggaagagctattaagtaaaacctattactctgtcggcggcgggtttattgtcgaagaagaacacttcggcctgtcgcacgatgtcgaaacgtccgtaccttacgatttccactcagcaggtgaactgctgaaaatgtgtgattacaacggcctgtctatatctggtctgatgatgcacaacgagctagcgctgcgcagcaaagcggaaattgacgccggttttgcccgtatctggcaagtgatgcatgacggtattgaacgtgggatgaacactgaaggcgtgctgcctggtccgctcaatgtgccgcgccgtgccgtagcgctgcgtcgtcagctggtttccagcgataacatctctaacgatccgatgaatgtcatcgactggatcaacatgtacgcgctggcggttagtgaagaaaacgcagctggcgggcgcgtggtaacggcaccgactaacggtgcgtgcggcattattccggcagtactggcttattacgataagttccgtcgtccggtaaacgagcg

SEQ ID NO: 121 - DNA, 1000 bp, Artificial Sequence
  Synthetic Construct
tggtttcaaaatggctgggatgaacagtatgttgaggctggcggccccggctcaccgaattatcataaatcgaacgccctgaaaaccatgcgtaatcagccgctgctgcaggggaaattgctgacgaaaggctcctgggattatcaactggtggatgaactgatgccgcagcctggcgatattgtgctgccgaagccgcgctacagcggtttcttcaatacgccgctggacagcattttgcgcagccgtggaatacgccatctggttttcaccggtatcgctaccaacgtctgcgtcgaatcgacgctacgcgacggcttttttctggagtatttcggcgtggtgctggaagacgcaacacaccaggcggggccggaatttgcacagaaagccgcgttgttcaatatcgaaaccttttttggctgggtcagcgacgtcgaaacgttctgcgacgcgctttcttccacgtcctttgctcgtatcgcttaaggagtttaacgggccgcgatgaaactttcactctcacctcccccttatgctgatgcgcccgtagtggtgttgatttcgggtcttggcggtagcggcagttactggttaccgcaactggcggtgctggatcaggagtatcaggtagtctgttacgaccagcgcggcaccggcaataatcccgacacgctggcagaagattacagtatcgcccagatggcagcggaactgcatcaggcgctggtagccgcagggattgagcgttacgcggtggtcggccatgcgctcggtgcgctggtgggaatgcagctggcgctggattatcccgcgtcggtaactgtgctggtcagcgttaacggctggctacgaataaacgcccatacgcgccgctgttttcaggttcgcgaacagttactacatagcggcggcgcgcaggcatgggtggaagcgctgccgttgttcctctatcccgccgactggatggcggcccgcgcacctcgccttgaggcagaag

SEQ ID NO: 122 - DNA, 29 bp, Artificial Sequence
  Synthetic Construct
cgcggatccgcataattttgtcagtggtg

SEQ ID NO: 123 - DNA, 40 bp, Artificial Sequence
  Synthetic Construct
acctgattcttctgatcgccctggcgcgcatccgcacgcg

SEQ ID NO: 124 - DNA, 41 bp, Artificial Sequence
  Synthetic Construct
cgcgtgcggatgcgcgccagggcgatcagaagaatcaggtg

SEQ ID NO: 125 - DNA, 30 bp, Artificial Sequence
  Synthetic Construct
cccaagctttagaacagcgcgctggccatc

SEQ ID NO: 126 - DNA, 29 bp, Artificial Sequence
  Synthetic Construct
cgcggatcccaacgacggcggagacatgc

SEQ ID NO: 127 - DNA, 43 bp, Artificial Sequence
  Synthetic Construct
ccatcggaaccacaccaacgccggccggcgcggtcgaaaagtg

SEQ ID NO: 128 - DNA, 43 bp, Artificial Sequence
  Synthetic Construct
cacttttcgaccgcgccggccggcgttggtgtggttccgatgg

SEQ ID NO: 129 - DNA, 30 bp, Artificial Sequence
  Synthetic Construct
cccaagcttggccggcgcggtcgaaaagtg

SEQ ID NO: 130 - DNA, 30 bp, Artificial Sequence
  Synthetic Construct
cgcggatccccttcccatcgccttaaaccc

SEQ ID NO: 131 - DNA, 41 bp, Artificial Sequence
  Synthetic Construct
cgaggtcttggggtttgtcgcctgggaagcgggtctcctgg

SEQ ID NO: 132 - DNA, 41 bp, Artificial Sequence
  Synthetic Construct
ccaggagacccgcttcccaggcgacaaaccccaagacctcg

SEQ ID NO: 133 - DNA, 31 bp, Artificial Sequence
  Synthetic Construct
cccaagcttgcctagagacaccgcatcgctg

SEQ ID NO: 134 - DNA, 1365 bp, Artificial Sequence
  Synthetic Construct
attagacctcagcgacccggcgtcggtagccgcagctatcaaacgcgcgaaggaaaaagaagggcgcattgatatcctggtgaataacgcaggcgtttgtcgtctgggcagtttcctcgatatgagcgatgacgatcgcgatttccatattgacatcaatattaaaggcgtatggaacgtcacgaaggcggtgctgccggagatgattgcccgcaaagatggtcgcattgtgatgatgtcttcagtcactggtgatatggtggccgatcctggcgaaacggcgtacgccttaacgaaagcggcgattgttggcctgacaaaatcgctggcggtggagtacgcgcagtctggtattcgcgttaacgccatttgcccgggatacgtgcgcacaccaatggcggaaagcattgcccgccagtcgaacccggaagatccagagtcggtgctgactgaaatggcgaaagcaatcccgatgcgtcgcctcgccgatccgctggaagtcggcgaactggcggccttcctcgcatcggatgaatccagctatttaaccggtacacagaatgtgattgatggcggcagcacactgccggagacggttagcgtcggtatctgattgacaattaatcatccggctcgtataatgtgtggaattgtgagcggataacaatttcacacaggaaacagaccatgtttgctgtctcctccagacgcgtgctgccgggctttaccttaagcctcggcaccagtctgctgtttgtgtgcctgattttgctgctgccgctctccgcgctggtgatgcaactggcccagatgagctgggcgcagtactgggaggtgatcaccaacccgcaggtggtcgcggcctacaaagtaacgctgctgtcggcgtttgtggcatcgatttttaacggcgttttcggtctgctgatggcgtggatcctaacccgctatcgcttcccaggccgcacgctgcttgatgcgctgatggatttaccctttgcgctgccaacggctgtcgccggtttaacgctggcctcgctcttttccgtaaacggtttttacggtgaatggctggcgaagtttgatatcaaagtcacctatacatggctggggattgcggtggctatggctttaccagcattccgtttgtggtgcgtaccgtgcagccggtgctggaagagttaggcccggaatatgaagaagcggcggaaacgcttggtgcaacgcgctggcagagtttctgcaaagtggtgctgccggagctttctccggcgctggtggcgggcgtggcgctgtcgtttacccgtagtcttggtgaatttggcgcggtgatttttatcgccggaaatatcgcgtggaagacgg

SEQ ID NO: 135 - DNA, 1100 bp, Artificial Sequence
  Synthetic construct
tcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaataggaaaaatgcctgatagcgcttcgcttatcaggcctacccgcgcgacaacgtcatttgtggttcggcaaaatcttccagaatgcctcaattagcggctcatgtagccgctttttctgcgcacacacgcccagctcaaacggcgttttctcatcgctgcgctctaaaatcatcacgcggttacgcaccggttcggggctgttttccagcaccacttccggcaacaatgccacgccacagccgagtgccaccatcgataccatcgcttcatgcccgccaaccgtggcgtaaatcatcgggttactgattttattgcgtcgaaaccacagttcaatgcggcggcgtaccggcccctgatcggccataataaacggcaccgttgaccagtccggcttctctaccgacacctgattacgcaccgggcagggcagcgcgggggcaatcagcactactgccagattctccagcatcgaaaacgccactgcgccgggcaaggtttccggtttacccgcaatcgccagatccgcttcaccagtgaccaccttttccatc

SEQ ID NO: 136 - DNA, 1545 bp, Escherichia coli
atggctgactcgcaacccctgtccggtgctccggaaggtgccgaatatttaagagcagtgctgcgcgcgccggtttacgaggcggcgcaggttacgccgctacaaaaaatggaaaaactgtcgtcgcgtcttgataacgtcattctggtgaagcgcgaagatcgccagccagtgcacagctttaagctgcgcggcgcatacgccatgatggcgggcctgacggaagaacagaaagcgcacggcgtgatcactgcttctgcgggtaaccacgcgcagggcgtcgcgttttcttctgcgcggttaggcgtgaaggccctgatcgttatgccaaccgccaccgccgacatcaaagtcgacgcggtgcgcggcttcggcggcgaagtgctgctccacggcgcgaactttgatgaagcgaaagccaaagcgatcgaactgtcacagcagcaggggttcacctgggtgccgccgttcgaccatccgatggtgattgccgggcaaggcacgctggcgctggaactgctccagcaggacgcccatctcgaccgcgtatttgtgccagtcggcggcggcggtctggctgctggcgtggcggtgctgatcaaacaactgatgccgcaaatcaaagtgatcgccgtagaagcggaagactccgcctgcctgaaagcagcgctggatgcgggtcatccggttgatctgccgcgcgtagggctatttgctgaaggcgtagcggtaaaacgcatcggtgacgaaaccttccgtttatgccaggagtatctcgacgacatcatcaccgtcgatagcgatgcgatctgtgcggcgatgaaggatttattcgaagatgtgcgcgcggtggcggaaccctctggcgcgctggcgctggcgggaatgaaaaaatatatcgccctgcacaacattcgcggcgaacggctggcgcatattctttccggtgccaacgtgaacttccacggcctgcgctacgtctcagaacgctgcgaactgggcgaacagcgtgaagcgttgttggcggtgaccattccggaagaaaaaggcagcttcctcaaattctgccaactgcttggcgggcgttcggtcaccgagttcaactaccgttttgccgatgccaaaaacgcctgcatctttgtcggtgtgcgcctgagccgcggcctcgaagagcgcaaagaaattttgcagatgctcaacgacggcggctacagcgtggttgatctctccgacgacgaaatggcgaagctacacgtgcgctatatggtcggcggacgtccatcgcatccgttgcaggaacgcctctacagcttcgaattcccggaatcaccgggcgcgctgctgcgcttcctcaacacgctgggtacgtactggaacatttctttgttccactatcgcagccatggcaccgactacgggcgcgtactggcggcgttcgaacttggcgaccatgaaccggatttcgaaacccggctgaatgagctgggctacgattgccacgacgaaaccaataacccggcgttcaggttctttttggcgggttag

SEQ ID NO: 137 - protein, 514 aa, Escherichia coli [sequence omitted]

SEQ ID NO: 138 - DNA, 1000 bp, Artificial Sequence
  Synthetic Construct
aaggtttcggtggctgcagtaagttgctgtcgggccgctacctggaggtgaatcagacgggacagcggaaggtagacttctgccacttcagcgaggtcaatgttttctccgatgcctcgaagttcaatgacttctttttgggtcagcacctgaggcattgagtttctcagctcgcgccattgtgcgcggtcgaaatcaaggtaggggctgaaatctggtgtgcgtggggaaggtttcacaccagttgtgcttgcagcgttttgctctgccatgaatccattgtgcaccttagctactccactagtgtgatcggggttattttttcacttcaatgggtggctaaaagacgtgggcacgtgagtaaactcatgcgcgcgaaacgatgggagtgaacccatacttttatatatgggtatcggcggtctatgcttgtgggcgtacctgtcccgcgagtgaggtcttacgcgcgggattcgtcttgtgaaaggttagctgacctggtttttctttgagttttcatatgtagaaggcatcgtcggcttcggcctggcggtgcttttctcgttgttttgtggttttgtcagaggatgtcatgcgcgttttaattattgataattatgattctttcacgtttaatctcgccacctatgtggaagaggttacgggtcaggcacctgtggtggtgcctaatgatcaagaaatagatgagatgcttttcgacgccgtcatcctctcacctggcccgggccacgccggcgttgcggctgattttggtatctgtgcaggcgtcattgagcgtgcacgcgttccgattttgggtgtgtgtttaggccaccagggcattgcgttggcctatggcggtgatgttgatttggcgcccaggccggtccacggtgaggtttcgcagatcacccatgatggttcaggtttatttgcaggcatccctgaaacgtttgaggcggtgcgttatcactcgatggtggcaacccgcttgccggag

SEQ ID NO: 139 - DNA, 1000 bp, Artificial Sequence
  Synthetic Construct
cgctgatttcatcgtcatcatcgctggtgtctttgccggaggagctcaagcttggggccgcaggaatgtcgccattgctgagcattgagctgccttcagagctgcctggccaggtttcgtttccatcgactggatttccatcatcatcaaggatctgtgatgaggtgatgttgtctgagagctgtgtcagtgcgtcagaggactgagcctgggcaactggagtgaacacggacaatgccacagcgcttgctgtaacaagggtcaaagtacttcgacgcaaagacaaaacttttctcctggcaataaatatgcggatttactatggaaacaagatagaagattggatagcgaaagctatcctcaactcgtggaaagtgtagtgccacaaccacagtattggctagaaaacaatctatagcattgttctacaaagagcttgttggaaataaaacctatgccaaagtaggtgcaattctaggagaagattacactagtcaaccacatagctgaaggccacctcaatcgaggtggcctttttctagtttcgggtcaggatcgcaaagccccacggctgaagggttgtggaggtgtcggtgacggtgggggaagtgaagctgtaaatcagctcgccgccaagcgggacggtgatggtgtcgtcggagaaattcgccagaattcggccgcgaccattggccatcgatagccagttctcgccgtgctcaacctcgagtgtgagcaagtttggttgggagaagcccaaggtgtgccgcaggtgcaacagctgcttgtaagcgtcgttgatgcggcgctgctccgcagtgaactcccaatcgagtttggaggaggtgaaggtggattccagctcgggggaggggatgtcgtcggcgttccagccaaggcgtgcgaattcccgtttgcggccctcggaggttaggcggttgagctcggggtcggtgtgggagcaaaagaaggcgaatggggtggtggctccgaattcttc

SEQ ID NO: 140 - DNA, 1311 bp, Corynebacterium glutamicum
atgagtgaaacatacgtgtctgagaaaagtccaggagtgatggctagcggagcggagctgattcgtgccgccgacattcaaacggcgcaggcacgaatttcctccgtcattgcaccaactccattgcagtattgccctcgtctttctgaggaaaccggagcggaaatctaccttaagcgtgaggatctgcaggatgttcgttcctacaagatccgcggtgcgctgaactctggagcgcagctcacccaagagcagcgcgatgcaggtatcgttgccgcatctgcaggtaaccatgcccagggcgtggcctatgtgtgcaagtccttgggcgttcagggacgcatctatgttcctgtgcagactccaaagcaaaagcgtgaccgcatcatggttcacggcggagagtttgtctccttggtggtcactggcaataacttcgacgaagcatcggctgcagcgcatgaagatgcagagcgcaccggcgcaacgctgatcgagcctttcgatgctcgcaacaccgtcatcggtcagggcaccgtggctgctgagatcttgtcgcagctgacttccatgggcaagagtgcagatcacgtgatggttccagtcggcggtggcggacttcttgcaggtgtggtcagctacatggctgatatggcacctcgcactgcgatcgttggtatcgaaccagcgggagcagcatccatgcaggctgcattgcacaatggtggaccaatcactttggagactgttgatccctttgtggacggcgcagcagtcaaacgtgtcggagatctcaactacaccatcgtggagaagaaccagggtcgcgtgcacatgatgagcgcgaccgagggcgctgtgtgtactgagatgctcgatctttaccaaaacgaaggcatcatcgcggagcctgctggcgcgctgtctatcgctgggttgaaggaaatgtcctttgcacctggttctgtcgtggtgtgcatcatctctggtggcaacaacgatgtgctgcgttatgcggaaatcgctgagcgctccttggtgcaccgcggtttgaagcactacttcttggtgaacttcccgcaaaagcctggtcagttgcgtcacttcctggaagatatcctgggaccggatgatgacatcacgctgtttgagtacctcaagcgcaacaaccgtgagaccggtactgcgttggtgggtattcacttgagtgaagcatcaggattggattctttgctggaacgtatggaggaatcggcaattgattcccgtcgcctcgagccgggcacccctgagtacgaatacttgacctaa

SEQ ID NO: 141 - protein, 436 aa, Corynebacterium glutamicum
MSETYVSEKSPGVMASGAELIRAADIQTAQARISSVIAPTPLQYCPRLSEETGAEIYLKREDLQDVRSYKIRGALNSGAQLTQEQRDAGIVAASAGNHAQGVAYVCKSLGVQGRIYVPVQTPKQKRDRIMVHGGEFVSLVVTGNNFDEASAAAHEDAERTGATLIEPFDARNTVIGQGTVAAEILSQLTSMGKSADHVMVPVGGGGLLAGVVSYMADMAPRTAIVGIEPAGAASMQAALHNGGPITLETVDPFVDGAAVKRVGDLNYTIVEKNQGRVHMMSATEGAVCTEMLDLYQNEGIIAEPAGALSIAGLKEMSFAPGSVVVCIISGGNNDVLRYAEIAERSLVHRGLKHYFLVNFPQKPGQLRHFLEDILGPDDDITLFEYLKRNNRETGTALVGIHLSEASGLDSLLERMEESAIDSRRLEPGTPEYEYLT

SEQ ID NO: 142 - DNA, 1000 bp, Artificial Sequence
  Synthetic Construct
tggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaactttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacccaatagagtgcgattgctgtggggaatggtcctgtgaggccaagggacagtgggaagatcggcgcgagg

SEQ ID NO: 143 - DNA, 246 bp, Escherichia coli
atgcgcattactatttacactcgtaacgattgcgttcagtgccacgccaccaaacgggcgatggaaaaccggggctttgattttgaaatgattaatgtcgatcgcgttcctgaagcggcagaagcgttgcgtgctcagggctttcgtcagttgccggtagtgattgctggcgatcttagctggtctggtttccgtccggacatgattaaccgtctgcatccagcgccacacgcggccagtgcatga

SEQ ID NO: 144 - protein, 81 aa, Escherichia coli
MRITIYTRNDCVQCHATKRAMENRGFDFEMINVDRVPEAAEALRAQGFRQLPVVIAGDLSWSGFRPDMINRLHPAPHAASA

SEQ ID NO: 145 - DNA, 1000 bp, Artificial Sequence
  Synthetic Construct
ctccagtgtgcaaggccttgggggcgcaaccgcgaaccaagacgctgacctttccttccgtcgcaccactggcgtgaccccgggagagaacttcagcagctttgccgatcttctcaaaggacacgacggaaacggctaaattcgcggatctccgtttaaggcattgaagcatttggaggccccaagacatgacccagaccctgtaaagcgcttaaacggcgttttagagggtcatagttttgggacaagtgggacaagtgtgaatcctgaaagcttccagggcaaggatccaccacaaaccggccatcgccctttggaatcggtccgaaaattgcaggtacagagccttttaccgagaaaatccaccacagattgctgaaatttcgtgatctgtggtggattcgtgcaacttcagactcttacggaggcgatggaccaaaaacaactacaatcaagcagatcaccttgtacaccaccatagaaaaggcccaccctcagcccggtacggctttaacacggcttggattttgtcttgcttggcgaggtaggactggcactgggcgtcgataagctcagctgaccacccggtgacctgcgccattgcttcggccgtcgcacgcacggcagccggttgcacataacccagcgtgccgagcacgatgcgacgatccagcacgtccgccaggtcgacggccgcctcctcggcgacagcaaaaacggcctgcgccgcgatatcaaggttgtctgggtcaagtcggcgccccaggtcgggttgctttgcgacgagatccagcactttttcatgctcagttccatacagtctggccagatgcacgcggatttcatcatccacatccagctcggggtggctgcgaagcgctgactcaaaggaatcagccacggactcatacgcgccaaaagaagtactcaacggcatctgcctggagtcaaagtggcgcagcggaggtaacaccgcagcttggtgctccaccacatgctc

SEQ ID NO: 146 - DNA, 1200 bp, Artificial Sequence
  Synthetic Construct
ctgcgtaaacacgctggacaagtggcattccctggaggtgcagtcgatgacacggacgcatcagctatcgccgccgcgctgcgcgaagctgaagaagaggtcgctataccgccttccgccgttgaagttatcggcgtgctgccgcccgtcgatagcgtcactggctaccaggtaaccccagtggtcggcattatcccgcccgatctgccgtatcgcgccagtgaagatgaagtctcggcggtgtttgaaatgccgctcgcccaggcattacatctgggtcgttatcaccctttagatatctaccgccgtggtgattcacatcgggtatggctgtcctggtacgaacagtattttgtatggggaatgaccgcaggcataattcgtgagctggcgctgcaaattggtgtgaaaccctgactatacttatctttacatctacaaaacactacttgagacaatcatcgcaatattagttaaatcgcggtttttgattagtttaattcatgtgaatagttaagccagtcgccgcgttccctcttacactatgcgctgttattagttcgttactggaagtccagtcaccttgtcaggagtattatctacttcttactcgcccatctgcaacggatgggcgaatttatacccgctttctcgtctgctgtaatattccccactacacttccactgttgcgtcaggcgtttgtcgccatacgcttacagggtggcccgcatgcaaaaagcacaacggatcattaaaacctatcgtcgtaatcgaatgattgtttgtacgatttgcgcactcgttacgctcgcttcgaccctgagcgtgcgatttatttcacagcgtaacttaaatcaacaacgggtagtacaattcgccaatcacgctgtagaggaattagataaagtactgcttcccctacaggcaggtagcgaagtcttgcttccgctgattggtctgccctgctctgtcgcccatttgccattacgtaaacaggcggcaaaactccaaactgtgcgatccattggcctggtgcaagacggcacactttattgctccagcatttttggttatcgcaatgtgcccgtcgtggacattctggctgaacttcctgcaccgcaaccacttttacgcctgacgatcgaccgtgccctgattaaaggcagtccggttttgattcaatggacgcctgcagcggg

SEQ ID NO: 147 - DNA, 936 bp, Corynebacterium glutamicum
atgggcaatgtgtacaacaacatcaccgaaaccatcggccacaccccactggtaaagctgaacaagctcaccgaaggcctcgacgcaactgtcctggtcaagcttgagtcattcaacccagcaaactccgtcaaggaccgtatcggtctggccatcgttgaagatgcagagaagtccggtgcactgaagccaggcggcaccatcgttgaagcaacctccggcaacaccggtatcgcactggcaatggtcggcgctgcacgcggatacaacgttgttctcaccatgccggagaccatgtccaacgagcgtcgcgttctcctccgcgcttacggtgcagagatcgttcttaccccaggtgcagcaggcatgcagggtgcaaaggacaaggcagacgaaatcgtcgctgaacgcgaaaacgcagtccttgctcgccagttcgagaacgaggcaaacccacgcgtccaccgcgacaccaccgcgaaggaaatcctcgaagacaccgacggcaacgttgatatcttcgttgcaagcttcggcaccggcggaaccgtcaccggcgttggccaggtcctgaaggaaaacaacgcagacgtacaggtctacaccgtcgagccagaagcgtccccacttctgaccgctggcaaggctggtccacacaagatccagggcatcggcgcaaacttcatccccgaggtcctggaccgcaaggttctcgacgacgtgctgaccgtctccaacgaagacgcaatcgcattctcccgcaagctcgctaccgaagagggcatcctcggcggtatctccaccggcgcaaacatcaaggcagctcttgaccttgcagcaaagccagagaacgctggcaaaaccatcgtcaccgttgtcaccgacttcggcgagcgctacgtctccaccgttctttacgaagacatccgcgactaa

SEQ ID NO: 148 - protein, 311 aa, Corynebacterium glutamicum
MGNVYNNITETIGHTPLVKLNKLTEGLDATVLVKLESFNPANSVKDRIGLAIVEDAEKSGALKPGGTIVEATSGNTGIALAMVGAARGYNVVLTMPETMSNERRVLLRAYGAEIVLTPGAAGMQGAKDKADEIVAERENAVLARQFENEANPRVHRDTTAKEILEDTDGNVDIFVASFGTGGTVTGVGQVLKENNADVQVYTVEPEASPLLTAGKAGPHKIQGIGANFIPEVLDRKVLDDVLTVSNEDAIAFSRKLATEEGILGGISTGANIKAALDLAAKPENAGKTIVTVVTDFGERYVSTVLYEDIRD

SEQ ID NO: 149 - DNA, 387 bp, Corynebacterium glutamicum
atgcctgtgcagctccctccaggacctgctgaacgcactgtttattcctacggtgttcgcgcagggggtcagttgcacatttcaggaatggtggcttttgattccgaggcgaatattgtcggcgaaggggacatcgaagcgcagactgaacaagtctttaagaacctgcaagcagtagtcgaggaggcaggtggaacgatcaacaacattgtttccaccactacttatcttgccgatgtcaccgatgctcctgtggttaatgccgctcgcgcccgctatttcaccggagatgtattgcctacccacaccgtaattggggttgctgctctggctcgcccacagtttctagtcgagatctcggcggtggcctatttgggggacctttag

SEQ ID NO: 150 - protein, 128 aa, Corynebacterium glutamicum
MPVQLPPGPAERTVYSYGVRAGGQLHISGMVAFDSEANIVGEGDIEAQTEQVFKNLQAVVEEAGGTINNIVSTTTYLADVTDAPVVNAARARYFTGDVLPTHTVIGVAALARPQFLVEISAVAYLGDL

SEQ ID NO: 151 - DNA, 396 bp, Corynebacterium glutamicum
atgcctgttccggcgcttcccttcggggaaacagctatggttggccacatgtcgaatgatcacccatactcccccgcaaaacgcgtcggaaatttcatcttcgtctccggcgctctttcagtagacaaggactaccaaccagtcgtcggtcgtaaagaagcagttgatgcagcacttgaacgcatgcgtgaacgcgtcgccaccgctggtggtgaactcaaagacgttgtgaaactcacttactttgtcaccgacatcagcctgcgcgaagaatgcaacgagcaattccgagagcatttcctcgaaggccgcccggcacgctctttcgtgggtgcatcatcgcttccttatggtgcaactgtggaaattgatgcgattgcgatgatcgaggactaa

SEQ ID NO: 152 - protein, 131 aa, Corynebacterium glutamicum
MPVPALPFGETAMVGHMSNDHPYSPAKRVGNFIFVSGALSVDKDYQPVVGRKEAVDAALERMRERVATAGGELKDVVKLTYFVTDISLREECNEQFREHFLEGRPARSFVGASSLPYGATVEIDAIAMIED

SEQ ID NO: 153 - DNA, 70 bp, Escherichia coli
ctgcgcgcgttacagcgccgcctgacgccctggcatggagaagtacaatgattccggggatccgtcgacc

SEQ ID NO: 154 - DNA, 70 bp, Escherichia coli
agggggcgaggggaccgtccactctcgtattaccccgcccgataaaacggtgtaggctggagctgcttcg

SEQ ID NO: 155 - DNA, 70 bp, Escherichia coli
attcgccagcgcatctggcagcccactcaactggaaggaaaacaattatgattccggggatccgtcgacc

SEQ ID NO: 156 - DNA, 70 bp, Escherichia coli
tcttcactggcgttgccattatttcttccttagctttgcgcgactttacgtgtaggctggagctgcttcg

SEQ ID NO: 157 - DNA, 70 bp, Escherichia coli
gttatcaatgttaacaaaaaaagaacaattggttataaggagagagtatgattccggggatccgtcgacc

SEQ ID NO: 158 - DNA, 70 bp, Escherichia coli
gcaatcccgccagcgccagtttaatgatgttacgcatgggcattacctcgtgtaggctggagctgcttcg

SEQ ID NO: 159 - DNA, 1200 bp, Artificial Sequence
  Synthtic Construct
tttgatctggaagctggtgtgcttctgcgcgattttttctaccgctggcagcagcgcaggcaggttttcaatattactggcgtaggcggcaatagcgttaaacagggcttcacgttgatcgccattacgctggttactcatgttaaaaatttctttgagttctgggttatgagtaaacatacggtcgtagaaatgggcggttaactttggccccgtttccaccagtaaagggatggtggcttttactgtagcgatggtttgagcgtcaagcatatggtcttcctttttttgcatcttaattgatgtatctcaaatgcatcttataaaaaatagccctgcaatgtaaatggttctttggtgtttttcagaaagaatgtgatgaagtgaaaaatttgcatcacaaacctgaaaagaaatccgtttccggttgcaagctctttattctccaaagccttgcgtagcctgaaggtaatcgtttgcgtaaattcctttgtcaagacctgttatcgcacaatgattcggttatactgttcgccgttgtccaacaggaccgcctataaaggccaaaaattttattgttagctgagtcaggagatgcgggcgaaacggtgatttgctgtcaatgtgctcgttgttcatgccggatgcggcgtgaacgccttatccggcctacaaaactttgcaaattcaatatattgcaatctccgtgtaggcctgataagcgtagcgcatcaggcaatttttcgtttatgatcatcaaggcttccttcgggaagcctttctacgttatcgcgccatcaaatctgtcgtaactgcgcctcaacatacaaatagccaattcccagcacctgttgtgcgcggcttaattgcccaaagccaatttgcgtcgctttcacgctcggtccttcgtcgcggtcgaacgggttaaatcctgtctggcgaataatagtattaagccagttctcaccaaaggcacaactgcgaccgtacaaccaaatctgattaatattgaggatgttgaggaagttatacagactcaaaccaatggcattggcagagcgatcaacccagctggtgatccacggttctccactttgccaggcagcgattaactgcgctgtagtcagtttttcaggatcaagttgagtactaaccggttgtgattttagccatacccgcgcctgtttttttaatgcgctt

SEQ ID NO: 160 - DNA, 990 bp, Escherichia coli
atgaacaagtggggcgtagggttaacatttttgctggcggcaaccagcgttatggcaaaggatattcagcttcttaacgtttcatatgatccaacgcgcgaattgtacgaacagtacaacaaggcattcagcgcccactggaaacagcaaactggtgataacgtggtgattcgtcagtcacacggtggctcaggtaaacaagcgacgtcggtaatcaacggtattgaagctgatgttgtcacgctggctctggcctatgacgtggacgcaattgcggaacgcgggcggattgataaagagtggatcaaacgtctgccggataactccgcaccgtacacttccaccattgttttcctggtacgtaagggaaatccgaagcagatccatgactggaacgatctgattaaaccgggtgtttcggtgatcacgcctaatccgaaaagctctggtggcgcgcgctggaactacctggcagcctggggctacgcgctgcatcacaacaacaacgatcaggcaaaagcacaggattttgttcgggcactgtataaaaacgtcgaagttctggattctggcgcgcgtggctccactaacacttttgtcgagcgcggaattggcgatgtactgattgcctgggaaaacgaagctctgctggcagcgaatgaactggggaaagataaattcgaaatcgtcacgccgagtgagtctatcctcgcagagccaaccgtgtcggtggtcgataaagtggtcgagaaaaaaggtactaaagaggtggcggaagcctacctgaaatatctctactcgccagaaggtcaggaaattgccgcgaaaaactactaccgtccgcgcgacgctgaggtggcgaaaaagtacgaaaatgcgtttccaaagctgaagttattcaccattgatgaagagttcggcggctggacgaaagcgcaaaaagagcattttgctaacggcggtacgttcgatcagatcagcaaacgctga

SEQ ID NO: 161 - protein, 329 aa, Escherichia coli
MNKWGVGLTFLLAATSVMAKDIQLLNVSYDPTRELYEQYNKAFSAHWKQQTGDNVVIRQSHGGSGKQATSVINGIEADVVTLALAYDVDAIAERGRIDKEWIKRLPDNSAPYTSTIVFLVRKGNPKQIHDWNDLIKPGVSVITPNPKSSGGARWNYLAAWGYALHHNNNDQAKAQDFVRALYKNVEVLDSGARGSTNTFVERGIGDVLIAWENEALLAANELGKDKFEIVTPSESILAEPTVSVVDKVVEKKGTKEVAEAYLKYLYSPEGQEIAAKNYYRPRDAEVAKKYENAFPKLKLFTIDEEFGGWTKAQKEHFANGGTFDQISKR

SEQ ID NO: 162 - DNA, 771 bp, Corynebacterium glutamicum
atgactacaaccttgactcgccccaaaatcgcgctgcccgcgcgcatctattcaccgcttgcggtgcttgttttctggcagctcggctcgagcctgggcgccatcccggagcggattctgccggcaccaaccacgatcttggccgccagctgggaggtcgccacaaatggcacgcttctcgacgccctcctcgtctcaagccaacgcgtccttctaggcttcgccctcggtgctgtcctaggcatttccctaggtgtattgacaggcatgtccagatttgcagacaccgccgttgatccgctcattcaagctgcccgcgcgctgcctcacctgggtcttgtgccgctgtttatcatctggttcggtatcggtgagctgccgaaagtactgattattagcctcggcgtgctgtatccgctgtacctcaacaccgccagcgggttcaggcaaattgatccaaagcttctggaagccggccacgtgatgggcttcggatttttccagaggttgcggaccatcatcattccttctgccgcgccgcaactttttgtcggcctgcgccaagcaagtgcggccgcctggctctcactgatcgtggcggaacaggtcaacgcccgcgaaggactcggcttcctcatcaacaatgcgcgcgatttttaccgcaccgacctcgttattttcggcctcattgtctacgccagcctcggtctgctgtctgaagcgctgatcagagcttgggaacgtcacaccttccgctaccgaaacgcataa

SEQ ID NO: 163 - protein, 256 aa, Corynebacterium glutamicum
MTTTLTRPKIALPARIYSPLAVLVFWQLGSSLGAIPERILPAPTTILAASWEVATNGTLLDALLVSSQRVLLGFALGAVLGISLGVLTGMSRFADTAVDPLIQAARALPHLGLVPLFIIWFGIGELPKVLIISLGVLYPLYLNTASGFRQIDPKLLEAGHVMGFGFFQRLRTIIIPSAAPQLFVGLRQASAAAWLSLIVAEQVNAREGLGFLINNARDFYRTDLVIFGLIVYASLGLLSEALIRAWERHTFRYRNA

SEQ ID NO: 164 - DNA, 732 bp, Corynebacterium glutamicum
atgactgccacattgtcactcaaacccgcagccactgtccgtggattgcgcaaatcatacggaactaaagaagtcctccaaggaatcgacctcaccatcaactgcggcgaagtaaccgcgctgatcggacgctcaggttcaggaaaatccaccatcctgcgcgtgttggcgggcctatctaaagagcattccggctctgtagaaatttccggaaacccggccgttgccttccaagagcctcgcctgttgccgtggaaaacggtgctcgataatgtgacctttggcctcaaccgcactgatatttcctggtcagaagcacaagaacgcgcctcggcactgcttgcagaagtcaaacttcccgactccgacgccgcctggcccctcacgctctccggcggccaagcccagcgcgtctcccttgcgcgagcgctcatctccgagccagagcttttgcttctcgacgaacccttcggcgccctcgatgctctgacaagactgacagcccaagacctgctgctcaaaaccgtgaacacccgaaacttgggagttctgctggtcacccatgatgtttccgaggccatcgccctggccgaccacgtccttcttcttgacgacggcgccatcacacacagtttgactgtagatatccccggcgatcgccgcacccacccctcctttgcctcctacaccgctcaactccttgagtggctcgaaatcaccacacctgcctag

SEQ ID NO: 165 - protein, 243 aa, Corynebacterium glutamicum
MTATLSLKPAATVRGLRKSYGTKEVLQGIDLTINCGEVTALIGRSGSGKSTILRVLAGLSKEHSGSVEISGNPAVAFQEPRLLPWKTVLDNVTFGLNRTDISWSEAQERASALLAEVKLPDSDAAWPLTLSGGQAQRVSLARALISEPELLLLDEPFGALDALTRLTAQDLLLKTVNTRNLGVLLVTHDVSEAIALADHVLLLDDGAITHSLTVDIPGDRRTHPSFASYTAQLLEWLEITTPA

SEQ ID NO: 166 - DNA, 960 bp, Corynebacterium glutamicum
atgaaatttaagaaaatcgccctcgttctcgccttcggtctaggccttgcatcctgctcatcagcttctggcgatcccgccaccaacgccgatggatccatcgatctgagcaaagtaacccttaacatcggtgatcaaatcgccggaacagaacaagtgctccaagcttcaggggagctagatgatgtcccttataaaatcgaatggtcatcatttacctctggaccaccccaaatcgaagcattaaacgcaggtcaaattgatttcgcgatcaccggaaacaccccaccgatcatcggcggccccaccaacaccaaagtggtctccgcctacaacaacgatgctttaggtgatgtcatcttggtcgccccggattcttcaataacctcggtggctgaccttgctggaaagaaagtggctgtcgcccgcggatccagcgcccacggacacctcatccaacaactagaaaaagcaggcgtgagcgttgacgacgtagaaatcaacctcctccaaccctccgacgccaaagccgctttccaaaacggccaggtagatgcgtgggcagtgtgggatccctacagctcacaggcggaactggaaggagctcaagttttggtcaggggagcgggactggtcagtgggcatggatttggtgtcgcaagtgatgaagcgctcgatgaccccgcaaaggaagccgccttggcagatttcctcgatcgcgtggccgactcttatgaatgggctgaagacaacaccgatgaatgggcgacgattttcagccaagaatccggctttgatccggaggcctctcaactgaacacccgcagcctgcgccatcaggtgccgctcgacgagtccgtcaacacctatcagaacgcgcttatcgacgctttcgtctccgcgggtctcgttgaggactttaatttcgaggacaccgtagacacccgatttgagggctaa

SEQ ID NO: 167 - protein, 319 aa, Corynebacterium glutamicum
MKFKKIALVLAFGLGLASCSSASGDPATNADGSIDLSKVTLNIGDQIAGTEQVLQASGELDDVPYKIEWSSFTSGPPQIEALNAGQIDFAITGNTPPIIGGPTNTKVVSAYNNDALGDVILVAPDSSITSVADLAGKKVAVARGSSAHGHLIQQLEKAGVSVDDVEINLLQPSDAKAAFQNGQVDAWAVWDPYSSQAELEGAQVLVRGAGLVSGHGFGVASDEALDDPAKEAALADFLDRVADSYEWAEDNTDEWATIFSQESGFDPEASQLNTRSLRHQVPLDESVNTYQNALIDAFVSAGLVEDFNFEDTVDTRFEG

SEQ ID NO: 168 - DNA, 3679 bp, Corynebacterium glutamicum
atgacattaactttccattggttcctatccacttcaggcgattcccgcggcatcatcggcggcggtcacggtgcagaaaaatccggcacctcccgcgaattgagccacagctacctcaagcagttggcgctagctgccgagaccaacggttttgaatctgtcctgacaccaacgggcacgtggtgcgaagatgcgtggattactgacgcttctttgattgaggcgacaaaacgcttgaagttcctcgttgcgcttcgccctgggcagattggacctacgctgtctgctcaaatggcttctactttccagcgtctgtctggcaaccgtttgctgatcaatgtggtcaccggtggggaagatgcggagcagcgtgcgtttggtgatttcttgaacaaggaggagcgctacgcccgtaccggagaattcttggatatcgtgagccgcttgtggcgaggcgaaaccgtcacgcaccacggtgaacacctgcaggtggagcaagctagccttgcgcatccgccagagattattccggagattctttttggtggatcgtcgccagctgcaggtgaggtggctgcacgttatgcggacacctatctcacgtggggtgaaactcccgatcaggtggcgcagaaaatcaactggatcaacgagctagcagcacagcgcggccgggaactgcgccatggaatccgcttccatgtgatcacccgcgatacgtctgaagaagcatgggtggtggcagagaagttgattagcggggtcactccagaacaggtcgctaaggctcaagccgggtttgcaacgtctaagtcggaggggcagcgccggatggctgagctgcacagcaagggtcgtgcctttactagtggctcaactgctcgtgatctggaggtgtatcccaatgtgtgggcaggcgtcggtttgcttcgcggaggtgcaggaacagcccttgtgggctcgcatgaagaggtcgccgatcgcatcgaagaatacgcagcactcggcttggatcagtttgtactgtcgggttatccaaacttggaggaggccttccacttcggtgagggtgtgattccggagctgctgcgccgcggtgtggatatcaaaaatcaagaatcacgagttttggaacctgttgggtaaacgggaagaacgagacgtcgataagcaaatttcttaaggaacctgacatgactacaaccttgactcgccccaaaatcgcgctgcccgcgcgcatctattcaccgcttgcggtgcttgttttctggcagctcggctcgagcctgggcgccatcccggagcggattctgccggcaccaaccacgatcttggccgccagctgggaggtcgccacaaatggcacgcttctcgacgccctcctcgtctcaagccaacgcgtccttctaggcttcgccctcggtgctgtcctaggcatttccctaggtgtattgacaggcatgtccagatttgcagacaccgccgttgatccgctcattcaagctgcccgcgcgctgcctcacctgggtcttgtgccgctgtttatcatctggttcggtatcggtgagctgccgaaagtactgattattagcctcggcgtgctgtatccgctgtacctcaacaccgccagcgggttcaggcaaattgatccaaagcttctggaagccggccacgtgatgggcttcggatttttccagaggttgcggaccatcatcattccttctgccgcgccgcaactttttgtcggcctgcgccaagcaagtgcggccgcctggctctcactgatcgtggcggaacaggtcaacgcccgcgaaggactcggcttcctcatcaacaatgcgcgcgatttttaccgcaccgacctcgttattttcggcctcattgtctacgccagcctcggtctgctgtctgaagcgctgatcagagcttgggaacgtcacaccttccgctaccgaaacgcataagaaagttgctcgccatgactgccacattgtcactcaaacccgcagccactgtccgtggattgcgcaaatcatacggaactaaagaagtcctccaaggaatcgacctcaccatcaactgcggcgaagtaaccgcgctgatcggacgctcaggttcaggaaaatccaccatcctgcgcgtgttggcgggcctatctaaagagcattccggctctgtagaaatttccggaaacccggccgttgccttccaagagcctcgcctgttgccgtggaaaacggtgctcgataatgtgacctttggcctcaaccgcactgatatttcctggtcagaagcacaagaacgcgcctcggcactgcttgcagaagtcaaacttcccgactccgacgccgcctggcccctcacgctctccggcggccaagcccagcgcgtctcccttgcgcgagcgctcatctccgagccagagcttttgcttctcgacgaacccttcggcgccctcgatgctctgacaagactgacagcccaagacctgctgctcaaaaccgtgaacacccgaaacttgggagttctgctggtcacccatgatgtttccgaggccatcgccctggccgaccacgtccttcttcttgacgacggcgccatcacacacagtttgactgtagatatccccggcgatcgccgcacccacccctcctttgcctcctacaccgctcaactccttgagtggctcgaaatcaccacacctgcctagaaagaaatcatgaaatttaagaaaatcgccctcgttctcgccttcggtctaggccttgcatcctgctcatcagcttctggcgatcccgccaccaacgccgatggatccatcgatctgagcaaagtaacccttaacatcggtgatcaaatcgccggaacagaacaagtgctccaagcttcaggggagctagatgatgtcccttataaaatcgaatggtcatcatttacctctggaccaccccaaatcgaagcattaaacgcaggtcaaattgatttcgcgatcaccggaaacaccccaccgatcatcggcggccccaccaacaccaaagtggtctccgcctacaacaacgatgctttaggtgatgtcatcttggtcgccccggattcttcaataacctcggtggctgaccttgctggaaagaaagtggctgtcgcccgcggatccagcgcccacggacacctcatccaacaactagaaaaagcaggcgtgagcgttgacgacgtagaaatcaacctcctccaaccctccgacgccaaagccgctttccaaaacggccaggtagatgcgtgggcagtgtgggatccctacagctcacaggcggaactggaaggagctcaagttttggtcaggggagcgggactggtcagtgggcatggatttggtgtcgcaagtgatgaagcgctcgatgaccccgcaaaggaagccgccttggcagatttcctcgatcgcgtggccgactcttatgaatgggctgaagacaacaccgatgaatgggcgacgattttcagccaagaatccggctttgatccggaggcctctcaactgaacacccgcagcctgcgccatcaggtgccgctcgacgagtccgtcaacacctatcagaacgcgcttatcgacgctttcgtctccgcgggtctcgttgaggactttaatttcgaggacaccgtagacacccgatttgagggctaa

SEQ ID NO: 169 - DNA, 492 bp, Corynebacterium glutamicum
atgacttcagcaatcaccaccgcaactgatcttcgctccgtactgcgaaacgtaccaaccccaattagcttcattgcaacccacaccgatcagcctctgggcatgatcgttggttcattcgtcagcattagcgccgaaccaccattggtaggcatcttcttgcagaagagctcttcttcatggccagctatcgagcaggcattagttaccggccaagagctaggcatttctatcctcggcggggcacacgcagaccatgtgcgtaagctttctggcccatccgaccagcgctttgaaaaccttgggtgggcatccaccgaaaacggtgcgattcaccttgaaggcgctgatgcacaactaaccacgaaacttcatgatctccaggaaatcggcgatcacttctttgcagttctagaagttattgacgcttccgctgaccaagacttcagctcagcgctggtgtaccaccgctcacaggtgtcctcgctgtag

SEQ ID NO: 170 - protein, 163 aa, Corynebacterium glutamicum
MTSAITTATDLRSVLRNVPTPISFIATHTDQPLGMIVGSFVSISAEPPLVGIFLQKSSSSWPAIEQALVTGQELGISILGGAHADHVRKLSGPSDQRFENLGWASTENGAIHLEGADAQLTTKLHDLQEIGDHFFAVLEVIDASADQDFSSALVYHRSQVSSL

SEQ ID NO: 171 - DNA, 599 bp, Artificial Sequence
  Synthetic Construct
tgagaattaaatacggctgtcacaagcttactcattgtgttgtcgcgaaacactggagtccacccctagagtgaggtgggtaatagaaagtgtgagaaataaggcacaatccactcctaaatgtgggggtgcaggcttgtttaacgtccaaaattcggcggtaggtgtacctaaaaacaggggtgggaaatacttggggattgctctcatcggttcgatatagactgaattgtctaggttaatatccaatatggaagaaaaactagacagttaagtagactgaatggcctactaggtgcgacactaaattttaagagggcgcgtcgaatcttcgacgtgccctcttttaaaattcgtggaacgctttgtggtgagtgtggagactttcctcttgttcggtgttaagtggttcgcgtcctcttttctcgcgaatgattctcacgtgattgataaaggcagcggagtcagggttttcgattgcggttttaagctccattccgtacagtttttgctcttctgttgtgaaataatctgttctgttgcggaagtccagtcccataagttcaaccagtgcgtcatcggataggaaaccgttaaaa

SEQ ID NO: 172 - DNA, 1464 bp, Corynebacterium glutamicum
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatga

SEQ ID NO: 173 - protein, 487 aa, Corynebacterium glutamicum
MATAENTTQENRKILFNAFDMNCVAHQSPGLWTHPKDKARDYNTLDYWVHLAKTLEKGLFDGLFIADVLGTYDVYGSSNEAALSSGAQVPVNDPILLVSAMAYATKNLGFGITAGTAYEHPYPFARRLATLDHLTNGRVGWNVVTGYLPSAAQNMGDTDQLPHDERYDKADEYLEVIYKLLEGSWEDDAVQNNTETSVFTDSSKVHAINHHGKYFDVPGIAITEPSVQRTPVIYQAGASPRGLKFAGENAEAVFINSSTVEAITKTVAKIRAAAVAAGRDPHAVKIFAMQTIITGETEADAQAKLEEYSRYIDPVGGLTLMSGWTGADLSQYDLDEPITNIESNAIQSTAATISNGTGEGAWTVRKLGEATGIGGFGPVLVGSGANVAAELARIQDLSDVDGFNLAYAITPGTFEDVVDFVVPELQKLSRYKTEYAPGSLRNKLLGKGDRLDDTHRGASYRLGARNSTATIDLSSISAQLVSQGAHS

SEQ ID NO: 174 - DNA, 1062 bp, Corynebacterium glutamicum
atgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcatgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatga

SEQ ID NO: 175 - protein, 360 aa, Corynebacterium glutamicum
MISPQTIIDNLAPVLAEIAATAAQREQDREFSRDLAKQLSAGGFTKLRIPVEFGGLGFSLPEAFEVLVAAAAADSNIAQGLRPHFLAVESLLIAPYSEHRTKWLRKIAEKGVVIGNALTEVGNKPGELKTKIRKEGESYVLSDTKFYSTGSLYADWIQVHAKDEEDQDVFAFVDRDASGVVLVDYWDEFGQQLFASGTSFFEKVVVDPLDIVTRDYTAPSAFQALAQSHHLSTLTGISQAITRDIVTYVQNRTRIFSHGSGDLPRFDPQVQQVVGEVKAKSYAVEKIFQGFAQELDLVVDKAKAGTATEVDLAAVDLSAYQAQLAVAPLVLSQATQAFEVGADALNGGHVAAQYTIGSLS

SEQ ID NO: 176 - DNA, 1200 bp, Corynebacterium glutamicum
atgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctag

SEQ ID NO: 177 - protein, 399 aa, Corynebacterium glutamicum
MSQEILSHFAPALERIRSGAVEREQQRALPVEEIKELVELGFTGLRVPEELGGAGASLESVVELLIEIAGADSNIAQALRGHFAFVELLLEAPESEFRTHWLREVATGRLVGNAESEKRGVYGDPQTFIDEVETENGPIFVLNGTKFYTTGTYFADYTWTTALLRNLNGQETLVSLPVDLHAPGVDVVDDWSGFGQKLTASGTTTFKDLEVDPRWIIPRTDAPTLVWTYLQLSLLTVLVGSAAAAVDEVVARAQSSTRNAWNPGVERRSDPAATIAIGDARSRVTVIRGALLDATRHVSNAATIVTPEAFNEADAIVAALWPIVSGQALVVTSNVFDAVGASAVLGEHSIDRHWRNVRTVSSNNPVFLAKNAVGEYALNGTPVGTNIGKALSRPVSLSS

SEQ ID NO: 178 - DNA, 1179 bp, Corynebacterium glutamicum
atgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 179 - protein, 392 aa, Corynebacterium glutamicum
MSENNPTTLHWFLPTYGDSRGITAGGHGFGFHSGSRTADLDYLSQIALAAERNGFESVLTPTGLWCEDAWITTAALLSRTSKLKFLVAIRPGQVSPTIIAQQGAAFQKFSNNRLLINVVVGGEDHEQRAFADYSSKEERYHKADETLEIIDHLWNSAEPLNFQGEFLSVENAVLKEQPEVSPPIYFGGSSQLGIEIAAQHSDVYLTWGEPAEKVEEKLARVRAEADKRNRELDYGIRLHVIARPTEDEAWSVAQNLLDQLDQEEVARIQEGLARSQSEGQRRMTELHGQGAAFTAGADARSLEIAPNLWAGVGLVRGGAGTALVGSYEQVAQAILRYRDIGLSHFIFSGYPHLEETYHVGEGVVPELLKLGVPVNNHEEQRNDVVATPFISR

SEQ ID NO: 180 - DNA, 5028 bp, Corynebacterium glutamicum
atggccacggccgagaacacaacacaggagaatcggaaaatcctgttcaacgcatttgatatgaactgcgttgcgcatcagtccccaggactgtggacacacccgaaggataaggcgcgagactacaacactcttgattactgggtgcaccttgccaagactttggagaagggccttttcgacggccttttcatcgcagatgtgcttggaacttacgatgtttatggttctagtaatgaagcggcgttgagcagtggtgcgcaggtgcctgtcaatgatccgatccttcttgtttctgcgatggcctatgccacaaagaacctcgggtttggcattactgcaggtactgcctatgagcacccgtatccttttgcgcggcgtctggccacacttgatcacctgactaatgggcgtgtggggtggaatgtggttactggctatcttccctctgctgctcaaaacatgggtgacaccgatcagctgccacatgatgagcgctatgacaaagcagatgaatacctggaagtgatctacaagcttctcgagggctcctgggaagacgatgctgttcaaaacaatacggagacgagtgtctttacggactcctccaaagtgcacgccattaatcatcatggcaagtactttgatgtgccgggcattgccatcactgagccgagtgtgcagcgtacgccggtgatctaccaggcgggtgcatcgccgcgcggattgaaattcgctggtgagaatgcagaagcagtgtttatcaattccagcaccgtggaggcaatcaccaagactgtcgcaaaaattcgcgctgctgcggtcgctgcgggacgtgatccacatgcggtgaagatctttgcgatgcaaaccatcatcactggtgaaacagaagcagatgcgcaggcaaagctggaggaatacagtcgctatatcgatcctgtcggtggtctgaccttgatgtctggatggaccggcgcggatctgtcgcagtatgacctggatgaaccgatcaccaatattgagtcaaacgctattcagtccactgcagccaccattagcaacggcaccggtgaaggtgcgtggacggtacgcaaactgggtgaggcaaccggcatcggcggcttcggaccagtgcttgtgggatctggcgctaacgttgccgcggaacttgcacgcatccaggatctcagcgatgttgatggtttcaaccttgcttatgccatcaccccaggaacttttgaagatgtcgtggactttgtggtgcctgagctgcaaaaacttagccgctacaagacggaatacgcgccgggttccttgcgcaacaaattgctcggtaaaggtgatcgcctggacgatacccaccgcggcgcaagctaccgcctaggcgctcggaactccaccgccactattgatctcagttccatatccgcccaactagtttcccagggagcccactcatgatctcaccgcaaacaatcatcgacaatcttgctccagtcctcgcggagatcgcagcaacggctgcgcagcgcgaacaggatcgagaattcagccgtgatttggcaaagcagcttagtgccggtgggttcactaaactccgcattccagttgaatttggcggtttgggttttagccttccagaagcatttgaggtgctggtggcagcggccgctgcagattcaaatatcgcgcagggattgcggcctcacttcttggcagtggagagcttattgatcgctccttattcagagcaccgcaccaagtggctgcgaaaaatcgctgagaaaggcgtggtcattggcaatgcgttgactgaagtgggaaacaagccaggtgagctgaaaaccaagatccgaaaggaaggcgagtcttacgttctcagcgacaccaagttctattccacgggcagtctttacgccgactggattcaagtacatgcgaaagatgaggaagatcaagatgttttcgcctttgttgatcgcgacgcatcaggcgttgttttggtggattactgggacgaatttgggcaacaactatttgcctccggaaccagcttctttgaaaaagtcgtggtagatccactggacattgtcacccgtgattacaccgcacccagcgctttccaggcgctggcgcagtcacatcatttgtctacgttgactggtatttcgcaggcaatcacccgtgacattgttacctatgtacaaaaccgcacccgtattttcagccacggaagtggtgaccttccacgctttgatccgcaggtgcaacaggtggtgggtgaggtgaaagccaagtcgtatgcagtcgagaaaatctttcagggtttcgcacaagaactggatcttgtcgtcgataaggcaaaagccggcactgctacggaggtcgatctggccgctgtcgacctgagcgcctaccaagcccagttagcggtggcacctttggtgctgagccaggccacccaggcctttgaggttggcgcagatgccttaaacggcggccacgtggctgcccaatacacgatcggatccttgtcatgagccaagaaattttgagccattttgcacccgcattagagcgcattcgaagcggcgccgtcgagcgcgaacagcagcgcgccttgccagtggaagagattaaagagctggtagagctaggttttactgggcttcgagtgcccgaagaactaggcggtgcgggcgcttccctggaaagcgtagttgagttactgatcgagatcgcgggcgccgattccaatatcgcccaagccctgcgcggacattttgccttcgtggaactactcctggaagcgccggagagcgaattccgcacccattggctgcgcgaagtcgccaccggaagacttgtgggcaacgccgaaagtgagaaacgcggcgtttacggcgatccgcagaccttcatcgatgaggtggagactgaaaacggaccgattttcgtgctcaacggcactaagttttataccaccggcacctattttgcggactacacctggaccaccgcgctgctgcgcaacctcaacggccaagaaactttggtcagtttgccggtcgatctgcacgcgccgggcgtggatgttgttgatgattggagcgggtttgggcaaaagctcaccgcctctggaacgaccacgtttaaagacctggaggtggatccgcggtggatcattccacgcactgatgcgcccacgctggtgtggacgtatctgcagctgagcctgctgaccgtgctggttggcagtgccgcagcagctgtcgatgaggtggttgcccgcgcccaatcctccaccagaaatgcgtggaaccctggcgtcgagcgccgcagcgatccggccgcaaccatagcgatcggcgacgcacgcagccgagtcaccgttattcgtggagcgcttctcgacgccacccgccacgtttccaacgccgccacgatcgtaacccccgaagccttcaacgaggcggacgctattgttgcagcgctctggcccatcgtctccggacaagctttggtggtgacatccaacgttttcgatgcggtgggtgcatctgcagtgcttggtgagcattccattgatcgccactggcgcaatgtgcgtaccgtgtcctcaaacaacccggtgttcctggccaagaatgcagtgggggagtatgccctcaacggcactcctgtgggtaccaacattggtaaagcactgagccgtccggtgagcctaagcagctagacgtgtgatttcgctggtttttagatggttttcaagccagcgagaccacattagtttcacgctggttgaaacctttgagatcaatatagaccgtgtggtctactcgaggaatgagtgaaaacaatcccactaccttgcactggttcctacccacctatggcgattctcgcggaatcacagccggcgggcatggcttcggcttccactccggaagccggacagcagacctcgattacctctcccaaattgccctggccgctgaacgaaacggttttgaatccgtcctgactcccactggattgtggtgcgaagatgcgtggatcaccaccgcagcgctgctgtctaggacatcaaaactgaaattcctcgttgctattcgaccaggccaagttagccccaccatcatcgcgcagcagggtgctgccttccagaaattctcaaataaccgcctgctcatcaacgtcgtggtgggtggcgaagaccatgaacagcgcgctttcgctgattattcttccaaagaggagcgctaccacaaggctgatgaaaccttagagatcatcgatcacctatggaacagcgcagaacctctaaatttccagggtgaattcctcagtgtggaaaacgcggtattgaaggaacagcccgaggtttccccaccgatttactttggcggatcctcacaactcggcatcgaaatcgcagcccaacattccgatgtttatctcacctggggtgaacctgcggaaaaggtagaggagaagcttgcccgggtgcgcgccgaagcagataagcgaaaccgcgaactagactatggcatccgcctgcatgtcattgctcgaccaactgaggatgaagcctggtcagtggctcaaaatcttcttgaccaacttgatcaggaagaggttgcccgcattcaggaagggcttgcgcgttctcaatcggaaggtcagcgtcgcatgacggaacttcatggacaaggggcagcattcacagcaggagcagatgctcgctcccttgaaattgcaccgaatctctgggcaggtgttgggctagtccgcggtggcgccggcacagcgttggtgggttcctatgagcaagtcgcgcaagcaattttgcgataccgcgatattggtctgagccacttcattttctccggctatccacatttggaggaaacctatcacgtgggcgaaggagtggtacctgagctcctcaaattgggtgttccggtgaacaaccatgaagaacaacgcaacgacgtggtagcgactccgtttatttccagatag

SEQ ID NO: 181 - DNA, 1181 bp, Artificial Sequence
  Synthetic Construct
ttgctgtggcagcgggacgccctctggctgatttccagggcggacagccagcggatgcactaaagcagtggactgatcaggtggttcaggctgtgggtggacagctggaaggactgccagaggaggccacagacggcgaacaagaagacgctcctgtggaagacccccgcttcgatgctgccactgatgctctaaaccgtggcgctttcgatgaggcgattgcggtttatgagtccattttggcgcaggagccaaacaacgctgatgcgaagcaggcacgcgataccgcaaagctgttgggccggcttgccacggtggatccttcggtggatgttgtcgctgctgcagatgctgatccaacaaacgttgatctggcctacacagcagctgacgcggctgttgttgcgggtgatcctgaggctgcctttgatcgtttaattgctctgctgaccatcagcgctggcgatcagaagaatcaggtgaaggaacgtttgctggagctgtttggcatgtttgagaccgccgatccccgtgtgctgcaggcgcgaggaaagatggccagcgcgctgttctaaaaccactctctatccagaaaaatatagaccgcttagtcttttccaggactaagtggtctacatttttacccaaaatgcagctcacgcaatagacatctcggtctatatacttgccaccttcgcgccctgcaaatccccacactacttatatccagcccgaaaataatacttctctctagacgaagcggtctgtttaagtatgtgccgtatgtctgagtatggcaaaggggcgttgaggtgctcagacggccatacaaagcatcgtagtttgtgcatatgagggctgctttttgccgctgtgagttggctgttcaggaatccgtcctcgggttggggagaggggctgaaaccgaacgaatattcgcataaatctttcgaattggcgcgaatattcgatcccgctggcctccgaactagccattttggtagaaaatttgcatccttgagccggtattggcccaaggatgcattttcctgtgtcctacttctgcagtgagtagaactgagcactcatcgctgggacatgcagtgagagtgagtaggcgaaattatcgcgtggtgtcgctgctgcttgaacggaagtagcgatatcgttttctgcaccga

SEQ ID NO: 182 - DNA, 851 bp, Artificial Sequence
  Synthetic Construct
gctgcccatgccttggtgtgctcgtttgcatactggccgtagccggctggcattgcttcgtcgaggacggggaagttgaaatcggcgatatcaacgagctcgtactgtgcatcgttgcgctcttgtgcgcgctcgagaacccagttggctacgtcaatgttgacgcggcctggacgggtggaaccggtgatgatggcgatcttgctcatggtgtactcctctaaaagttaattaagttgatgtatcaacaacttgacgtacctagcctatgttaactttccgcgtcatgcaagcgctttgggattccagtctatttctcaaggtcaacagggtgggggagtcgtcgaggttttcattcagggtgaatgctctccttgtttcagatgttcaacgctccataaagtagaccgcaatctagacaaagatgtctattttaattaaggagcagaacatcacggatcggctgctttagcagaggcaaaagccgggattcagttggtgcggcttccagaggttgtcacaaaccgtcttatatgaccaacggccaggagatgtgaggttggtcggtcacctagtttggtcggtgcttggacgccagaaaaactggccgggccccaccaaaagtgacctctcagattcgcctctaagcgcccaaagccctgaaccatacaaatactcaatcttgatcttcgacctcttaaatcgcatttgcactggcatccctcgtagggatccccaagcaaaaaaccgccgaccacaatggccggcggtttttcgaactaagagctcttaggtcgcggtggtgtagtcaaccacggagtattcctgcagcttggacagctggtggacag

SEQ ID NO: 183 - DNA, 599 bp, Artificial Sequence; identical to SEQ ID NO: 171
  Synthetic Construct

SEQ ID NO: 184 - DNA, 1536 bp, Escherichia coli
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaaggcaaagcaaataccggcgtcactctggagccaatcaacagtcagaacgcaccaaaaggtcacttcttcctgcacccgcgtgcacgttcaccacactatattgtgatgaatgacaagaaacactaa

SEQ ID NO: 185 - protein, 511 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRKGKANTGVTLEPINSQNAPKGHFFLHPRARSPHYIVMNDKKH

SEQ ID NO: 186 - DNA, 1413 bp, Escherichia coli
atggctacatcagtacagacaggtaaagctaagcagctcacattacttggattctttgccataacggcatcgatggtaatggctgtttatgaataccctaccttcgcaacatcgggcttttcattagtcttcttcctgctattaggcgggattttatggtttattcccgtgggactttgtgctgcggaaatggccaccgtcgacggctgggaagaaggtggtgtcttcgcctgggtatcaaatactctggggccgagatggggatttgcagcgatctcatttggctatctgcaaatcgccattggttttattccgatgctctatttcgtgttaggggcactctcctacatcctgaaatggccagcgctgaatgaagaccccattaccaaaactattgcagcactcatcattctttgggcgctggcattaacgcagtttggtggcacgaaatacacggcgcgaattgctaaagttggcttcttcgccggtatcctgttacctgcatttattttgatcgcattagcggctatttatctgcactccggtgcccccgttgctatcgaaatggattcgaagaccttcttccctgacttctctaaagtgggcaccctggtagtatttgttgccttcattttgagttatatgggcgtagaagcatccgcaacccacgtcaatgaaatgagcaacccagggcgcgactatccgttggctatgttactgctgatggtggcggcaatctgcttaagctctgttggtggtttgtctattgcgatggtcattccgggtaatgaaatcaacctctccgcaggggtaatgcaaacctttaccgttctgatgtcccatgtggcaccagaaattgagtggacggttcgcgtgatctccgcactgctgttgctgggtgttctggcggaaatcgcctcctggattgttggtccttctcgcgggatgtatgtaacagcgcagaaaaacctgctgccagcggcattcgctaaaatgaacaaaaatggcgtaccggtaacgctggtcatttcgcagctggtgattacgtctatcgcgttgatcatcctcaccaataccggtggcggtaacaacatgtccttcctgatcgcactggcgctgacggtggtgatttatctgtgtgcttatttcatgctgtttattggctacattgtgttggttcttaaacatcctgacttaaaacgcacatttaatatccctggtggtaaaggggtgaaactggtcgtggcaattgtcggtctgctgacttcaattatggcgtttattgtttccttcctgccgccggataacatccagggtgattctaccgatatgtatgttgaattactggttgttagtttcctggtggtacttgccctgccctttattctctatgctgttcatgatcgtaaataa

SEQ ID NO: 187 - protein, 470 aa, Escherichia coli
MATSVQTGKAKQLTLLGFFAITASMVMAVYEYPTFATSGFSLVFFLLLGGILWFIPVGLCAAEMATVDGWEEGGVFAWVSNTLGPRWGFAAISFGYLQIAIGFIPMLYFVLGALSYILKWPALNEDPITKTIAALIILWALALTQFGGTKYTARIAKVGFFAGILLPAFILIALAAIYLHSGAPVAIEMDSKTFFPDFSKVGTLVVFVAFILSYMGVEASATHVNEMSNPGRDYPLAMLLLMVAAICLSSVGGLSIAMVIPGNEINLSAGVMQTFTVLMSHVAPEIEWTVRVISALLLLGVLAEIASWIVGPSRGMYVTAQKNLLPAAFAKMNKNGVPVTLVISQLVITSIALIILTNTGGGNNMSFLIALALTVVIYLCAYFMLFIGYIVLVLKHPDLKRTFNIPGGKGVKLVVAIVGLLTSIMAFIVSFLPPDNIQGDSTDMYVELLVVSFLVVLALPFILYAVHDRK

SEQ ID NO: 188 - DNA, 1507 bp, Lactobacillus brevis
agtggatgaaaataagtctgaacagcaaattgatacgcaaaaggcaaatcgaataacgggctttcagttattttccatgacgacttccatggtcatgacggtttacggttttgcggcatttgctaagcaaggaccaaccgctttcttttacctgtttttagccgggatcttatggtttttaccggtgactcggacctcaggagagatggcgtccatcgatggctggagtaaaggcggtatctttacttggtcacggaatatgttaggtgaacgcgcaggttggtcggcattattctaccaatggattcacattacggttgggatgaatacaatgatgtatttcattatcggctgtttttcagtaacgtttggcttacccattatcacggataacccgttagttaagtttattttgatgatgatcattttatggggccttatctttctacagcaaaaaggaacgagtgttaccggtaagattgcccaatggtgtttcacgttaggggtgattatcccagtcttcttcttactctttctatttattttgtacctcgtacaaggaaacccagcgatgattcacgtcaacatgcacaccatttttccatcgagttggaatgggagtgtcttagtgggatttgttccgtttatcttggcttttgccggtgccgaagggtcggcaccacacgttaaggacttggataaacctagcatctatcctaaggttatgatggcactagcagttgctgcgatctgttccgatattatcggcagtatggcgattgctatgacgattccgaataatcagattcagttaagtaatgggattgtttacgcctatggggccttagttgcccgctacggtgtgggtgtggtctttgtcgaaaagctaacagggttcttattagccgttggtgtcttgggtgaaatcagcagttgggtcgttggtcctaacgcggggatgtttgaagcagctaaagctggctacttaccaccgcgcttctctaaggccaataaatatgggattgaaaccaatgtcatggtccttcaaggggtcatcgtgtcgattgttggggcgctgttgacgtttggggctggcgggaacaaggcgagcctttccttccagactgcaatgagtttgacggtagccttatacaccttgatgtatatgctgatgtttattagttatttggtcttacagttcaagtatgtcgatttgcatcgggactttgtggccgctaagtcgcgttggttgcggatcacttacggtatcctagggtttattttatcggcctttggatttgtcgtgaccttcttcccgccagcagatttatccgttgcctcaaaacacacgtacctaatgttgttagtgagtgcctttgtggtcatgctcgttttgccatttatcttgtaccgtttccacgacaaatgggccttgcagctaggtgtcaatgtggatgaagtggcagcaccgaccggtcatgaggcgttggaaaaagaaaccaagtag

SEQ ID NO: 189 - protein, 501 aa, Lactobacillus brevis
MDENKSEQQIDTQKANRITGFQLFSMTTSMVMTVYGFAAFAKQGPTAFFYLFLAGILWFLPVTRTSGEMASIDGWSKGGIFTWSRNMLGERAGWSALFYQWIHITVGMNTMMYFIIGCFSVTFGLPIITDNPLVKFILMMIILWGLIFLQQKGTSVTGKIAQWCFTLGVIIPVFFLLFLFILYLVQGNPAMIHVNMHTIFPSSWNGSVLVGFVPFILAFAGAEGSAPHVKDLDKPSIYPKVMMALAVAAICSDIIGSMAIAMTIPNNQIQLSNGIVYAYGALVARYGVGVVFVEKLTGFLLAVGVLGEISSWVVGPNAGMFEAAKAGYLPPRFSKANKYGIETNVMVLQGVIVSIVGALLTFGAGGNKASLSFQTAMSLTVALYTLMYMLMFISYLVLQFKYVDLHRDFVAAKSRWLRITYGILGFILSAFGFVVTFFPPADLSVASKHTYLMLLVSAFVVMLVLPFILYRFHDKWALQLGVNVDEVAAPTGHEALEKETK

SEQ ID NO: 190 - DNA, 1054 bp, Escherichia coli
agtgaacatatatatcgggtggcttttcaaattaatccctttgattatgggcttaatttgtatcgcgttaggtggctttgtgctggaaagttcagggcaaagcgagtatttcgtcgcgggtcatgtgctgatttctctggcggccatatgcctggcattattcactaccgcatttattatcatttcgcagctcacgcgcggcgttaatacgttttacaatacattgttccccattattggctatgcggggtcaattatcaccatgatatggggttgggcactgttagcaggcaatgatgtgatggcagacgagtttgtcgccggccatgttattttcggcgttggtatgattgccgcctgtgtatcgacggtggcagcgtcatccggtcactttctgctcattcccaaaaatgcagcggggagcaagagcgacggaacaccggtacaggcttattcttcattaatcggtaactgcctcattgccgttcccgttttactcaccctgctcggtttcatttggtctattacgctgttacgtagtgctgacataactccgcattatgtcgcgggtcacgtattgcttgggttaaccgcaatctgtgcctgtctaattggccttgttgccacaattgtccatcaaacacgtaatacgttttcaactaaagaacactggctgtggtgttattgggttatttttctcggctcaatcacggtactgcaggggatatacgtcttagtcagttccgatgcaagcgcccgactggctcccggcattattcttatttgcctcggaatgatctgttacagcatattctcaaaagtctggctactggcactggtatggagacgtacctgttcgttagccaacagaataccgatgattcccgtcttcacctgcctgttttgccttttcctggcatcgtttcttgcggaaatggcgcagaccgacatgggatattttattccttcgcgagttctggtcggtttgggagcggtatgctttacgttgttctcaatcgtttcaatattagaagcgggttctgctaaaaaataa

SEQ ID NO: 191 - protein, 350 aa, Escherichia coli
MNIYIGWLFKLIPLIMGLICIALGGFVLESSGQSEYFVAGHVLISLAAICLALFTTAFIIISQLTRGVNTFYNTLFPIIGYAGSIITMIWGWALLAGNDVMADEFVAGHVIFGVGMIAACVSTVAASSGHFLLIPKNAAGSKSDGTPVQAYSSLIGNCLIAVPVLLTLLGFIWSITLLRSADITPHYVAGHVLLGLTAICACLIGLVATIVHQTRNTFSTKEHWLWCYWVIFLGSITVLQGIYVLVSSDASARLAPGIILICLGMICYSIFSKVWLLALVWRRTCSLANRIPMIPVFTCLFCLFLASFLAEMAQTDMGYFIPSRVLVGLGAVCFTLFSIVSILEAGSAKK

SEQ ID NO: 192 - DNA, 1422 bp, Cyanobium sp.
atgatcagtgatcaagttgcagcaccaagagtgcttggcctgtttggccttgcaatgatcaacatcgttgccatcgccagtcttcgcgatcttccacagatggcaagctatggcgttggggccatttttttctatgtgttagccgcgctcgtctttttcgtgccggtctctttggtagcagcggagttggcaacagcgtggcccgaacggggaggcgtctatatctgggtaagagaagcttttggaaaacgctggggctttgttgccgtttttctgcaatggtttcagaacctttgctggtttccagttgtactcacctttggagccgcatcactggcctatgccatcgcgcctggagattccgaagcactggccaataacaaaacctttattgtcagcatcgttctcactacttattgggctgcagttctttttaacttccgaggcctctccggttcagcaaaattaagcgccctcggtgctttctctggtgtatttattcctggcataacactgatcatcttggcaggagttgggctggcgacaggctatccatcacgattgctagagaatggcaaccaggtcattccagatatcagccagttttccaatatcacttttgcagtcagtgtctggcttgcttttgccgggatggaaatgaccgccgcccacgcgcgcgaagtcaaatcaccaggcagaaactttccgctaggaatcatgcttgcggcgatgatcatattgactatttttattcttggtgctctttcaattgcaacagctctggctccaggcgaatacgaactgcaaagtggagtgatcaaagcgctcaagcatatgctgagtcgctacaaacttgacgggtgggctcggctgtttgcacttgggatggcattgggtgttttcggaagcgtcaatgcctggatcgttggacccagcaaaggcatgctggcttcaactgaagacggcgctttgccgccaactctgctgaaagtgaaccagaacaacgtgccaactcatatcctgctgattcaaagtgcagttgtctcaatcatctgcttggcatttctattacaaccaacagtcgcttcagcctatttcatgattagcgttctgaccattcagatgtatctttgcatgtacgtgatgatgttccttgccgccttgagactaaggcaaaaacatccaaatcattcaggcagatttcgcatccccggaggtaaatttggcctgtggagcacttgcatcataggaggccttggttcactttttgcaatcatccttggcttctttccccctgaccaacttgagagttccggaatgaattctcaatccttcattttgtttttagccacaggattatcagtggcaatagcatctccgcacctgataggcttcaggaggcagggtcgacatgcacattaa

SEQ ID NO: 193 - protein, 473 aa, Cyanobium sp.
MISDQVAAPRVLGLFGLAMINIVAIASLRDLPQMASYGVGAIFFYVLAALVFFVPVSLVAAELATAWPERGGVYIWVREAFGKRWGFVAVFLQWFQNLCWFPVVLTFGAASLAYAIAPGDSEALANNKTFIVSIVLTTYWAAVLFNFRGLSGSAKLSALGAFSGVFIPGITLIILAGVGLATGYPSRLLENGNQVIPDISQFSNITFAVSVWLAFAGMEMTAAHAREVKSPGRNFPLGIMLAAMIILTIFILGALSIATALAPGEYELQSGVIKALKHMLSRYKLDGWARLFALGMALGVFGSVNAWIVGPSKGMLASTEDGALPPTLLKVNQNNVPTHILLIQSAVVSIICLAFLLQPTVASAYFMISVLTIQMYLCMYVMMFLAALRLRQKHPNHSGRFRIPGGKFGLWSTCIIGGLGSLFAIILGFFPPDQLESSGMNSQSFILFLATGLSVAIASPHLIGFRRQGRHAH

SEQ ID NO: 194 - DNA, 1494 bp, Chlorella desiccata
atgctaaacgtagaatctccacgtgcttcaaacgcccctttgcatccgttacagaaggccaaaccattaactctactccctttagttgctcttattttctacgacgtctctggaggtccctttggaattgaagatgcagtcagcaagggagcaccttttcttgccataactgggtttttaatattaccatttgcatggagtatccctgaagcattagtcacagctgaattagctactacattccctgaaaatagtggatttgtcgcttgggtgacggcagcgtttggaccttggtggggttttcaagaaggattttggaagtggttgagtggagtaacagataatgcagtttatccagtgatttttttgacttatctacagcaattagtgccagtattagagactggattgcctcgaacgctgtttttagtaggacttaatttggcactgacgtacatgaattacagagggttgcatgttgtcggtaaagtagctgtagctatgacagcatttactctactgccctttattctcatgtgtctgttaggtgctcctcatgtggatccttcaaattggctggttgttgactggaaagcagtgcaatgggttcctttcttaaatgttatgttttggtcattgaattactgggactctgtatctacgcttgctggtgaagttagtgatcccgcaaggacctttccacgtgcgttgagtattgctgttgttcttgtcattagtgcttaccttctcccattattgataggagtaggagtggagactggatcgacgagtttagattggacattgggctactttgccaccgttgcggaacacgtcggtgggaaatggctcgcttggtggattgttgtagcagcagcaatcagtcagttgggtcagtttgaagctgaaatgagcactgactcttatcagctattaggaatggccgaacgcggttttatcccttcgatttttgcacagcgatcttctcacggcaccccaacctatgctatattagctagtagtctcggtattgcagcaatggcaagctttaatttcttagagattgtagaactgctgaatgttgtctattgcatggctgaattgcttgaatttacagcgttcctgtatctacgcttgaggtacccaaatcttcacagaccatttcgagtgcctttgtcaactttaggatgtgccatcatgctcgcccctgcgtttatcctgctttccatcatgttaatgttgcctattttcagaggcgattggcgcgtcgtcggatttacttttttggctttcgttttgggtgctgtcctgtaccctttaatgcagtacgctcgtcaggaaggctggtgtgcctttgaaggtgtgacccctgatgagtttaaggagcatttgtatactacatatacccctgtacgacccgttggtagcagcagagaaagcggtgatatgatgactgttatggaagatcagcaacagggagaacaaattaattga

SEQ ID NO: 195 - protein, 497 aa, Chlorella desiccata
MLNVESPRASNAPLHPLQKAKPLTLLPLVALIFYDVSGGPFGIEDAVSKGAPFLAITGFLILPFAWSIPEALVTAELATTFPENSGFVAWVTAAFGPWWGFQEGFWKWLSGVTDNAVYPVIFLTYLQQLVPVLETGLPRTLFLVGLNLALTYMNYRGLHVVGKVAVAMTAFTLLPFILMCLLGAPHVDPSNWLVVDWKAVQWVPFLNVMFWSLNYWDSVSTLAGEVSDPARTFPRALSIAVVLVISAYLLPLLIGVGVETGSTSLDWTLGYFATVAEHVGGKWLAWWIVVAAAISQLGQFEAEMSTDSYQLLGMAERGFIPSIFAQRSSHGTPTYAILASSLGIAAMASFNFLEIVELLNVVYCMAELLEFTAFLYLRLRYPNLHRPFRVPLSTLGCAIMLAPAFILLSIMLMLPIFRGDWRVVGFTFLAFVLGAVLYPLMQYARQEGWCAFEGVTPDEFKEHLYTTYTPVRPVGSSRESGDMMTVMEDQQQGEQIN

SEQ ID NO: 196 - DNA, 1473 bp, Cyanidiococcus yangmingshanensis
atgaccgttcctttttcagagcaggccatggtaagcacaagttccaaactcacgaatacagcgtttgatggtctggcaattgaggaaggtgccgagacggatgctactgcagttgctgtggtggcaccggacaaggtcagtccgccccgctcgctcggcctgctccagctcgttggcatgttattccttttaacctctggtggcggttatggcttagaacctctggtagggtccgcaggtccacgatggtcgattcttgctttggtggttgcgccttggctctgggcgttaccgcaggcgttgatggcatcggagttggcgacgttgataccagaggacggtggctatgtgatctgggtggaggcagcgctcggttcgttctggggattccaacaaggttggtggagttttgtggactcacttgtggacaatgctcttttcccgcgtcttttcagtgattatacgctgcgtgttgccccagcattcggtatgcttggcagctggatggtcggggtattggtattggtgatctgcaccttggtcaatattctcggactgggtcttgtaggctgggcagcggtgctctttacagtgcttgtcgtgtccccctttatgttcatttgcatcgtcggctttcgaaggacggtgttgagttccttgtggcaggcgcgacctcttcacgagatcaactggcgtttgtttttggcctcgttgctatggaactggtgcggattcgattcctgctcgacggttgctggtgaaattcgcgatgtacatcgatcgtttcctcgagcaatggtcatcgtcctatcactgaccacaatggttttcacgttgccgatagctgcctcggtgacggtcaatcacgtctggagtgagtggcgtgatgcgttctggccgactgctgcgaatcgtctcgctggcggccactggctcggtattctcgtctgtgttggtggtatgtgcagtgctgcaggcatgttgagcggcttggttgcgacctcatcgcgggcgttgtatgggatgacccgcaagaatatgttgccgccagagctcggtgcgttgcatcaacgatttcgtaccccttggatttgtatcctcttgattgggctgggatcagcctgcttcactgcgctaccctttaacgtactaatccagatagactcgacgttgtactgtctgaaagtcgcattggagttcatcgcgcttgcggtactgcgacggaaatggcccgatcgacagcgaccattccggattggcggaggtatctgggggctctcctatgttgtcggcagtgggctagtctgttgcctggcgatggcggtgctcagtggtctctggtcagcgatttcagcagcagtgaccatcgtgaccggcctcgtgctttattcggtgatcgggcgagttctcaacggtcgatggatgcccagcaccatcgctccgacacctacctcaactccagtggagatataa

SEQ ID NO: 197 - protein, 490 aa, Cyanidiococcus yangmingshanensis
MTVPFSEQAMVSTSSKLTNTAFDGLAIEEGAETDATAVAVVAPDKVSPPRSLGLLQLVGMLFLLTSGGGYGLEPLVGSAGPRWSILALVVAPWLWALPQALMASELATLIPEDGGYVIWVEAALGSFWGFQQGWWSFVDSLVDNALFPRLFSDYTLRVAPAFGMLGSWMVGVLVLVICTLVNILGLGLVGWAAVLFTVLVVSPFMFICIVGFRRTVLSSLWQARPLHEINWRLFLASLLWNWCGFDSCSTVAGEIRDVHRSFPRAMVIVLSLTTMVFTLPIAASVTVNHVWSEWRDAFWPTAANRLAGGHWLGILVCVGGMCSAAGMLSGLVATSSRALYGMTRKNMLPPELGALHQRFRTPWICILLIGLGSACFTALPFNVLIQIDSTLYCLKVALEFIALAVLRRKWPDRQRPFRIGGGIWGLSYVVGSGLVCCLAMAVLSGLWSAISAAVTIVTGLVLYSVIGRVLNGRWMPSTIAPTPTSTPVEI

(19) United States
(12) Patent Application Publication
(10) Pub. No.: US 2025/0137018 A1
(43) Pub. Date: May 1, 2025

(54) BIO-BASED TAURINE PRODUCTION

(71) Applicant: Natáur LLC, Baltimore, MD (US)

(72) Inventors: Frank TURANO, Baltimore, MD (US); Kathleen TURANO, Baltimore, MD (US)

(73) Assignee: Natáur LLC, Baltimore, MD (US)

(21) Appl. No.: 18/834,802

(22) PCT Filed: Jan. 31, 2022
(86) PCT No.: PCT/US2022/014507

§ 371 Date: Jul. 31, 2024

(51) Int. Cl. C12P 13/04; C12N 15/52; C12N 15/70; C12R 1/19

(52) U.S. Cl. CPC C12P 13/04; C12N 15/52; C12N 15/70; C12R 2001/19

(57) ABSTRACT

The present invention describes methods for the fermentative production of taurine-containing products from unicellular organisms. More particularly, the invention relates to genetic modifications of the taurine and/or substrate biosynthetic pathways in unicellular organisms that include bacteria, algae, microalgae, diatoms, yeast, or fungi. The invention also relates to fermentation and processing methods for the production of various taurine-containing products. The invention also relates to the use of the cells, fermentation broth or extracts that contain taurine to produce products for use in food, feed, beverages, dietary and health supplements, cosmetics, personal care, pharmaceuticals, or agricultural production.

### SEQUENCE SUBMISSION

The present application is being filed along with a Sequence Listing in electronic format. The Sequence Listing is entitled Bio-based Taurine Production_2.txt, created on Jan. 30, 2022 and is 443 kb in size. The information in the electronic format of the Sequence Listing is incorporated herein by reference in their entirety.

### FIELD OF THE INVENTION

The present invention is in the field of production of taurine by unicellular organisms.

### BACKGROUND OF THE INVENTION

### Taurine, an Essential Nutrient for Humans and Animals

Taurine, a sulfonic acid, is an essential nutrient for humans and animals (1-6); it is needed for cardiovascular, skeletal muscle, vision, and nervous system function (7) and has been linked with overall human wellness and longevity (1). Taurine is used as an ingredient, required in some cases by the FDA, in numerous products including infant formula, pet food, animal feed, energy drinks, nutraceuticals, pharmaceuticals, personal care/cosmetics, and plant growth enhancers. Taurine is naturally occurring in meat and other animal products, (8) but as we shift to more plant-based food and feed diets, taurine must be added as an ingredient or taken as a supplement (5, 9, 10).

Currently, nearly all supplemental taurine is made from a petroleum-based process (11). A real need exists for a biologically synthesized, safe and sustainable source of taurine that can be economically produced on a commercial scale.

The present invention provides methods for a cost-effective fermentative production of taurine by unicellular organisms. Methods are presented for the optimization of taurine production through genetic improvements of unicellular organisms, growth and fermentation conditions, cost-effective nutrient media and downstream processing for taurine purification.

### Taurine Biosynthetic Pathways

Several taurine biosynthetic pathways have been identified. The genes and their corresponding gene products and methods for the use of genes and the corresponding peptides to make taurine in cells have been described in the literature (12-20). In brief, Pathway 1: Cysteine and oxygen are converted into 3-sulfinoalanine by cysteine dioxygenase (CDO) or CDO homologues (21). 3-sulfinoalanine is converted into hypotaurine by sulfinoalanine decarboxylase (SAD), glutamate decarboxylase (GAD) (22, 23), or by a portion of the cysteine synthetase/PLP decarboxylase (partCS/PLP-DC) (16). Hypotaurine is converted into taurine by a spontaneous conversion or by the activity of a yet to be identified hypotaurine dehydrogenase (HTDeHase). Pathway 2: Cysteamine and oxygen are converted into hypotaurine by cysteamine dioxygenase (ADO), and hypotaurine is converted into taurine. Pathway 3: Cysteine and sulfite are converted into cysteate and hydrogen sulfide by cysteine lyase. Cysteate is converted into taurine by SAD (24) or cysteine sulfonic acid decarboxylase (CAD). Pathway 4: O-phosphoserine and sulfite are converted into cysteate by threonine synthase (TS) (25). Cysteate is then converted into taurine by either SAD or GAD. Pathway 5: Serine can be converted into 2-aminoacrylate by serine dehydratase (SDH) (26). Then 2-aminoacrylate and 3′-phosphoadenosine-5′-phosphosulfate (PAPS) are converted into cysteate by 3′-phosphoadenylyl sulfate: 2′-aminoacrylate C-sulfotransferase (PAPS-AS). Cysteate is converted into taurine by either SAD or GAD (26, 27). Pathway 6: Cysteine synthetase/PLP decarboxylase (CS/PLP-DC) converts O-acetylserine and hydrogen sulfide or 2-aminoacrylate and PAPS into taurine.

The genes and corresponding peptides involved in taurine synthesis in the algal and microalgal species (28) include cysteine dioxygenase ((DO)), glutamate decarboxylase (GAD), sulfinoalanine decarboxylase (SAD), cysteate synthase (('S), cysteine synthetase/PLP decarboxylase (('S PLP-DC) or a portion of the cysteine synthetase/PLP decarboxylase (partCS PLP-DC).

### Precursor Pathways

Similarities among the taurine biosynthetic pathways arise from the requirement of carbon, nitrogen, and sulfur in taurine production. Carbon and nitrogen are supplied from components of the serine-based pathways. Sulfur (sulfate and thiosulfate) is supplied to the cell through a series of reactions that involve uptake, reduction and assimilation. Carbon from glucose enters the serine biosynthetic pathway by conversion of glycerate1,3-bisphosphate into glycerate 3-phosphate by the pgk gene product phosphoglycerate kinase (29). Glycerate 3-phosphate is converted into 3-phosphohydroxypyruvate by the product of serA, 3-phosphoglycerate dehydrogenase. The serA gene product is sensitive to feedback inhibition by serine, however, the inhibition can be removed by the deletion of the last 197 amino acids (serA.\197) (30). 3-phosphohydroxypyruvate is converted into O-phospho-serine by the product of ser (′, phosphoserine aminotransferase, and O-phospho-serine is converted into serine by the product of serB, phosphoserine phosphatase. Serine and acetyl-CoA are converted into O-acetyl-serine by the product of cysE, serine acetyltransferase. The cysE gene product is sensitive to feedback inhibition by cysteine, however, a mutated cysE_{M201R} is insensitive to cysteine inhibition (31). O-acetyl-serine is converted into cysteine by the product of cysK, cysteine synthase. Cysteine can be degraded by the product of tna (32). Other serine-based taurine precursors are derived from the above-named compounds. The precursor, 2-aminoacrylate, is produced from serine by threonine dehydratase, a product of ilvA (28) or serine dehydratase (26). The ilvA gene product is sensitive to feedback inhibition by isoleucine, however, a mutated ilvA_{L447F} is insensitive to isoleucine inhibition (33). 2-aminoacrylate is converted into 2-ketobutyate by the products of RidA or tdcF, 2-iminobutanoate/2-iminopropanoate deaminases or the product of rutC, aminoacrylate peracid reductase.

Sulfur-based precursors for taurine biosynthesis come from the sulfur (sulfate and thiosulfate) uptake and reduction pathways. The sulfate-thiosulfate uptake pathway is controlled by the products of sbp, cysP, cyst), cysW, and cysA. Sulfate and thiosulfate are bound by the products of sbp and cysP, respectively, and transported into the cell by the products of cyst), cysW, and cysA (34). Sulfate is converted into 3′-phosphoadenosine-5′-phosphosulfate (PAPS) by the products of cysDNC, ATP sulfurylase and APS kinase. PAPS is converted into adenosine-3′,5′-diphosphate (PAP) and sulfite by the product of cysH, PAPS reductase. The product of cysQ, PAP nucleotidase, is involved in PAPS regeneration. Sulfite is converted into sulfide by the products of cysIJ. O-acetyl-L-serine and sulfide are converted into cysteine by CysK and CysM. CysM also synthesizes S-sulfocysteine from O-acetyl-L-serine and thiosulfate (35). The S-sulfocysteine is converted into cysteine by glutaredoxin (NrdH) or Grx.

### Taurine and Sulfonic Acid Degradation

In the absence of sulfur, bacteria utilize the sulfonic acid uptake and degradation pathway or the taurine uptake and degradation pathway to mobilize carbon, nitrogen or sulfur (36-39). Genes and their corresponding peptides involved in the uptake and degradation of taurine are usually on the same operon, tauABCD (40) and ssuEADCB (41), and induced in the absence of nitrogen (42, 43) or sulfur (36) or in the presence of taurine (39, 44). In other bacteria, such as C. glutamicum, the genes and their corresponding peptides involved in sulfonic acid, taurine, uptake and degradation are in the ssuDICBA and sueABCD2 operons (45).

The genes for the degradation enzymes, tauX and tauY, encode taurine dehydrogenase (TDH) (43). tauD encodes taurine dioxygenase (TDO) (36), tpa encodes taurine-pyruvate aminotransferase (TPAT) (46), and ssuD and ssuE encode the two-component alkanesulfonate monooxygenase, 2CASM (37).

### Transcriptional Regulators

Several global regulators of sulfur metabolism exist in bacteria. The cysB gene product is a LysR-type transcriptional activator of genes involved in sulfur uptake and reduction and cysteine metabolism. CysB is highly conserved in gram-negative bacteria (47). In Corynebacterium glutamicum, a transcriptional regulator, methionine/cysteine biosynthetic repressor (McbR) (48), represses the expression of genes involved in sulfur assimilation and cysteine biosynthesis. The translational regulators, Cbl and TauR, control the expression and induction of the taurine degradation pathways in bacteria (36, 46). Cbl is a LysR-type transcriptional regulator of the sulfonic acid uptake and degradation pathway or the taurine uptake and degradation pathway in several bacteria (41, 49). The cbl gene is found in Proteobacteria including members of the Alphaproteobacteria, Betaproteobacteria, and Gammaproteobacteria. Bacteria that lack Cbl transcriptional regulators have a McbR subfamily of activators, which include TauR, that control the taurine uptake and degradation system. TauR is found in Rhizobiales and Rhodobacterales of the Alphaproteobacteria, in Burkholderiaceae and Comamonadaceae of the Betaproteobacteria, in Enterobacteriales, Oceanospirillales and Psychromonadales from the Gammaproteobacteria, and in Rhizobiales and Rhodobacter of the Alphaproteobacteria.

### Taurine Exporters

Taurine can be exported outside the cell by the products of gadC, yhiM, or AAperm.

### Fermentation Conditions and Nutrient Media

In the described invention, taurine is produced by fermentation. Methods to produce chemical compounds by batch fermentation, fed-batch fermentation, continuous fermentation or in tanks or ponds are well known to one with ordinary skill in the art (50-60).

The culture medium to be used in the present invention is dependent upon the requirements of the microorganism used in production. Descriptions of defined media for various microorganisms are found in the literature (61-63) Carbon sources can be used individually or combined and can include sugar and carbohydrates such as glucose, sucrose, lactose, fructose, maltose, molasses, starch and cellulose, oils and fats, fatty acids, alcohols, and organic acids. Nitrogen sources can be used individually or as a mixture and can include organic nitrogen-containing compounds such as peptones, tryptone, casein amino acids, yeast extract, meat extract, malt extract, corn steep liquor, soybean meal and urea or inorganic compounds such as ammonium sulfate, ammonium chloride, ammonium phosphate, ammonium carbonate and ammonium nitrate Potassium and phosphate sources can include potassium chloride, monopotassium phosphate, dipotassium phosphate, monosodium phosphate, and disodium phosphate. Magnesium sulfate or iron sulfate, micronutrients, amino acids and vitamins are also necessary for growth.

To control the pH of the culture, compounds such as sodium hydroxide, potassium hydroxide, ammonia, ammonium hydroxide or acids such as phosphoric acid or sulfuric acid are used. To control the foaming, anti-foaming agents are used. Aerobic conditions are maintained by mixing or introducing air or oxygen into the culture. The dissolved oxygen is 15% to 40%, depending on the growth phase and microorganism. The temperature of the culture is 25° C. to 40° C., preferably at 30° C. to 37° C., depending on the microorganism. Growth of the cell culture is maintained until maximum taurine production is reached, typically within 10 hours to 100 hours, preferably 15 hours to 30 hours.

In the described invention, the fermentation broth contains taurine, the cell mass of the microorganism, organic byproducts of the fermentative process, and any remaining components of the medium.

The concentration of the synthesized taurine can be determined at various times throughout fermentation using thin layer chromatography (TLC), amino acid analyzers, high-performance liquid chromatography (HPLC), mass spectrometry (MS), electrospray ionization mass spectrometry (ESI-MS), and liquid chromatography tandem mass spectrometry (LC-MS/MS).

### Downstream Processing: Separation and Purification

In the described invention, taurine is processed or purified to make a product. The specific downstream processing to be used is dependent upon several factors including whether taurine exists in the cells (or biomass) or in the liquid, the form of the desired final taurine product such as liquid or powder, and the desired purity and/or moisture level. In some product applications, the processing may include drying the cells and media to the appropriate concentration and dryness. In some product applications, the processing may include purifying or partially purifying the taurine. To decrease cost and increase efficiency, the volume can be decreased at various times throughout downstream processing by concentrating or removing water by evaporation, using e.g. a falling film evaporator, reverse osmosis or nanofiltration.

If the taurine is in the liquid of the fermentation broth, the liquid can be separated from the biomass by centrifugation, filtration, decantation or a combination thereof. Additional processing of the taurine-containing liquid may include concentration or drying or a purification step for the manufacturing of a taurine product according to the invention. The purification step may be selected from the group consisting of chromatographic techniques (54) or membrane-based processes (64) including ion exchange chromatography (64), ultra-filtration, precipitation, pH adjustment and nanofiltration (65), treatment with activated carbon (66) or crystallization. The purification step or any combination thereof may be repeated until the taurine is purified to the desired specification such as for purity and moisture.

If the taurine is in the cells of the fermentation broth, the cells can be separated from the liquid by centrifugation, filtration, decantation or a combination thereof. The taurine-containing cells can be concentrated and used as a product or the cells can be disrupted by chemical agents, pressure, mechanical force, or ultrasonification to release their contents. The disrupted cells with their contents can be concentrated or dried and used as a product or the contents can be further processed to produce single cell proteins that can be concentrated or dried for use as a product. Alternatively, taurine in the disrupted cells can be separated from the cellular debris by centrifugation, filtration or decantation or a combination thereof, followed by further purification as described above.

If the taurine is in both the liquid and the cells in the fermentation broth, the liquid and cells can be separated, and treated separately, as described above or concentrated together. The taurine-containing concentrate can be used for the manufacturing of a product according to the invention or further processed by purification as described above.

The taurine-containing product can be in different forms such as liquid, powder, paste, capsule or tablet.

### SUMMARY OF THE INVENTION

The invention provides methods for the fermentative production of taurine-containing products in unicellular organisms. More particularly, the invention encompasses the use of polynucleotides for taurine biosynthetic enzymes in combination with polynucleotides for serine biosynthesis and sulfur (sulfate or thiosulfate) uptake, reduction and assimilation and/or the use of polynucleotides for peptides that degrade or transport taurine to increase taurine in cells or export taurine into the media. The invention also relates to fermentation and processing methods for the production of various products produced from the cells, fermentation broth or extracts that contain taurine.

For purposes of promoting an understanding of the principles of the invention, reference will now be made to particular embodiments of the invention and specific language will be used to describe the same. The materials, methods and examples are illustrative only and not limiting.

In some embodiments, the unicellular organisms contain one or more exogenous polynucleotides that is operably linked to a promoter. In other embodiments, the expression of the endogenous polynucleotides of the unicellular organisms is modified with an exogenous promoter.

In one embodiment, the invention consists of unicellular organisms that have a taurine biosynthetic pathway containing the exogenous polynucleotides, CDO and SAD, and a modified serine-based pathway to have increased expression of pgk, ser_{Δ4197}, serC, serB, cysE, and cysK, and a modified sulfur-based pathway to have increased expression of cysPUWA, cysDNC, cysQ, cysH and cysIJ, and knock-outs of tauD, ssuD, and ssuE to inhibit taurine degradation or knock-outs of tauABCD, ssuEADCB, ssuDICBA or sueABCD2 to inhibit taurine degradation and reuptake of taurine into the cell.

In another embodiment, the invention consists of unicellular organisms that have a taurine biosynthetic pathway containing the exogenous polynucleotide, CS PLP-DC, and a modified serine-based pathway to have increased expression of pgk, serA_{Δ197}, serC, and serB, and a modified sulfur-based pathway to have increased expression of cysDNC and cysQ, and knock-outs of tauD, ssuD, and ssuE to inhibit taurine degradation or knockouts of tauABCD, ssuEADCB, ssuDICBA or sueABCD2 to inhibit taurine degradation and reuptake of taurine into the cell.

In another embodiment, the invention consists of unicellular organisms that have a taurine biosynthetic pathway containing the exogenous polynucleotide, CS PLP-DC, and a modified serine-based pathway to have increased expression of serA 4197, and knockouts of tauABCD, ssuEADCB, ssuDICBA or sueABCD2 to inhibit taurine degradation and reuptake of taurine into the cell.

In another embodiment, the invention consists of unicellular organisms that have a taurine biosynthetic pathway containing the exogenous polynucleotides, TS and SAD, and a modified serine-based pathway to have increased expression of pgk, serA_{Δ197}, and serC, and a modified sulfur-based pathway to have increased expression of sbp, cysUWA, cysDNC, cysQ, and cysH, and knock-outs of tauD, SsuD, and SsuE to inhibit taurine degradation and knock-out of cuyA to inhibit cysteate degradation.

In another embodiment, the invention consists of unicellular organisms that have a taurine biosynthetic pathway containing the exogenous polynucleotides, ilvA and PAPS-AS, and a modified serine-based pathway to have increased expression of serA_{Δ197}, serC, serB, and a modified sulfur-based pathway to have increased expression of sbp, cysUWA, cysDNC, and cysQ, and knock-outs of tauD, ssuD, and ssuE to inhibit taurine degradation.

In another embodiment, the invention consists of unicellular organisms that have a taurine biosynthetic pathway containing the exogenous polynucleotides, ilvA_{L447F} and PAPS-AS, taurine exporters, gadC, yhiM, and AAperm, a modified serine-based pathway to have increased expression of serA_{Δ197}, serC, serB, a modified sulfur-based pathway to have increased expression of cysPUWA, cysDNC, and cysQ, knock-outs of tauD, ssuD, and ssuE to inhibit taurine degradation, and knock-outs of ridA, tdcF, and rutC to inhibit 2-aminoacrylate degradation.

In certain embodiments, the invention includes modified or mutant unicellular organisms including bacteria, yeast, fungi, or unicellular algae that produce taurine for use in food, feed, beverages, dietary and health supplements, cosmetics, personal care, pharmaceuticals, or agricultural production.

In certain embodiments, the invention also describes methods to grow the cells by fermentation and describes media formulations in which to grow the cells for the production of taurine or a taurine product that may be a liquid, powder, paste, capsule or tablet.

In certain embodiments, the unicellular organism is E. coli, which is grown in a media that contains at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, at least 0.05 g/L yeast extract, and at least 0.25 mg/L pyridoxal 5′-phosphate (PLP).

In certain embodiments, the invention relates to methods to process the cells or the media in which the cells were grown to make a range of products that include pure taurine or a taurine-containing product. The method can include isolating the taurine to produce taurine having a purity level of greater than 10% purity, greater than 25% purity, greater than 50% purity, greater than 75% purity, or greater than 98% purity.

### BRIEF DESCRIPTION OF THE FIGURES

FIG. 1 exemplifies pathways for taurine production in a unicellular organism (outer dotted rectangle). Genes are designated in bold text and molecules are in normal text. The taurine pathways are indicated by bold lines, and the serine, cysteine, sulfur and degradative pathways are indicated by thin lines. Genes that encode for taurine uptake and degradation are shown in the square. The spontaneous conversion of hypotaurine to taurine is indicated by an *.

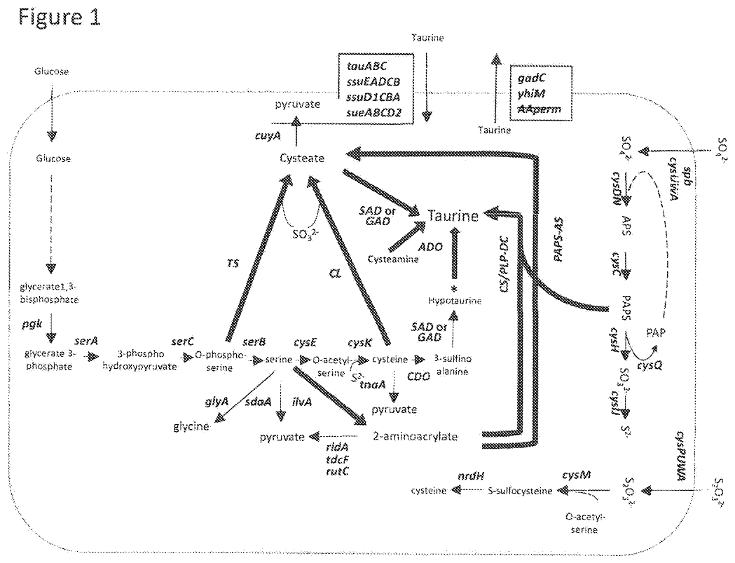

FIG. 2. Chromatogram from HPLC illustrating purified taurine.

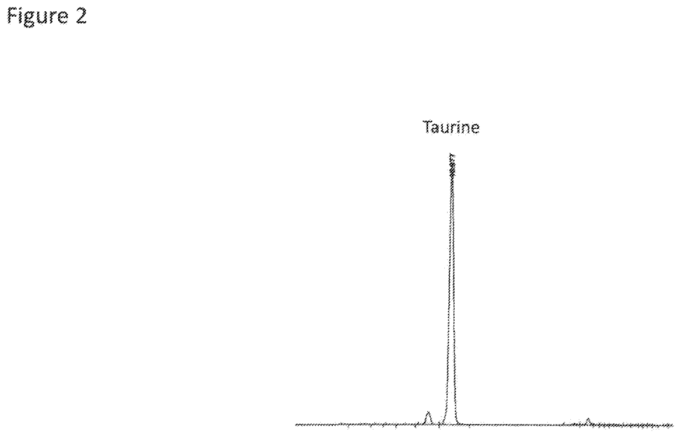

### DETAILED DESCRIPTION OF THE INVENTION

The present invention provides methods for the production of taurine (2-aminoethanesulfonic acid) in unicellular organisms. In preferred embodiments, the invention provides methods for the genetic modification of unicellular organisms using genes that encode proteins in the taurine biosynthetic pathway, the serine biosynthetic pathway, and for the increased transport, reduction and assimilation of sulfur together with silenced or knocked-out genes for the degradation of taurine or precursors or knocked-out operons for taurine uptake and degradation. The invention also provides methods of using unicellular organisms including bacteria, microalgae, fungi, yeast, and algae with increased levels of endogenous taurine or taurine derivatives such as hypotaurine for use in food, feed, beverages, dietary and health supplements, cosmetics, personal care, pharmaceuticals, or agricultural production.

This invention presents methods for the modification of unicellular organisms by including one or more exogenous polynucleotides for peptides from one or more taurine biosynthetic pathway consisting of the groups: Group 1: CDO and SAD, GAD or partCS/PLP-DC; Group 2: ADO; Group 3: cysteine lyase and SAD or GAD; Group 4: TS and SAD or GAD; Group 5: ilvA_{L447F} and PAPS-AS and SAD; or Group 6: CS/PLP-DC.

This invention presents methods for the modification of unicellular organisms that increase the expression of one or more polynucleotides for peptides in serine-based or sulfate-based pathways comprising of: pgk, serA_{Δ197}, serC, serB, cys_{EM201R}, cysK, cysM, nrdH, sbp, cysUWA, cysPUWA, cysDNC, cysQ, cysH, and cysIJ.

This invention presents methods for the modification of unicellular organisms that block taurine uptake and degradation by silencing, mutating or knocking out one or more of the following operons: tauABC, ssuEADCB, ssuDICBA or sueABCD2.

This invention presents methods for the modification of unicellular organisms that block taurine by methods of silencing, mutating or knocking out one or more of the following genes: tauX, tauY, tauD, tpa, ssuD, ssuE, or ssu1.

This invention presents methods for the modification of unicellular organisms that block precursor degradation by methods of silencing, mutating or knocking out one or more of the following: genes for the 2-aminoacrylate degradation enzymes: ridA, tdcF and rutC, gene for the cysteate degradation enzyme: cuyA, and genes for the serine degradation enzymes: glyA, sdaA, and ilvA.

This invention presents methods for the modification of unicellular organisms to control the expression of one or more translational regulator genes, cbl, cysB, tauR, or mcbR, in the serine-based, sulfate-based, or taurine pathways.

This invention presents methods for the modification of unicellular organisms by including one or more exogenous polynucleotides from the group consisting of the following genes: gadC, yhiM, and AAperm, for peptides that transport taurine out of the cell.

Below is a list of suitable polynucleotides that are suitable for each gene in certain embodiments. Other suitable polynucleotides for use in accordance with the invention may be obtained by the identification of polynucleotides by selective hybridize to the polynucleotides to the named polypeptide by hybridization under low stringency conditions, moderate stringency conditions, or high stringency conditions. Still other suitable polynucleotides for use in accordance with the invention may be obtained by the identification of similar polynucleotides that have substantial identity of the nucleic acid of or encode polypeptides that have substantial identity to amino acid sequence of when it used as a reference for sequence comparison.

Suitable polynucleotides for CDO are provided in SEQ ID NO:1; SEQ ID NO:3; SEQ ID NO: 5; SEQ ID NO:7 and encode the peptides with amino acid sequences of SEQ ID NO:2; SEQ ID NO:4; SEQ ID NO:6; SEQ ID NO:8, respectively.

Suitable polynucleotides for SAD are provided in SEQ ID NO:9; SEQ ID NO:11; SEQ ID NO: 13 and encode the peptides with amino acid sequences of SEQ ID NO: 10; SEQ ID NO: 12; SEQ ID NO:14, respectively.

A suitable polynucleotide for GAD is provided in SEQ ID NO:15 and encodes the peptide with amino acid sequence of SEQ ID NO:16.

Suitable polynucleotides for CS PL_DC are provided in SEQ ID NO: 17; SEQ ID NO: 78 and encode the peptides with amino acid sequences of SEQ ID NO: 18; SEQ ID NO: 79, respectively.

A suitable polynucleotide for ADO is provided in SEQ ID NO: 19 and encodes the peptide with amino acid sequence of SEQ ID NO:20.

Suitable polynucleotides for CL are provided in SEQ ID NO:21; SEQ ID NO:23 and encode the peptides with amino acid sequences of SEQ ID NO:22; SEQ ID NO:24, respectively.

Suitable polynucleotides for TS are provided in SEQ ID NO:25; SEQ ID NO:27 and encode the peptides with amino acid sequences of SEQ ID NO:26; SEQ ID NO:28, respectively.

Suitable polynucleotides for ilvA are provided in SEQ ID NO: 136; SEQ ID NO: 140 and encode the peptides with amino acid sequences of SEQ ID NO: 137; SEQ ID NO:141, respectively.

A suitable polynucleotide for ilvA_{L447F} is provided in SEQ ID NO:29 and encodes the peptide with amino acid sequence of SEQ ID NO:30.

Suitable polynucleotides for PAPS-AS are provided in SEQ ID NO:31; SEQ ID NO: 33 and encode the peptides with amino acid sequences of SEQ ID NO:32; SEQ ID NO:34, respectively.

A suitable polynucleotide for pgk is provided in SEQ ID NO:35 and encodes the peptide with amino acid sequence of SEQ ID NO:36.

A suitable polynucleotide for serA 4197 is provided in SEQ ID NO:37 and encodes the peptide with amino acid sequence of SEQ ID NO:38.

A suitable polynucleotide for serB is provided in SEQ ID NO:39 and encodes the peptide with amino acid sequence of SEQ ID NO:40.

A suitable polynucleotide for serC is provided in SEQ ID NO:41 and encodes the peptide with amino acid sequence of SEQ ID NO:42.

A suitable polynucleotide for cysE_{M201R} is provided in SEQ ID NO:43 and encodes the peptide with amino acid sequence of SEQ ID NO:44.

Suitable polynucleotides for cysk are provided in SEQ ID NO:45; SEQ ID NO:147 and encode the peptides with amino acid sequences of SEQ ID NO:46; SEQ ID NO:148, respectively.

A suitable polynucleotide for cysDNC is provided in SEQ ID NO:47 and encodes the peptides with amino acid sequences of SEQ ID NO:48; SEQ ID NO:49; SEQ ID NO:50.

A suitable polynucleotide for cysQ is provided in SEQ ID NO:51 and encodes the peptide with amino acid sequence of SEQ ID NO:52.

A suitable polynucleotide for cysH is provided in SEQ ID NO:53 and encodes the peptide with amino acid sequence of SEQ ID NO:54.

A suitable polynucleotide for cysIJ is provided in SEQ ID NO:55 and encodes the peptides with amino acid sequences of SEQ ID NO:57; SEQ ID NO:56.

A suitable polynucleotide for cysB is provided in SEQ ID NO:58 and encodes the peptide with amino acid sequence of SEQ ID NO:59.

A suitable polynucleotide for tauX is provided in SEQ ID NO:60 and encodes the peptide with amino acid sequence of SEQ ID NO:61.

A suitable polynucleotide for tauY is provided in SEQ ID NO:62 and encodes the peptide with amino acid sequence of SEQ ID NO:63.

A suitable polynucleotide for tauD is provided in SEQ ID NO:64 and encodes the peptide with amino acid sequence of SEQ ID NO:65.

A suitable polynucleotide for tpa is provided in SEQ ID NO:66 and encodes the peptide with amino acid sequence of SEQ ID NO:67.

A suitable polynucleotide for tauABCD is provided in SEQ ID NO:68.

A suitable polynucleotide for ssuEADCB is provided in SEQ ID NO:69.

Suitable polynucleotides for ssuD are provided in SEQ ID NO: 70; SEQ ID NO: 72 and encode the peptides with amino acid sequences of SEQ ID NO:71; SEQ ID NO:73, respectively.

Suitable polynucleotides for ssuE are provided in SEQ ID NO:74; SEQ ID NO:76 and encode the peptides with amino acid sequences of SEQ ID NO:75; SEQ ID NO:77, respectively.

Suitable polynucleotides for ridA are provided in SEQ ID NO:80; SEQ ID NO: 149; SEQ ID NO:151 and encode the peptides with amino acid sequences of SEQ ID NO:81; SEQ ID NO: 150; SEQ ID NO:152, respectively.

A suitable polynucleotide for tdcF is provided in SEQ ID NO:82 and encodes the peptide with amino acid sequence of SEQ ID NO:83.

A suitable polynucleotide for rutC is provided in SEQ ID NO:84 and encodes the peptide with amino acid sequence of SEQ ID NO:85.

A suitable polynucleotide for cuyA is provided in SEQ ID NO: 86 and encodes the peptide with amino acid sequence of SEQ ID NO:87.

Suitable polynucleotides for cbl are provided in SEQ ID NO:88; SEQ ID NO:90 and encode the peptides with amino acid sequences of SEQ ID NO:89; SEQ ID NO:91, respectively.

Suitable polynucleotides for tauR are provided in SEQ ID NO:92; SEQ ID NO:94 and encode the peptides with amino acid sequences of SEQ ID NO:93; SEQ ID NO:95, respectively.

A suitable polynucleotide for mcbR is provided in SEQ ID NO:96 and encodes the peptide with amino acid sequence of SEQ ID NO:97.

A suitable polynucleotide for cysM is provided in SEQ ID NO:98 and encodes the peptide with amino acid sequence of SEQ ID NO:99.

Suitable polynucleotides for sdaA are provided in SEQ ID NO:100; SEQ ID NO:102 and encode the peptides with amino acid sequences of SEQ ID NO:101; SEQ ID NO:103, respectively.

Suitable polynucleotides for glyA are provided in SEQ ID NO: 104; SEQ ID NO:106 and encode the peptides with amino acid sequences of SEQ ID NO: 105; SEQ ID NO:107, respectively.

A suitable polynucleotide for tnaA is provided in SEQ ID NO: 108 and encodes the peptide with amino acid sequence of SEQ ID NO:109.

A suitable polynucleotide for cysPUWA is provided in SEQ ID NO: 110 and encodes the peptides with amino acid sequences of SEQ ID NO:111; SEQ ID NO:112; SEQ ID NO:113; SEQ ID NO:114.

A suitable polynucleotide for nrdh is provided in SEQ ID NO: 143 and encodes the peptide with amino acid sequence of SEQ ID NO:144.

A suitable polynucleotide for sbp is provided in SEQ ID NO: 160 and encodes the peptide with amino acid sequence of SEQ ID NO:161.

A suitable polynucleotide for ssuC is provided in SEQ ID NO: 162 and encodes the peptide with amino acid sequence of SEQ ID NO: 163.

A suitable polynucleotide for ssuB is provided in SEQ ID NO: 164 and encodes the peptide with amino acid sequence of SEQ ID NO:165.

A suitable polynucleotide for ssuA is provided in SEQ ID NO: 166 and encodes the peptide with amino acid sequence of SEQ ID NO:167.

A suitable polynucleotide for ssuDICBA is provided in SEQ ID NO:168.

A suitable polynucleotide for ssu1 is provided in SEQ ID NO:169 and encodes the peptide with amino acid sequence of SEQ ID NO:170.

A suitable polynucleotide for sueA is provided in SEQ ID NO: 172 and encodes the peptide with amino acid sequence of SEQ ID NO:173.

A suitable polynucleotide for sueB is provided in SEQ ID NO:174 and encodes the peptide with amino acid sequence of SEQ ID NO:175.

A suitable polynucleotide for sueC is provided in SEQ ID NO:176 and encodes the peptide with amino acid sequence of SEQ ID NO:177.

A suitable polynucleotide for sueD2 is provided in SEQ ID NO:178 and encodes the peptide with amino acid sequence of SEQ ID NO:179.

A suitable polynucleotide for sueABCD2 is provided in SEQ ID NO:180.

Suitable polynucleotides for gadC are provided in SEQ ID NO:184; SEQ ID NO:186; SEQ ID NO: 188 and encode the peptides with amino acid sequences of SEQ ID NO:185; SEQ ID NO: 187, SEQ ID NO: 189, respectively.

A suitable polynucleotide for yhiM is provided in SEQ ID NO: 190 and encodes the peptide with amino acid sequence of SEQ ID NO:191.

Suitable polynucleotides for amino acid permeases, AAperm, are provided in SEQ ID NO: 192; SEQ ID NO:194; SEQ ID NO:196 and encode the peptides with amino acid sequences of SEQ ID NO: 193; SEQ ID NO: 195; SEQ ID NO: 197, respectively.

The invention is not limited to the use of these amino acid sequences. Amino acid sequences comprising a variation of the enzymes and transcription factors listed are included within the scope of the present invention and are considered substantially or sufficiently similar to a reference amino acid sequence. Although it is not intended that the present invention be limited by any theory by which it achieves its advantageous result, it is believed that the identity between amino acid sequences that is necessary to maintain proper functionality is related to maintenance of the tertiary structure of the polypeptide such that specific interactive sequences will be properly located and will have the desired activity, and it is contemplated that a polypeptide including these interactive sequences in proper spatial context will have activity.

Another manner in which similarity may exist between two amino acid sequences is where there is conserved substitution between a given amino acid of one group. The process of encoding a specific amino acid sequence may involve DNA sequences having one or more base changes (i.e., insertions, deletions, substitutions) that do not cause a change in the encoded amino acid, or which involve base changes which may alter one or more amino acids, but do not eliminate the functional properties of the polypeptide encoded by the DNA sequence.

One of ordinary skill in the art will recognize that changes in the amino acid sequences, such as individual substitutions, deletions or additions to a nucleic acid, peptide, polypeptide, or protein sequence which alters, adds or deletes a single amino acid or a small percentage of amino acids in the encoded sequence is “sufficiently similar” when the alteration results in the substitution of an amino acid with a chemically similar amino acid.

It is therefore understood that the invention encompasses more than the specific polynucleotides encoding the proteins described herein. For example, modifications to a sequence, such as deletions, insertions, or substitutions in the sequence, which produce “silent” changes that do not substantially affect the functional properties of the resulting polypeptide are expressly contemplated by the present invention. It is known by those of ordinary skill in the art, “universal” code is not completely universal. Some mitochondrial and bacterial genomes diverge from the universal code, e.g., some termination codons in the universal code specify amino acids in the mitochondria or bacterial codes. Thus, each silent variation of a nucleic acid, which encodes a polypeptide of the present invention, is implicit in each described polypeptide sequence and incorporated in the descriptions of the invention.

It is understood that alterations in a nucleotide sequence, which reflect the degeneracy of the genetic code, or which result in the production of a chemically equivalent amino acid at a given site, are contemplated. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a biologically equivalent product.

When the nucleic acid is prepared or altered synthetically, one of ordinary skill in the art can take into account the known codon preferences for the intended host where the nucleic acid is to be expressed. For example, although nucleic acid sequences of the present invention may be expressed in different species, sequences can be modified to account for the specific codon preferences and GC-content preferences of the organism, as these preferences have been shown to differ (67-72).

### Cloning Techniques

Unless mentioned otherwise, the techniques employed or contemplated herein are standard methodologies well known to one of ordinary skill in the art. Specific terms, while employed below and defined at the end of this section, are used in a descriptive sense only and not for purposes of limitation. The practice of the present invention will employ, unless otherwise indicated, conventional techniques of botany, microbiology, mycology, phycology, tissue culture, molecular biology, chemistry, biochemistry, biotechnology, and recombinant DNA technology, which are within the skill of the art (73-80).

A suitable polynucleotide for use in accordance with the invention may be obtained by cloning techniques using cDNA or genomic libraries, DNA, or cDNA from bacteria, algae, microalgae, diatoms, yeast or fungi which are available commercially or which may be constructed using standard methods known to persons of ordinary skill in the art. Suitable nucleotide sequences may be isolated from DNA libraries obtained from a wide variety of species by means of nucleic acid hybridization or amplification methods, such as polymerase chain reaction (PCR) procedures, using as probes or primers nucleotide sequences selected in accordance with the invention.

Furthermore, nucleic acid sequences may be constructed or amplified using chemical synthesis. The product of amplification is termed an amplicon. Moreover, if the particular nucleic acid sequence is of a length that makes chemical synthesis of the entire length impractical, the sequence may be broken up into smaller segments that may be synthesized and ligated together to form the entire desired sequence by methods known in the art. Alternatively, individual components or DNA fragments may be amplified by PCR and adjacent fragments can be amplified together using fusion-PCR (81), overlap-PCR (82) or chemical (de novo) synthesis (83-87) using a vendor (e.g. DNA2.0, GE life technologies, GENEART, Gen9, GenScript) by methods known in the art.

The recombinant expression cassette or DNA construct includes a promoter that directs transcription in a unicellular organism, operably linked to the polynucleotide of the invention described herein. A variety of different types of promoters are described and used. As used herein, a polynucleotide is “operably linked” to a promoter or other nucleotide sequence when it is placed into a functional relationship with the promoter or other nucleotide sequence. The functional relationship between a promoter and a desired polynucleotide insert typically involves the polynucleotide and the promoter sequences being contiguous such that transcription of the polynucleotide sequence will be facilitated. Two nucleic acid sequences are further said to be operably linked if the nature of the linkage between the two sequences does not (1) result in the introduction of a frame-shift mutation; (2) interfere with the ability of the promoter region sequence to direct the transcription of the desired nucleotide sequence, or (3) interfere with the ability of the desired nucleotide sequence to be transcribed by the promoter sequence region. Typically, the promoter element is generally upstream (i.e., at the 5′ end) of the nucleic acid insert coding sequence.

While a promoter sequence can be ligated to a coding sequence prior to insertion into a vector, in other embodiments, a vector is selected that includes a promoter operable in the host cell into which the vector is to be inserted. In addition, certain preferred vectors have a region that codes a ribosome binding site positioned between the promoter and the site at which the DNA sequence is inserted so as to be operatively associated with the DNA sequence of the invention to produce the desired polypeptide, i.e., the DNA sequence of the invention in-frame.

Gene expression cassettes may contain one or more polynucleotides (genes), each operably linked with a promoter and terminator to form a series of monocistronic mRNAs or the genes can be arranged with one promoter and terminator to form a single polycistronic mRNA. A wide variety of operable cassettes are known to those of ordinary skill in the art.

### Suitable Promoters

A wide variety of promoters are known to those of ordinary skill in the art, as are other regulatory elements that can be used alone or in combination with promoters. A wide variety of promoters that direct transcription in unicellular organisms can be used in connection with the present invention (88-90). The features (binding sites and regulatory elements) necessary for the identification and use of functional bacterial promoters are known to those of ordinary skill in the art (91-93). For purposes of describing the present invention, promoters are divided into two types, namely, constitutive promoters and non-constitutive promoters (89, 94). Constitutive promoters are classified as providing for a range of constitutive expression. Some are weak constitutive promoters, and others are strong constitutive promoters (95). Other promoters are considered non-constitutive promoters (96-100).

### Terminators

In addition to the selection of a suitable promoter, the DNA constructs require an appropriate transcriptional terminator to be attached downstream (3′), after the stop codon (TGA, TAG or TAA) of the desired gene of the invention for proper expression in unicellular organisms. Several such terminators are available and known to persons of ordinary skill in the art. Terminators play an important role in the processing and stability of RNA as well as in translation and may also control gene expression (101-110). The identification and use of terminators that are required to express genes in unicellular organisms are known to those of ordinary skill in the art.

### Selectable Markers

Selectable markers usually confer resistance to an antibiotic, herbicide or chemical or provide color change, which aid the identification of transformed organisms. The vectors may also include a RNA stability signal, which are 3′-regulatory sequence elements that increase the stability of the transcribed RNA (111, 112).

### Plastid Transit Peptides

The invention can be targeted for transformation into the chloroplast. Chloroplast targeted transformation systems for algae are known by those of ordinary skill in the art (97, 99, 113-115).

A wide variety of plastid transit peptides are known to those of ordinary skill in the art that can be used in connection with the present invention. Suitable transit peptides which can be used to target any CDO, SAD, GAD, CS/PLP-DC, partCS/PLP-DC, TauA, or TauK polypeptide to a plastid include, but are not limited, to those described herein and in U.S. Pat. Nos. 8,779,237 (116), 8,674,180 (117), 8,420,888 (118), and 8,138,393 (119), and in Lee et al. (120) and von Heijne et al. (121). Identification and use of chloroplast plastid targeting sequences for algae are known to those of ordinary skill in the art (122-125). Cloning a nucleic acid sequence that encodes a transit peptide upstream and in-frame of a nucleic acid sequence that encodes a polypeptide involves standard molecular techniques that are known to those of ordinary skill in the art.

### Suitable Vectors

A wide variety of vectors may be employed to transform a unicellular organism with a construct made or selected in accordance with the invention, including high- or low-copy number plasmids, phage vectors and cosmids. Vector systems, expression cassettes, culture methods, and transformation methods are known by those of ordinary skill in the art. The vectors can be chosen such that operably linked promoter and polynucleotides that encode the desired polypeptide of the invention are incorporated into the genome of the unicellular organism. Other vectors that can operably link promoter and polynucleotides that encode the polypeptide of the invention are incorporated are not incorporated into the host genome but the vector DNA with the clone polynucleotides are autonomously or semi autonomously replicated in the cell. Although the preferred embodiment of the invention is expressed in unicellular organisms, other embodiments may include expression in prokaryotic or unicellular eukaryotic organisms including, but not limited to, yeast, fungi, algae, microalgae, or microbes.

It is known by those of ordinary skill in the art that there exist numerous expression systems available for expression of a nucleic acid encoding a protein of the present invention. There are many commercially available recombinant vectors to transform a unicellular organism. Standard molecular and cloning techniques (77, 80, 126) are available to make a recombinant expression cassette that expresses the polynucleotide that encodes the desired polypeptide of the invention. No attempt will be made to describe in detail the various methods known for the expression of proteins in prokaryotes or eukaryotes. In brief, the expression of isolated nucleic acids encoding a protein of the present invention will typically be achieved by operably linking, for example, the DNA or cDNA to a promoter, followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high-level expression of a cloned gene, it is desirable to construct expression vectors that contain, at the minimum, a strong promoter, to direct transcription, a ribosome-binding site for translational initiation, and a transcription/translation terminator.

### Expression in Prokaryotes

Protocols for transformation as well as commonly used vectors with control sequences including promoters for transcription initiation (some with an operator), together with ribosome binding site sequences for use in prokaryotes are known to those of ordinary skill in the art. Those of ordinary skill in the art know the molecular techniques and DNA vectors that are used in bacterial systems (127-131). In bacteria one messenger RNA can encode for one peptide (referred to as monocistronic) or several independent peptides (referred to as polycistronic). It is known to those of ordinary skill in the art that a portion of a polycistronic messenger RNA can be knocked-out (132) or that heterologous or exogenous genes can be expressed on a monocistronic or polycistronic messenger RNA (130, 131). Genes can be expressed by modification of bacterial DNA (genomic) through the use of knock-in, gene insertion, or by allelic exchange (133-138). Specific gene targeting has been used in bacteria using PCR-based methods (139), and CRISPR/Cas (140-142).

### Expression in Algae and Microalgae

Protocols for transformation as well as commonly used vectors with control sequences include promoters for transcription initiation, optionally with an operator, together with ribosome binding site sequences for use in algae and microalgae are known to those of ordinary skill in the art (89, 113, 143-153). Specific gene targeting systems have been used in algae including ZFNs (154) and transcription activator-like effector nucleases (TALENs) (155).

### Expression in Non-Plant Eukaryotes

Protocols for transformation, as well as commonly used vectors, are known to those of ordinary skill in the art. Also known to those of ordinary skill in the art are control sequences that include promoters for transcription initiation and ribosome binding site sequences for use in unicellular eukaryotes. The present invention can be expressed in a variety of eukaryotic expression systems such as yeast and protozoa. The vectors usually have expression control sequences, such as promoters, an origin of replication, enhancer sequences, termination sequences, ribosome binding sites, RNA splice sites, polyadenylation sites, transcriptional terminator sequences, and selectable markers (156, 157). There are numerous vectors that can be used with the invention that are known to those of ordinary skill in the art and include, but are not limited to, pREP, pRIP, pD912, pD1201, pD1211, pD1221, pD1231, pYES2/NT, pYSG-IBA, or pESC-TRP. Synthesis of heterologous proteins and fermentation of products in yeast is known to those of ordinary skill in the art (158, 159). Protozoa that can be used include, but are not limited to, ciliates, amoebae and flagellates. Yeast and fungi that can be used with the invention and the molecular protocols for transformation, and the vectors required for expression of genes in these systems, are known to those of ordinary skill in the art (160-165). A range of vectors is available. Also available are plasmid vectors, which may be integrative, autonomously replicating high copy-number vectors, or autonomously replicating low copy number vectors (166, 167). The most common vectors that complement a chromosomal mutation in the host include functional genes such as URA3, HIS3, LEU2, TRP1 and LYS2. Specific gene editing or targeting has been used in unicellular fungi using PCR-based methods (168-170). Zinc-finger nucleases (ZFNs), 171 transcription activator-like effector nucleases (TALENs) (172), and clustered regularly interspaced short palindromic repeats/Cas (CRISPR/Cas) (173, 174).

One of ordinary skill in the art recognizes that modifications could be made to a protein of the present invention without diminishing its biological activity. Some modifications may be made to facilitate the cloning, expression, targeting or to direct the location of the polypeptide in the host, or for the purification. Such modifications are known to those of ordinary skill in the art and include, for example, a methionine added at the amino terminus to provide an initiation site, additional nucleic acids to insert a restriction site or a termination.

In addition, polynucleotides can be placed in the appropriate vector used to transform unicellular organisms. The polypeptide can be expressed and then isolated from transformed cells, or metabolites can be synthetized and isolated from the transformed cells. Such transgenic organisms can be harvested, and subjected to large-scale protein or metabolite (taurine) extraction and purification techniques.

The vector may include another polynucleotide that encodes a signal polypeptide or signal sequence (“subcellular location sequence”) to direct the desired polypeptide in the host cell, so that the polypeptide accumulates in a specific cellular compartment, subcellular compartment, or membrane. The specific cellular compartments include the vacuole, chloroplast (not in fungi), mitochondrion, peroxisomes, secretory pathway, lysosome, endoplasmic reticulum, nucleus or Golgi apparatus in fungi or algae. There are specific signal polypeptides or signal sequences to direct peptide transport to the periplasmic space in bacteria (175-177). A signal polypeptide or signal sequence is usually at the amino terminus and normally absent from the mature protein due to protease that removes the signal peptide when the polypeptide reaches its final destination. Signal sequences can be a primary sequence located at the N-terminus (121, 178-180), C-terminus (181, 182) or internal (183-185) or tertiary structure (185). If a signal polypeptide or signal sequence to direct the polypeptide does not exist on the vector, it is expected that those of ordinary skill in the art can incorporate the extra nucleotides necessary to encode a signal polypeptide or signal sequence by the ligation of the appropriate nucleotides or by PCR. Those of ordinary skill in the art can identify the nucleotide sequence of a signal polypeptide or signal sequence using computational tools. There are numerous computational tools available for the identification of targeting sequences or signal sequence. These include, but are not limited to, TargetP (186, 187), iPSORT (188), SignalP (189), PrediSi (190), ELSpred (191), HSLpred (192) and PSLpred (193), MultiLoc (194), SherLoc (195), ChloroP (196), MITOPROT (197), Predotar (198) 3D-PSSM (199) and PredAlgo (125). Additional methods and protocols are discussed in the literature (194).

### Transformation of Host Cells

Transformation of an unicellular organism can be accomplished in a wide variety of ways within the scope of a person of ordinary skill in the art (88, 90, 151, 200). Those of ordinary skill in the art can use different algal, diatom, fungal, yeast and bacteria gene transfer techniques that include, but not limited to, Agrobacterium-mediated (201) glass beads and polyethylene glycol (PEG) (202, 203), electroporation (204-207), microprojectile bombardment or ballistic particle acceleration (208-212), silicon carbide whisker methods (213, 214), viral infection (215, 216), or transposon/transposase complexes (217). Transformation can be targeted to organellular genomes (115). Other methods to edit, incorporate or move genes into bacteria, fungal algal genomes include, but are not limited to, Zinc-finger nucleases (ZFNs), transcription activator-like effector nucleases (TALENs), or clustered regularly interspaced short palindromic repeats/Cas (CRISPR/Cas).

### Gene Silencing by Mutagenesis or Recombinant Technologies

Genetic modification to silence or inactivate genes or their corresponding gene products of unicellular organisms can be conducted by radiation-, chemical- or UV-based mutagenesis followed by specific screening for biochemical traits or pathways (200, 218-222). Radiation-based mutations can silence or inactive a gene or the corresponding gene product by DNA breakage and repair. Chemical- or UV-based mutations usually result in single DNA basepair changes. Mutations can silence or inactive a gene or the corresponding gene product by one of the following: (1) introduction of a frame-shift mutation; (2) introduction of premature stop codon; (3) interference with the ability of the promoter region sequence to direct the transcription of the desired nucleotide sequence, (4) interference with the ability of the desired nucleotide sequence to be transcribed by the promoter sequence region or (5) introduction of an amino acid substitution in the gene product to reduce or inhibit activity (enzymatic activity or binding) or interfere with the function of the gene product.

Targeted gene silencing or knockouts can be made in unicellular organisms using phage or viruses (94, 223-227), transposons (217, 228-231), PCR-assisted targeting (168-170, 232), recombinases or by allelic exchange (133-138). Targeted and random bacterial gene disruptions can be made using a group II intron (Targetron) (233, 234), ZNFs (171), TALENs (172), CRISPER-Cas9 or clustered regularly interspaced short palindromic repeats interference (CRISPi) (140-142, 173, 174, 235, 236). In addition, RNA-mediated methods (237-242), or regulatory RNAs (243-245) have been used to silence or suppress gene expression in unicellular organisms and these techniques and protocols are well known to one with ordinary skill in the art.

### Suitable Unicellular Organisms

A wide variety of unicellular host cells may be used in the invention, including prokaryotic and unicellular eukaryotic host cells. These cells or organisms may include yeast, fungi, algae, microalgae, microbes, or unicellular photosynthetic organisms. Preferred host cells for this invention are bacteria including, archaebacteria and eubacteria. Proteobacteria such as members of Alphaproteobacteria, Betaproteobacteria, Gammaproteobacteria, Deltaproteobacteria, and Epsilonproteobacteria can host the invention. Other bacteria including Methanotrophs and Methylobacterium (246) can be used with the invention. Other bacterial genera that can host the invention include, but are not limited to Escherichia, Bacillus, Salmonella, Lactococcus, Lactobacillus, Streptococcus, Brevibacterium and Coryneform bacteria. Some specific bacterial species that can be used for the invention include, but are not limited to, Bacillus subtilis, Brevibacterium ammoniagene, Corynebacterium crenatum, Corynebacterium pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans, and Saccharomyces cerevisiae (50).

Unicellular algae, unicellular photosynthetic organisms, and microscopic algae (microphytes or microalgae) cells may be used in the invention. These include, but are not limited to diatoms, green algae (Chlorophyta), and members of the Euglenophyta, Dinoflagellata, Chrysophyta, Phaeophyta, red algae (Rhodophyta), Heterokontophyta, and Cyanobacteria. The invention can also be used to increase the taurine by binding taurine with a taurine binding protein or knocking out genes for taurine degradation in algae that have been shown to synthesize taurine (28) or may have the capability to synthesize taurine (28). These include but are not limited to Coccomyxa species, Chlorella species, Trebouxia impressa, Tetraselmis species, Chlamydomonas reinhardtii, Micromonas pusilla, Ostreococcus tauri, Navicula radiosa, Phaeodactylum tricornutum, Pseudo-nitzschia multiseries, Fragilariopsis cylindrus, Thalassiosira weissflogii, Nannochloropsis oceanica, Aureococcus anophagefferens, Saccharina japonica, Sargassum species and Bigelowiella natans.

Protozoa that may be used in the invention include, but are not limited, to ciliates, amoebae and flagellates. Yeast and unicellular fungi that can be used include, but are not limited to Ashbya gossypii, Blakeslea trispora, Candida flareri, Eremothecium ashbyii, Mortierella isabellina, Pichia pastoris, Saccharomyces cerevisiae, and Saccharomyces pombe.

Once transformed, the unicellular organism may be treated with other “active agents” either prior to or during the growth to further increase production of taurine. “Active agent,” as used herein, refers to an agent that has a beneficial effect on the taurine production by the unicellular organism. Sulfur containing compounds such as sulfite, sulfide, hydrogen sulfide, sulfate, taurine, hypotaurine, cysteate, 2-sulfacetaldehyde, homotaurine, homocysteine, cystathionine, N-acetyl thiazolidine 4 carboxylic acid (ATCA), glutathione, or bile, or other non-protein amino acids, such as GABA, citrulline and ornithine, or other nitrogen containing compounds such as polyamines may also be used to promote taurine production. Depending on the type of gene construct or recombinant expression cassette, other metabolites and nutrients may be used. These include, but are not limited to, sugars, carbohydrates, lipids, oligopeptides, mono-(glucose, arabinose, fructose, xylose, and ribose) di-(sucrose and trehalose) and polysaccharides, carboxylic acids (succinate, malate and fumarate), vitamins, and nutrients such as phosphate, molybdate, or iron.

In some embodiments properties of a transgenic unicellular organism are altered using an agent which increases sulfur concentration in the cell, such as sulfur, sulfite, sulfide, hydrogen sulfide, sulfate, taurine, hypotaurine, homotaurine, cysteate, 2-sulfacetaldehyde, N-acetyl thiazolidine 4 carboxylic acid (ATCA), glutathione, and bile. In other embodiments, the agent increases nitrogen concentration. Amino acids either naturally occurring in proteins (e.g., cysteine, methionine, glutamate, glutamine, serine, alanine, or glycine) or which do not naturally occur in proteins (e.g., GABA, citrulline, or ornithine) and/or polyamines can be used for this purpose.

### Pharmaceutical Compositions

The invention provides pharmaceutical compositions that comprise extracts of one or more modified unicellular organisms described above. Extracts containing hypotaurine or taurine can be used to synthesize or manufacture taurine derivatives (247, 248), taurine-conjugates (249) or taurine-polymers (250) that may have a wide range of commercial and medicinal applications (251). Some taurine derivatives can function as organogelators (252) or dyes (253) and can be used in nanosensor synthesis (254). Some taurine derivatives have anticonvulsant (247) or anti-cancer (255) properties. Other taurine derivatives are used in the treatment of alcoholism (256, 257). Taurine-conjugated carboxyethylester-polyrotaxanes increase anticoagulant activity (258). Taurine-containing polymers may increase wound healing (259, 260). Taurine linked polymers such as poly gamma-glutamic acid-sulfonates are biodegradable and may have applications in the development of drug delivery systems, environmental materials, tissue engineering, and medical materials (261). Extracts from taurine-containing cells may be used in pharmaceutical or medicinal compositions to deliver taurine, hypotaurine, taurine-conjugates, or taurine-polymers for use in the treatment of congestive heart failure, high blood pressure, hepatitis, high cholesterol, fibrosis, epilepsy, autism, attention deficit-hyperactivity disorder, retinal degeneration, diabetes, and alcoholism. It is also used to improve mental performance and as an antioxidant.

Pharmaceutically acceptable vehicles of taurine, taurine derivatives, taurine-conjugates, or taurine-polymers are tablets, capsules, gel, ointment, film, patch, powder or dissolved in liquid form.

### Nutritional Supplements and Feeds

Transgenic cells containing hypotaurine or taurine may be consumed or used to make extracts for nutritional supplements. Transgenic cells that contain hypotaurine or taurine may be used for human consumption. Extracts from transgenic cells containing hypotaurine or taurine may be used as nutritional supplements, as an antioxidant or to improve physical or mental performance. The extracts may be used in the form of a liquid, powder, capsule or tablet.

Transgenic cells containing hypotaurine or taurine may be used as fish or animal feed or used to make extracts for the supplementation of animal feed. Transgenic cells that contain hypotaurine or taurine may be used as animal or fish feed. Extracts from transgenic cells containing taurine may be used as feed supplements in the form of a liquid, powder, capsule or tablet.

### Enhancer of Plant Growth or Yield

Transgenic cells that contain hypotaurine or taurine may be used as an enhancer for plant growth or yield. Extracts from transgenic cells containing hypotaurine or taurine may be used as plant enhancers in the form of a liquid, powder, capsule or tablet.

### Definitions

The term “polynucleotide” refers to a natural or synthetic linear and sequential array of nucleotides and/or nucleosides, including deoxyribonucleic acid, ribonucleic acid, and derivatives thereof. It includes chromosomal DNA, self-replicating plasmids, infectious polymers of DNA or RNA and DNA or RNA that performs a primarily structural role. Unless otherwise indicated, nucleic acids or polynucleotide are written left to right in 5′ to 3′ orientation, Nucleotides are referred to by their commonly accepted single-letter codes. Numeric ranges are inclusive of the numbers defining the range.

The terms “amplified” and “amplification” refer to the construction of multiple copies of a nucleic acid sequence or multiple copies complementary to the nucleic acid sequence using at least one of the nucleic acid sequences as a template. Amplification can be achieved by chemical synthesis using any of the following methods, such as solid-phase phosphoramidate technology or the polymerase chain reaction (PCR). Other amplification systems include the ligase chain reaction system, nucleic acid sequence based amplification, Q-Beta Replicase systems, transcription-based amplification system, and strand displacement amplification. The product of amplification is termed an amplicon.

As used herein “promoter” includes reference to a region of DNA upstream from the start of transcription and involved in recognition and binding of RNA polymerase, either I, II or III, and other proteins to initiate transcription. Promoters include necessary nucleic acid sequences near the start site of transcription, such as, in the case of a polymerase II type promoter, a TATA element. A promoter also optionally includes distal enhancer or repressor elements, which can be located as far as several thousand base pairs from the start site of transcription. In bacteria, the promoter includes a Shine-Dalgarno or ribosomal binding site that can include the sequence AGGAGG (−35 box) and a Pribnow box or RNA polymerase binding site that can include the sequence TATAAT (−10 box).

The term “algal promoter” refers to a promoter capable of initiating transcription in algal cells.

The term “foreign promoter” refers to a promoter, other than the native, or natural, promoter, which promotes transcription of a length of DNA of viral, bacterial or eukaryotic origin, including those from microbes, plants, plant viruses, invertebrates or vertebrates.

The term “microbe” refers to any microorganism (including both eukaryotic and prokaryotic microorganisms), such as bacteria, fungi, yeast, bacteria, algae and protozoa, as well as other unicellular organisms.

The term “constitutive” refers to a promoter that is active under most environmental and developmental conditions, such as, for example, but not limited to, the CaMV 35S promoter.

The term “inducible promoter” refers to a promoter that is under chemical (including biomolecules such as sugars, organic acids or amino acids) or environmental control.

The terms “encoding” and “coding”” refer to the process by which a polynucleotide, through the mechanisms of transcription and translation, provides the information to a cell from which a series of amino acids can be assembled into a specific amino acid sequence to produce a functional polypeptide, such as, for example, an active enzyme or ligand binding protein.

The terms “polypeptide,” “peptide,” “protein” and “gene product” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. Amino acids may be referred to by their commonly known three-letter or one-letter symbols. Amino acid sequences are written left to right in amino to carboxy orientation, respectively. Numeric ranges are inclusive of the numbers defining the range.

The terms “residue,” “amino acid residue,” and “amino acid” are used interchangeably herein to refer to an amino acid that is incorporated into a protein, polypeptide, or peptide. The amino acid may be a naturally occurring amino acid and may encompass known analogs of natural amino acids that can function in a similar manner as the naturally occurring amino acids.

The term “degradation” in reference to the “taurine degradation pathway”, “taurine degradation enzymes”, “taurine degradation system”, and “taurine degradation proteins” refers to the process of breakdown, catabolismor dissimilation of taurine.

The terms “cysteine dioxygenase” and “CDO” refer to the protein that catalyzes the following reaction:

cysteine+oxygen=3-sulfinoalanine

NOTE: 3-sulfinoalanine is another name for cysteine sulfinic acid, cysteine sulfinate, 3-sulphino-L-alanine, 3-sulfino-alanine, 3-sulfino-L-alanine, L-cysteine sulfinic acid, L-cysteine sulfinic acid, cysteine hydrogen sulfite ester or alanine 3-sulfinic acid.

The terms “sulfinoalanine decarboxylase” and “SAD” refer to the protein that catalyzes the following reaction:

3-sulfinoalanine=hypotaurine+CO₂

NOTE: SAD is another name for cysteine-sulfinate decarboxylase, L-cysteine sulfinic acid decarboxylase, cysteine-sulfinate decarboxylase, CADCase/CSADCase, CSAD, cysteic decarboxylase, cysteine sulfinic acid decarboxylase, cysteine sulfinate decarboxylase, sulfoalanine decarboxylase, sulphinoalanine decarboxylase, cysteate decarboxylase (CAD), cysteic acid decarboxylase, and 3-sulfino-L-alanine carboxy-lyase.

NOTE: the SAD reaction is also catalyzed by some glutamic acid decarboxylases (GAD). Although called GAD the enzyme has been shown to catalyze the SAD reaction (22, 23).

Other names for hypotaurine are 2-aminoethane sulfinate, 2-aminoethylsulfinic acid, and 2-aminoethanesulfinic acid.

Other names for taurine are 2-aminoethane sulfonic acid, aminoethanesulfonate, L-taurine, taurine ethyl ester, and taurine ketoisocaproic acid 2-aminoethane sulfinate.

The terms “threonine synthase” and “TS” refer to the protein that catalyzes the following reaction:

O-phosphoserine and sulfite=cysteate

NOTE: TS is another name for cysteate synthase

The terms “ilvA” or “ilvA gene product” refer to the protein that catalyzes the following reaction:

Serine=2-aminoacrylate

NOTE: ilvA is another name for serine/threonine dehydratase, threonine dehydratase, Ser/Thr dehydratase, threonine deaminase, serine ammonia lyase, serine dehydratase or SDH.

Other names for 2-aminoacrylate are 2-aminoacrylic acid, dehydroalanine and 2-aminoprop-2-enoic acid

The terms “3′-phosphoadenylyl sulfate: 2′-aminoacrylate C-sulfotransferase” or “PAPS-AS” refer to the protein that catalyzes the following reaction:

2-aminoacrylate+3′-phosphoadenosine-5′-phosphosulfate=cysteate]

The terms “cysteamine dioxygenase” and “ADO” refer to the protein that catalyzes the following reaction:

2-aminoethanethiol+O₂=hypotaurine

ADO is another name for 2-aminoethanethiol:oxygen oxidoreductase, persulfurase, cysteamine oxygenase, and cysteamine:oxygen oxidoreductase.

Other names for 2-aminoethanethiol are cysteamine or 2-aminoethane-1-thiol, b-mercaptoethylamine, 2-mercaptoethylamine, decarboxycysteine, and thioethanolamine.

The terms “cysteine lyase” and “CL” refer to the protein that catalyzes the following reaction:

Cysteine+sulfite=cysteate+hydrogen sulfide

Other names for cysteine lyase are cysteine sulfite lyase and cysteine hydrogen-sulfide-lyase.

The terms “taurine-pyruvate aminotransferase” and “TPAT” refer to the protein that catalyzes the following reaction:

taurine+pyruvate=L-alanine+2-sulfoacetaldehyde

TPAT is another name for taurine transaminase or taurine transaminase aminotransferase. The term “Tpa” refers to the gene that encodes TPAT.

The terms “taurine dehydrogenase” and “TDH” refer to the protein that catalyzes the following reaction:

taurine+water=ammonia+2-sulfoacetaldehyde

TDH is another name for taurine: oxidoreductase, taurine: ferricytochrome-c oxidoreductase,

The term “tauX” or “tauY” refers to the genes that encode for the small and large subunits of TDH, respectively.

The terms “taurine dioxygenase” and “TDO” refer to the protein that catalyzes the following reaction:

taurine+2-oxoglutarate+O₂=sulfite+aminoacetaldehyde+succinate+CO₂

TDO is another name for 2-aminoethanesulfonate dioxygenase, alpha-ketoglutarate-dependent taurine dioxygenase, taurine, or 2-oxoglutarate:O₂ oxidoreductase.

The term “tauD” refers to the gene that encodes TDO.

The term “two-component alkanesulfonate monooxygenase” or “2CASM” catalyzes the following reaction:

taurine+O₂+FMNH₂=Aminoacetaldehyde+SO₃²+H₂O+FMN

or

taurine+O₂+Thioredoxinred-Aminoacetaldehyde+SO₃²+H₂O+Thioredoxinox

The term “ssuDE”, “ssuD” or “ssuE” refers to the genes that encode the two-component alkanesulfonate monooxygenase (2CASM).

The terms “cysteine synthetase/PLP decarboxylase” and “CS/PLP-DC” refer to the protein that catalyzes the following reactions:

2-aminocrylate+PAPS=taurine

O-phosphoserine+PAPS=taurine

O-acetyl-L-serine+hydrogen sulfide=taurine

The terms “portion of the cysteine synthetase/PLP decarboxylase” and “partCS/PLP-DC” refers to the protein that catalyzes a decarboxylase reaction which cleaves carbon-carbon bonds and includes, but is not limited to, the following substrate and end-products:

Cysteic acid=2-aminoethane sulfonate+CO₂

3-sulfinoalanine=hypotaurine+CO₂

Glutamate=4-aminobutanoate+CO₂

Another name for 4-aminobutanoate is gamma-aminobutyric acid (GABA).

Other names for pyridoxal 5′-phosphate (PLP) are vitamin B6 and P-5-P.

The term “recombinant” includes reference to a cell or vector that has been modified by the introduction of a heterologous nucleic acid. Recombinant cells express genes that are not normally found in that cell or express native genes that are otherwise abnormally expressed, underexpressed, or not expressed at all as a result of deliberate human intervention, or expression of the native gene may have reduced or eliminated as a result of deliberate human intervention.

The term “recombinant expression cassette” refers to a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “transgenic” includes reference to a unicellular, which comprises within its genome a heterologous polynucleotide. Generally, the heterologous polynucleotide is integrated within the genome such that the polynucleotide is passed on to successive generations. The heterologous polynucleotide may be integrated into the genome alone or as part of a recombinant expression cassette. “Transgenic” is also used to include any cell the genotype of which has been altered by the presence of heterologous nucleic acid including those cells altered or created by budding or conjugation propagation from the initial transgenic cell.

The term “vector” includes reference to a nucleic acid used in transfection or transformation of a host cell and into which can be inserted a polynucleotide.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity, and most preferably 100% sequence identity (i.e., complementary) with each other.

The terms “stringent conditions” and “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt solution. Low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Moderate stringency conditions include hybridization in 40 to 45% formamide, 1 M NaCl, 1% SDS at 37° C., and a wash in 0.5× to 1×SSC at 55 to 60° C. High stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated (262), where the Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill in the art will understand that variations in the stringency of hybridization and/or wash solutions are inherently described. An extensive guide to the hybridization of nucleic acids is found in the scientific literature (126, 263). Unless otherwise stated, in the present application high stringency is defined as hybridization in 4×SSC, 5×Denhardt solution (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

The following terms are used to describe the sequence relationships between two or more nucleic acids or polynucleotides or polypeptides: “reference sequence,” “comparison window,” “sequence identity,” “percentage of sequence identity,” and “substantial identity.”

The term “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “comparison window” includes reference to a contiguous and specified segment of a polynucleotide sequence, where the polynucleotide sequence may be compared to a reference sequence and the portion of the polynucleotide sequence in the comparison window may comprise additions or deletions (i.e., gaps) when it is compared to the reference sequence for optimal alignment. The comparison window is usually at least 20 contiguous nucleotides in length, and optionally can be 30, 40, 50, 100 or longer. Those of ordinary skill in the art understand that the inclusion of gaps in a polynucleotide sequence alignment introduces a gap penalty, and it is subtracted from the number of matches.

Methods of alignment of nucleotide and amino acid sequences for comparison are well known to those of ordinary skill in the art. The local homology algorithm, BESTFIT (264), can perform an optimal alignment of sequences for comparison using a homology alignment algorithm called GAP (265), search for similarity using Tfasta and Fasta,²⁶⁶ by computerized implementations of these algorithms widely available on-line or from various vendors (Intelligenetics, Genetics Computer Group). CLUSTAL allows for the alignment of multiple sequences (267-269) and program PileUp can be used for optimal global alignment of multiple sequences (270). The BLAST family of programs can be used for nucleotide or protein database similarity searches. BLASTN searches a nucleotide database using a nucleotide query. BLASTP searches a protein database using a protein query. BLASTX searches a protein database using a translated nucleotide query that is derived from a six-frame translation of the nucleotide query sequence (both strands). TBLASTN searches a translated nucleotide database using a protein query that is derived by reverse-translation. TBLASTX search a translated nucleotide database using a translated nucleotide query.

GAP (265) maximizes the number of matches and minimizes the number of gaps in an alignment of two complete sequences. GAP considers all possible alignments and gap positions and creates the alignment with the largest number of matched bases and the fewest gaps. It also calculates a gap penalty and a gap extension penalty in units of matched bases. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (271).

Unless otherwise stated, sequence identity or similarity values refer to the value obtained using the BLAST 2.0 suite of programs using default parameters (272). As those of ordinary skill in the art understand that BLAST searches assume that proteins can be modeled as random sequences and that proteins comprise regions of nonrandom sequences, short repeats, or enriched for one or more amino acid residues, called low-complexity regions. These low-complexity regions may be aligned between unrelated proteins even though other regions of the protein are entirely dissimilar. Those of ordinary skill in the art can use low-complexity filter programs to reduce number of low-complexity regions that are aligned in a search. These filter programs include, but are not limited to, the SEG (273, 274) and XNU (275).

The terms “sequence identity” and “identity” are used in the context of two nucleic acid or polypeptide sequences and include reference to the residues in the two sequences, which are the same when aligned for maximum correspondence over a specified comparison window. When the percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conserved substitutions, the percent sequence identity may be adjusted upwards to correct for the conserved nature of the substitution. Sequences, which differ by such conservative substitutions, are said to have “sequence similarity” or “similarity.” Scoring for a conservative substitution allows for a partial rather than a full mismatch (276), thereby increasing the percentage sequence similarity.

The term “percentage of sequence identity” means the value determined by comparing two optimally aligned sequences over a comparison window, wherein the portion of the polynucleotide sequence in the comparison window may comprise gaps (additions or deletions) when compared to the reference sequence for optimal alignment. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90%, and most preferably at least 95%, compared to a reference sequence using one of the alignment programs described using standard parameters. One of ordinary skill in the art will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 50-100%. Another indication that nucleotide sequences are substantially identical is if two molecules hybridize to each low stringency conditions, moderate stringency conditions or high stringency conditions. Yet another indication that two nucleic acid sequences are substantially identical is if the two polypeptides immunologically cross-react with the same antibody in a western blot, immunoblot or ELISA assay.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm (265). Thus, a peptide is substantially identical to a second peptide, for example, where the two peptides differ only by a conserved substitution. Another indication that amino acid sequences are substantially identical is if two polypeptides immunologically cross-react with the same antibody in a western blot, immunoblot or ELISA assay. In addition, a peptide can be substantially identical to a second peptide when they differ by a non-conservative change if the epitope that the antibody recognizes is substantially identical.

The invention provides isolated cells comprising DNA which does not express a functional taurine degradation enzyme, some isolated cells of the invention comprise (i) exogenous DNA which disrupts the expression of the gene or renders the corresponding peptide for the degradation enzyme non-functional (ii) a basepair mutation that disrupts the expression of the gene or renders the corresponding peptide for the degradation enzyme non-functional, or (iii) a deletion of the entire polynucleotide or a portion of the polynucleotide which disrupts the expression of the gene or renders the corresponding peptide for the degradation enzyme non-functional. The non-functional DNA could be due to changes in the promoter, a portion of the coding region or terminator to a polynucleotide which encodes taurine degradation enzyme, that includes tauX, tauY, tauD), tpa, ssuD), or ssuE or in genes that encode translational activators of those genes including chl or tauR in a manner where the gene products are not functional. The invention also provides isolated cells comprising non-functional genes or gene products of taurine degradation enzymes from the suppression or decreased accumulation of the corresponding RNA due to antisense RNA or RNA interference.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

The publications and other materials used herein to illuminate the background of the invention or provide additional details respecting the practice, are incorporated by reference, and for convenience are respectively grouped in the References.

### REFERENCES

1. Ames B N. Prolonging healthy aging: Longevity vitamins and proteins. Proc Natl Acad Sci USA. 2018; 115 (43): 10836.
2. Lourenco R, Camilo M E. Taurine: a conditionally essential amino acid in humans? An overview in health and disease. Nutr Hosp. 2002; 17 (6): 262-70.
3. Markwell P J, Earle K E. Taurine: An essential nutrient for the cat. A brief review of the biochemistry of its requirement and the clinical consequences of deficiency. Nutrition Research. 1995; 15:53-8.
4. Ripps H, Shen W. Review: taurine: a “very essential” amino acid. Molecular Vision. 2012; 18:2673-86.
5. Salze G P, Davis D A. Taurine: a critical nutrient for future fish feeds. Aquaculture. 2015; 437:215-29.
6. Stapleton P P, Charles R P, Redmond H P, Bouchier-Hayes D J. Taurine and human nutrition. Clinical Nutrition. 1997; 16:103-8.
7. Yamori Y, Taguchi T, Hamada A, Kunimasa K, Mori H, Mori M. Taurine in health and diseases: consistent evidence from experimental and epidemiological studies. J Biomed Sci. 2010; 17 Suppl 1: S6.
8. Huxtable R J. Physiological actions of taurine. Physiological Reviews. 1992; 72:101-63.
9. Wu G. Important roles of dietary taurine, creatine, carnosine, anserine and 4-hydroxyproline in human nutrition and health. Amino Acids. 2020; 52 (3): 329-60.
10. Zafalon R V A, Risolia L W, Vendramini T H A, Ayres Rodrigues R B, Pedrinelli V, Teixeira F A, et al. Nutritional inadequacies in commercial vegan foods for dogs and cats. PLOS One. 2020; 15: e0227046.
11. Bondareva O M, Lopatik D V, Kuvaeva Z I, Vinokurova L G, Markovich M M, Prokopovich I P. Synthesis of taurine. Pharmaceutical Chemistry Journal. 2008:142-4.
12. Honjoh K I, Matsuura K, Machida T, Nishi K, Nakao M, Yano T, et al. Enhancement of menadione stress tolerance in yeast by accumulation of hypotaurine and taurine: co-expression of cDNA clones, from Cyprinus carpio, for cysteine dioxygenase and cysteine sulfinate decarboxylase in Saccharomyces cerevisiae. Amino Acids. 2010; 38:1173-83.
13. Turano F J, Turano K A, Carlson P S, Kinnersley A M, inventors; Plant Sensory Systems, LLC, assignee. Methods for the biosynthesis of taurine or hypotaurine in cells. U.S. Pat. No. 9,267,148. 2012.
14. Turano F J, Price M B, Turano K A, inventors; Plant Sensory Systems, LLC, assignee. Methods to Improve Plant-Based Food and Feed. USA2014.
15. Turano F J, Turano K A, Carlson P S, Kinnersley A M, inventors; Plant Sensory. Systems, LLC, assignee. Methods for the biosynthesis of taurine or hypotaurine in cells. U.S. Pat. No. 10,874,625. 2020.
16. Turano F J, inventor; Plant Sensory Systems, LLC, assignee. Algal and fungal genes and their uses for taurine biosynthesis in cells. U.S. Pat. No. 11,078,547. 2021.
17. Turano F J, Price M S, inventors; Plant Sensory Systems, LLC, assignee. Methods for High Taurine Production in Unicellular Organisms. U.S. Pat. No. 11,220,691. 2022.
18. Joo Y-C, Ko Y J, You S K, Shin S K, Hyeon J E, Musaad A S, et al. Creating a new pathway in Corynebacterium glutamicum for the production of taurine as a food additive. Journal of Agricultural and Food Chemistry 2018; 66:13454-63.
19. Tevatia R, Allen J, Rudrappa D, White D, Clemente T, Cerutti H, et al. The taurine biosynthetic pathway of microalgae. Algal Research. 2015; 9:21-6.
20. Feinberg L F, Marx C J, Wall M A, Smith D R, Pujol-Baxley C J, McAvoy B D, inventors; KnipBio, Inc., assignee. Heterologous expression of taurine in microorganisms2016.

21 Tchesnokov E P, Fellner M, Siakkou E, Kleffmann T, Martin L W, Aloi S, et al. The Cysteine Dioxygenase Homologue from Pseudomonas aeruginosa Is a 3-Mercaptopropionate Dioxygenase *. J Biol Chem. 2015; 290 (40): 24424-37.
22. Liu P, Ge X, Ding H, Jiang H, Christensen B M, Li J. Role of Glutamate Decarboxylase-like Protein 1 (GADL1) in Taurine Biosynthesis. J Biol Chem. 2012; 287 (49): 40898-906.
23. Winge I, Teigen K, Fossbakk A, Mahootchi E, Kleppe R, Sköldberg F, et al. Mammalian CSAD and GADL1 have distinct biochemical properties and patterns of brain expression. Neurochemistry International. 2015; 90:173-84.
24. Goto T, Matsumoto T, Murakami S, Takagi S, Hasumi F. Conversion of cysteate into taurine in liver of fish. Fisheries science. 2003; 69 (1): 216-8.
25. Graham D E, Taylor S M, Wolf R Z, Namboori S C. Convergent evolution of coenzyme M biosynthesis in the Methanosarcinales: cysteate synthase evolved from an ancestral threonine synthase. The Biochemical Journal. 2009; 424:467-78.
26. Sass N L, Martin W G. The synthesis of taurine from sulfate III. Further evidence for the enzymatic pathway in chick liver. Proceedings of the Society for Experimental Biology and Medicine. 1972; 139:755-61.
27. Machlin L J, Pearson B, Denton C A. The utilization of sulfate-sulfur for the synthesis of taurine in the developing chick embryo. J Biol Chem. 1955; 212:469-75.
28. Tevatia R, Allenc J, Rudrappa D, White D, Clemente T E, Cerutti H, et al. The taurine biosynthetic pathway of microalgae. Algal Research. 2015; 9:21-6.
29. Rojas-Pirela M, Andrade-Alviárez D, Rojas V, Kemmerling U, Cáceres AJ, Michels P A, et al. Phosphoglycerate kinase: structural aspects and functions, with special emphasis on the enzyme from Kinetoplastea. Open Biology. 2020.
30. Peters-Wendisch P, Stolz M, Etterich H, Kennerknecht N, Sahm H, Eggeling L. Metabolic engineering of Corynebacterium glutamicum for L-serine production. Applied and Environmental Microbiology. 2005; 71:7139-44.
31. Wei L, Wang H, Xu N, Wei Z, Ju J, Liu J, et al. Metabolic engineering of Corynebacterium glutamicum for l-cysteine production. Applied Microbiology and Biotechnology. 2019; 103.
32. Awano N, Wada M, Mori H, Nakamori S, Takagi H. Identification and functional analysis of Escherichia coli cysteine desulfhydrases. Applied and Environmental Microbiology. 2005; 71:4149-52.
33. Borchert A J, Downs D M. Analyses of variants of the Ser/Thr dehydratase IlvA provide insight into 2-aminoacrylate metabolism in Salmonella enterica. The Journal of biological chemistry. 2018; 293 (50): 19240-9.
34. Hryniewicz M, Sirko A, Pałucha A, Böck A, Hulanicka D. Sulfate and thiosulfate transport in Escherichia coli K-12: identification of a gene encoding a novel protein involved in thiosulfate binding. Journal of bacteriology. 1990; 172 (6): 3358-66.
35. Zhao C, Kumada Y, Imanaka H, Imamura K, Nakanishi K. Cloning, overexpression, purification, and characterization of O-acetylserine sulfhydrylase-B from Escherichia coli . . . . Protein expression and purification. 2006; 47:607-13.
36. van der Ploeg J R, Weiss M A, Saller E, Nashimoto H, Saito N, Kertesz M A, et al. Identification of sulfate starvation-regulated genes in Escherichia coli: a gene cluster involved in the utilization of taurine as a sulfur source. Journal of Bacteriology. 1996; 178 (18): 5438-46.
37. van der Ploeg J R, Cummings N J, Leisinger T, Connerton I F. Bacillus subtilis genes for the utilization of sulfur from aliphatic sulfonates. Microbiology. 1998; 144 (9): 2555-61.
38. Brüggemann C, Denger K, Cook A M, Ruff J. Enzymes and genes of taurine and isethionate dissimilation in Paracoccus denitrificans. Microbiology. 2004; 150 (4): 805-16.
39 Denger K, Ruff J, Schleheck D, Cook A M. Rhodococcus opacus expresses the xsc gene to utilize taurine as a carbon source or as a nitrogen source but not as a sulfur source. Microbiology. 2004; 150 (6): 1859-67.
40. van der Ploeg J R, Weiss M A, Saller E, Nashimoto H, Saito N, Kertesz M A, et al. Identification of sulfate starvation-regulated genes in Escherichia coli: A gene cluster involved in the utilization of taurine as a sulfur source. Journal of Bacteriology. 1996; 178:5438-46.
41. van der Ploeg J R, Iwanicka-Nowicka R, Bykowski T, Hryniewicz M M, Leisinger T. The Escherichia coli ssuEADCB Gene Cluster Is Required for the Utilization of Sulfur from Aliphatic Sulfonates and Is Regulated by the Transcriptional Activator Cbl. J Biol Chem. 1999; 274 (41): 29358-65.
42. Denger K, Smits T H M, Cook A M. Genome-enabled analysis of the utilization of taurine as sole source of carbon or of nitrogen by Rhodobacter sphaeroides 2.4.1. Microbiology. 2006; 152 (11): 3197-206.
43. Krejcik Z, Schleheck D, Hollemeyer K, Cook A M. A five-gene cluster involved in utilization of taurine-nitrogen and excretion of sulfoacetaldehyde by Acinetobacter radioresistens SH164. Archives of microbiology. 2012; 194 (10): 857-63.
44. Gorzynska A K, Denger K, Cook A M, Smits T H M. Inducible transcription of genes involved in taurine uptake and dissimilation by Silicibacter pomeroyi DSS-3T. Archives of microbiology. 2006; 185 (5): 402-6.
45. Koch D J, Rückert C, Rey D A, Mix A, Pühler A, Kalinowski J. Role of the ssu and seu genes of Corynebacterium glutamicum ATCC 13032 in utilization of sulfonates and sulfonate esters as sulfur sources. Applied and environmental microbiology. 2005; 71 (10): 6104-14.
46. Novak R T, Gritzer R F, Leadbetter E R, Godchaux W. Phototrophic utilization of taurine by the purple nonsulfur bacteria Rhodopseudomonas palustris and Rhodobacter sphaeroides. Microbiology. 2004; 150 (6): 1881-91.
47. Song Y, Yang C, Chen G, Zhang Y, Seng Z, Cai Z, et al. Molecular insights into the master regulator CysB-mediated bacterial virulence in Pseudomonas aeruginosa. Molecular Microbiology. 2019; 111 (5): 1195-210.
48. Rey D A, Pühler A, Kalinowski J. The putative transcriptional repressor McbR, member of the TetR-family, is involved in the regulation of the metabolic network directing the synthesis of sulfur containing amino acids in Corynebacterium glutamicum. Journal of Biotechnology. 2003; 103:61-5.
49. van der Ploeg J R, Iwanicka-Nowicka R, Kertesz M A, Leisinger T, Hryniewicz M M. Involvement of CysB and Cbl regulatory proteins in expression of the tauABCD operon and other sulfate starvation-inducible genes in Escherichia coli. J Bacteriol. 1997; 179 (24): 7671-8.
50. Demain A L. The business of biotechnology. Industrial Biotechnology. 2007; 3:269-83.
51. Roubos J A, van Straten G, van Boxtel A J B. An evolutionary strategy for fed-batch bioreactor optimization; concepts and performance. Journal of Biotechnology. 1999; 67 (2-3): 173-87.
52. Oka T. Amino acids, production processes. In: Flickinger M C, Drew S W, editors. Encyclopedia of Bioprocess Technology: Fermentation, Biocatalysis, and Bioseparation. London: Wiley; 1999.
53. Borowitzka M A. Commercial production of microalgae: ponds, tanks, tubes and fermenters. Journal of Biotechnology. 1999; 70 (1-3): 313-21.
54. Hermann T. Industrial production of amino acids by Coryneform bacteria. Journal of Biotechnology. 2003; 104:155-72.
55. Ikeda M. Amino acid production processes. Advances in biochemical engineering/biotechnology. 2003; 79:1-35.
56. Richmond A, Hu Q, editors. Handbook of Microalgal Culture: Biotechnology and Applied Phycology. 2nd ed. Hoboken, New Jersey: Wiley-Blackwell; 2013.
57. Cardozo K H, Guaratini T, Barros M P, Falcao V R, Tonon A P, Lopes N P, et al. Metabolites from algae with economical impact. Comparative biochemistry and physiology Toxicology & pharmacology: CBP. 2007; 146 (1-2): 60-78.
58. Milledge J J. Commercial application of microalgae other than as biofuels: a brief review. Reviews in Environmental Science and Biotechnology. 2011; 10:31-41.
59. Xu Q, Li S, Huang H, Wen J. Key technologies for the industrial production of fumaric acid by fermentation. Biotechnol Adv. 2012; 30 (6): 1685-96.
60. Dufossé L, Fouillaud M, Caro Y, Mapari S A S, Sutthiwong N. Filamentous fungi are large-scale producers of pigments and colorants for the food industry. Current Opinion in Biotechnology. 2014; 26:56-61.
61. Jones D M. Manual of Methods for General Bacteriology. J Clin Pathol. 1981; 34 (9): 1069-.
62. Oberhardt M A, Zarecki R, Gronow S, Lang E, Klenk H-P, Gophna U, et al. Harnessing the landscape of microbial culture media to predict new organism-media pairings. Nature Communications. 2015; 6 (1): 8493.
63. Richards M A, Cassen V, Heavner B D, Ajami N E, Herrmann A, Simeonidis E, et al. MediaDB: A database of microbial growth conditions in defined media. PLOS ONE. 2014; 9: e103548.
64. Kumar R, Vikramachakravarthi D, Pal P. Production and purification of glutamic acid: A critical review towards process intensification. Chemical Engineering and Processing. 2014; 81:59-71.
65. Ecker J, Raab T, Harasek M. Nanofiltration as key technology for the separation of LA and AA. Fuel and Energy Abstracts. 2011; 389.
66. Li H, Qiu T, Chen Y, Cao Y. Separation of gamma-aminobutyric acid from fermented broth. Journal of Indian Microbiology Biotechnology. 2011; 38:1955-9.
67. Bennetzen J L, Hall B D. Codon selection in yeast. J Biol Chem. 1982; 257 (6): 3026-31.
68. Gouy M, Gautier C. Codon usage in bacteria: correlation with gene expressivity. Nucleic Acids Research. 1982; 10 (22): 7055-74.
69. Campbell W H, Gowri G. Codon Usage in Higher Plants, Green Algae, and Cyanobacteria. Plant Physiol. 1990; 92 (1): 1-11.
70. Douglas E S, Penny L S. The Plastid Genome of the Cryptophyte Alga, Guillardia theta: Complete Sequence and Conserved Synteny Groups Confirm Its Common Ancestry with Red Algae. Journal of Molecular Evolution. 48 (2): 236-44.
71. Yoon H S, Müller KM, Sheath R G, Ott F D, Bhattacharya D. Defining the major lineages of red algae (rhodophyta). Journal of Phycology. 2006; 42 (2): 482-92.
72. Fletcher S P, Muto M, Mayfield S P. Optimization of Recombinant Protein Expression in the Chloroplasts of Green Algae. In: León R, Galván A, Fernández E, editors. Transgenic Microalgae as Green Cell Factories. New York, NY: Springer New York; 2007. p. 90-8.
73. Langenheim J H, Thimann K V. Botany: Plant Biology and its Relation to Human Affairs. New York: John Wiley & Sons Inc.; 1982.
74. Vasil I K. Cell Culture and Somatic Cell Genetics of Plants: Laboraory Procedures and Their Applications. Vasil I K, editor. Orlando: Academic Press; 1984.
75. Stanier R, Ingrahm J, Wheelis M, Painter P. The Microbial World. 5 ed. New Jersey: Prentice-Hall; 1986.
76. Dhringra O D, Sinclair J B. Basic plant pathology methods. Boca Raton, FL: CRC Press; 1985.
77. Maniatis T, Fritsch E F, Sambrook J. Molecular Cloning: A Laboratory Manual: DNA Cloning. Glover D M, editor. New York: Cold Spring Harbor; 1985.
78. Gait. Oligonucleotide Synthesis-A Practical Approach. Gait, editor. Washington, D.C.: IRL Press; 1984.
79. Hames D D, Higgins S J. Nucleic Acid Hybridization: A Practical Approach. Hames D D, Higgins S J, editors. Washington D.C.: IRL Press; 1984.
80. Watson J D, Gilman M, Witowski J, Zoller M. Recombinant DNA. New York: Scientific American Books; 1992.
81. Szewczyk E, Nayak T, Oakley C E, Edgerton H, Xiong Y, Taheri-Talesh N, et al. Fusion P C R and gene targeting in Aspergillus nidulans. Nature Protocols. 2006; 1:3111-21.
82. Ho S N, Hunt H D, Horton R M, Pullen J K, Pease L R. Site-directed mutagenesis by overlap extension using the polymerase chain reaction. Gene. 1989; 77:51-9.
83. Fuhrmann M, Oertel W, Hegemann P. A synthetic gene coding for the green fluorescent protein (GFP) is a versatile reporter in Chlamydomonas reinhardtii. Plant Journal. 1999; 19:353-61.
84. Mandecki W, Bolling T J. FokI method of gene synthesis. Gene. 1988; 68:101-7.
85. Stemmer W P, Crameri, A., Ha, K. D., Brennan, T. M. and Heyneker, H. L., Single-step assembly of a gene and entire plasmid from large numbers of oligodeoxyribonucleotides. Gene. 1995; 164:49-53.
86. Gao X, Yo P, Keith A, Ragan T J, Harris T K. Thermodynamically balanced inside-out (TBIO) PCR-based gene synthesis: a novel method of primer design for high-fidelity assembly of longer gene sequences. Nucleic Acids Research. 2003; 31: e143.
87. Young L, Dong Q. Two-step total gene synthesis method. Nucleic Acids Research. 2004; 32: e59.
88 Rosano G L, Ceccarelli E A. Recombinant protein expression in microbial systems. Frontiers in microbiology. 2014; 5:341.
89. Hlavova M, Turoczy Z, Bisova K. Improving microalgae for biotechnology—From genetics to synthetic biology. Biotechnology Advances. 2015; 33:1194-203.
90. çelik E, çalιk P. Production of recombinant proteins by yeast cells. Biotechnology Advances. 2012; 30 (5): 1108-18.
91. de Jong A, Pietersma H, Cordes M, Kuipers O P, Kok J. PePPER: a webserver for prediction of prokaryote promoter elements and regulons. BMC Genomics. 2012; 13:299.
92. Lee D J, Minchin S D, Busby S J W. Activating Transcription in Bacteria. Annual review of microbiology. 2012; 66 (1): 125-52.
93. Meysman P, Collado-Vides J, Morett E, Viola R, Engelen K, Laukens K. Structural Properties of Prokaryotic Promoter Regions Correlate with Functional Features. PLOS ONE. 2014; 9 (2): e88717.
94. Fujiwara T, Ohnuma M, Yoshida M, Kuroiwa T, Hirano T. Gene Targeting in the Red Alga Cyanidioschyzon merolae: Single- and Multi-Copy Insertion Using Authentic and Chimeric Selection Markers. PLOS ONE. 2013; 8 (9): e73608.
95. Mikami K, Hirata K, Takahashi M, Uji T, Saga N. Transient transformation of red algal cells: Breakthrough toward genetic transformation of marine crop porphyra species. In: Alvarez M, editor. Genetic Transformation: InTech; 2011.
96. Manuell A L, Beligni M V, Elder J H, Siefker D T, Tran M, Weber A, et al. Robust expression of a bioactive mammalian protein in Chlamydomonas chloroplast. Plant biotechnology journal. 2007; 5 (3): 402-12.
97. Cui Y, Qin S, Jiang P. Chloroplast Transformation of Platymonas (Tetraselmis) subcordiformis with the bar Gene as Selectable Marker. PLOS ONE. 2014; 9 (6): e98607.
98. Oey M, Ross I L, Stephens E, Steinbeck J, Wolf J, Radzun K A, et al. RNAi Knock-Down of LHCBM1, 2 and 3 Increases Photosynthetic H2 Production Efficiency of the Green Alga Chlamydomonas reinhardtii. PLOS ONE. 2013; 8 (4): e61375.
99. Oey M, Ross I L, Hankamer B. Gateway-Assisted Vector Construction to Facilitate Expression of Foreign Proteins in the Chloroplast of Single Celled Algae. PLOS ONE. 2014; 9 (2): e86841.
100. Wang B, Wang J, Zhang W, Meldrum D R. Application of synthetic biology in cyanobacteria and algae. Frontiers in microbiology. 2012; 3:344.
101. Ingelbrecht I L, Herman L M, Dekeyser R A, Van Montagu M C, Depicker A G. Different 3′ end regions strongly influence the level of gene expression in plant cells. The Plant cell. 1989; 1:671-80.
102. Zaret K S, Sherman F. DNA sequence required for efficient transcrition termination in yeast. Cell. 1982; 28:563-73.
103. Helden Jv, Rios A F, Collado-Vides J. Discovering regulatory elements in non-coding sequences by analysis of spaced dyads. Nucleic Acids Research. 2000; 28 (8): 1808-18.
104. Graber J H. Variations in yeast 3′-processing cis-elements correlate with transcript stability. Trends in Genetics. 2003; 19 (9): 473-6.
105. Wodniok S, Simon A, Glöckner G, Becker B. Gain and loss of polyadenylation signals during evolution of green algae. BMC Evolutionary Biology. 2007; 7 (1): 1-12.
106. Shen Y, Liu Y, Liu L, Liang C, Li Q Q. Unique Features of Nuclear mRNA Poly (A) Signals and Alternative Polyadenylation in Chlamydomonas reinhardtii. Genetics. 2008; 179 (1): 167-76.
107. Schlackow M, Marguerat S, Proudfoot N J, Bahler J, Erban R, Gullerova M. Genome-wide analysis of poly (A) site selection in Schizosaccharomyces pombe. RNA (New York, NY). 2013; 19 (12): 1617-31.
108. Yamanishi M, Ito Y, Kintaka R, Imamura C, Katahira S, Ikeuchi A, et al. A Genome-Wide Activity Assessment of Terminator Regions in Saccharomyces cerevisiae Provides a “Terminatome” Toolbox. ACS Synthetic Biology. 2013; 2 (6): 337-47.
109. Chen Y-J, Liu P, Nielsen A A K, Brophy J A N, Clancy K, Peterson T, et al. Characterization of 582 natural and synthetic terminators and quantification of their design constraints. Nat Meth. 2013; 10 (7): 659-64.
110. Leavitt J M, Alper H S. Advances and current limitations in transcript-level control of gene expression. Curr Opin Biotechnol. 2015; 34:98-104.
111. Newman T C, Ohme-Takagi M, Taylor C B, Green P J. DST sequences, highly conserved among plant SAUR genes, target reporter transcripts for rapid decay in tobacco. The Plant cell. 1993; 5 (6): 701-14.
112. Ohme-Takagi M, Taylor C B, Newman T C, Green P J. The effect of sequences with high AU content on mRNA stability in tobacco. Proceedings of the National Academy of Sciences of the United States of America. 1993; 90 (24): 11811-5.
113. Rasala B A, Muto M, Lee P A, Jager M, Cardoso R M F, Behnke C A, et al. Production of therapeutic proteins in algae, analysis of expression of seven human proteins in the chloroplast of Chlamydomonas reinhardtii. Plant biotechnology journal. 2010; 8 (6): 719-33.
114. Doetsch N A, Favreau M R, Kuscuoglu N, Thompson M D, Hallick R B. Chloroplast transformation in Euglena gracilis: splicing of a group III twintron transcribed from a transgenic psbK operon. Current genetics. 2001; 39 (1): 49-60.
115. Lapidot M, Raveh D, Sivan A, Arad S M, Shapira M. Stable chloroplast transformation of the unicellular red alga Porphyridium species. Plant Physiol. 2002; 129 (1): 7-12.
116. Hatzfeld Y, inventor; BASF Plant Science Gmbh, assignee. Plants having enhanced yield-related traits and a method for making the same. USA patent U.S. Pat. No. 8,779,237. 2014.
117. Franklin S, Somanchi A, Espina K, Rudenko G, Chua P, inventors; Solazyme, Inc., assignee. Nucleic acids useful in the manufacture of oil. USA patent U.S. Pat. No. 8,674,180. 2014.
118. Feng P C C, Malven M, Flasinski S, inventors; Monsanto Technology Llc, assignee. Chloroplast transit peptides for efficient targeting of DMO and uses thereof patent U.S. Pat. No. 8,420,888. 2013.
119. Manjunath S, Navarro S X, Rapp W D, Shi X, Varagona M J, Winson J L, et al., inventors; Monsanto Technology L L C, assignee. Production of high tryptophan maize by chloroplast targeted expression of anthranilate synthase. USA patent U.S. Pat. No. 8,138,393. 2012.
120. Lee D W, Kim J K, Lee S, Choi S, Kim S, Hwang I. Arabidopsis Nuclear-Encoded Plastid Transit Peptides Contain Multiple Sequence Subgroups with Distinctive Chloroplast-Targeting Sequence Motifs. The Plant cell. 2008; 20 (6): 1603-22.
121. von Heijne G, Hirai T, Klösgen RB, Steppuhn J, Bruce B D, Keegstra K, et al. CHLPEP: a database of chloroplast transit peptides. Plant Mol Biol Rep. 1991; 9:104-26.
122. Waller R F, Reed M B, Cowman A F, McFadden G I. Protein trafficking to the plastid of Plasmodium falciparum is via the secretory pathway. The EMBO Journal. 2000; 19 (8): 1794-802.
123. Minge M A, Shalchian-Tabrizi K, Tørresen OK, Takishita K, Probert I, Inagaki Y, et al. A phylogenetic mosaic plastid proteome and unusual plastid-targeting signals in the green-colored dinoflagellate Lepidodinium chlorophorum. BMC Evolutionary Biology. 2010; 10 (1): 1-11.
124. Li H-m, Teng Y-S. Transit peptide design and plastid import regulation. Trends in Plant Science. 2013; 18 (7): 360-6.
125. Tardif M, Atteia A, Specht M, Cogne G, Rolland N, Brugière S, et al. PredAlgo: A New Subcellular Localization Prediction Tool Dedicated to Green Algae. Molecular Biology and Evolution. 2012; 29 (12): 3625-39.
126. Ausubel F M, Brent R, Kingston R E, Moore D D, Seidman J, Smith J, et al. Current Protocols in Molecular Biology. New York: Greene Publishing and Wiley-Interscience; 1995.
127. Marx C J, Lidstrom M E. Development of improved versatile broad-host-range vectors for use in methylotrophs and other Gram-negative bacteria. Microbiology. 2001; 147:2065-75.
128 Atomi H, Imanaka T, Fukui T. Overview of the genetic tools in the Archaea. Frontiers in microbiology. 2012; 3:337.
129. Farkas J A, Picking J W, Santangelo T J. Genetic techniques for the archaea. Annu Rev Genet. 2013; 47:539-61.
130. Tan S. A modular polycistronic expression system for overexpressing protein complexes in Escherichia coli. Protein expression and purification. 2001; 21 (1): 224-34.
131. Tan S, Kern R C, Selleck W. The pST44 polycistronic expression system for producing protein complexes in Escherichia coli. Protein expression and purification. 2005; 40 (2): 385-95.
132. Baba T, Ara T, Hasegawa M, Takai Y, Okumura Y, Baba M, et al. Construction of Escherichia coli K-12 in-frame, single-gene knockout mutants: the Keio collection. Molecular Systems Biology. 2006; 2:2006.0008-2006.0008.
133. Reyrat J M, Pelicic V, Gicquel B, Rappuoli R. Counterselectable markers: untapped tools for bacterial genetics and pathogenesis. Infection and immunity. 1998; 66 (9): 4011-7.
134. Nakashima N, Miyazaki K. Bacterial cellular engineering by genome editing and gene silencing. International journal of molecular sciences. 2014; 15 (2): 2773-93.
135 Ried J L, Collmer A. An nptI-sacB-sacR cartridge for constructing directed, unmarked mutations in gram-negative bacteria by marker exchange-eviction mutagenesis. Gene. 1987; 57 (2-3): 239-46.
136 Murphy K C, Campellone K G, Poteete A R. PCR-mediated gene replacement in Escherichia coli. Gene. 2000; 246 (1-2): 321-30.
137. Sun W, Wang S, Curtiss R. Highly efficient method for introducing successive multiple scarless gene deletions and markerless gene insertions into the Yersinia pestis chromosome. Appl Environ Microbiol. 2008; 74:4241-5.
138. Costantino N, Court D L. Enhanced levels of λ Red-mediated recombinants in mismatch repair mutants. Proc Natl Acad Sci USA. 2003; 100 (26): 15748-53.
139. Datsenko K A, Wanner B L. One-step inactivation of chromosomal genes in Escherichia coli K-12 using PCR products. Proc Natl Acad Sci USA. 2000; 97 (12): 6640-5.
140 Lv L, Ren Y-L, Chen J-C, Wu Q, Chen G-Q. Application of CRISPRi for prokaryotic metabolic engineering involving multiple genes, a case study: Controllable P (3HB-co-4HB) biosynthesis. Metabolic Engineering. 2015; 29:160-8.
141. Peters J M, Silvis M R, Zhao D, Hawkins J S, Gross C A, Qi L S. Bacterial CRISPR: accomplishments and prospects. Current opinion in microbiology. 2015; 27:121-6.
142. Selle K, Barrangou R. Harnessing CRISPR-Cas systems for bacterial genome editing. Trends in Microbiology. 2015; 23 (4): 225-32.
143. Rehnstam-Holm A-S, Godhe A. Genetic engineering of algal species. In: Doelle H W, editor. Biotechnology. Oxford, UK: UNESCO, Eolss Publishers; 2003.
144. Rosa L, Galván-Cejudo A, Fernández E, editors. Transgenic Microalgae as Green Cell Factories. New York, NY: Springer Science+Business Media, LLC; 2007.
145. Leon R, Fernandez E. Nuclear transformation of eukaryotic microalgae: historical overview, achievements and problems. Adv Exp Med Biol. 2007; 616:1-11.
146. Mikami K, Hirata R, Takahashi M, Uji T, Saga N. Transient Transformation of Red Algal Cells: Breakthrough Toward Genetic Transformation of Marine Crop Porphyra Species. In: Alvarez M, editor. Genetic Transformation: InTech; 2011.
147. Umen J G, Olson B J. Genomics of Volvocine Algae. Advances in botanical research. 2012; 64:185-243.
148. Liu L, Wang Y, Zhang Y, Chen X, Zhang P, Ma S. Development of a new method for genetic transformation of the green alga Chlorella ellipsoidea. Molecular biotechnology. 2013; 54 (2): 211-9.
149. Gimpel J A, Specht E A, Georgianna D R, Mayfield S P. Advances in microalgae engineering and synthetic biology applications for biofuel production. Current opinion in chemical biology. 2013; 17 (3): 489-95.
150. Rasala B A, Chao S-S, Pier M, Barrera D J, Mayfield S P. Enhanced genetic tools for engineering multigene traits into green algae. PLOS ONE. 2014.
151. Potvin G, Zhang Z. Strategies for high-level recombinant protein expression in transgenic microalgae: a review. Biotechnol Adv. 2010; 28 (6): 910-8.
152. León-Bañares R, González-Ballester D, Galván A, Fernández E. Transgenic microalgae as green cell-factories. Trends in Biotechnology. 2004; 22 (1): 45-52.
153. Heitzer M, Zschoernig B. Construction of modular tandem expression vectors for the green alga Chlamydomonas reinhardtii using the Cre/lox-system. Biotechniques. 2007; 43 (3): 324, 6, 8 passim.
154. Sizova I, Greiner A, Awasthi M, Kateriya S, Hegemann P. Nuclear gene targeting in Chlamydomonas using engineered zinc-finger nucleases. The Plant Journal. 2013; 73 (5): 873-82.
155. Daboussi F, Leduc S, Maréchal A, Dubois G, Guyot V, Perez-Michaut C, et al. Genome engineering empowers the diatom Phaeodactylum tricornutum for biotechnology. Nat Commun. 2014; 5.
156. Romanos M A, Scorer C A, Clare J J. Foreign gene expression in yeast: a review. Yeast (Chichester, England). 1992; 8 (6): 423-88.
157. Agmon N, Mitchell L A, Cai Y, Ikushima S, Chuang J, Zheng A, et al. Yeast Golden Gate (yGG) for the Efficient Assembly of S. cerevisiae Transcription Units. ACS Synthetic Biology. 2015; 4 (7): 853-9.
158 Sherman F. Getting started with yeast. In: Guthrie C, Fink G R, editors. Methods in Enzymology, Guide to Yeast Genetics and Molecular Biology. 194. New York: Acad. Press; 1991. p. 3-21.
159. Sherman F, Fink G R, Hick J B. Methods in Yeast Genetics. New York: Cold Spring Harbor Laboratory; 1982.
160. Olmedo-Monfil V, CortEs-Penagos C, Herrera-Estrella A. Three Decades of Fungal Transformation. 2672004. p. 297-313.
161. Weld R J, Plummer K M, Carpenter M A, Ridgway H J. Approaches to functional genomics in filamentous fungi. Cell Res. 2006; 16 (1): 31-44.
162. Kawai S, Hashimoto W, Murata K. Transformation of Saccharomyces cerevisiae and other fungi: Methods and possible underlying mechanism. Bioengineered Bugs. 2010; 1 (6): 395-403.
163. van den Berg M A, Maruthachalam K, editors. Genetic Transformation Systems in Fungi, Volume 1. New York, NY: Springer; 2015.
164. Rivera A L, Magana-Ortiz D, Gomez-Lim M, Fernandez F, Loske A M. Physical methods for genetic transformation of fungi and yeast. Physics of life reviews. 2014; 11 (2): 184-203.
165. Vickers C E, Bydder S F, Zhou Y, Nielsen L K. Dual gene expression cassette vectors with antibiotic selection markers for engineering in Saccharomyces cerevisiae. Microbial Cell Factories. 2013; 12 (1): 1-11.
166. Sherman F. Yeast genetics. In: Meyers R A, editor. The Encyclopedia of Molecular Biology and Molecular Medicine. 6. Weinheim, Germany: VCH Publisher; 1997. p. 302-25.
167. Romanos M A, Scorer C A, Clare J J. Foreign gene expression in yeast: a review. Yeast (Chichester, England). 1992; 8.
168. Baudin A, Ozier-Kalogeropoulos O, Denouel A, Lacroute F, Cullin C. A simple and efficient method for direct gene deletion in Saccharomyces cerevisiae. Nucleic Acids Research. 1993; 21 (14): 3329-30.
169. Longtine M S, Mckenzie Iii A, Demarini D J, Shah N G, Wach A, Brachat A, et al. Additional modules for versatile and economical PCR-based gene deletion and modification in Saccharomyces cerevisiae. Yeast (Chichester, England). 1998; 14 (10): 953-61.
170. Krawchuk M D, Wahls W P. High-efficiency gene targeting in Schizosaccharomyces pombe using a modular, PCR-based approach with long tracts of flanking homology. Yeast (Chichester, England). 1999; 15 (13): 1419-27.
171. Epinat J-C, Arnould S, Chames P, Rochaix P, Desfontaines D, Puzin C, et al. A novel engineered meganuclease induces homologous recombination in yeast and mammalian cells. Nucleic Acids Research. 2003; 31 (11): 2952-62.
172. Li T, Huang S, Zhao X, Wright D A, Carpenter S, Spalding M H, et al. Modularly assembled designer TAL effector nucleases for targeted gene knockout and gene replacement in eukaryotes. Nucleic Acids Research. 2011.
173. DiCarlo J E, Norville J E, Mali P, Rios X, Aach J, Church G M. Genome engineering in Saccharomyces cerevisiae using CRISPR-Cas systems. Nucleic Acids Research. 2013; 41 (7): 4336-43.
174. Jacobs J Z, Ciccaglione K M, Tournier V, Zaratiegui M. Implementation of the CRISPR-Cas9 system in fission yeast. Nat Commun. 2014; 5.
175. Nakai K, Kanehisa M. Expert system for predicting protein localization sites in gram-negative bacteria. Proteins: Structure, Function, and Bioinformatics. 1991; 11 (2): 95-110.
176. Bendtsen J D, Nielsen H, von Heijne G, Brunak S. Improved prediction of signal peptides: SignalP 3.0. J Mol Biol. 2004; 340 (4): 783-95.
177. Bendtsen J D, Kiemer L, Fausbøll A, Brunak S. Non-classical protein secretion in bacteria. BMC Microbiology. 2005; 5 (1): 1-13.
178. Swinkels B W, Gould S J, Bodnar A G, Rachubinski R A, Subramani S. A novel, cleavable peroxisomal targeting signal at the amino-terminus of the rat 3-ketoacyl-CoA thiolase. EMBO (European Molecular Biology Organization) Journal. 1991; 10 (11): 3255-62.
179. Rusch S L, Kendall D A. Protein transport via amino-terminal targeting sequences: Common themes in diverse systems. Molecular Membrane Biology. 1995; 12 (4): 295-307.
180. Soll J, Tien R. Protein translocation into and across the chloroplastic envelope membranes. Plant Molecular Biology. 1998; 38:191-207.
181. Gould S J, Keller G A, Subramani S. Identification of peroxisomal targeting signals located at the carboxy terminus of four peroxisomal proteins. Journal of Cell Biology. 1988; 107 (3): 897-905.
182. Gould S J, Keller G A, Hosken N, Wilkinson J, Subramani S. A conserved tripeptide sorts proteins to peroxisomes. Journal of Cell Biology. 1989; 108 (5): 1657-64.
183. McCammon M T, McNew J A, Willy P J, Goodman J M. An internal region of the peroxisomal membrane protein PMP47 is essential for sorting to peroxisomes. Journal of Cell Biology. 1994; 124 (6): 915-25.
184. Cokol M, Nair R, Rost B. Finding nuclear localization signals. EMBO Reports. 2000; 1 (5): 411-5.
185. Helenius A, Aebi M. Intracellular functions of N-linked glycans. Science. 2001; 291 (5512): 2364-9.
186. Emanuelsson O, Brunak S, von Heijne G, Nielsen H. Locating proteins in the cell using TargetP, SignalP and related tools. Nature Protocols. 2007; 2 (4): 953-71.
187. Emanuelsson O, Nielsen H, Brunak S, von Heijne G. Predicting subcellular localization of proteins based on their N-terminal amino acid sequence. Journal of Molecular Biology. 2000; 300 (4): 1005-16.
188. Bannai H, Tamada Y, Maruyama O, Nakai K, Miyano S. Extensive feature detection of N-terminal protein sorting signals. Bioinformatics. 2002; 18 (2): 298-305.
189. Bendtsen J D, Nielsen H, von Heijne G, Brunak S. Improved prediction of signal peptides: SignalP 3.0. Journal of Molecular Biology. 2004; 340 (4): 783-95.
190. Hiller K, Grote A, Scheer M, Munch R, Jahn D. PrediSi: prediction of signal peptides and their cleavage positions. Nucleic Acids Research. 2004; 32 (Web Server issue): W375-9.
191. Bhasin M, Raghava G P. ESLpred: SVM-based method for subcellular localization of eukaryotic proteins using dipeptide composition and PSI-BLAST. Nucleic Acids Research. 2004; 32 (Web Server issue): W414-9.
192. Garg A, Bhasin M, Raghava G P. Support vector machine-based method for subcellular localization of human proteins using amino acid compositions, their order, and similarity search. J Biol Chem. 2005; 280 (15): 14427-32.
193. Bhasin M, Garg A, Raghava G P. PSLpred: prediction of subcellular localization of bacterial proteins. Bioinformatics. 2005; 21 (10): 2522-4.
194. Hoglund A, Donnes P, Blum T, Adolph H W, Kohlbacher O. MultiLoc: prediction of protein subcellular localization using N-terminal targeting sequences, sequence motifs and amino acid composition. Bioinformatics. 2006; 22 (10): 1158-65.
195. Shatkay H, Hoglund A, Brady S, Blum T, Donnes P, Kohlbacher O. SherLoc: high-accuracy prediction of protein subcellular localization by integrating text and protein sequence data. Bioinformatics. 2007; 23 (11): 1410-7.
196. Emanuelsson O, Nielsen H, von Heijne G. ChloroP, a neural network-based method for predicting chloroplast transit peptides and their cleavage sites. Protein Science. 1999; 8 (5): 978-84.
197. Claros M G, Vincens P. Computational method to predict mitochondrially imported proteins and their targeting sequences. European Journal of Biochemistry. 1996; 241 (3): 779-86.
198. Small I, Peeters N, Legeai F, Lurin C. Predotar: A tool for rapidly screening proteomes for N-terminal targeting sequences. Proteomics. 2004; 4 (6): 1581-90.
199. Kelley L A, MacCallum R M, Sternberg M J. Enhanced genome annotation using structural profiles in the program 3D-PSSM. Journal of Molecular Biology. 2000; 299 (2): 499-520.
200. Hlavova M, Turoczy Z, Bisova K. Improving microalgae for biotechnology—From genetics to synthetic biology. Biotechnology advances. 2015; 33 (6 Pt 2): 1194-203.
201. Pratheesh P T, Vineetha M, Kurup G M. An Efficient Protocol for the Agrobacterium-mediated Genetic Transformation of Microalga Chlamydomonas reinhardtii. Molecular biotechnology. 2013; 56 (6): 507-15.
202. Kindle K L. Nuclear Transformation: Technology and Applications. In: Rochaix J D, Goldschmidt-Clermont M, Merchant S, editors. The Molecular Biology of Chloroplasts and Mitochondria in Chlamydomonas. Dordrecht: Springer Netherlands; 1998. p. 41-61.
203. Ohnuma M, Yokoyama T, Inouye T, Sekine Y, Tanaka K. Polyethylene Glycol (PEG)-Mediated Transient Gene Expression in a Red Alga, Cyanidioschyzon merolae 10D. Plant and Cell Physiology. 2008; 49 (1): 117-20.
204. Shimogawara K, Fujiwara S, Grossman A, Usuda H. High-efficiency transformation of Chlamydomonas reinhardtii by electroporation. Genetics. 1998; 148 (4): 1821-8.
205. Hayashi M, Hirono M, Kamiya R. Recovery of flagellar dynein function in a Chlamydomonas actin/dynein-deficient mutant upon introduction of muscle actin by electroporation. Cell Motility and the Cytoskeleton. 2001; 49 (3): 146-53.
206. van Ooijen G, Knox K, Kis K, Bouget F-Y, Millar A J. Genomic Transformation of the Picoeukaryote Ostreococcus tauri. Journal of Visualized Experiments: JoVE. 2012 (65): 4074.
207. Vieler A, Wu G, Tsai C—H, Bullard B, Cornish A J, Harvey C, et al. Genome, Functional Gene Annotation, and Nuclear Transformation of the Heterokont Oleaginous Alga Nannochloropsis oceanica CCMP1779. PLOS Genetics. 2012; 8 (11): e1003064.
208. Boynton J E, Gillham N W, Harris E H, Hosler J P, Johnson A M, Jones A R, et al. Chloroplast transformation in Chlamydomonas with high velocity microprojectiles. Science. 1988; 240 (4858): 1534-8.
209. Apt K E, Kroth-Pancic P G, Grossman A R. Stable nuclear transformation of the diatom Phaeodactylum tricornutum. Mol Gen Genet. 1996; 252 (5): 572-9.
210. Dunahay T G, Jarvis E E, Roessler P G. GENETIC TRANSFORMATION OF THE DIATOMS CYCLOTELLA CRYPTICA AND NAVICULA SAPROPHILA. Journal of Phycology. 1995; 31 (6): 1004-12.
211. Falciatore A, Casotti R, Leblanc C, Abrescia C, Bowler C. Transformation of Nonselectable Reporter Genes in Marine Diatoms. Marine biotechnology (New York, NY). 1999; 1 (3): 239-51.
212. Zaslavskaia L A, Lippmeier J C, Kroth P G, Grossman A R, Apt K E. Transformation of the diatom Phaeodactylum tricornutum (Bacillariophyceae) with a variety of selectable marker and reporter genes. Journal of Phycology. 2000; 36 (2): 379-86.
213. Dunahay T G. Transformation of Chlamydomonas reinhardtii with silicon carbide whiskers. Biotechniques. 1993; 15 (3): 452-5, 7-8, 60.
214. Te M R, Lohuis, Miller D J. Genetic transformation of dinoflagellates (Amphidinium and Symbiodinium): expression of GUS in microalgae using heterologous promoter constructs. The Plant Journal. 1998; 13 (3): 427-35.
215. Henry E C, Meints R H. Recombinant viruses as transformation vectors of marine macroalgae. Journal of Applied Phycology. 6 (2): 247-53.
216. Van Etten J L, Meints R H. Giant viruses infecting algae. Annual review of microbiology. 1999; 53:447-94.
217. Kojima H, Kawata Y. A mini-transposon/transposase complex as a new tool for the genetic transformation of microalgae. In: Kojima H, Lee Y K, editors. Photosynthetic Microorganisms in Environment Biotechnology. Berlin, Germany: Springer-Verlag; 2001. p. 41-61.
218. Miller J H. A short course in bacterial genetics: a laboratory manual and handbook for Escherichia coli and related bacteria. Plainview, N.Y.: Cold Spring Harbor Laboratory Press; 1992.
219. Parekh S, Vinci V A, Strobel R J. Improvement of microbial strains and fermentation processes. Appl Microbiol Biotechnol. 2000; 54 (3): 287-301.
220. Forsburg S L. The art and design of genetic screens: yeast. Nature reviews Genetics. 2001; 2 (9): 659-68.
221. Flynn T, Ghirardi M L, Seibert M. Accumulation of 02-tolerant phenotypes in H2-producing strains of Chlamydomonas reinhardtii by sequential applications of chemical mutagenesis and selection. International Journal of Hydrogen Energy. 2002; 27 (11-12): 1421-30.
222. Doan T T Y, Obbard J P. Enhanced intracellular lipid in Nannochloropsis sp. via random mutagenesis and flow cytometric cell sorting. Algal Research. 2012; 1 (1): 17-21.
223. Bernheim A G, Libis V K, Lindner A B, Wintermute E H. Phage-mediated Delivery of Targeted sRNA Constructs to Knock Down Gene Expression in E. coli. 2016 (109): e53618.
224. Zhang R, Patena W, Armbruster U, Gang S S, Blum S R, Jonikas M C. High-Throughput Genotyping of Green Algal Mutants Reveals Random Distribution of Mutagenic Insertion Sites and Endonucleolytic Cleavage of Transforming DNA. The Plant Cell. 2014; 26 (4): 1398-409.
225. Dent R M, Haglund C M, Chin B L, Kobayashi M C, Niyogi K K. Functional genomics of Eukaryotic photosynthesis using insertional mutagenesis of Chlamydomonas reinhardtii. Plant Physiol. 2005; 137 (2): 545-56.
226. Colombo S L, Pollock S V, Eger K A, Godfrey A C, Adams J E, Mason C B, et al. Use of the bleomycin resistance gene to generate tagged insertional mutants of Chlamydomonas reinhardtii that require elevated CO2 for optimal growth. Functional Plant Biology. 2002; 29 (3): 231-41.
227. Gonzalez-Ballester D, Pootakham W, Mus F, Yang W, Catalanotti C, Magneschi L, et al. Reverse genetics in Chlamydomonas: a platform for isolating insertional mutants. Plant Methods. 2011; 7 (1): 1-13.
228. Kleckner N, Bender J, Gottesman S. Uses of transposons with emphasis on Tn10. Methods Enzymol. 1991; 204:139-80.
229. Wu-Scharf D, Jeong B-r, Zhang C, Cerutti H. Transgene and transposon silencing in Chlamydomonas reinhardtii by a DEAH-Box RNA helicase. Science. 2000; 290 (5494): 1159-62.
230. Casas-Mollano J A, Rohr J, Kim E J, Balassa E, van Dijk K, Cerutti H. Diversification of the core RNA interference machinery in Chlamydomonas reinhardtii and the role of DCL 1 in transposon silencing. Genetics. 2008; 179 (1): 69-81.
231. Goryshin I Y, Jendrisak J, Hoffman L M, Meis R, Reznikoff W S. Insertional transposon mutagenesis by electroporation of released Tn5 transposition complexes. Nat Biotech. 2000; 18 (1): 97-100.
232. Datsenko K A, Wanner B L. One-step inactivation of chromosomal genes in Escherichia coli K-12 using PCR products. Proc Natl Acad Sci USA. 2000; 97.
233. Zhong J, Karberg M, Lambowitz A M. Targeted and random bacterial gene disruption using a group II intron (targetron) vector containing a retrotransposition-activated selectable marker. Nucleic Acids Research. 2003; 31 (6): 1656-64.
234. Minoda A, Sakagami R, Yagisawa F, Kuroiwa T, Tanaka K. Improvement of culture conditions and evidence for nuclear transformation by homologous recombination in a red alga, Cyanidioschyzon merolae 10D. Plant & cell physiology. 2004; 45 (6): 667-71.
235. Qi Lei S, Larson Matthew H, Gilbert Luke A, Doudna Jennifer A, Weissman Jonathan S, Arkin Adam P, et al. Repurposing CRISPR as an RNA-guided platform for sequence-specific control of gene expression. Cell. 2013; 152 (5): 1173-83.
236. Jiang W, Bikard D, Cox D, Zhang F, Marraffini L A. RNA-guided editing of bacterial genomes using CRISPR-Cas systems. Nature biotechnology. 2013; 31 (3): 233-9.
237. Zhao T, Wang W, Bai X, Qi Y. Gene silencing by artificial microRNAs in Chlamydomonas. The Plant Journal. 2009; 58 (1): 157-64.
238. Si T, HamediRad M, Zhao H. Regulatory RNA-assisted genome engineering in microorganisms. Current opinion in biotechnology. 2015; 36:85-90.
239. Meng J, Kanzaki G, Meas D, Lam C K, Crummer H, Tain J, et al. A genome-wide inducible phenotypic screen identifies antisense RNA constructs silencing Escherichia coli essential genes. FEMS microbiology letters. 2012; 329 (1): 45-53.
240. Xiao H, Zhao H. Genome-wide RNAi screen reveals the E3 SUMO-protein ligase gene SIZI as a novel determinant of furfural tolerance in Saccharomyces cerevisiae. Biotechnology for Biofuels. 2014; 7 (1): 1-11.
241. Bao Z, Xiao H, Liang J, Zhang L, Xiong X, Sun N, et al. Homology-Integrated CRISPR-Cas (HI-CRISPR) System for One-Step Multigene Disruption in Saccharomyces cerevisiae. ACS Synthetic Biology. 2015; 4 (5): 585-94.
242. De Backer M D, Nelissen B, Logghe M, Viaene J, Loonen I, Vandoninck S, et al. An antisense-based functional genomics approach for identification of genes critical for growth of Candida albicans. Nat Biotech. 2001; 19 (3): 235-41.
243. Na D, Yoo S M, Chung H, Park H, Park J H, Lee S Y. Metabolic engineering of Escherichia coli using synthetic small regulatory RNAs. Nat Biotech. 2013; 31 (2): 170-4.
244. Ohnuma M, Misumi O, Fujiwara T, Watanabe S, Tanaka K, Kuroiwa T. Transient gene suppression in a red alga, Cyanidioschyzon merolae 10D. Protoplasma. 2009; 236 (1-4): 107-12.
245. Molnar A, Bassett A, Thuenemann E, Schwach F, Karkare S, Ossowski S, et al. Highly specific gene silencing by artificial microRNAs in the unicellular alga Chlamydomonas reinhardtii. Plant J. 2009; 58:165-74.
246. Jiang H, Chen Y, Jiang P, Zhang C, Smith T J, Murrell J C, et al. Methanotrophs: Multifunctional bacteria with promising applications in environmental bioengineering. Biochemical Engineering Journal. 2010; 49 (3): 277-88.
247 Andersen L, Sundman L-O, Inge-Britt Linden I-B, Kontro P, Simo S O. Synthesis and anticonvulsant properties of some 2-Aminoethanesulfonic acid (Taurine) derivatives. Journal of Pharmaceutical Sciences. 1984; 73:106-8.
248. Herdeis C, Weis C E, inventorsß-Aminoethanesulphonylazide their use for the preparation of 2-aminoethane-sulphonamide (taurylamide), taurolidine or taurultam and their acid addition salts. United States patent U.S. Pat. No. 5,889,183. 1999.
249. Tserng K-Y, Hachey D L, Klein P D. An improved procedure for the synthesis of glycine and taurine conjugates of bile acids. Journal of Lipid Research. 1977; 18:404-7.
250. Fong D W, Hoots J E, inventors; Nalco Chemical Company, assignee. Synthesis of tagged polymers by post-polymerization (trans) amidation reaction. United States patent U.S. Pat. No. 5,128,419. 1992.
251. Seeberger S, Griffin R J, Hardcastle I R, Golding B T. A new strategy for the synthesis of taurine derivatives using the ‘safety-catch’ principle for the protection of sulfonic acids. Org Biomol Chem. 2007; 5:132-8.
252. Suzuki M, Nakajima Y, Sato T, Shirai H, Hanabusa K. Fabrication of TiO2 using L-lysine-based organogelators as organic templates: control of the nanostructures. Chem Commun. 2006:377-9.
253. Mikhalenko S A, Soloveva L I, Lukyanets E A. Phthalocyanines and related compounds: XXXVIII. Synthesis of symmetric taurine- and choline-substituted phthalocyanines. Russ J Gen Chem. 2004; 74:1775-800.
254. Capone R, Blake S, Restrepo M R, Yang J, Mayer M. Designing Nanosensors Based on Charged Derivatives of Gramicidin A. Journal of the American Chemical Society. 2007; 129:9737-45.
255. Gupta R C, Win T, Bittner S. Taurine analogues; A new class of therapeutics: Retrospect and prospects Current Medicinal Chemistry. 2005; 12:2021-39.
256. Johnson B A. Update on neuropharmacological treatments for alcoholism: Scientific basis and clinical findings. Biochemical Pharmacology. 2008; 75:34-56.
257. Tambour S, Quertemont E. Preclinical and clinical pharmacology of alcohol dependence. Fundamental and Clinical Pharmacology. 2007; 21:9-28.
258. Joung Y K, Sengoku Y, Ooya T, Park K D, Yui N. Anticoagulant supramolecular-structured polymers: Synthesis and anticoagulant activity of taurine-conjugated carboxyethylester-polyrotaxanes. Science and Technology of Advanced Materials. 2005; 6:484-90.
259. Özmeriç N, Özcan G, Haytaç CM, Alaaddinoglu E E, Sargon M F, Senel S. Chitosan film enriched with an antioxidant agent, taurine, in fenestration defects. Journal of Biomedical Materials Research Part A. 2000; 51:500-3.
260. Degim Z, çelebi N, Sayan H, Babül A, Erdoğan D, Take G. An investigation on skin wound healing in mice with a taurinechitosan gel formulation. Amino Acids. 2002; 22:187-98.
261. Matsusaki M, Serizawa T, Kishida A, Endo T, Akashi M. Novel functional biodegradable polymer: Synthesis and anticoagulant activity of poly (y-Glutamic Acid) sulfonate (y-PGA-sulfonate). Bioconjugate Chemistry. 2002; 13:23-8.
262. Meinkoth J, G. W. Hybridization of nucleic acids immobilized on solid supports. Analytical Biochemistry. 1984; 138:267-84.
263. Tijssen P. Overview of principles of hybridization and the strategy of nucleic acid probe assays. Laboratory Techniques in Biochemistry and Molecular Biology-Hybridization with Nucleic Acid Probes: Part I. New York: Elsevier; 1993.
264. Smith T F, Waterman M S. Comparison of biosequences. Advances in Applied Mathematics. 1981; 2:482-9.
265. Needleman S B, Wunsch C D. A general method applicable to the search for similarities in the amino acid sequence of two proteins. Journal of Molecular Biology. 1970; 48:443-53.
266. Pearson W R, Lipman D J. Improved tools for biological sequence comparison. Proceedings of the National Academy of Sciences of the United States of America. 1988; 85:2444-8.
267. Higgins D G, Bleasby A J, Fuchs R. CLUSTAL V: improved software for multiple sequence alignment. Computer Applications in the Biosciences. 1992; 8 (2): 189-91.
268. Higgins D G, Sharp P M. CLUSTAL: a package for performing multiple sequence alignment on a microcomputer. Gene. 1988; 73 (1): 237-44.
269. Higgins D G, Sharp P M. Fast and sensitive multiple sequence alignments on a microcomputer. Computer Applications in the Biosciences. 1989; 5 (2): 151-3.
270. Feng D F, Doolittle R F. Progressive sequence alignment as a prerequisite to correct phylogenetic trees. Journal of Molecular Evolution. 1987; 25 (4): 351-60.
271. Henikoff S, Henikoff J. Amino acid substitution matrices from protein blocks Proceedings of the National Academy of Sciences of the United States of America. 1989; 89:10915-9.
272. Altschul S F, Madden T L, Schaffer A A, Zhang J, Zhang Z, Miller W, et al. Gapped BLAST and PSI-BLAST: a new generation of protein database search programs. Nucleic Acids Research. 1997; 25:3389-402.
273. Wootton J C, Federhen S. Statistics of local complexity in amino acid sequences and sequence databases. Comput Chem. 1993; 17:149-63.
274. Wootton J C, Federhen S. Analysis of compositionally biased regions in sequence databases. Methods Enzymol. 1996; 266:554-71.
275. Claverie J-M, States D J. Information enhancement methods for large scale sequence analysis. Comput Chem. 1993; 17:191-201.
276. Myers E W, Miller W. Optimal alignments in linear-space. Computer Applications in the Biological Sciences. 1988; 4:11-7.
277. Merlin C, McAteer S, Masters M. Tools for characterization of Escherichia coli genes of unknown function. Journal of Bacteriology. 2002; 184:4573-81.
278. Buchholz J, Schwentner A, Brunnenkan B, Gabris C, Grimm S, Gerstmeir R, et al. Platform engineering of Corynebacterium glutamicum with reduced pyruvate dehydrogenase complex activity for improved production of L-lysine, L-valine, and 2-ketoisovalerate. Appl Environ Microbiol. 2013; 79 (18): 5566-75.

### EXAMPLES

### Example 1

### Development of a High Taurine-Producing Microbe

Step 1: Use chemical synthesis to make a ΔtauABCD polynucleotide (SEQ ID NO: 116). Clone the polynucletide into the vector pTOF25 and transform into an E. coli K12 strain to knockout tauABCD (SEQ ID NO:68) using the recombination methods of Merlin et al. (277).

Step 2: Use chemical synthesis to make a ΔssuEADCB polynucleotide (SEQ ID NO: 115). Clone the polynucleotide into the vector pTOF25 and transform into the ΔtauABCD strain (from Step 1 EXAMPLE 1) to knockout ssuEADCB (SEQ ID NO:69) using the recombination methods of Merlin et al. (277).

Step 3: Use chemical synthesis to make a trcPUWA polynucleotide (SEQ ID NO: 118). Clone the polynucleotide into the vector pTOF25 and transform into the ΔtauABCD/ΔssuEADCB strain (from Step 2 EXAMPLE 1) to knockin a constitutive promoter to replace the native promoter for cysPUWA (SEQ ID NO:110) using the recombination methods of Merlin et al. (277).

Step 4: Use chemical synthesis to make a trcDNC polynucleotide (SEQ ID NO:117). Clone the polynucleotide into the vector pTOF25 and transform into the ΔtauABCD/ΔssuEADCB/trcPUWA strain (from Step 3 EXAMPLE 1) to knockin a constitutive promoter to replace the native promoter for cysDNC (SEQ ID NO:47) using the recombination methods of Merlin et al. (277)

Step 5: Use chemical synthesis to make an operable polycistronic CDO/SAD cysQ cysH/cysIJ polynucleotide optimized for expression in the host cell line as follows:
a. The CDO gene is derived from SEQ ID NO:3 by removing nucleotides 4 through 159 (corresponding to the native transit peptide) and encodes a CDO peptide from Chlamydomonas reinhardtii (SEQ ID NO:4 minus amino acids 2 through 53); and
b. The SAD gene is derived from SEQ ID NO:9 and encodes a SAD peptide from Danio rerio (SEQ ID NO:10); and
c. The cysQ gene is derived from SEQ ID: 51 and encodes a cysQ peptide from E. coli (SEQ ID NO:52); and
d. The cysH gene is derived from SEQ ID: 53 and encodes a cysH peptide from E. coli (SEQ ID NO:54); and
e. The cysIJ gene is derived from SEQ ID: 55 and encodes the cys/peptide from E. coli (SEQ ID NO:57) and the cys peptide from E. coli (SEQ ID NO:56).

Step 6: Clone the polynucleotide into a bacterial expression vector so it is functional.

Step 7: Use chemical synthesis to make an operable polycistronic pgk/serA_{Δ197}/serC/serB/cysE_{M201R}/cysK/cysM polynucleotide.
a. The pgk gene is derived from SEQ ID NO:35 and encodes a pgk peptide from C. glutamicum (SEQ ID NO: 36); and
b. The serA_{Δ197} gene is derived from SEQ ID NO:37 and encodes the serA_{Δ}197 peptide from C. glutamicum (SEQ ID NO:38); and
c. The serC gene is derived from SEQ ID NO:41 and encodes the serC peptide from C. glutamicum (SEQ ID NO:42); and
d. The serB gene is derived from SEQ ID NO:39 and encodes the serB peptide from C. glutamicum (SEQ ID NO:40); and
e. The cysE_{M201R} gene is derived from SEQ ID NO: 43 and encodes the cysE_{M201R} peptide from E. coli (SEQ ID NO: 44); and
f. The cysK gene is derived from SEQ ID NO: 45 and encodes the cysK peptide from E. coli (SEQ ID NO: 46); and
g. The cysM gene is derived from SEQ ID NO: 98 and encodes the cysM peptide from E. coli (SEQ ID NO: 99).

Step 8: Clone the polycistronic pgk/serA_{Δ197}/serC/serB/cysE_{M1201R}/cySK/cysM polynucleotide into a bacterial expression vector, with a different selectable marker from the vector in Step 6, EXAMPLE 1, so it is functional.

Step 9: Co-transform the vectors with the CDO-SAD cysQ cysH/cysIJ construct (from Step 6, EXAMPLE 1) and pgk/serA_{Δ197}/serC/serB/cysE_{M201R} cysK (from Step 8, EXAMPLE 1) into the ΔtauABCD/ΔssuEADCB/trcPUWA/trcDNC strain (from Step 4, EXAMPLE 1) and confirm the presence of both DNA constructs.

### Example 2

### Development of Another High Taurine-Producing Microbe

Step 1: Make a ΔsdaA in the ΔtauABCD ΔssuEADCB strain (from Step 2, EXAMPLE 1) using the synthetic polynucleotide (SEQ ID NO: 146) and recombination methods of Merlin et al. (277).

Step 2: Make a ΔglyA in the ΔtauABCD/ΔssuEADCB/ΔsdaA strain (from Step 1, EXAMPLE 2) using the synthetic polynucleotide (SEQ ID NO:159) and recombination methods of Merlin et al. (277).

Step 3: Make a trcDNC in the ΔtauABCD/ΔssuEADCB/ΔsdaA/ΔglyA strain (from Step 2, EXAMPLE 2) using the synthetic polynucleotide (SEQ ID NO:117) and recombination methods of Merlin et al. (277).

Step 4: Make a trcUWA in the ΔtauABCD/ΔssuEADCB/ΔsdaA/ΔglyA/trcDNC strain (from Step 3, EXAMPLE 2) using the synthetic polynucleotide (SEQ ID NO:134) and recombination methods of Merlin et al. (277).

Step 5: Make a ΔilvA in the ΔtauABCD/ΔssuEADCB/ΔsdaA/ΔglyA/trcDNC/trcUWA strain (from Step 4, EXAMPLE 2) using the synthetic polynucleotide (SEQ ID NO:135) and recombination methods of Merlin et al. (277).

Step 6: Use chemical synthesis to make an operable polycistronic pgk/serA_{Δ197}/serC/serB/cysE_{M201R}/cysK polynucleotide as described in Steps 7a through 7f, EXAMPLE 1.

Step 7: Clone the polynucleotide into a bacterial expression vector so it is functional.

Step 8: Use chemical synthesis to make an operable polycistronic CDO/SAD/cysQ/cysH/cysIJ/sbp polynucleotide optimized for expression in the host cell line as follows:
a. CDO, SAD, cysQ, cysH, and cysIJ are derived as described in Steps 5a through 5e, EXAMPLE 1; and
b. sbp is derived from SEQ ID NO: 160 and encodes the sbp peptide from E. coli (SEQ ID NO: 161).

Step 9: Clone the polycistronic CDO SAD/cysQ/cysH/cysIJ/sbp polynucleotide into a bacterial expression vector, with a different selectable marker from the vector in Step 7, EXAMPLE 2, so it is functional.

Step 10: Co-transform the vectors with CDO SAD/cysQ/cysH/cysIJ/sbp construct (from Step 9, EXAMPLE 2) and pgk/serA_{Δ197}/serC/serB/cysE_{M201R}/cysK (from Step 7, EXAMPLE 2) into the ΔtauABCD/ΔssuEADCB/ΔsdaA/ΔglyA/trcDNC/trcUWA strain (from Step 5, EXAMPLE 2) and confirm the presence of both DNA constructs.

### Example 3

### Development of Another High Taurine-Producing Microbe

Step 1: Make a ΔridA in the ΔtauABCD/ΔssuEADCB/ΔsdaA/ΔglyA/trcDNC strain (from Step 3, EXAMPLE 2) using the synthetic polynucleotide (SEQ ID NO:119) and recombination methods of Merlin et al. (277).

Step 2: Make a ΔtdcF in the ΔtauABCD/ΔssuEADCB/ΔsdaA/ΔglyA/trcDNC/ΔridA strain (from Step 1, EXAMPLE 3) using the synthetic polynucleotide (SEQ ID NO:120) and recombination methods of Merlin et al. (277).

Step 3: Make a ΔrutC in the ΔtauABCD/ΔssuEADCB/ΔsdaA/ΔglyA/trcDNC/ΔridA/ΔdcF strain (from Step 2, EXAMPLE 3) using the synthetic polynucleotide (SEQ ID NO: 121) and recombination methods of Merlin et al. (277).

Step 4: Use chemical synthesis to make an operable polycistronic pgk/serA_{Δ197}/serC/serB polynucleotide as described in Steps 7a through 7d, EXAMPLE 1.

Step 5: Clone the polycistronic pgk/serA_{Δ197}/serC/serB polynucleotide into a bacterial expression vector so it is functional.

Step 6: Use chemical synthesis to make an operable polycistronic CS PLP-DC/IlvA_{L447F} polynucleotide optimized for expression in the host cell line as follows:
a. The CS PLP-DC gene is derived from SEQ ID NO: 17 and encodes the pgk peptide from Micromonas pusilla (SEQ ID NO: 18); and
b. The IlvA_{L447F} gene is derived from SEQ ID NO:29 and encodes the IlvA_{L447F} peptide from E. coli (SEQ ID NO:30).

Step 7: Clone the polycistronic CS PLP-DC IlvA_{L447F} polynucleotide into a bacterial expression vector, with a different selectable marker from the vector in Step 5, EXAMPLE 3, so it is functional.

Step 8: Co-transform the vectors with the functional pgk/serA_{Δ197}/serC/serB (from Step 5, EXAMPLE 3) and CS/PLP-DC/IlvA_{L447F} constructs (from Step 7, EXAMPLE 3) into the ΔtauABCD/ΔssuEADCB/ΔsdaA/ΔglyAltrcDNC/ΔridA/ΔtdcF/ΔrutC strain (from Step 3, EXAMPLE 3) and confirm the presence of both DNA constructs.

### Example 4

### Development of Another High Taurine-Producing Microbe

Step 1: Use chemical synthesis to make an operable polycistronic TS/partCS/PLP-DC polynucleotide optimized for expression in the host cell line as follows:
a. The TS gene is derived from SEQ ID NO:27 and encodes the TS peptide from Euryarchaeota archaeon (SEQ ID NO: 28); and
b. The partCS/PLP-DC gene is derived from SEQ ID NO: 17 by removing nucleotides 4 through 1413 (which removes the native transit and cysteine synthetase peptide sequences but retains the start codon) and encodes a partCS/PLP-DC peptide from Micromonas pusilla (SEQ ID NO:18 minus amino acids 2 through 471).

Step 2: Clone the polycistronic TS/partCS/PLP-DC polynucleotide into a bacterial expression vector so it is functional.

Step 3: Use chemical synthesis to make an operable polycistronic pgk/serA_{Δ197}/serC polynucleotide as described in Steps 7a through 7c, EXAMPLE 1.

Step 4: Clone the polycistronic pgk serA_{Δ197}/serC polynucleotide into a bacterial expression vector, with a different selectable marker from the vector in Step 2, EXAMPLE 4, so it is functional.

Step 5: Co-transform the vectors with the functional pgk/serA_{Δ197}/serC (from Step 4, EXAMPLE 4) and TS/partCS/PLP-DC constructs (from Step 2, EXAMPLE 4) into the ΔtauABCD/ΔssuEADCB/ΔsdaA/ΔglyA/trcDNC strain (from Step 3, EXAMPLE 2) and confirm the presence of both DNA constructs.

### Example 5

### Development of Another High Taurine-Producing Microbe

Step 1: Generate a DNA fragment using genomic DNA from C. glutamicum and the primer pairs, SEQ ID NO:122 and SEQ ID NO:123. Generate a second DNA fragment using genomic DNA from C. glutamicum and the primer pairs, SEQ ID NO:124 and SEQ ID NO:125. Purify each DNA fragment and use them in overlap PCR with primers SEQ ID NO: 122 and SEQ ID NO: 125 to make a knockout fragment for ssuE (SEQ ID NO:76). Clone the resulting fragment into the pK19mobsacB vector and transform into C. glutamicum to replace ssuE with the ssuE knockout fragment by homologous recombination as described by Buchholz et al. (278).

Step 2: Make a ΔmcbR in the ΔssuE strain (from Step 1, EXAMPLE 5) using the synthetic polynucleotide (SEQ ID NO:142) and recombination methods as described by Buchholz et al. (278).

Step 3: Make a ΔilvA in the ΔssuE/ΔmcbR strain (from Step 2, EXAMPLE 5) using the synthetic polynucleotide (SEQ ID NO: 139) and recombination methods as described by Buchholz et al. (278).

Step 4: Make a ΔglyA in the ΔssuE/ΔmcbR/ΔilvA strain (from Step 3, EXAMPLE 5) using the synthetic polynucleotide (SEQ ID NO: 138) and recombination methods as described by Buchholz et al. (278).

Step 5: Clone the polycistronic pgk serA_{Δ197}/serC/serB polynucleotide from Step 5: EXAMPLE 3 into a bacterial expression vector so it is functional.

Step 6: Use chemical synthesis to make an operable polycistronic CDO/SAD/gadC polynucleotide optimized for expression in the host cell line as follows:
a. The CDO gene is derived from SEQ ID NO:1 and encodes a CDO peptide from Danio rerio (SEQ ID NO:2); and
b. The SAD gene is derived from SEQ ID NO:9 and encodes a SAD peptide from Danio rerio (SEQ ID NO:10).
c. The gadC gene is derived from SEQ ID NO:184 and encodes a GadC peptide from E. coli (SEQ ID NO:185).

Step 7: Clone the CDO/SAD/gadC polynucleotide into a bacterial expression vector, with a different selectable marker from the vector in Step 5, EXAMPLE 5, so it is functional.

Step 8: Co-transform the vectors with the functional CDO/SAD/gadC (from Step 7, EXAMPLE 5) and pgk/serA_{Δ197}/serC/serB (from Step 5, EXAMPLE 5) into the ΔssuE/ΔmcbR/ΔilvA/glyA strain (from Step 4, EXAMPLE 5) and confirm the presence of the DNA construct.

### Example 6

### Microbial Fermentation Process to Produce at Least 1.0 g L Taurine in a Shaker Flask

Step 1: Grow a seed culture of taurine-producing bacteria (from EXAMPLES 1, 2, 3, or 4) in LB broth with the appropriate antibiotic(s) for 12-20 hours on a rotary shaker at 37° C. and 250 rpm.

Step 2: Inoculate production media with 1/50 volume of seed culture. The production media contains ammonium sulfate (5 g/L), dibasic potassium phosphate (6 g/L), monobasic sodium phosphate (3 g/L), magnesium sulfate (0.5 g/L), glucose (6 g/L), typtone (0.1 g/L), yeast extract (0.05 g/L), and PLP (2.4 mg/L), with or without antibiotic(s), pH 7.0. Grow taurine-producing bacteria in production media in beveled flasks for 20-30 hours in a rotary shaker at 250 rpm and 30° C.

Step 3: Separate cells from broth by centrifugation.

Step 4: Determine the taurine concentration in the cells and cleared broth by HPLC.

### Example 7

### Microbial Fermentation Process to Produce at Least 25 g L Taurine in a Fermentor

Step 1: Grow the seed culture of taurine-producing bacteria (from EXAMPLES 1, 2, 3, or 4) in LB broth with the appropriate antibiotic(s) for 12-20 hours on a rotary shaker at 250 rpm and 37° C.

Step 2: Conduct batch fermentation in a 1.5 L bioreactor using production media from Step 2 and EXAMPLE 6 plus an antifoaming agent. Maintain pH at 7.0 with ammonium hydroxide, temperature at 30° C., and dissolved oxygen above 20% by adjusting the agitation speed and air-flow.

Step 3: Separate cells from broth by centrifugation.

Step 4: Determine the taurine concentration in the cells and cleared broth by HPLC.

### Example 8

### Another Microbial Fermentation Process to Produce at Least 1.0 g L Taurine in a Shaker Flask

Step 1. Grow the seed culture of taurine-producing bacteria (from EXAMPLE 5) in LB broth with 0.5% glucose with the appropriate antibiotic(s) for 24 hours on a rotary shaker at 200 rpm and 30° C. for 48 hours.

Step 2: Inoculate production media with 1/10 volume of seed culture. The production media contains yeast extract (2 g/L), glucose (40 g/L), calcium carbonate (10 g/L), ammonium sulfate (15 g/L), dibasic potassium phosphate (1 g/L), monobasic potassium phosphate (1 g/L), sodium chloride (2 g/L), calcium chloride (80 mg/L), ferric chloride (3 mg/L), zinc sulfate heptahydrate (0.9 mg/L), cupric sulfate (0.2 mg/L), manganese sulfate (0.4 mg/L), sodium molybdate (0.1 mg/L), sodium borate (0.3 mg/L), magnesium sulfate (1 g/L), thiamine hydrochloride (0.2 mg/L), biotin (0.2 mg/L), and PLP (2.4 mg/L), with or without antibiotic(s), pH 7.0. Grow taurine-producing bacteria in production media in beveled flasks for 24 hours in a rotary shaker at 250 rpm and 30° C.

Step 3: Separate cells from broth by centrifugation,

Step 4: Determine the taurine concentration in the cells and cleared broth by HPLC.

### Example 9

### Another Microbial Fermentation Process to Produce at Least 25 g L Taurine in a Fermentor

Step 1: Grow the seed culture of taurine-producing bacteria (from EXAMPLE 5) in LB broth with the appropriate antibiotic(s) for 24 hours on a rotary shaker at 200 rpm and 30° C.

Step 2: Conduct batch fermentation and 1.5 L bioreactor with production media from Step 2, EXAMPLE 8 plus an antifoaming agent. Maintain pH at 7.0 with potassium hydroxide and phosphoric acid, temperature at 30° C., and dissolved oxygen above 20% by adjusting the agitation speed and air-flow.

Step 3: Separate cells from broth by centrifugation.

Step 4: Determine the taurine concentration in the cells and cleared broth by HPLC.

### Example 10

### Purification of Taurine of at Least 90% from Cleared Broth

Step 1: Purify taurine from the cleared broth (Step 3, EXAMPLES 6-9) by cation exchange as follows:
a. Concentrate solution with ultrafiltration membrane; and
b. Adjust pH of the cleared broth solution to pH 4.0 with HCl; and
c. Add solution to an activated cation-exchange column; and
d. Wash column with 0.1N HCl; and
e. Elute taurine with deionized water.

Step 2: Dry down solution to crystal or powder form.

Step 3: Determine taurine concentration by HPLC.

### Example 11

### Purification of Taurine of at Least 90% from Fermented Cells

Step 1: Suspend cells (from Step 3, EXAMPLES 6, 7, 8, OR 9) in 0.1N HCl.

Step 2: Disrupt cells by chemical agents, pressure, mechanical force, or ultrasonification to release their contents.

Step 3: Separate cellular debris from supernatant by centrifugation.

Step 4: Purify taurine from the supernatant (Step 3, EXAMPLES 6-9) by cation exchange as described in Steps 1a through 1e, EXAMPLE 10.

Step 5: Dry down solution to crystal or powder form.

Step 6: Determine taurine concentration by HPLC.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method for taurine production comprising:
growing a unicellular organism in a shaker flask to produce at least 1 g/liter of taurine from the unicellular organism.

2. A method for taurine production comprising:
growing a unicellular organism in a fermentor or bioreactor to produce at least 20 g/liter of taurine from the unicellular organism.

3. The method of claim 1, wherein the unicellular organism expresses one or more exogenous taurine biosynthetic pathways and contains one or more of the following:
a deletion of at least one operon for taurine or sulfonate uptake and degradation;
an exogenous taurine exporter;
increased expression of genes in sulfate or thiosulfate transport, or sulfur reduction or sulfur assimilation;
increased expression of a gene in the serine biosynthetic pathway;
increased expression of a gene in the cysteine biosynthetic pathway;
increased expression of a gene in the 2-aminoacrylate biosynthetic pathway;
a deletion of at least one gene in the degradation of taurine, serine, cysteate or 2-aminoacrylate; or
modified expression of a gene for a transcriptional regulator or activator for taurine, sulfur, or cysteine metabolism.

4. The method of claim 1, wherein the unicellular organism is selected from the group consisting of Proteobacteria, Alphaproteobacteria, Betaproteobacteria, Deltaproteobacteria, Epsilonproteobacteria, Methanotrophs, Methylobacterium, Escherichia, Bacillus, Salmonella, Lactococcus, Lactobacillus Streptococcus, Brevibacterium, coryneform bacteria, Bacillus subtilis, Brevibacterium ammoniagene, Corynebacterium crenatum, Corynebacterim pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans and Saccharomyces cerevisiae.

5. The method of claim 1, further comprising:
isolating the taurine to produce taurine having a purity level of greater than 10% purity, greater than 25% purity, greater than 50% purity, greater than 75% purity, or greater than 98% purity.

6. The method of claim 1, wherein the unicellular organism is E. coli and the growing step is conducted in a media that contains at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, and at least 0.05 g/L yeast extract.

7. The method of claim 1, wherein the growing step is conducted in a media that contains at least 0.25 mg/L pyridoxal 5′-phosphate (PLP).

8. The method of claim 1, wherein the cells of the unicellular organism are chemically, physically or mechanically disrupted, dried, and used in food, feed, beverages, dietary and health supplements, cosmetics, personal care, pharmaceuticals, or agricultural production.

9. A unicellular organism capable of producing at least 1 g/liter of taurine when grown in a shaker flask or 20 g/liter of taurine when grown in a fermentor or bioreactor.

10. The method of claim 2, wherein the unicellular organism expresses one or more exogenous taurine biosynthetic pathways and contains one or more of the following:
a deletion of at least one operon for taurine or sulfonate uptake and degradation;
an exogenous taurine exporter;
increased expression of genes in sulfate or thiosulfate transport, or sulfur reduction or sulfur assimilation;
increased expression of a gene in the serine biosynthetic pathway;
increased expression of a gene in the cysteine biosynthetic pathway;
increased expression of a gene in the 2-aminoacrylate biosynthetic pathway;
a deletion of at least one gene in the degradation of taurine, serine, cysteate or 2-aminoacrylate; or
modified expression of a gene for a transcriptional regulator or activator for taurine, sulfur, or cysteine metabolism.

11. The method of claim 2, wherein the unicellular organism is selected from the group consisting of Proteobacteria, Alphaproteobacteria, Betaproteobacteria, Deltaproteobacteria, Epsilonproteobacteria, Methanotrophs, Methylobacterium, Escherichia, Bacillus, Salmonella, Lactococcus, Lactobacillus Streptococcus, Brevibacterium, coryneform bacteria, Bacillus subtilis, Brevibacterium ammoniagene, Corynebacterium crenatum, Corynebacterim pekinese, Corynebacterium glutamicum, Erwinia citreus, Erwinia herbicola, Escherichia coli, Fusarium venenatum, Gluconobacter oxydans, Propionibacterium freudenreicheii, Propionibacterium denitrificans and Saccharomyces cerevisiae.

12. The method of claim 2, further comprising:
isolating the taurine to produce taurine having a purity level of greater than 10% purity, greater than 25% purity, greater than 50% purity, greater than 75% purity, or greater than 98% purity.

13. The method of claim 2, wherein the unicellular organism is E. coli and the growing step is conducted in a media that contains at least 5 g/L ammonium sulfate, at least 6 g/L dibasic potassium phosphate, at least 3 g/L monobasic sodium phosphate, at least 0.5 g/L magnesium sulfate, at least 6 g/L glucose, at least 0.1 g/L typtone, and at least 0.05 g/L yeast extract.

14. The method of claim 2, wherein the growing step is conducted in a media that contains at least 0.25 mg/L pyridoxal 5′-phosphate (PLP).

15. The method of claim 2, wherein the cells of the unicellular organism are chemically, physically or mechanically disrupted, dried, and used in food, feed, beverages, dietary and health supplements, cosmetics, personal care, pharmaceuticals, or agricultural production.
